(19) United States
(12) Patent Application Publication
(10) Pub. No.: US 2024/0139728 A1
(43) Pub. Date: May 2, 2024

(54) DEVICES, ASSAYS AND METHODS FOR DETECTION OF NUCLEIC ACIDS

(71) Applicant: Mammoth Biosciences, Inc., Brisbane, CA (US)

(72) Inventors: Maria-Nefeli TSALOGLOU, San Francisco, CA (US); Janice Sha CHEN, San Francisco, CA (US); James Paul BROUGHTON, South San Francisco, CA (US); Daniel Thomas DRZAL, Pacifica, CA (US); Sarah Jane SHAPIRO, Pacifica, CA (US); Clare Louise FASCHING, Redwood City, CA (US); Carley Gelenter HENDRIKS, Burlingame, CA (US); Jesus CHING, Saratoga, CA (US); Sonal JAIN, San Mateo, CA (US)

(21) Appl. No.: 18/055,814

(22) Filed: Nov. 15, 2022

Related U.S. Application Data
(63) Continuation of application No. PCT/US21/33271, filed on May 19, 2021.
(60) Provisional application No. 63/181,131, filed on Apr. 28, 2021.
(60) Provisional application No. 63/114,977, filed on Nov. 17, 2020.
(60) Provisional application No. 63/113,799, filed on Nov. 13, 2020.
(60) Provisional application No. 63/027,309, filed on May 19, 2020.

(51) Int. Cl. B01L 3/00; G01N 33/53

(52) U.S. Cl. CPC B01L 3/5023; G01N 33/5308; B01L 2300/069; B01L 2400/06

(57) ABSTRACT

The application provides devices, kits and methods for a streamlined lateral flow assay for sample preparation, optional amplification, and detection of a target nucleic acid using programmable nuclease reagents.

### CROSS REFERENCE

This application claims the benefit of U.S. Provisional Application No. 63/027,309, filed on May 19, 2020, U.S. Provisional Application No. 63/113,799, filed on Nov. 13, 2020, U.S. Provisional Application No. 63/114,977 filed on Nov. 17, 2020, and U.S. Provisional Application No. 63/181,131 filed on Apr. 28, 2021, each of which is incorporated herein by reference in its entirety.

### STATEMENT AS TO FEDERALLY SPONSORED RESEARCH

This invention was made with government support under Contract No. N66001-21-C-4048 awarded by the Department of Defense, Defense Advanced Research Projects Agency (DARPA). The U.S. government has certain rights in the invention.

### SEQUENCE LISTING

The instant application contains a Sequence Listing which has been submitted electronically in XML format and is hereby incorporated by reference in its entirety. Said XML copy, created on Nov. 22, 2022, is named 203477-733301US_SL.xml and is 222,422 bytes in size.

### BACKGROUND

Streamlined devices are needed for rapid, sensitive, and specific detection of nucleic acids using CRISPR/Cas systems. Devices that can service point-of-need (PON) markets are especially needed. In these cases, lateral flow assays may be used, where they combine various reagents and process steps in one assay strip. This in turn provides a sensitive and rapid means for detection.

### SUMMARY

Described herein are methods, systems, and devices for detection of a target nucleic acids. In a first aspect, a device is provided. The device comprises: a chamber configured to contain a sample comprising one or more nucleic acids; a programmable nuclease detection reagent and a reporter; a locking-mechanism coupled to the chamber and having a locked configuration and an unlocked configuration, wherein the locking-mechanism is configured to form a fluid-tight seal between a sample collector comprising the sample and the chamber when in the locked configuration; a reagent release mechanism configured to release one or more reagents therefrom when the locking-mechanism is in the locked configuration; and a detection reagent system configured to detect a presence of a target nucleic acid when the target nucleic acid is present in the one or more nucleic acids of the sample, wherein the presence of the target nucleic acid is indicated by a signal produced upon reaction of the target nucleic acid with a programmable nuclease of the programmable nuclease detection reagent and subsequent cleavage and release of at least a portion of the reporter, wherein at least a portion of the device is configured to be is sealed in an enclosure once the locking mechanism has been engaged in the locked configuration. In some embodiments, the reagent release mechanism is configured to prevent amplicon contamination by at least 2-fold as compared to running the detection reaction outside the enclosure of the device. In some embodiments, where the detection system is configured to remain external to the body when the portion of the device is sealed in the body. In some embodiments, the detection system is disposed on a lateral flow assay strip or within a detection chamber.

In another aspect, a device for detection of a target nucleic acid is provided, the device comprising: a chamber configured to contain a sample provided by way of a sample collection apparatus, wherein the sample collection apparatus comprises a sample collector at a distal end thereof and an interlock at a proximal end thereof; a programmable nuclease; and a detection system configured to detect a presence of the target nucleic acid when the target nucleic acid is present in the sample, wherein the presence of the target nucleic acid is indicated by a signal produced upon reaction of the target nucleic acid with the programmable nuclease and subsequent cleavage and release of at least a portion of a reporter, wherein the interlock, integrated into the enclosure, is configured to form a fluid-tight seal, enclosing the sample collector and reagents, comprising the programmable nuclease and the reporter, wherein the interlock comprises a release mechanism configured to release the reagents enclosed within the enclosure to prevent the reaction from amplicon contamination by at least 2 fold as compared to detection outside the enclosure, and wherein at least a portion of the device is configured to be sealed in the enclosure once the interlock has been engaged to form the fluid-tight seal. In some embodiments, a “child safety lock” seal is formed between the interlock and the enclosure upon sealing. In some embodiments, wherein the interlock comprises a screw-top lid configured to screw onto corresponding threads on an external surface of the device. In some embodiments, the screw-top lid is configured to form the fluid-tight seal at a first position, and wherein continuing to screw on the screw-top lid to a second position will puncture one or more reagent blister packs disposed within the chamber, there upon releasing one or more reagents inside the chamber, wherein the chamber is inside the sealed enclosure. In some embodiments, the enclosed reagents comprise the reporter. In some embodiments, the reporter comprises an RNA sequence. In some embodiments, the reporter is suspended or immobilized in the chamber. In some embodiments, the reporter is cleaved by the programmable nuclease. In some embodiments, the target nucleic acid is a target deoxyribonucleic acid and wherein the target deoxyribonucleic acid lacks a guanine at the 3′ end. In some embodiments, the target nucleic acid is a target deoxyribonucleic acid and wherein the terminal 3′ nucleotide in the segment of the target deoxyribonucleic acid is A, C or T. In some embodiments, the target nucleic acid is a target deoxyribonucleic acid and wherein the target deoxyribonucleic acid is a single-stranded deoxyribonucleic acid. In some embodiments, the target nucleic acid is a target deoxyribonucleic acid and wherein the target deoxyribonucleic acid is a single stranded deoxyribonucleic acid oligonucleotide. In some embodiments, the target nucleic acid is a target deoxyribonucleic acid and wherein the target deoxyribonucleic acid is genomic single stranded deoxyribonucleic acids. In some embodiments, the target nucleic acid is a target deoxyribonucleic acid and wherein the target deoxyribonucleic acid has a length of from 18 to 100 nucleotides. In some embodiments, the target nucleic acid is a target deoxyribonucleic acid and wherein the target deoxyribonucleic acid has a length of from 18 to 30 nucleotides. In some embodiments, the target nucleic acid is a target deoxyribonucleic acid and wherein the target deoxyribonucleic acid has a length of 20 nucleotides. In some embodiments, the device is battery powered. In some embodiments, the device is not battery powered. In some embodiments, the device comprises a chemical heating element. In some embodiments, the device comprises a sample pad region, a nucleic acid amplification region, a conjugate pad region, a detection region, a collection pad region, or any combination thereof. In some embodiments the enclosure encases one or more support medium. In some embodiments, the enclosure is made from cardboard, plastics, polymers, or materials that provide mechanical protection for the support medium. In some embodiments, the support medium is configured to present the signal in a detectable format. In some embodiments, the support medium is a lateral flow assay strip. In some embodiments, the programmable nuclease is selected from the group consisting of a Cas13, Mad7, Mad2, Csm1, Cas9, C2c4, C2c8, C2c5, C2c10, C2c9, and CasZ. In some embodiments, the programmable nuclease is a type V CRISPR-Cas system, a type VI CRISPR Cas system, or a type III CRISPR Cas system In some embodiments, the Cas13 is Cas13a, Cas13b, Cas13c, Cas13d, or Cas13e. In some embodiments, the enclosure contains a filtration device. In some embodiments, the enclosure comprises a filtration device configured to filter the sample after a lysis step. In some embodiments, the enclosure houses an amplification chamber. In some embodiments, the amplification chamber comprises amplification reagents. In some embodiments, the amplification reagents are configured for amplification of the target nucleic acid. In some embodiments, the amplification comprises an isothermal amplification. In some embodiments, the isothermal amplification comprises LAMP amplification. In some embodiments, the isothermal amplification is performed at 62 Celsius. In some embodiments, the isothermal amplification is performed for no more than 5 minutes. In some embodiments, the device comprises both amplification reagents and DETECTR™ reagents in one chamber. In such embodiments, the device can be configured for a one pot DETECTR™ reaction, wherein a) sample preparation, amplification, and detection, b) sample preparation and detection, or c) amplification and detection are carried out within a single chamber of the device. In some embodiments, the device is configured for a two pot reaction in which amplification and detection are carried out in separate regions of the device. In some embodiments, the device is configured for a one pot reaction, wherein amplification, reverse transcription, amplification and reverse transcription, or amplification and in vitro transcription are carried out simultaneously with detection. In some embodiments, the device is configured for a two pot reaction, wherein any combination of reverse transcription, amplification, and in vitro transcription is performed as a first reaction in a first chamber, followed by the DETECTR™ assay in a second chamber. In some embodiments detection of the reaction products are detected in the second chamber. In other embodiments, detection of the reaction products are detected in a third chamber. In other embodiments, the amplification comprises a PCR amplification. In other embodiments, a reagent for the amplification comprises a recombinase, an oligonucleotide primer, a single-stranded DNA binding (SSB) protein, a polymerase, or a combination thereof. In other embodiments, the amplification is performed for no greater than 1 minute. In other embodiments, the amplification is performed at a temperature no greater than 20 Celsius. In other embodiments, the support medium comprises a nucleic acid amplification region. In other embodiments, the device is a lateral flow device. In other embodiments, the lateral flow device is capable of delivering results in less than 60 minutes. In other embodiments, the amplification is performed for no more than 5 minutes. In other embodiments, the enclosure contains a lateral flow strip. In other embodiments, the lateral flow strip comprises multiple layers. In other embodiments, the lateral flow strip is configured to interface with a sample preparation device. In other embodiments, the support medium is a lateral flow assay strip. In other embodiments, the enclosure contains at least two lateral flow strips, wherein the device is configured for multiplexed detection of target nucleic acids. In other embodiments, one or more reporters are present on the lateral flow strip. In other embodiments, a first reporter of the one or more reporters is a biotin-FAM reporter and a second of the one or more reporters is a biotin-DIG reporter. In other embodiments, the device is configured to detect at least two different single-stranded target nucleic acids with two different programmable nucleases and two different single-stranded reporters in a sample where the sample is contacted with the reagents for a predetermined length of time sufficient for trans cleavage of the at least two different single stranded reporters. In other embodiments, the device is configured for multiplexed detection, wherein multiple individual target nucleic acids are detected simultaneously at multiple and spatially separated detection spots located in a detection region of the lateral flow strip, further wherein each detection spot is complimentary toward an individual target nucleic acid. In other embodiments, a different reporter of one or more reporters corresponds to a different of target nucleic acid of two or more nucleic acids. In other embodiments, the device comprises multiple support mediums in a single enclosure. In other embodiments, the multiple support mediums share a single sample pad. In other embodiments, the sample pad is connected to the multiple support mediums in various configurations comprising branching or radial formation. In other embodiments, the reagents enclosed by the enclosure comprise multiple guide nucleic acids, multiple programmable nucleases, and multiple single stranded reporters, wherein a combination of one of the guide nucleic acids, one of the programmable nucleases, and one of the single stranded reporters detects one target nucleic acid and can provide a detection spot on the detection region. In other embodiments, the device comprising the multiple support mediums, further wherein each support medium comprises multiple detection spots, is configured to detect at least 6, 7, 8, 9, or 10 different target nucleic acids in a single reaction.

Described herein are various embodiments of a kit for detection of target nucleic acid. In some embodiments, the kit comprises any of the devices described herein, a sample collector, and instructions for the use thereof.

Described here are various methods for assaying target nucleic acids comprising: inserting a sample comprising the target nucleic acids into a chamber of a device comprising an enclosure and a locking mechanism for sealing the enclosure; engaging the locking mechanism to seal the chamber within the enclosure; releasing at least one reagent using a reagent release mechanism; and detecting a presence or absence of the target nucleic acids.

Described herein are methods for assaying a target nucleic acid, comprising: inserting a sample comprising the target nucleic acid into a chamber located in an enclosure of a device, wherein the chamber comprises a programmable nuclease; the enclosure comprises a locking-mechanism to form a fluid-tight seal between a sample collector and the device; and the enclosure comprises a reagent release mechanism capable of preventing the reaction from amplicon contamination by 2 fold as compared to detection outside the device; and a detection system configured to detect a presence of the target nucleic acid indicated by a signal produced via a reaction of the target nucleic acid with the programmable nuclease and release of a reporter; engaging the locking mechanism, thereby sealing the device in a body; releasing at least one reagent using the reagent release mechanism; and detecting the presence of the target nucleic acid on the detection system. In some embodiments, the reagent is an amplification reagent, as described herein. In some embodiments, the reagent is a DETECTR™ reagent as described herein. In other embodiments, the target nucleic acid is from a subject at risk of a disease that is contained in the sample of the subject is indicative of a disease. In other embodiments, the target nucleic acid comprises a portion of a nucleic acid from a virus or a bacterium or other agents responsible for a disease in the sample. In other embodiments, the target nucleic acid is indicative of the presence of cancer. In other embodiments, the sample comprises blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, or tissue. In other embodiments, the chamber contains a reporter. In other embodiments, the reporter comprises an RNA sequence. In other embodiments, wherein the reporter is an RNA reporter. In other embodiments, the reporter is suspended or immobilized in the chamber. In other embodiments, the reporter is cleaved by the programmable nuclease. In other embodiments, the chamber comprises a target deoxyribonucleic acid lacking a guanine at the 3′ end. In some embodiments, the terminal 3′ nucleotide in the segment of the target deoxyribonucleic acid is A, C or T. In some embodiments, the target deoxyribonucleic acid is a single-stranded deoxyribonucleic acid. In some embodiments, the target deoxyribonucleic acid is single stranded deoxyribonucleic acid oligonucleotides. In some embodiments, the target deoxyribonucleic acid is genomic single stranded deoxyribonucleic acids. In some embodiments, the target deoxyribonucleic acid has a length of from 18 to 100 nucleotides. In some embodiments, the target deoxyribonucleic acid has a length of from 18 to 30 nucleotides. In some embodiments, the target deoxyribonucleic acid has a length of 20 nucleotides. In some embodiments, the device is battery powered. In some embodiments, the device is not battery powered. In some embodiments, the device comprises a chemical heating element. In some embodiments, the detection system comprises a support medium configured to present the datable signal in a detectable format. In some embodiments, the support medium is a lateral flow assay strip. In some embodiments, the programmable nuclease is selected from the group consisting of a Cas13, Mad7, Mad2, Csm1, Cas9, C2c4, C2c8, C2c5, C2c10, C2c9, and CasZ. In some embodiments, the programmable nuclease is a type V CRISPR-Cas system, a type VI CRISPR Cas system, or a type III CRISPR Cas system. In some embodiments, the Cas13 is Cas13a, Cas13b, Cas13c, Cas13d, or Cas13e.

Described herein are various methods for programmable nuclease based-detection preventing contamination and utilizing a spin-through assay, comprising: providing an enclosure configured to contain contents comprising programmable nuclease-based detection reagents, amplification reagents, and a sample, wherein the detection reagents are physically separated from a mixture comprising amplification reagents and the sample by a filter; heating the enclosure until at least a portion of the sample is amplified by the amplification reagents; centrifuging the enclosure after the portion of the sample is amplified; heating the enclosure until the sample has reacted with the programmable nuclease-based detection reagents; and visualizing the contents of the enclosure. In some embodiments, the amplification is an isothermal amplification. In some embodiments, the heating of the enclosure the contents for amplification are heated to about 37 to 67 degrees Celsius. In some embodiments, the duration of the centrifuging is about 10 to 30 seconds. In some embodiments, the incubation of the contents for detection occurs at about 37 degrees Celsius. In some embodiments, the visualizing of the contents comprises visualizing fluorescence emitted from the contents. In some embodiments, the visualizing comprises viewing by eye, imaging by a device, or any combination thereof.

Described herein are various devices for programmable nuclease based-detection preventing contamination and utilizing a spin-through assay, comprising: an enclosure comprising programmable nuclease-based detection reagents, amplification reagents, and a sample, wherein the detection reagents are physically separated from a mixture comprising the amplification reagents and the sample by a filter, wherein the filter is configured to maintain separation between the detection reagents and the mixture until the enclosure is centrifuged, thereby allowing for amplification of the sample by the amplification reagents; wherein the filter is configured to facilitate transfer of the detection reagents in to the mixture upon centrifugation of the enclosure; and wherein the enclosure is configured to allow for visualization of one or more signals produced the mixture. In some embodiments, the enclosure comprises a transparent or translucent material configured to allow fluorescent light to pass therethrough. In some embodiments, the enclosure comprises a transparent or translucent material configured to allow visible light to pass therethrough.

Described herein are systems devices and methods for detection of a target nucleic acid. Described herein is a system for detection of a target nucleic acid, comprising a reagent chamber; a programmable nuclease disposed within the reagent chamber, the programmable nuclease comprising a guide nucleic acid that is complementary to the target nucleic acid or a portion thereof, wherein the programmable nuclease is configured to be activated through binding of the guide nucleic acid to the target nucleic acid; a reporter disposed within the reaction chamber, wherein the reporter comprises a cleavable nucleic acid and a detection moiety, wherein cleavage of the cleavable nucleic acid by the activated programmable nuclease releases the detection moiety from the cleavable nucleic acid; and a detection region, wherein the detection region comprises a stationary capture probe disposed thereon and configured to capture the released detection moiety, wherein release of the detection moiety directly or indirectly produces a detectable signal at a location corresponding to the stationary capture probe. In some embodiments, the guide nucleic acid comprises at least 10 nucleotides reverse complementary to the target nucleic acid or portion thereof. In some embodiments, a lateral flow assay strip comprising the detection region. In some embodiments, the system further comprises amplification reagents. In some embodiments, the amplification reagents are located within the reagent chamber. In some embodiments, the amplification reagents are LAMP reagents. In some embodiments, the lateral flow assay strip further comprises a sample pad. In some embodiments, the sample pad is the reagent chamber. In some embodiments, the lateral flow assay strip comprises a flowing capture probe. In some embodiments, the flowing capture probe is an anti-biotin functionalized gold nanoparticle. In some embodiments, the lateral flow assay strip further comprises a conjugation pad, wherein the sample pad is in fluid communication with the conjugation pad. In some embodiments, the sample pad is located in between the conjugation pad and the detection region. In some embodiments, the conjugation pad comprises a flowing capture probe. In some embodiments, the flowing capture probe comprises an antibody. In some embodiments, wherein the sample pad and/or the conjugation pad is in fluid communication with the detection region. In some embodiments, the lateral flow assay strip further comprises an absorption pad. In some embodiments, the sample pad, the conjugation pad, and/or the detection region are in fluid communication with the absorption pad. In some embodiments, the absorption pad is configured to draw a sample through the lateral flow assay strip by capillary action, wherein the sample contains the target nucleic acid. In some embodiments, a sample is driven through the lateral flow assay strip by an external pressure, wherein the sample contains the target nucleic acid. In some embodiments, the external pressure is applied by a pump in fluid communication with the lateral flow assay strip In some embodiments, the pump is a manually driven syringe or automated syringe pump. In some embodiments, the stationary capture probe comprises an antibody. In some embodiments, the antibody is selected from the group consisting of a nucleic acid capture sequence, anti-FITC, anti-DIG, anti-TAMRA, anti-Cy5, and anti-AF594. In some embodiments, the detection moiety comprises a chemical functional group. In some embodiments, the chemical functional group is biotin. In some embodiments, the detection moiety comprises a label. In some embodiments, the label is selected from the group consisting of a nucleic acid sequence complimentary to the nucleic acid capture sequence, FITC, DIG, TAMRA, Cy5™, and AF594™. In some embodiments, the programmable nuclease probe further comprises a Cas Enzyme. In some embodiments, the Cas enzyme is selected from the group consisting of Cas 12, Cas 13, Cas 14, and CasPhi. In some embodiments, the system further comprises a plurality of programmable nucleases and a plurality of different guide nucleic acids, wherein each programmable nuclease of the plurality of programmable nucleases comprises a guide nucleic acid of the plurality of different guide nucleic acids. In some embodiments, each guide nucleic acid is complimentary toward a corresponding target nucleic acid. In some embodiments, the system further comprises a plurality of reporters. In some embodiments, the system further comprises a plurality of labels, wherein each reporter of the plurality of reporters comprises a detection moiety and a label of the plurality of labels. In some embodiments, the system further comprises a plurality of different stationary capture probes, wherein each label of the plurality of labels is complimentary toward a corresponding stationary the plurality of different stationary capture probes. In some embodiments, an enclosure contains a sample inlet, the reagent chamber, and a lateral flow strip. In some embodiments, the system further comprises an amplification chamber located between the reagent chamber and the detection region. In some embodiments, the signal is a visual signal. In some embodiments, the detection region comprises one or more detection spots. In some embodiments, each detection spot of the one or more detection spots comprises a corresponding stationary capture probe of a plurality of stationary capture probes. In some embodiments, the detection spot is selected from the group of shapes consisting of a line, a circle, an oval, a rectangle, a triangle, and a plus sign. In some embodiments, the reagent chamber comprises a surface. In some embodiments, the programmable nuclease and/or the reporter is immobilized to the surface. In some embodiments, the programmable nuclease and/or the reporter is immobilized to the surface by covalent, non-covalent, or electrostatic force. In some embodiments, the programmable nuclease probe and/or the reporter is immobilized to the surface by a linker. In some embodiments, the Cas enzyme of the programmable nuclease is immobilized to a surface of the reagent chamber by the linker. In some embodiments, the linker is attached to the guide nucleic acid of the programmable nuclease. In some embodiments, the system further comprises a control spot. In some embodiments, the detection spot is located between the reagent chamber and the control spot. In some embodiments, the control spot is located between the reagent chamber and the detection spot. In some embodiments, the detectable signal is a visibly detectable signal. In some embodiments, the programmable nuclease is configured to cleave the cleavable nucleic acid in a cis-cleavage configuration or a trans-cleavage configuration. In some embodiments, the system further comprises a plurality of programmable nuclease probes, wherein each programmable nuclease probe of the plurality of programmable nuclease probe is complimentary to a plurality of corresponding segments of the target nucleic acid. In some embodiments, each corresponding segment of the target nucleic acid is non-overlapping, partially overlapping or fully overlapping.

Described herein is a lateral flow assay strip system for detection of a target nucleic acid, comprising: a sample pad comprising; a programmable nuclease disposed on the sample pad, the programmable nuclease comprising a guide nucleic acid that is complementary to the target nucleic acid or a portion thereof, wherein the programmable nuclease is configured to be activated through binding of the guide nucleic acid to the target nucleic acid, a reporter, wherein the reporter comprises a cleavable nucleic acid and a detection moiety, wherein cleavage of the cleavable nucleic acid by the activated programmable nuclease releases the detection moiety from the cleavable nucleic acid; and a detection region fluidly coupled to the sample pad, wherein the detection region comprises a stationary capture probe disposed thereon and configured to capture the released detection moiety, wherein release of the detection moiety directly or indirectly produces a detectable signal at a location corresponding to the stationary capture probe. In some embodiments, wherein the guide nucleic acid comprises at least 10 nucleotides reverse complementary to the target nucleic acid or portion thereof. In some embodiments, the system further comprises amplification reagents. In some embodiments, the amplification reagents are located within the reagent chamber. In some embodiments, the amplification reagents are LAMP reagents. In some embodiments, the lateral flow assay strip comprises a flowing capture probe. In some embodiments, lateral flow assay strip further comprises a conjugation pad, wherein the sample pad is in fluid communication with the conjugation pad, wherein the sample pad is located in between the conjugation pad and the detection region. In some embodiments, the conjugation pad comprises a flowing capture antibody. In some embodiments, the sample pad and/or the conjugation pad is in fluid communication with the detection region. In some embodiments, the lateral flow assay strip further comprises an absorption pad. In some embodiments, the sample pad, the conjugation pad, and/or the detection region are in fluid communication with the absorption pad. In some embodiments, the absorption pad is configured to draw a sample through the lateral flow assay strip by capillary action, wherein the sample contains the target nucleic acid. In some embodiments, a sample is driven through the lateral flow assay strip by an external pressure, wherein the sample contains the target nucleic acid. In some embodiments, the external pressure is applied by a pump in a in fluid communication with the lateral flow assay strip In some embodiments, the pump is a manually driven syringe or automated syringe pump. In some embodiments, the flowing capture probe is an anti-biotin functionalized gold nanoparticle. In some embodiments, the stationary capture probe comprises an antibody. In some embodiments, the antibody is selected from the group consisting of a nucleic acid capture sequence, anti-FITC, anti-DIG, anti-TAMRA, anti-Cy5, and anti-AF594. In some embodiments, the detection moiety comprises a chemical functional group. In some embodiments, the chemical functional group is biotin. In some embodiments, the detection moiety comprises a label. In some embodiments, the label is selected from the group consisting of a nucleic acid sequence complimentary to the nucleic acid capture sequence, FITC, DIG, TAMRA, Cy5™, and AF594™. In some embodiments, the programmable nuclease probe further comprises a Cas Enzyme. In some embodiments, the Cas enzyme is selected from the group consisting of Cas 12, Cas 13, Cas 14 and CasPhi. In some embodiments, the sample comprises a plurality of target nucleic acids. In some embodiments, the system further comprises a plurality of programmable nucleases and a plurality of different guide nucleic acids, wherein each programmable nuclease of the plurality of programmable nucleases comprises a guide nucleic acid of the plurality of different guide nucleic acids. In some embodiments, each guide nucleic acid is complimentary toward a corresponding target nucleic acid. In some embodiments, the system further comprises a plurality of reporters. In some embodiments, the system further comprises a plurality of labels, wherein each reporter of the plurality of reporters comprises a detection moiety and a label of the plurality of labels. In some embodiments, the system further comprises a plurality of different stationary capture probes, wherein each label of the plurality of labels is complimentary towards a stationary capture probe of the plurality of different stationary capture probes. In some embodiments, an enclosure contains a sample inlet, and the lateral flow strip. In some embodiments, the system further comprises an amplification chamber located between the reagent chamber and the detection region. In some embodiments, the signal is a visual signal. In some embodiments, the detection region comprises one or more detection spots. In some embodiments, each detection spot of the one or more detection spots comprises a corresponding stationary capture probe of a plurality of stationary capture probes. In some embodiments, where each detection spot of the one or more detection spot is selected from the group of shapes consisting of a line, a circle, an oval, a rectangle, a triangle, and a plus sign. In some embodiments, the programmable nuclease and/or the reporter is immobilized to the sample pad. In some embodiments, the programmable nuclease and/or the reporter is immobilized to the sample pad by covalent, non-covalent, or electrostatic force. In some embodiments, the programmable nuclease and/or the reporter immobilized to the sample pad is immobilized by a linker. In some embodiments, a Cas enzyme of the programmable nuclease is immobilized to the sample pad by the linker. In some embodiments, the linker is attached to the guide nucleic acid of the programmable nuclease. In some embodiments, the system further comprises a control spot. In some embodiments, the detection spot is located between the reagent chamber and the control spot. In some embodiments, the control spot is located between the reagent chamber and the detection spot. In some embodiments, the detectable signal is a visibly detectable signal. In some embodiments, the programmable nuclease is configured to cleave the cleavable nucleic acid in a cis-cleavage configuration or a trans-cleavage configuration. In some embodiments, the system further comprises a plurality of programmable nuclease probes, wherein each programmable nuclease probe of the plurality of programmable nuclease probe is complimentary to a plurality of corresponding segments of the target nucleic acid. In some embodiments, each corresponding segment of the target nucleic acid is non-overlapping, partially overlapping or fully overlapping.

Described herein is a lateral flow assay strip system for detection of a target nucleic acid, comprising: a sample pad comprising; a programmable nuclease disposed on the sample pad, the programmable nuclease comprising 1) a Cas 13 enzyme or a Cas 14 enzyme, and 2) a guide nucleic acid that is complementary to the target nucleic acid or a portion thereof, wherein the programmable nuclease is configured to be activated through binding of the guide nucleic acid to the target nucleic acid, and a reporter, wherein the reporter comprises a cleavable nucleic acid and a detection moiety, wherein cleavage of the cleavable nucleic acid by the activated programmable nuclease releases the detection moiety from the cleavable nucleic acid; and a detection region fluidly coupled to the sample pad, wherein the detection region comprises a stationary capture probe disposed thereon and configured to capture the released detection moiety, wherein release of the detection moiety directly or indirectly produces a detectable signal at a location corresponding to the stationary capture probe. In some embodiments, the guide nucleic acid comprises at least 10 nucleotides reverse complementary to the target nucleic acid or portion thereof. In some embodiments, the system further comprises a lateral flow assay strip comprising the detection region. In some embodiments, the system further comprises amplification reagents. In some embodiments, the amplification reagents are located within the reagent chamber. In some embodiments, the amplification reagents are LAMP reagents. In some embodiments, the lateral flow assay strip further comprises a sample pad. In some embodiments, the sample pad is the reagent chamber. In some embodiments, the lateral flow assay strip comprises a flowing capture probe. In some embodiments, the flowing capture probe is an anti-biotin functionalized gold nanoparticle. In some embodiments, the lateral flow assay strip further comprises a conjugation pad, wherein the sample pad is in fluid communication with the conjugation pad. In some embodiments, the sample pad is located in between the conjugation pad and the detection region. In some embodiments, the conjugation pad comprises a flowing capture probe. In some embodiments, the flowing capture probe comprises an antibody. In some embodiments, the sample pad and/or the conjugation pad is in fluid communication with the detection region. In some embodiments, the lateral flow assay strip further comprises an absorption pad. In some embodiments, the sample pad, the conjugation pad, and/or the detection region are in fluid communication with the absorption pad. In some embodiments, the absorption pad is configured to draw a sample through the lateral flow assay strip by capillary action, wherein the sample contains the target nucleic acid. In some embodiments, a sample is driven through the lateral flow assay strip by an external pressure, wherein the sample contains the target nucleic acid. In some embodiments, the external pressure is applied by a pump in fluid communication with the lateral flow assay strip In some embodiments, the pump is a manually driven syringe or automated syringe pump. In some embodiments, the stationary capture probe comprises an antibody. In some embodiments, the antibody is selected from the group consisting of a nucleic acid capture sequence, anti-FITC, anti-DIG, anti-TAMRA, anti-Cy5, and anti-AF594. In some embodiments, the detection moiety comprises a chemical functional group. In some embodiments, the chemical functional group is biotin. In some embodiments, the detection moiety comprises a label. In some embodiments, the label is selected from the group consisting of a nucleic acid sequence complimentary to the nucleic acid capture sequence, FITC, DIG, TAMRA, Cy5™, and AF594™. In some embodiments, the programmable nuclease probe further comprises a Cas Enzyme. In some embodiments, the Cas enzyme is Cas 12 or CasPhi. In some embodiments, the system further comprises a plurality of programmable nucleases and a plurality of different guide nucleic acids, wherein each programmable nuclease of the plurality of programmable nucleases comprises a guide nucleic acid of the plurality of different guide nucleic acids. In some embodiments, each guide nucleic acid is complimentary toward a corresponding target nucleic acid. In some embodiments, the system further comprises a plurality of reporters. In some embodiments, the system further comprises a plurality of labels, wherein each reporter of the plurality of reporters comprises a detection moiety and a label of the plurality of labels. In some embodiments, the system further comprises a plurality of different stationary capture probes, wherein each label of the plurality of labels is complimentary toward a corresponding stationary the plurality of different stationary capture probes. In some embodiments, an enclosure contains a sample inlet, the reagent chamber, and a lateral flow strip. In some embodiments, the system further comprises an amplification chamber located between the reagent chamber and the detection region. In some embodiments, the signal is a visual signal. In some embodiments, the detection region comprises one or more detection spots. In some embodiments, each detection spot of the one or more detection spots comprises a corresponding stationary capture probe of a plurality of stationary capture probes. In some embodiments, the detection spot is selected from the group of shapes consisting of a line, a circle, an oval, a rectangle, a triangle, and a plus sign. In some embodiments, the reagent chamber comprises a surface. In some embodiments, the programmable nuclease and/or the reporter is immobilized to the surface. In some embodiments, the programmable nuclease and/or the reporter is immobilized to the surface by covalent, non-covalent, or electrostatic force. In some embodiments, the programmable nuclease probe and/or the reporter is immobilized to the surface by a linker. In some embodiments, the Cas enzyme of the programmable nuclease is immobilized to a surface of the reagent chamber by the linker. In some embodiments, the linker is attached to the guide nucleic acid of the programmable nuclease. In some embodiments, the system further comprises a control spot. In some embodiments, the detection spot is located between the reagent chamber and the control spot. In some embodiments, control spot is located between the reagent chamber and the detection spot. In some embodiments, the detectable signal is a visibly detectable signal. In some embodiments, the programmable nuclease is configured to cleave the cleavable nucleic acid in a cis-cleavage configuration or a trans-cleavage configuration. In some embodiments, the system further comprises a plurality of programmable nuclease probes, wherein each programmable nuclease probe of the plurality of programmable nuclease probe is complimentary to a plurality of corresponding segments of the target nucleic acid. In some embodiments, each corresponding segment of the target nucleic acid is non-overlapping, partially overlapping or fully overlapping.

Described herein is a system for detection of a target nucleic acid, comprising: a reagent chamber; a programmable nuclease disposed within the reagent chamber, the programmable nuclease comprising 1) a Cas 14 enzyme or a Cas 13 enzyme, and 2) a guide nucleic acid that is complementary to the target nucleic acid or a portion thereof, wherein the programmable nuclease is configured to be activated through binding of the guide nucleic acid to the target nucleic acid; a reporter disposed within the reagent chamber, wherein the reporter comprises a cleavable nucleic acid and a detection moiety, wherein cleavage of the cleavable nucleic acid by the activated programmable nuclease releases the detection moiety from the cleavable nucleic acid; and a lateral flow assay strip comprising a detection region, wherein the detection region comprises a stationary capture probe disposed thereon and configured to capture the released detection moiety, wherein release of the detection moiety directly or indirectly produces a detectable signal at a location corresponding to the stationary capture probe. In some embodiments, the reagent chamber is fluidly connected to the detection chamber when a valve is open. In some embodiments, the guide nucleic acid comprises at least 10 nucleotides reverse complementary to the target nucleic acid or portion thereof. In some embodiments, the system further comprises a lateral flow assay strip comprising the detection region. In some embodiments, the system further comprises amplification reagents. In some embodiments, the amplification reagents are located within the reagent chamber. In some embodiments, the amplification reagents are LAMP reagents. In some embodiments, the lateral flow assay strip further comprises a sample pad. In some embodiments, the sample pad is the reagent chamber. In some embodiments, the lateral flow assay strip comprises a flowing capture probe. In some embodiments, the flowing capture probe is an anti-biotin functionalized gold nanoparticle. In some embodiments, the lateral flow assay strip further comprises a conjugation pad, wherein the sample pad is in fluid communication with the conjugation pad. In some embodiments, the sample pad is located in between the conjugation pad and the detection region. In some embodiments, the conjugation pad comprises a flowing capture probe. In some embodiments, the flowing capture probe comprises an antibody. In some embodiments, the sample pad and/or the conjugation pad is in fluid communication with the detection region. In some embodiments, the lateral flow assay strip further comprises an absorption pad. In some embodiments, the sample pad, the conjugation pad, and/or the detection region are in fluid communication with the absorption pad. In some embodiments, the absorption pad is configured to draw a sample through the lateral flow assay strip by capillary action, wherein the sample contains the target nucleic acid. In some embodiments, a sample is driven through the lateral flow assay strip by an external pressure, wherein the sample contains the target nucleic acid. In some embodiments, the external pressure is applied by a pump in fluid communication with the lateral flow assay strip In some embodiments, the pump is a manually driven syringe or automated syringe pump. In some embodiments, the stationary capture probe comprises an antibody. In some embodiments, the antibody is selected from the group consisting of a nucleic acid capture sequence, anti-FITC, anti-DIG, anti-TAMRA, anti-Cy5, and anti-AF594. In some embodiments, the detection moiety comprises a chemical functional group. In some embodiments, the chemical functional group is biotin. In some embodiments, the detection moiety comprises a label. In some embodiments, the label is selected from the group consisting of a nucleic acid sequence complimentary to the nucleic acid capture sequence, FITC, DIG, TAMRA, Cy5™, and AF594™. In some embodiments, the programmable nuclease probe further comprises a Cas Enzyme. In some embodiments, the Cas enzyme is selected from the group consisting of Cas 12, Cas 13, Cas 14, and CasPhi. In some embodiments, the system further comprises a plurality of programmable nucleases and a plurality of different guide nucleic acids, wherein each programmable nuclease of the plurality of programmable nucleases comprises a guide nucleic acid of the plurality of different guide nucleic acids. In some embodiments, each guide nucleic acid is complimentary toward a corresponding target nucleic acid. In some embodiments, the system further comprises a plurality of reporters. In some embodiments, further comprises a plurality of labels, wherein each reporter of the plurality of reporters comprises a detection moiety and a label of the plurality of labels. In some embodiments, the system further comprises a plurality of different stationary capture probes, wherein each label of the plurality of labels is complimentary toward a corresponding stationary the plurality of different stationary capture probes. In some embodiments, wherein an enclosure contains a sample inlet, the reagent chamber, and a lateral flow strip. In some embodiments, the system further comprises an amplification chamber located between the reagent chamber and the detection region. In some embodiments, the signal is a visual signal. In some embodiments, the detection region comprises one or more detection spots. In some embodiments, each detection spot of the one or more detection spots comprises a corresponding stationary capture probe of a plurality of stationary capture probes. In some embodiments, the detection spot is selected from the group of shapes consisting of a line, a circle, an oval, a rectangle, a triangle, and a plus sign. In some embodiments, the reagent chamber comprises a surface. In some embodiments, wherein the programmable nuclease and/or the reporter is immobilized to the surface. In some embodiments, the programmable nuclease and/or the reporter is immobilized to the surface by covalent, non-covalent, or electrostatic force. In some embodiments, the programmable nuclease probe and/or the reporter is immobilized to the surface by a linker. In some embodiments, the Cas enzyme of the programmable nuclease is immobilized to a surface of the reagent chamber by the linker. In some embodiments, the linker is attached to the guide nucleic acid of the programmable nuclease. In some embodiments, the system further comprises a control spot. In some embodiments, the detection spot is located between the reagent chamber and the control spot. In some embodiments, the control spot is located between the reagent chamber and the detection spot. In some embodiments, the detectable signal is a visibly detectable signal. In some embodiments, the programmable nuclease is configured to cleave the cleavable nucleic acid in a cis-cleavage configuration or a trans-cleavage configuration. In some embodiments, the system further comprises a plurality of programmable nuclease probes, wherein each programmable nuclease probe of the plurality of programmable nuclease probe is complimentary to a plurality of corresponding segments of the target nucleic acid. In some embodiments, each corresponding segment of the target nucleic acid is non-overlapping, partially overlapping or fully overlapping.

Described herein is a method for detecting a target nucleic acid, the method comprising the steps of: providing a system, comprising: a reagent chamber, a programmable nuclease disposed within the reagent chamber, the programmable nuclease comprising a guide nucleic acid that is complementary to the target nucleic acid or a portion thereof, wherein the programmable nuclease is configured to be activated through binding of the guide nucleic acid to the target nucleic acid, a reporter disposed within the reaction chamber, wherein the reporter comprises a cleavable nucleic acid and a detection moiety, wherein cleavage of the cleavable nucleic acid by the activated programmable nuclease releases the detection moiety from the cleavable nucleic acid, and a lateral flow assay strip comprising a detection region, wherein the detection region comprises a stationary capture probe disposed thereon and configured to capture the released detection moiety, wherein release of the detection moiety directly or indirectly produces a detectable signal at a location corresponding to the stationary capture probe contacting a sample with the reagent chamber; and identifying a presence of the target nucleic acid in the sample via the detectable signal produced at the location of the stationary capture probe. In some embodiments, the method further comprises amplifying sample comprising the target nucleic acid. In some embodiments, the guide nucleic acid comprises at least 10 nucleotides reverse complementary to the target nucleic acid or portion thereof. In some embodiments, the method further comprises a lateral flow assay strip comprising the detection region. In some embodiments, the method further comprises amplification reagents. In some embodiments, the amplification reagents are located within the reagent chamber. In some embodiments, wherein the amplification reagents are LAMP reagents. In some embodiments, the lateral flow assay strip further comprises a sample pad. In some embodiments, the sample pad is the reagent chamber. In some embodiments, the lateral flow assay strip comprises a flowing capture probe. In some embodiments, the flowing capture probe is an anti-biotin functionalized gold nanoparticle. In some embodiments, the lateral flow assay strip further comprises a conjugation pad, wherein the sample pad is in fluid communication with the conjugation pad. In some embodiments, the sample pad is located in between the conjugation pad and the detection region. In some embodiments, the conjugation pad comprises a flowing capture probe. In some embodiments, the flowing capture probe comprises an antibody. In some embodiments, the sample pad and/or the conjugation pad is in fluid communication with the detection region. In some embodiments, the lateral flow assay strip further comprises an absorption pad. In some embodiments, the sample pad, the conjugation pad, and/or the detection region are in fluid communication with the absorption pad. In some embodiments, the absorption pad is configured to draw a sample through the lateral flow assay strip by capillary action, wherein the sample contains the target nucleic acid. In some embodiments, a sample is driven through the lateral flow assay strip by an external pressure, wherein the sample contains the target nucleic acid. In some embodiments, the external pressure is applied by a pump in fluid communication with the lateral flow assay strip. In some embodiments, the pump is a manually driven syringe or automated syringe pump. In some embodiments, the stationary capture probe comprises an antibody. In some embodiments, the antibody is selected from the group consisting of a nucleic acid capture sequence, anti-FITC, anti-DIG, anti-TAMRA, anti-Cy5, and anti-AF594. In some embodiments, the detection moiety comprises a chemical functional group. In some embodiments, the chemical functional group is biotin. In some embodiments, the detection moiety comprises a label. In some embodiments, the label is selected from the group consisting of a nucleic acid sequence complimentary to the nucleic acid capture sequence, FITC, DIG, TAMRA, Cy5™, and AF594™. In some embodiments, the programmable nuclease probe further comprises a Cas Enzyme. In some embodiments, the Cas enzyme is selected from the group consisting of Cas 12, Cas 13, Cas 14, and CasPhi. In some embodiments, the method further comprises a plurality of programmable nucleases and a plurality of different guide nucleic acids, wherein each programmable nuclease of the plurality of programmable nucleases comprises a guide nucleic acid of the plurality of different guide nucleic acids. In some embodiments, each guide nucleic acid is complimentary toward a corresponding target nucleic acid. In some embodiments, the method further comprises a plurality of reporters. In some embodiments, the method further comprises a plurality of labels, wherein each reporter of the plurality of reporters comprises a detection moiety and a label of the plurality of labels. In some embodiments, further comprises a plurality of different stationary capture probes, wherein each label of the plurality of labels is complimentary toward a corresponding stationary the plurality of different stationary capture probes. In some embodiments, an enclosure contains a sample inlet, the reagent chamber, and a lateral flow strip. In some embodiments, the method further comprises an amplification chamber located between the reagent chamber and the detection region. In some embodiments, the signal is a visual signal. In some embodiments, the detection region comprises one or more detection spots. In some embodiments, each detection spot of the one or more detection spots comprises a corresponding stationary capture probe of a plurality of stationary capture probes. In some embodiments, the detection spot is selected from the group of shapes consisting of a line, a circle, an oval, a rectangle, a triangle, and a plus sign. In some embodiments, the reagent chamber comprises a surface. In some embodiments, the programmable nuclease and/or the reporter is immobilized to the surface. In some embodiments, the programmable nuclease and/or the reporter is immobilized to the surface by covalent, non-covalent, or electrostatic force. In some embodiments, the programmable nuclease probe and/or the reporter is immobilized to the surface by a linker. In some embodiments, the Cas enzyme of the programmable nuclease is immobilized to a surface of the reagent chamber by the linker. In some embodiments, the linker is attached to the guide nucleic acid of the programmable nuclease. In some embodiments, further comprises a control spot. In some embodiments, the detection spot is located between the reagent chamber and the control spot. In some embodiments, the control spot is located between the reagent chamber and the detection spot. In some embodiments, the detectable signal is a visibly detectable signal. In some embodiments, the programmable nuclease is configured to cleave the cleavable nucleic acid in a cis-cleavage configuration or a trans-cleavage configuration. In some embodiments, the method further comprises a plurality of programmable nuclease probes, wherein each programmable nuclease probe of the plurality of programmable nuclease probe is complimentary to a plurality of corresponding segments of the target nucleic acid. In some embodiments, each corresponding segment of the target nucleic acid is non-overlapping, partially overlapping or fully overlapping.

Described herein is a device for detection of a target nucleic acid, comprising: a reagent chamber comprising: a programmable nuclease disposed within the reagent chamber, the programmable nuclease comprising a guide nucleic acid that is complementary to the target nucleic acid or a portion thereof, wherein the programmable nuclease is configured to be activated through binding of the guide nucleic acid to the target nucleic acid, and a reporter disposed within the reaction chamber, wherein the reporter comprises a cleavable nucleic acid and a detection moiety, wherein cleavage of the cleavable nucleic acid by the activated programmable nuclease releases the detection moiety from the cleavable nucleic acid; a detection region in fluid communication with the reagent chamber, wherein the detection region comprises a stationary capture probe disposed thereon and configured to capture the released detection moiety, wherein release of the detection moiety directly or indirectly produces a detectable signal at a location corresponding to the stationary capture probe; and a housing containing the reagent chamber and the detection region. In some embodiments, the further comprises a valve located between the reagent chamber and the detection region, wherein the valve in an open position allows for fluid to pass therethrough and from the reagent chamber to the detection region. In some embodiments, the detection region is located on a lateral flow assay strip that is located within the housing. In some embodiments, the device further comprises a sample pad located on the lateral flow assay strip and located between the reagent chamber and the detection region. In some embodiments, the device further comprises an amplification chamber located between the reagent chamber and the detection region. In some embodiments, the programmable nuclease further comprises a Cas13 enzyme or a Cas14 enzyme. In some embodiments, the guide nucleic acid comprises at least 10 nucleotides reverse complementary to the target nucleic acid or portion thereof. In some embodiments, further comprises a lateral flow assay strip comprising the detection region. In some embodiments, the device further comprises amplification reagents. In some embodiments, the amplification reagents are located within the reagent chamber. In some embodiments, the amplification reagents are LAMP reagents. In some embodiments, the lateral flow assay strip further comprises a sample pad. In some embodiments, the sample pad is the reagent chamber. In some embodiments, the lateral flow assay strip comprises a flowing capture probe. In some embodiments, the flowing capture probe is an anti-biotin functionalized gold nanoparticle. In some embodiments, the lateral flow assay strip further comprises a conjugation pad, wherein the sample pad is in fluid communication with the conjugation pad. In some embodiments, the sample pad is located in between the conjugation pad and the detection region. In some embodiments, the conjugation pad comprises a flowing capture probe. In some embodiments, the flowing capture probe comprises an antibody. In some embodiments, the sample pad and/or the conjugation pad is in fluid communication with the detection region. In some embodiments, the lateral flow assay strip further comprises an absorption pad. In some embodiments, the sample pad, the conjugation pad, and/or the detection region are in fluid communication with the absorption pad. In some embodiments, the absorption pad is configured to draw a sample through the lateral flow assay strip by capillary action, wherein the sample contains the target nucleic acid. In some embodiments, a sample is driven through the lateral flow assay strip by an external pressure, wherein the sample contains the target nucleic acid. In some embodiments, the external pressure is applied by a pump in fluid communication with the lateral flow assay strip In some embodiments, the pump is a manually driven syringe or automated syringe pump. In some embodiments, the stationary capture probe comprises an antibody. In some embodiments, the antibody is selected from the group consisting of a nucleic acid capture sequence, anti-FITC, anti-DIG, anti-TAMRA, anti-Cy5, and anti-AF594. In some embodiments, the detection moiety comprises a chemical functional group. In some embodiments, the chemical functional group is biotin. In some embodiments, the detection moiety comprises a label. In some embodiments, the label is selected from the group consisting of a nucleic acid sequence complimentary to the nucleic acid capture sequence, FITC, DIG, TAMRA, Cy5™, and AF594™. In some embodiments, the programmable nuclease probe further comprises a Cas Enzyme. In some embodiments, the Cas enzyme is selected from the group consisting of Cas 12, Cas 13, Cas 14, and CasPhi. In some embodiments, the device further comprises a plurality of programmable nucleases and a plurality of different guide nucleic acids, wherein each programmable nuclease of the plurality of programmable nucleases comprises a guide nucleic acid of the plurality of different guide nucleic acids. In some embodiments, each guide nucleic acid is complimentary toward a corresponding target nucleic acid. In some embodiments, the device further comprises a plurality of reporters. In some embodiments, the device further comprises a plurality of labels, wherein each reporter of the plurality of reporters comprises a detection moiety and a label of the plurality of labels. In some embodiments, the device further comprises a plurality of different stationary capture probes, wherein each label of the plurality of labels is complimentary toward a corresponding stationary the plurality of different stationary capture probes. In some embodiments, an enclosure contains a sample inlet, the reagent chamber, and a lateral flow strip. In some embodiments, the device further comprises an amplification chamber located between the reagent chamber and the detection region. In some embodiments, the signal is a visual signal. In some embodiments, the detection region comprises one or more detection spots. In some embodiments, each detection spot of the one or more detection spots comprises a corresponding stationary capture probe of a plurality of stationary capture probes. In some embodiments, the detection spot is selected from the group of shapes consisting of a line, a circle, an oval, a rectangle, a triangle, and a plus sign. In some embodiments, the reagent chamber comprises a surface. In some embodiments, the programmable nuclease and/or the reporter is immobilized to the surface. In some embodiments, the programmable nuclease and/or the reporter is immobilized to the surface by covalent, non-covalent, or electrostatic force. In some embodiments, the programmable nuclease probe and/or the reporter is immobilized to the surface by a linker. In some embodiments, the Cas enzyme of the programmable nuclease is immobilized to a surface of the reagent chamber by the linker. In some embodiments, the linker is attached to the guide nucleic acid of the programmable nuclease. In some embodiments, comprising a control spot. In some embodiments, the detection spot is located between the reagent chamber and the control spot. In some embodiments, the control spot is located between the reagent chamber and the detection spot. In some embodiments, the detectable signal is a visibly detectable signal. In some embodiments, the programmable nuclease is configured to cleave the cleavable nucleic acid in a cis-cleavage configuration or a trans-cleavage configuration. In some embodiments, the device further comprises a plurality of programmable nuclease probes, wherein each programmable nuclease probe of the plurality of programmable nuclease probe is complimentary to a plurality of corresponding segments of the target nucleic acid. In some embodiments, each corresponding segment of the target nucleic acid is non-overlapping, partially overlapping or fully overlapping.

Described herein is a method for detecting a target nucleic acid, the method comprising the steps of: providing a device, comprising: a reagent chamber, comprising: a programmable nuclease disposed within the reagent chamber, the programmable nuclease comprising a guide nucleic acid that is complementary to the target nucleic acid or a portion thereof, wherein the programmable nuclease is configured to be activated through binding of the guide nucleic acid to the target nucleic acid, and a reporter molecule comprising a cleavable nucleic acid and a detection moiety, wherein cleavage of the cleavable nucleic acid by the activated programmable nuclease releases the detection moiety from the cleavable nucleic acid; a detection region in fluid communication with the reagent chamber, wherein the detection region comprises a stationary capture probe, to capture the released detection moiety, wherein release of the detection moiety directly or indirectly produces a detectable signal at a location corresponding to the stationary capture probe, a housing containing the reagent chamber and detection region, introducing a sample into the reagent chamber, thereby enabling the detection moiety to be released via the cleavage of the nucleic acid to produce a detection moiety solution; contacting the detection moiety solution with the detection region; and identifying a presence of the target nucleic acid in the sample via the detectable signal produced at the location corresponding to the stationary capture probe. The system of claim 1, wherein the guide nucleic acid comprises at least 10 nucleotides reverse complementary to the target nucleic acid or portion thereof. In some embodiments, the method further comprises a lateral flow assay strip comprising the detection region. In some embodiments, the method further comprises amplification reagents. In some embodiments, the amplification reagents are located within the reagent chamber. In some embodiments, the amplification reagents are LAMP reagents. In some embodiments, the lateral flow assay strip further comprises a sample pad. In some embodiments, the sample pad is the reagent chamber. In some embodiments, the lateral flow assay strip comprises a flowing capture probe. In some embodiments, the flowing capture probe is an anti-biotin functionalized gold nanoparticle. In some embodiments, the lateral flow assay strip further comprises a conjugation pad, wherein the sample pad is in fluid communication with the conjugation pad. In some embodiments, the sample pad is located in between the conjugation pad and the detection region. In some embodiments, the conjugation pad comprises a flowing capture probe. In some embodiments, the flowing capture probe comprises an antibody. In some embodiments, the sample pad and/or the conjugation pad is in fluid communication with the detection region. In some embodiments, the lateral flow assay strip further comprises an absorption pad. In some embodiments, the sample pad, the conjugation pad, and/or the detection region are in fluid communication with the absorption pad. In some embodiments, the absorption pad is configured to draw a sample through the lateral flow assay strip by capillary action, wherein the sample contains the target nucleic acid. In some embodiments, a sample is driven through the lateral flow assay strip by an external pressure, wherein the sample contains the target nucleic acid. In some embodiments, the external pressure is applied by a pump in fluid communication with the lateral flow assay strip In some embodiments, the pump is a manually driven syringe or automated syringe pump. In some embodiments, the stationary capture probe comprises an antibody. In some embodiments, the antibody is selected from the group consisting of a nucleic acid capture sequence, anti-FITC, anti-DIG, anti-TAMRA, anti-Cy5, and anti-AF594. In some embodiments, the detection moiety comprises a chemical functional group. In some embodiments, the chemical functional group is biotin. In some embodiments, the detection moiety comprises a label. In some embodiments, the label is selected from the group consisting of a nucleic acid sequence complimentary to the nucleic acid capture sequence, FITC, DIG, TAMRA, Cy5™, and AF594™. In some embodiments, the programmable nuclease probe further comprises a Cas Enzyme. In some embodiments, the Cas enzyme is selected from the group consisting of Cas 12, Cas 13, Cas 14, and CasPhi. In some embodiments, the method further comprises a plurality of programmable nucleases and a plurality of different guide nucleic acids, wherein each programmable nuclease of the plurality of programmable nucleases comprises a guide nucleic acid of the plurality of different guide nucleic acids. In some embodiments, each guide nucleic acid is complimentary toward a corresponding target nucleic acid. In some embodiments, the method further comprises a plurality of reporters. In some embodiments, the method further comprises a plurality of labels, wherein each reporter of the plurality of reporters comprises a detection moiety and a label of the plurality of labels. In some embodiments, the method further comprises a plurality of different stationary capture probes, wherein each label of the plurality of labels is complimentary toward a corresponding stationary the plurality of different stationary capture probes. In some embodiments, an enclosure contains a sample inlet, the reagent chamber, and a lateral flow strip. In some embodiments, the method further comprises an amplification chamber located between the reagent chamber and the detection region. In some embodiments, the signal is a visual signal. In some embodiments, the detection region comprises one or more detection spots. In some embodiments, each detection spot of the one or more detection spots comprises a corresponding stationary capture probe of a plurality of stationary capture probes. In some embodiments, the detection spot is selected from the group of shapes consisting of a line, a circle, an oval, a rectangle, a triangle, and a plus sign. In some embodiments, the reagent chamber comprises a surface. In some embodiments, the programmable nuclease and/or the reporter is immobilized to the surface. In some embodiments, the programmable nuclease and/or the reporter is immobilized to the surface by covalent, non-covalent, or electrostatic force. In some embodiments, the programmable nuclease probe and/or the reporter is immobilized to the surface by a linker. In some embodiments, the Cas enzyme of the programmable nuclease is immobilized to a surface of the reagent chamber by the linker. In some embodiments, the linker is attached to the guide nucleic acid of the programmable nuclease. In some embodiments, the method further comprises a control spot. In some embodiments, the detection spot is located between the reagent chamber and the control spot. In some embodiments, the control spot is located between the reagent chamber and the detection spot. In some embodiments, the detectable signal is a visibly detectable signal. In some embodiments, the programmable nuclease is configured to cleave the cleavable nucleic acid in a cis-cleavage configuration or a trans-cleavage configuration. In some embodiments, the method further comprises a plurality of programmable nuclease probes, wherein each programmable nuclease probe of the plurality of programmable nuclease probe is complimentary to a plurality of corresponding segments of the target nucleic acid. In some embodiments, each corresponding segment of the target nucleic acid is non-overlapping, partially overlapping or fully overlapping.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a workflow for the streamlined lateral flow devices of the present disclosure include sample preparation, optional amplification of target nucleic acids, CRISPR-based detection with programmable nucleases, guide nucleic acids, and reporters, and qualitative results. All of said processes can occur in a single streamlined lateral flow device disclosed herein.

FIG. 2 shows an example of streamlined lateral flow device of the present disclosure.

FIG. 3 shows individual parts of sample preparation devices of the present disclosure.

FIG. 4 shows a sample work flow using a sample processing device.

FIG. 5 shows the layout of a Milenia commercial strip with a typical reporter.

FIG. 6 shows the layout of a Milenia HybridDetect 1 strip with an amplicon.

FIG. 7 shows the layout of a Milenia HybridDetect 1 strip with a standard Cas reporter.

FIG. 8 shows a modified Cas reporter comprising a DNA linker to biotin-dT (808) bound to a FAM molecule (804).

FIG. 9 shows the layout of Milenia HybridDetect strips with the modified Cas reporter as described in FIG. 8.

FIG. 10 shows an example of a single target assay format (to left) and a multiplexed assay format (to right).

FIG. 11 shows another variation of an assay prior to use (top), an assay with a positive result (middle left), an assay with a negative result (middle right), and a failed test (bottom).

FIG. 12 shows one design of a tethered lateral flow Cas reporter.

FIG. 13 shows a workflow for CRISPR diagnostics using the tethered reporter using magnetic beads.

FIG. 14 shows a schematic for an enzyme-reporter system that is filtered by streptavidin-biotin before reaching the reaction chamber.

FIG. 15 shows a schematic for sample input module for sample preparation on a point-of-need CRISPR device.

FIG. 16 shows a schematic diagram of amplification and DETECTR™ channels on a point-of-need CRISPR device.

FIG. 17 shows detailed steps of process flow on a point-of-need CRISPR device.

FIG. 18 shows the workflow of the device. In Step 1, the reaction cassette containing tubes A (nucleic acid amplification reaction) and B (CRISPR enzyme reagents) is inserted into the device that contains the puncture and mixing chamber, the lateral flow cassette, and the lateral flow wick. In Step 2, the device is closed and locked. This moves the reaction cassette down and onto the projections from the puncture and mixing chamber. In Step 3, the contents of tubes A and B mix together after being punctured. The mixed reagents flow to the bottom of the device where they are wicked up into the lateral flow cassette.

FIG. 19 shows an example device format using existing USTAR lateral flow cassette hardware. (A) The contents of the chase buffer bulb (Tube B) were replaced by CRISPR enzyme reagents, and a RT-LAMP nucleic acid amplification reaction was used for (Tube A). (B) The closed reaction cassette next to the USTAR lateral flow device. (C) The side view of the reaction cassette before Tube A and Tube B are punctured when the device is closed. (D) Example readout of the lateral flow after the device has been run.

FIG. 20 shows workflow for CRISPR diagnostics using a spin-through assay format. The entire process is sealed and does not require the amplification tube to be opened which reduces the potential for contamination.

FIG. 21 illustrates an assay design for a point-of-need (PON) 5-plex respiratory panel.

FIG. 22 illustrates a PON disposable device, according to an embodiment.

FIG. 23 illustrates an embodiment of a multiplex lateral flow strip, as described herein.

FIG. 24 illustrates an embodiment of a workflow with multiplex “HotPot” as described herein.

FIG. 25 illustrates an embodiment for HRP paper-based detection, as described herein.

FIG. 26 illustrates an embodiment for an HRP-based multiplex lateral flow assay, as described herein.

FIG. 27 illustrates an embodiment for Multiplexed Cas13 immobilization approach to an HRP-based multiplex lateral flow assay, as described herein.

FIG. 28 shows results for both DNAse and DETECTR™ based assays for two replicate runs a week apart.

FIG. 29 illustrates the use of multiple Cas-complex probes guide pooling enhanced signal detection to a lateral flow assay, as described herein

FIG. 30 depicts results of a DETECTR™ assay showing enhanced Cas12a-based detection of the GF184 target using a pooled-guide (pooled-gRNA) format compared to DETECTR™ Cas12a-based assay using an individual gRNA format.

FIG. 31 depicts results of a DETECTR™ assay showing enhanced sensitivity of the Cas13a-based detection of the SC2 target using a pooled-guide format compared to the Cas13a-based assays using an individual guide format.

FIG. 32 shows images corresponding to each chamber, used to count the number of positive droplets, showing that the Cas13a-DETECTR™ assay samples containing the pooled guide RNAs generated more crystals containing the amplified products per starting copy of the target RNA than the Cas13a-DETECTR™ assay samples containing the guide RNAs in individual format.

FIG. 33 shows that measurement of signal intensity following amplification showed that the Cas13a-DETECTR™ assay samples containing the pooled guide RNAs generated more signal intensity per starting copy of the target template RNA than the Cas13a-DETECTR™ assay samples containing the guide RNAs in individual format.

FIG. 34 shows that measurement of signal intensity following amplification showed that the Cas13a-DETECTR™ assay samples containing the pooled guide RNAs generated more signal intensity per starting copy of the target template RNA than the Cas13a-DETECTR™ assay samples containing the guide RNAs in individual format. FIG. 34 also shows that relative quantification performed by counting the number of positive droplets showed that the Cas13a-DETECTR™ assay samples containing the pooled guide RNAs generated more crystals containing the amplified products per starting copy of the target template RNA than the Cas13a-DETECTR™ assay samples containing the guide RNAs in individual format.

FIG. 35 shows that Cas13a DETECTR™ assay samples containing the pooled guides (R4637, R4638, R4667, R4676, R4684, R4689, R4691) did not exhibit higher target detection sensitivity per starting copy of the target than the Cas13a DETECTR™ samples containing the single guides R4684, R4667, or R4785 (RNAseP guide) in individual format.

### DETAILED DESCRIPTION

The present disclosure provides various streamlined lateral flow devices for rapid lab tests, which may quickly assess whether a target nucleic acid is present in a sample by using a programmable nuclease that can interact with functionalized surfaces of the devices to generate a detectable signal.

The present disclosure provides streamlined lateral flow devices for analyzing a sample comprising a nucleic acid of interest to be detected. The device may be configured to perform sample preparation. The device may be configured to optionally perform amplification of a target nucleic acid sequence. The device may be configured to determine whether the nucleic acid from the sample comprises the target nucleic acid sequence. The device may be configured to receive the sample, perform sample preparation, amplify a target nucleic acid sequence, and determine whether the nucleic acid from the sample comprises the target nucleic acid sequence. The device may be configured to receive the sample, perform sample preparation, and determine whether the nucleic acid from the sample comprises the target nucleic acid sequence.

Advantageously, the device may be configured to minimize contamination of the sample. In some cases, the device is configured to receive or uptake the sample. The sample uptake may be accomplished by inserting a swab containing the sample into the device. The device may be configured so that the swab cannot be removed once inserted. This may prevent backflow or leakage of the sample and subsequent cross-contamination.

The lateral flow assays and reagents disclosed herein can be used as a companion diagnostic with any of the diseases disclosed herein, or can be used in reagent kits, point-of-care diagnostics, or over-the-counter diagnostics. The systems may be used as a point of care diagnostic or as a lab test for detection of a target nucleic acid and, thereby, detection of a condition in a subject from which the sample was taken. The systems may be used in various sites or locations, such as in laboratories, in hospitals, in physician offices/laboratories (POLs), in clinics, at remotes sites, or at home. Sometimes, the present disclosure provides various streamlined lateral flow devices for consumer genetic use or for over the counter use.

Described herein are lateral flow devices and methods for detecting the presence of a target nucleic acid in a sample. The devices and methods for detecting the presence of a target nucleic acid in a sample can be used in a rapid lab test for detection of a target nucleic acid of interest. In particular, provided herein are lateral flow devices, wherein the rapid lab test can be performed in a streamlined, single system. The target nucleic acid may be a portion of a nucleic acid from a virus or a bacterium or other agents responsible for a disease in the sample. The target nucleic acid may be a portion of an RNA or DNA from any organism in the sample. In some embodiments, programmable nucleases disclosed herein are activated by RNA or DNA to initiate trans cleavage activity of an RNA reporter. A programmable nuclease as disclosed herein, in some cases, binds to a target RNA to initiate trans cleavage of an RNA reporter. In some instances, a programmable nuclease as disclosed herein binds to a target DNA to initiate trans cleavage of an RNA reporter. In some embodiments, the Cas13 binds to a target ssDNA to initiates trans cleavage of RNA detector nucleic acids. In some embodiments, Cas14 binds to target dsDNA or ssDNA to initiate cleavage of DNA detector nucleic acids. In some embodiments, DNA detector nucleic acids are referred to as cleavable nucleic acids of a reporter.

The detection of the target nucleic acid in the sample may indicate the presence of the disease in the sample and may provide information for taking action to reduce the transmission of the disease to individuals in the disease-affected environment or near the disease-carrying individual. The detection of the target nucleic acid in the sample may indicate the presence of a disease mutation, such as a single nucleotide polymorphism (SNP) that provide antibiotic resistance to one or more disease-causing bacteria.

The detection of the target nucleic acid is facilitated by a programmable nuclease. The programmable nuclease can become activated after binding of a guide nucleic acid with a target nucleic acid, in which the activated programmable nuclease can cleave the target nucleic acid and can have trans cleavage activity, which can also be referred to as “collateral” or “transcollateral” cleavage. Trans cleavage activity can be non-specific cleavage of nearby single-stranded nucleic acids by the activated programmable nuclease, such as trans cleavage of reporters with a detection moiety. Once the reporter is cleaved by the activated programmable nuclease, the detection moiety is released from the reporter and generates a detectable signal that is immobilized on a support medium. Often the detection moiety is at least one of a fluorophore, a dye, a polypeptide, or a nucleic acid. Sometimes the detection moiety binds to a capture molecule on the support medium to be immobilized. The detectable signal can be visualized on the support medium to assess the presence or level of the target nucleic acid associated with a condition or state, such as a disease. The programmable nuclease can be a CRISPR-Cas (clustered regularly interspaced short palindromic repeats—CRISPR associated) nucleoprotein complex with trans cleavage activity, which can be activated by binding of a guide nucleic acid with a target nucleic acid.

A biological sample from an individual or an environmental sample can be tested to determine whether the individual has a communicable disease. At least one target nucleic acid from a pathogen responsible for the disease that is detected can also indicate that the pathogen is wild-type or comprises a mutation that confers resistance to treatment, such as antibiotic treatment. A sample from an individual or from an environment is applied to the reagents described herein. The reaction between the sample and the reagents may be performed in the reagent chamber provided in the kit or on a support medium provided in the kit. If the target nucleic acid is present in the sample, the target nucleic acid binds to the guide nucleic acid to activate the programmable nuclease. The activated programmable nuclease cleaves the reporter and generates a detectable signal that can be visualized on the support medium. If the target nucleic acid is absent in the sample or below the threshold of detection, the guide nucleic acid remains unbound, the programmable nuclease remains inactivated, and the reporter remains uncleaved.

If the sample is positive for the target nucleic acid, a test marker for the detectable signal can also be visualized. The results in the detection region can be visualized by eye or using a mobile device. In some instances, an individual can open a mobile application for reading of the test results on a mobile device having a camera and take an image of the support medium, including the detection region, barcode, reference color scale, and fiduciary markers on the housing, using the camera of the mobile device and the graphic user interface (GUI) of the mobile application. The mobile application can identify the test, visualize the detection region in the image, and/or analyze to determine the presence or absence or the level of the target nucleic acid responsible for the disease. In some embodiments, the mobile application may communicate with a remote device (e.g., via a cloud-based encrypted communication system) to transfer the image to a remote practitioner or cloud-based analysis tool for analysis of the presence, absence, or level of the target nucleic acid. The mobile application can present the results of the test to the individual, store the test results in the mobile application, or communicate with a remote device and transfer the data of the test results.

Such streamlined lateral flow devices described herein may allow for detection of target nucleic acid, and in turn the viral infection associated with the target nucleic acid, in remote regions or low resource settings without specialized equipment. Also, such streamlined lateral flow devices and methods described herein may allow for detection of target nucleic acid, and in turn the pathogen and disease associated with the target nucleic acid, in healthcare clinics or doctor offices without specialized equipment. In some cases, this provides a point of care testing for users to easily test for a disease or infection at home or quickly in an office of a healthcare provider. Assays that deliver results in under an hour, for example, in 15 to 60 minutes, are particularly desirable for at-home testing for many reasons. Antivirals can be most effective when administered within the first 48 hours, thus quick and accurate results may be desired to improve the efficacy of antivirals. Similarly, at-home rapid testing may improve antibiotic stewardship by reducing unnecessary antibiotic use (e.g., in the case where the diagnostic test indicates a viral infection not a bacterial infection) and/or ensuring patient compliance in taking the full course of antibiotics even after symptoms have resolved. Thus, the streamlined lateral flow devices disclosed herein, which are capable of delivering results in under an hour can allow for the delivery of anti-viral therapy at an optimal time. Additionally, the streamlined lateral flow devices provided herein, which are capable of delivering quick diagnoses and results, can help keep or send a patient home, improve comprehensive disease surveillance, and/or prevent the spread of an infection. In other cases, this provides a test, which can be used in a lab to detect a nucleic acid of interest in a sample from a subject. In particular, provided herein are streamlined lateral flow devices, wherein the rapid lab tests can be performed in a single system. In some cases, this may be valuable in detecting diseases in a developing country and as a global healthcare tool to detect the spread of a disease or efficacy of a treatment or provide early detection of a viral infection.

### Streamlined Lateral Flow Device for Detection of a Target Nucleic Acid

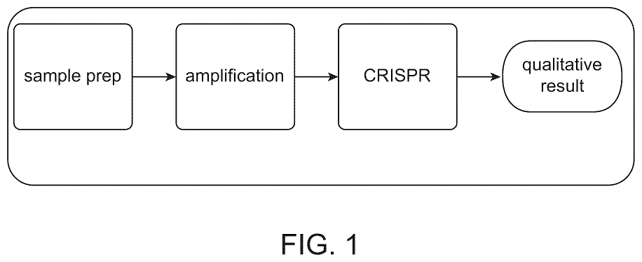

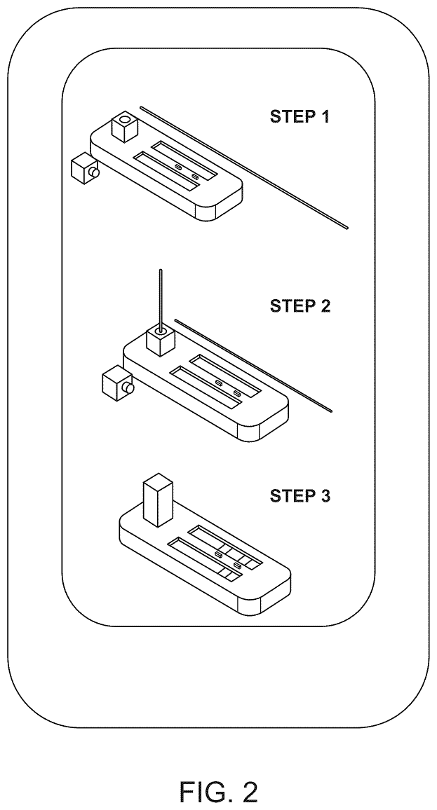

The present disclosure provides a streamlined lateral flow device for detection of a target nucleic acid. A schematic of the device is shown in FIG. 2. A workflow performed by the streamlined lateral flow device is shown in FIG. 1. The device may be configured to determine whether the nucleic acid from the sample comprises one or more of a plurality of target nucleic acid sequences. Optionally, the device may be configured to amplify one or more of a plurality of target nucleic acid sequences. The streamlined lateral flow device performs sample preparation steps comprising purifying nucleic acids from the sample on-device. The sample preparation may comprise the removal of species that are not nucleic acids (e.g., lipids, polypeptides). The sample preparation may comprise nucleic acid fragmentation. The sample preparation may comprise cell lysis. The lysis may comprise chemical lysis. The lysis may comprise physical lysis. The lysis may comprise osmotic lysis. The lysis may also be carried out by exposing the sample to heat.

The streamlined lateral flow device optionally performs amplification on-device. The amplification may comprise isothermal amplification. The isothermal amplification may be loop mediated isothermal amplification (LAMP). The isothermal amplification may be performed at 62° C. The isothermal amplification may be performed for at least 2 minutes. The isothermal amplification may be performed for no more than 5 minutes. The temperature of the amplification reaction and the time of the detection reaction can be controlled or tuned within the streamlined lateral flow device disclosed herein.

The streamlined lateral flow device may perform detection on-device. The detection may include determining whether the nucleic acid from the sample comprises the target nucleic acid sequence and may comprise detecting the target nucleic acid sequence. The detecting may comprise the use of a programmable nuclease (e.g., any Cas protein disclosed herein). The detecting may comprise the use a guide nucleic acid. In some cases, the detecting may comprise the use of reporters. The detecting may comprise a DETECTR™ reaction. The detecting may comprise the use of a lateral flow strip within the streamlined lateral flow device. A region of the lateral flow strip may be configured to change color when the nucleic acid from the sample comprises the target nucleic acid sequence.

Determining whether the nucleic acid from the sample comprises the target nucleic acid sequence using the streamlined lateral flow devices disclosed herein may require that at least 5000 copies of the target nucleic acid sequence be present in the sample. Determining whether the nucleic acid from the sample comprises the target nucleic acid sequence may require that at least from 10 to 50 copies of the target nucleic acid sequence be present in the sample. Determining whether the nucleic acid from the sample comprises the target nucleic acid sequence may require that at least from 50 to 100 copies of the target nucleic acid sequence be present in the sample. Determining whether the nucleic acid from the sample comprises the target nucleic acid sequence may require that at least from 100 to 150 copies of the target nucleic acid sequence be present in the sample. Determining whether the nucleic acid from the sample comprises the target nucleic acid sequence may require that at least from 150 to 200 copies of the target nucleic acid sequence be present in the sample. Determining whether the nucleic acid from the sample comprises the target nucleic acid sequence may require that at least from 200 to 250 copies of the target nucleic acid sequence be present in the sample. Determining whether the nucleic acid from the sample comprises the target nucleic acid sequence may require that at least from 250 to 300 copies of the target nucleic acid sequence be present in the sample. Determining whether the nucleic acid from the sample comprises the target nucleic acid sequence may require that at least from 300 to 350 copies of the target nucleic acid sequence be present in the sample. Determining whether the nucleic acid from the sample comprises the target nucleic acid sequence may require that at least from 350 to 400 copies of the target nucleic acid sequence be present in the sample. Determining whether the nucleic acid from the sample comprises the target nucleic acid sequence may require that at least from 400 to 450 copies of the target nucleic acid sequence be present in the sample. Determining whether the nucleic acid from the sample comprises the target nucleic acid sequence may require that at least from 450 to 500 copies of the target nucleic acid sequence be present in the sample. Determining whether the nucleic acid from the sample comprises the target nucleic acid sequence may require that at least from 500 to 550 copies of the target nucleic acid sequence be present in the sample. Determining whether the nucleic acid from the sample comprises the target nucleic acid sequence may require that at least from 550 to 600 copies of the target nucleic acid sequence be present in the sample. Determining whether the nucleic acid from the sample comprises the target nucleic acid sequence may require that at least from 600 to 650 copies of the target nucleic acid sequence be present in the sample. Determining whether the nucleic acid from the sample comprises the target nucleic acid sequence may require that at least from 650 to 700 copies of the target nucleic acid sequence be present in the sample. Determining whether the nucleic acid from the sample comprises the target nucleic acid sequence may require that at least from 700 to 750 copies of the target nucleic acid sequence be present in the sample. Determining whether the nucleic acid from the sample comprises the target nucleic acid sequence may require that at least from 750 to 800 copies of the target nucleic acid sequence be present in the sample. Determining whether the nucleic acid from the sample comprises the target nucleic acid sequence may require that at least from 800 to 850 copies of the target nucleic acid sequence be present in the sample. Determining whether the nucleic acid from the sample comprises the target nucleic acid sequence may require that at least from 850 to 900 copies of the target nucleic acid sequence be present in the sample. Determining whether the nucleic acid from the sample comprises the target nucleic acid sequence may require that at least from 900 to 950 copies of the target nucleic acid sequence be present in the sample. Determining whether the nucleic acid from the sample comprises the target nucleic acid sequence may require that at least from 950 to 1000 copies of the target nucleic acid sequence be present in the sample. Determining whether the nucleic acid from the sample comprises the target nucleic acid sequence may require that at least from 1000 to 1500 copies of the target nucleic acid sequence be present in the sample. Determining whether the nucleic acid from the sample comprises the target nucleic acid sequence may require that at least from 1500 to 2000 copies of the target nucleic acid sequence be present in the sample. Determining whether the nucleic acid from the sample comprises the target nucleic acid sequence may require that at least from 2000 to 2500 copies of the target nucleic acid sequence be present in the sample. Determining whether the nucleic acid from the sample comprises the target nucleic acid sequence may require that at least from 2500 to 3000 copies of the target nucleic acid sequence be present in the sample. Determining whether the nucleic acid from the sample comprises the target nucleic acid sequence may require that at least from 3000 to 3500 copies of the target nucleic acid sequence be present in the sample. Determining whether the nucleic acid from the sample comprises the target nucleic acid sequence may require that at least from 3500 to 4000 copies of the target nucleic acid sequence be present in the sample. Determining whether the nucleic acid from the sample comprises the target nucleic acid sequence may require that at least from 4000 to 4500 copies of the target nucleic acid sequence be present in the sample. Determining whether the nucleic acid from the sample comprises the target nucleic acid sequence may require that at least from 4500 to 5000 copies of the target nucleic acid sequence be present in the sample. Determining whether the nucleic acid from the sample comprises the target nucleic acid sequence may require that at least from 50 to 200 copies of the target nucleic acid sequence be present in the sample. Determining whether the nucleic acid from the sample comprises the target nucleic acid sequence may require that at least from 50 to 2000 copies of the target nucleic acid sequence be present in the sample. Determining whether the nucleic acid from the sample comprises the target nucleic acid sequence may require that at least from 1000 to 3000 copies of the target nucleic acid sequence be present in the sample. Determining whether the nucleic acid from the sample comprises the target nucleic acid sequence may require that at least from 2000 to 5000 copies of the target nucleic acid sequence be present in the sample. Determining whether the nucleic acid from the sample comprises the target nucleic acid sequence may require no more than 5000 copies of the target nucleic acid sequence be present in the sample. Determining whether the nucleic acid from the sample comprises the target nucleic acid sequence may require no more than 4500 copies of the target nucleic acid sequence be present in the sample. Determining whether the nucleic acid from the sample comprises the target nucleic acid sequence may require no more than 4000 copies of the target nucleic acid sequence be present in the sample. Determining whether the nucleic acid from the sample comprises the target nucleic acid sequence may require no more than 3500 copies of the target nucleic acid sequence be present in the sample. Determining whether the nucleic acid from the sample comprises the target nucleic acid sequence may require no more than 3000 copies of the target nucleic acid sequence be present in the sample. Determining whether the nucleic acid from the sample comprises the target nucleic acid sequence may require no more than 2500 copies of the target nucleic acid sequence be present in the sample. Determining whether the nucleic acid from the sample comprises the target nucleic acid sequence may require no more than 2000 copies of the target nucleic acid sequence be present in the sample. Determining whether the nucleic acid from the sample comprises the target nucleic acid sequence may require no more than 1500 copies of the target nucleic acid sequence be present in the sample. Determining whether the nucleic acid from the sample comprises the target nucleic acid sequence may require no more than 1000 copies of the target nucleic acid sequence be present in the sample. Determining whether the nucleic acid from the sample comprises the target nucleic acid sequence may require no more than 500 copies of the target nucleic acid sequence be present in the sample. Determining whether the nucleic acid from the sample comprises the target nucleic acid sequence may require no more than 100 copies of the target nucleic acid sequence be present in the sample. Determining whether the nucleic acid from the sample comprises the target nucleic acid sequence may require no more than 50 copies of the target nucleic acid sequence be present in the sample. Determining whether the nucleic acid from the sample comprises the target nucleic acid sequence may require no more than 10 copies of the target nucleic acid sequence be present in the sample.

Samples consistent with the streamlined lateral flow devices disclosed herein may include biological samples, environmental samples, and/or agricultural samples, or the like. The sample may comprise plasma, saliva, tissue, a cell, cell lysate, a protein, a virus, a lipid, a metabolite, or any combination thereof. The biological sample may comprise a cell. The sample may comprise a plurality of nucleic acids.

Devices consistent with the streamlined lateral flow devices disclosed herein include single use device. In some aspects, the device is battery powered, or otherwise electrically-powered (e.g., via an electrical outlet). In some aspects, the device does not require battery power. In some aspects, the device comprises a chemical heating element. In some aspects, the chemical heating element comprises magnesium oxide, or iron pellets, which allows for an electricity free lateral flow device.

In addition, the streamlined lateral flow devices disclosed herein may carry out “one-pot” or “two-pot” reactions. In one-pot reactions—a) sample preparation, amplification, and detection, b) sample preparation and detection, or c) amplification and detection are carried out within the device in the same region. In two-pot reactions, amplification and detection are carried out within the device in separate regions. In two-pot reactions, sample preparation, amplification and detection, or combinations thereof, are carried out within the device in separate regions.

Disclosed herein are lateral flow devices for detection of a target nucleic acid of interest in a biological sample. The lateral flow devices described in detail herein can be used to monitor the reaction of target nucleic acids in samples with a programmable nuclease, thereby allowing for the detection of said target nucleic acid. All samples and reagents disclosed herein are compatible for use with a lateral flow device disclosed herein. Any programmable nuclease, such as any Cas nuclease described herein, is compatible for use with a lateral flow device disclosed below. Support mediums and housing disclosed herein are also compatible for use in conjunction with the lateral flow devices disclosed herein. Multiplexing detection, as described throughout the present disclosure, can be carried out within the lateral flow devices disclosed herein. Compositions and methods for detection and visualization disclosed herein are also compatible for use within the herein described lateral flow devices.

In the herein described lateral flow devices, any programmable nuclease (e.g., CRISPR-Cas) reaction can be monitored. For example, any programmable nuclease disclosed herein can be used to cleave the reporters to generate a detection signal. In some cases, the programmable nuclease is Cas13. Sometimes the Cas13 is Cas13a, Cas13b, Cas13c, Cas13d, or Cas13e. In some cases, the programmable nuclease is Mad7 or Mad2. In some cases, the programmable nuclease is Cas12. Sometimes the Cas12 is Cas12a, Cas12b, Cas12c, Cas12d, or Cas12e. In some cases, the programmable nuclease is Csm1, Cas9, C2c4, C2c8, C2c5, C2c10, C2c9, or CasZ. Sometimes, the Csm1 is also called smCms1, miCms1, obCms1, or suCms1. Sometimes Cas13a is also called C2c2. Sometimes CasZ is also called Cas14a, Cas14b, Cas14c, Cas14d, Cas14e, Cas 14f, Cas14g, or Cas14h. Sometimes, the programmable nuclease is a type V CRISPR-Cas system. In some cases, the programmable nuclease is a type VI CRISPR-Cas system. Sometimes the programmable nuclease is a type III CRISPR-Cas system. In some cases, the programmable nuclease is from at least one of Leptotrichia shahii (Lsh), Listeria seeligeri (Lse), Leptotrichia buccalis (Lbu), Leptotrichia wadei (Lwa), Rhodobacter capsulatus (Rca), Herbinix hemicellulosilytica (Hhe), Paludibacter propionicigenes (Ppr), Lachnospiraceae bacterium (Lba), [Eubacterium] rectale (Ere), Listeria newyorkensis (Lny), Clostridium aminophilum (Cam), Prevotella sp. (Psm), Capnocytophaga canimorsus (Cca Lachnospiraceae bacterium (Lba), Bergeyella zoohelcum (Bzo), Prevotella intermedia (Pin), Prevotella buccae (Pbu), Alistipes sp. (Asp), Riemerella anatipestifer (Ran), Prevotella aurantiaca (Pau), Prevotella saccharolytica (Psa), Prevotella intermedia (Pint), Capnocytophaga canimorsus (Cca), Porphyromonas gulae (Pgu), Prevotella sp. (Psp), Porphyromonas gingivalis (Pig), Prevotella intermedia (Pin3), Enterococcus italicus (Ei), Lactobacillus salivarius (Ls), or Thermus thermophilus (Tt). Sometimes the Cas13 is at least one of LbuCas13a, LwaCas13a, LbaCas13a, HheCas13a, PprCas13a, EreCas13a, CamCas13a, or LshCas13a.

Lateral flow devices disclosed herein are compatible with one-pot reactions and two-pot reactions. In a one-pot reaction, amplification, reverse transcription, amplification and reverse transcription, or amplification and in vitro transcription, and detection can be carried out simultaneously in the lateral flow device. In other words, in a one-pot reaction, any combination of reverse transcription, amplification, and in vitro transcription can be performed in the same reaction as detection. In a two-pot reaction, any combination of reverse transcription, amplification, and in vitro transcription can be performed in a first reaction, followed by detection in a second reaction. The one-pot or two-pot reactions can be carried out in the lateral flow devices disclosed herein.

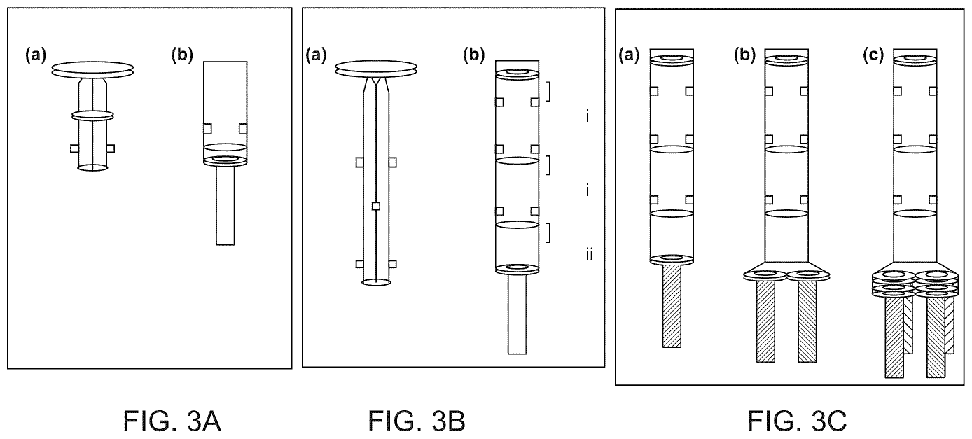

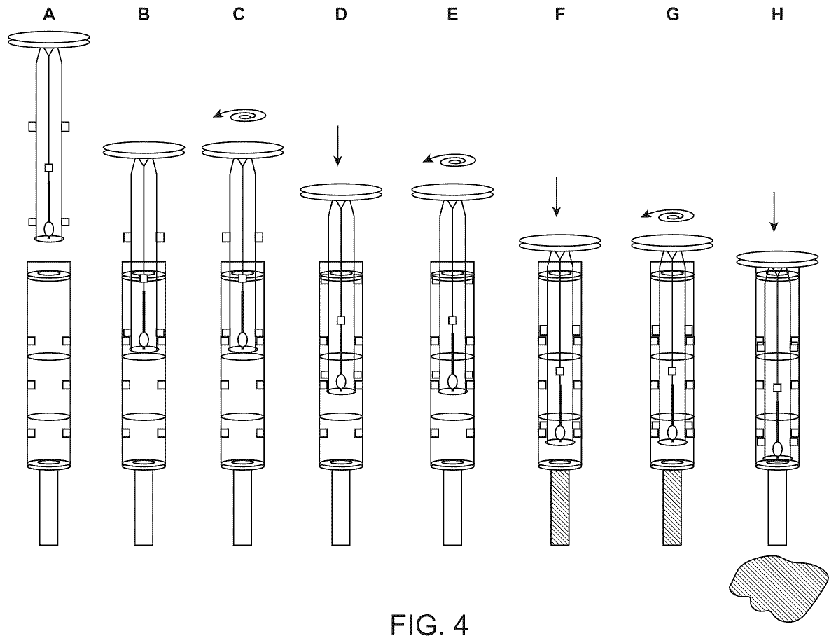

A lateral flow device disclosed herein can include a filtration device. In some embodiments, the lateral flow device is located within an enclosure of the device. In some embodiments, the enclosure comprises a filtration device configured to filter the sample after a lysis step. In some embodiments, the filtration device for sample preparation resembles a syringe or, comprises, similar functional elements to a syringe, e.g., as shown in FIGS. 3 and 4. For example, the filtration device for sample preparation may include a narrow tip for collection of liquid samples. Liquid samples can include blood, saliva, urine, or any other biological fluid. Liquid samples can also include liquid tissue homogenates. The tip, for collection of liquid samples, can be manufactured from glass, metal, plastic, or other biocompatible materials. The tip may be replaced with a glass capillary that may serve as a metering apparatus for the amount of biological sample added downstream to the lateral flow device. For some samples, e.g., blood, the capillary may be the only fluidic device required for sample preparation. Another functional element of the filtration device for sample preparation may include a channel that can carry volumes on the order from nL to mL, containing lysis buffers compatible with the programmable nuclease reaction downstream of this process. The channel may be manufactured from metal, plastic, or other biocompatible materials. The channel may be large enough to hold an entire fecal, buccal, or other biological sample collection swab. The filtration device may further contain a solution of reagents that will lyse the cells in each type of sample and release the nucleic acids so that they are accessible to the programmable nuclease. Active ingredients of the solution may be chaotropic agents, detergents, salts, and can be of high osmolality, ionic strength, and/or pH. In some embodiments, the pH is low in value. Chaotropic agents or chaotropes are substances that disrupt the three-dimensional structure in macromolecules such as proteins, DNA, or RNA. Alkaline buffers may also be used for cells with hard shells, particularly for environmental samples. Detergents such as sodium dodecyl sulphate (SDS) and cetyl trimethylammonium bromide (CTAB) may also be implemented to chemical lysis buffers. Cell lysis may also be performed by physical, mechanical, thermal or enzymatic means, in addition to or instead of chemically-induced cell lysis mentioned previously. The syringe-based device may include more complex architecture depending on the type of sample, such as nanoscale barbs, nanowires, sonication capability in a separate chamber of the device, integrated laser, integrated heater, for example, a Peltier-type heater, or a thin-film planar heater, and/or microcapillary probes for electrical lysis. Any samples described herein can be used in this workflow. For example, samples may include liquid samples collected from a subject being tested for a condition of interest.

In some embodiments, a lateral flow strip can be additionally interfaced with a sample preparation device, as shown in FIG. 3. FIG. 3 shows individual parts of sample preparation devices of some embodiments in the present disclosure. FIG. 3A shows a single chamber sample extraction device with (a) an insert that holds a sample collection device and regulates a step between sample extraction and dispensing the sample into another reaction or detection device, (b) a single chamber that contains extraction buffer. FIG. 3B of the figure shows an embodiment where a dispensing chamber is filled with material that further purifies the nucleic acid as it is dispensed wherein: (a) an insert holds the sample collection device and regulates “stages” of sample extraction and nucleic acid amplification. Each set of notches (red, blue and green) are offset 90° from the preceding set, (b) a reaction module contains multiple chambers separated by substrates that allow for independent reactions to occur. (e.g., i. a nucleic acid separation chamber, ii. a nucleic acid amplification chamber and iii. a DETECTR reaction chamber or dispensing chamber). Each chamber has notches (black) that prevent the insert from progressing into the next chamber without a deliberate 90° turn. The first two chambers may be separated by material that removes inhibitors between the extraction and amplification reactions. FIG. 3C shows additional embodiments for the reaction/dispensing chamber with: (a) a single dispensing chamber may release only extracted sample or extraction/amplification extraction/amplification/DETECTR reactions, (b) a duel dispensing chamber may release extraction/multiplex amplification products, and (c) a quadruple dispensing chamber would allow for multiplexing amplification and single DETECTR or four single amplification reactions. In some embodiments, syringe-based sample preparation devices are set up for readout with only one lateral flow assay strip, as shown in FIGS. 3A, 3B and 3C(a). In some embodiments, syringe-based sample preparation devices are set up for readout with more than one lateral flow assay strip, as shown in FIG. 3C(b) and 3C(c). In such embodiments, sample multiplexing is enabled by allowing for readout of more than one target nucleic acids per sample, wherein each different lateral flow assay strip can detect a correspondingly different one target nucleic acid, as described herein. In some embodiments, as described herein more than one target nucleic acid can be detected on a single lateral flow assay strip.

FIG. 4 shows a sample work flow using a sample processing device as described herein. The sample collection device is attached to the insert portion of the sample processing device (A). The insert is placed into the device chamber and pressed until the first stop (lower tabs on top portion meet upper tabs on bottom portion) (B). This step allows the sample to come into contact with the nucleic acid extraction reagents. After the appropriate amount of time, the insert is turned 90° (C.) and depressed (D) to the next set of notches. These actions transfer the sample into the amplification chamber. The sample collection device is no longer in contact with the sample or amplification products. After the appropriate incubation, the insert is rotated 90° (E) and depressed (F) to the next set of notches. These actions release the sample into the DETECTR (green reaction). The insert is again turned 90° (G) and depressed (H) to dispense the reaction.

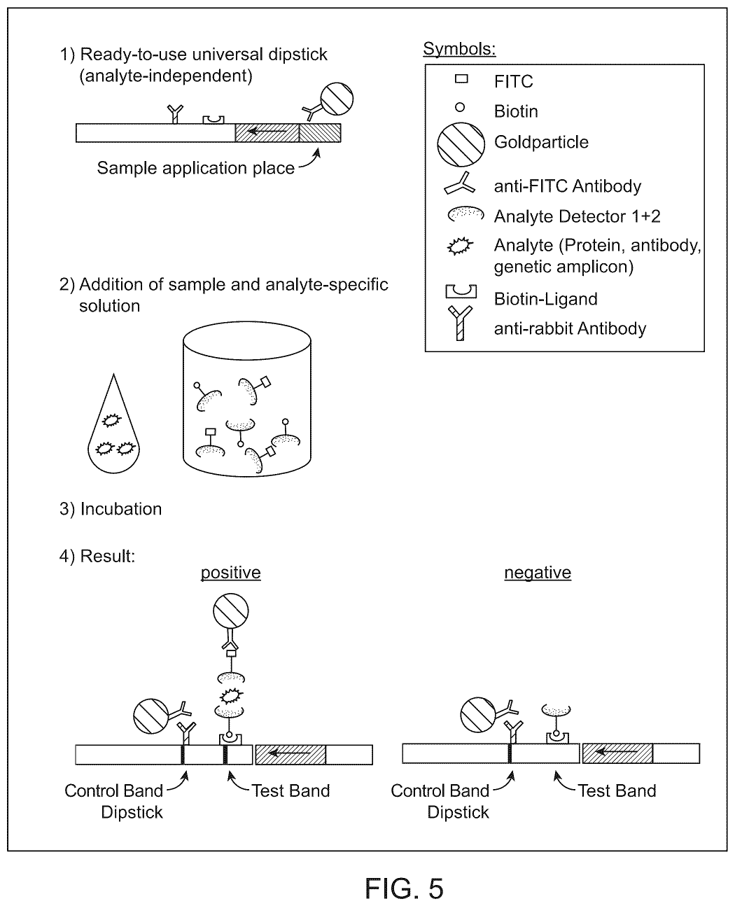

Described herein are various methods, devices and systems for DETECTR™ assays for readout on lateral flow assay strips. In some embodiments, DETECTR™ assays are read out on lateral flow assay strips as shown in FIG. 5. FIG. 5 shows the layout of a Milenia™ commercially available strip with a typical reporter. This schematic shows an analyte-independent universal dipstick with a sample application region at right followed by a wicking region immediately to the left, followed to the left by a region containing a biotin ligand, followed to the left by a region spotted with anti-rabbit antibody. The sample and analyte-specific solution are incubated with analyte detectors bearing a biotin or FITC. Samples are run on the strip. A positive result shows two bands—the left-most band is from the control band and is due to binding of anti-FITC antibody coated gold nanoparticles to an anti-rabbit antibody. The right band is from the test band itself and is due to binding by the biotin ligand to an analyte detector bearing biotin, where the detector complexes the analyte and wherein the analyte is further complexed to another detector bearing FITC, which is then bound to the anti-FITC antibody coated gold particle. In the negative result—only one band is seen at the control line. In such embodiments, workflow comprises four steps: (1) sample application to lateral flow assay strip; (2) addition of sample and analyte-specific solution; (3) incubation; and (4) readout of result.

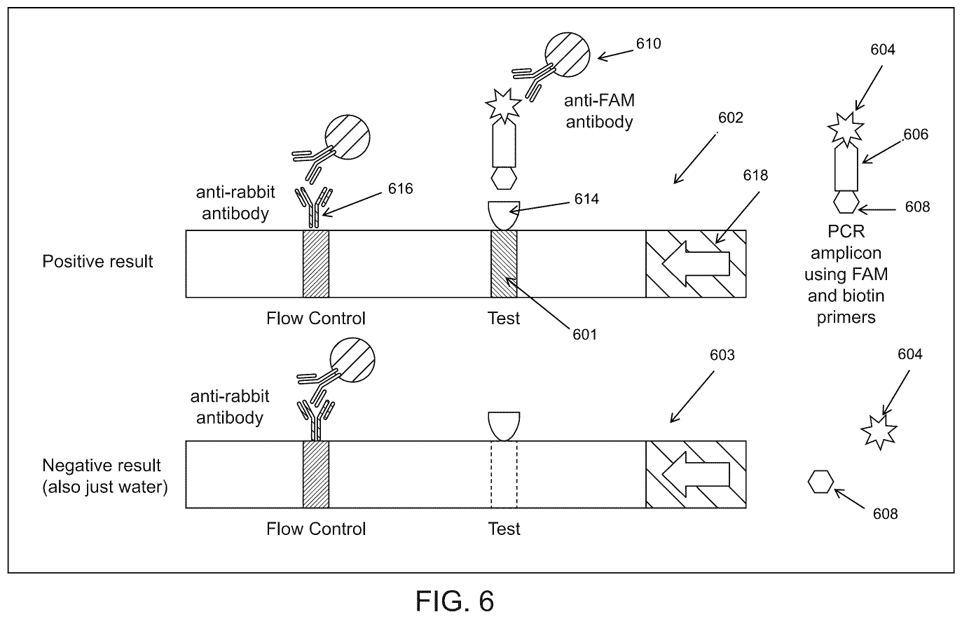

FIG. 6 shows the layout of a Milenia HybridDetect 1 strip with a PCR amplicon (606) comprising using FAM and biotin primers (604, 608). This schematic shows at top PCR amplicon using FAM and biotin primers at the right end of the top figure. In the case of a positive result, the strip shows two bands—this PCR amplicon binds to a moiety immobilized at the test line, and the FAM molecule (shown as a start) binds to an anti-FAM antibody coated particle. To the left of the test line is a flow control line, containing anti-rabbit antibody which binds to anti-FAM antibody coated nanoparticles. In the case of a negative result, the strip shows one band—that is, just binding of anti-FAM antibody coated nanoparticles bound to anti-rabbit antibody immobilized on the test strip. A Milenia HybridDetect 1 strip showing both positive result chemistry (602) and negative result chemistry (603) are displayed. A positive result occurs when a sample contains PCR amplicons (606) comprising FAM (604) and biotin (608). The sample placed on the sample pad (618) and is drawn toward the test line (601) and flow control line. Streptavidin (614) acting as a capture probe, located on the test line (601) captures the biotin (608) end of the PCR amplicon (606) that was in turn captured by the flowing capture probe (610), also known as a conjugate particle, which comprises a gold nanoparticle and anti-FAM antibody. Any flowing capture probe (610) that does not capture a PCR amplicon (606) is drawn further down the lateral flow assay strip and is then captured at the flow control line by anti-rabbit antibody (616). A negative result occurs when not enough PCR amplicon is present to generate a visible signal. When a sample that does not contain enough the PCR amplicon (606), even if biotin (608) and FAM (604) a visible signal at the test line does not occur. This is because the flowing capture probe (610) which are present just downstream of the sample pad do not have any biotin labeled PCR product to bind to and therefore are not functionalized with biotin. This in turn does not allow for the flowing capture probe to bind to the streptavidin (614) at the test line (601). Without the gold nanoparticles present the test line does not exhibit a color change.

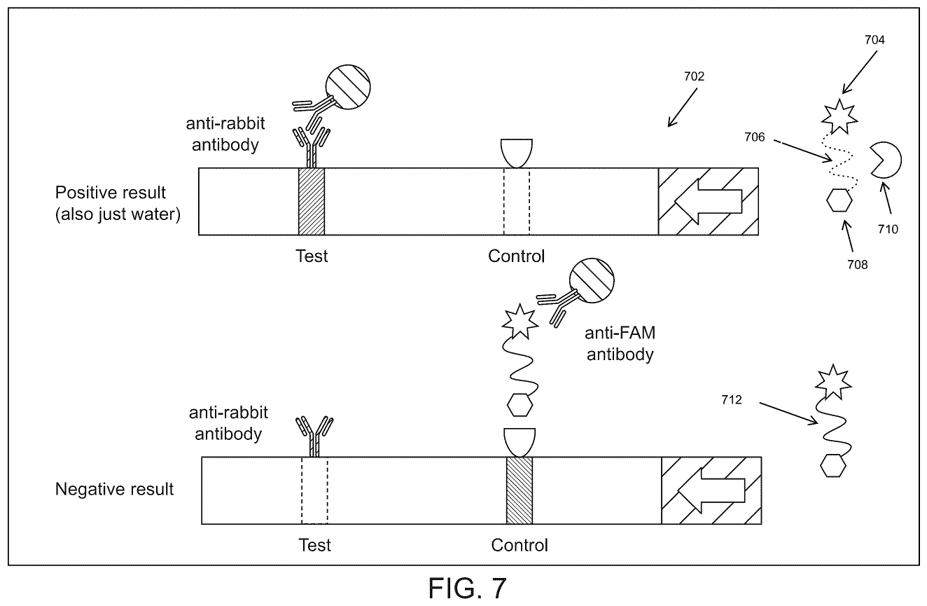

In some embodiments, Milenia HybridDetect 1 Strip™ is used with a standard Cas reporter where the cleavable nucleic acid (706) is located between the FAM (704) and biotin (708), as shown in FIG. 7. A positive result is shown at top where a Cas protein cleaves the standard reporter, and only one band is seen—due to binding of the anti-FAM antibody coated nanoparticles to anti-rabbit antibody spotted on the strip. A negative result is shown at bottom where the intact reporter binds to a moiety immobilized on the strip, and all of the anti-FAM antibody coated nanoparticles bind at the control line to the FAM molecule on the intact Cas reporter. Results of running samples with target nucleic acids and with a water only control showed that even with the water only control, a false positive band appeared at the test line.

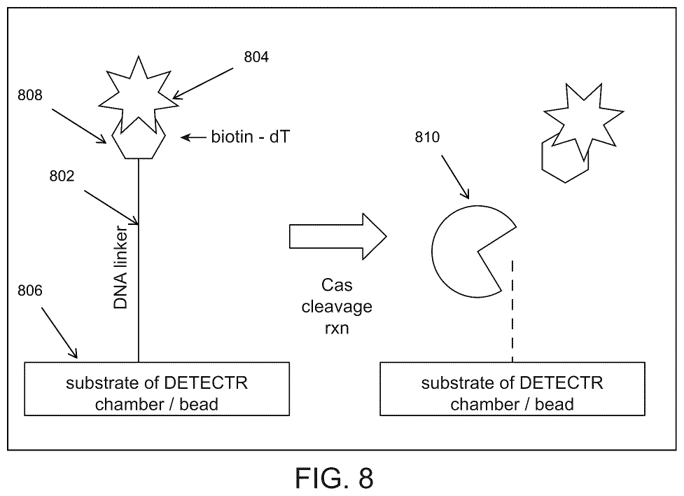

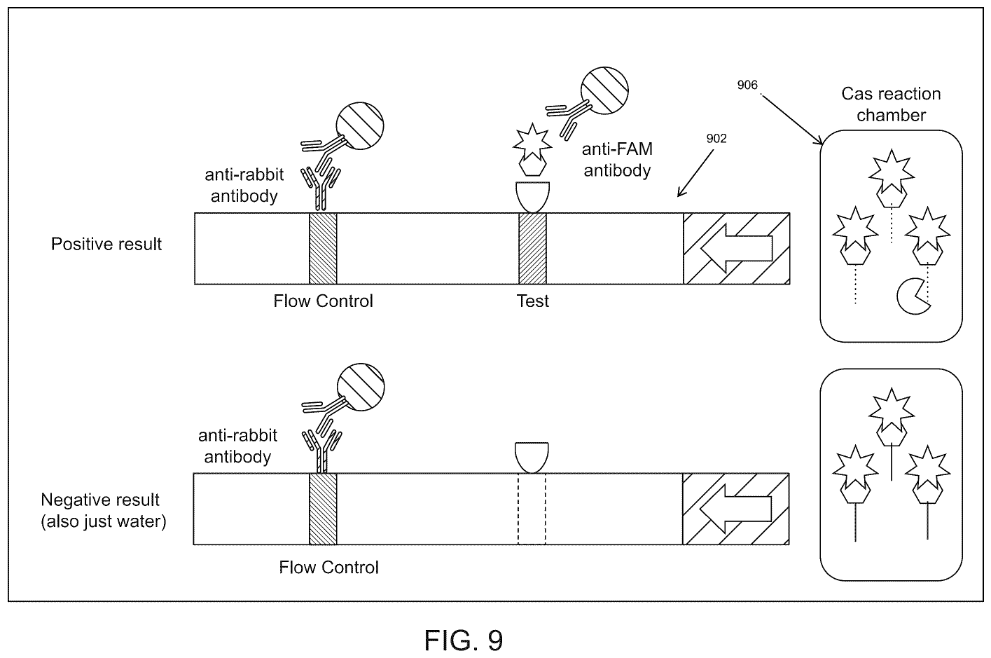

In some embodiments, a device of the present disclosure comprises a chamber (906) and a lateral flow strip (902) as shown in FIG. 9. FIGS. 8-9 show a particularly advantageous layout for the lateral flow strip (902) and a corresponding suitable reporter. FIG. 8 shows a modified Cas reporter comprising a DNA linker to biotin-dT (shown as a pink hexagon) bound to a FAM molecule (shown as a green star). This entire modified Cas reporter is conjugated to magnetic beads or the surface of a reaction chamber, which is upstream of the strip. This is shown in the schematic as immobilization of the modified Cas reporter to the substrate of the DETECTR chamber/bead. During cleavage by a Cas (shown as a yellow pac-man), the biotin-FAM molecule is released from the DNA linker. Unlike other assay formats, this particular assay format contains the entire Cas cleavage reaction to the reaction chamber. In this assay format, the test-line is the actual test line and the control line is a true control line. FIG. 9 shows the layout of Milenia HybridDetect strips with the modified Cas enzyme and reporter. At top, a positive result is shown, where in the Cas reaction chamber, the Cas protein cleaves the DNA linker segment of the modified Cas reporter. The biotin-dT/FAM molecule is released and flows down the test strip binding to streptavidin coated on the test line. An anti-FAM antibody coated gold nanoparticle binds to the biotin-DT/FAM reporter at the test line. Additionally the anti-FAM antibody coated gold nanoparticle binds to anti-rabbit antibody coated at the flow control line. At bottom, a negative result is shown where only the anti-FAM antibody coated gold nanoparticle binds to anti-rabbit antibody coated at the flow control line. This particular layout improves the test result by generating higher signal in the case of a positive result, while also minimizing false positives. In this assay layout, the reporter comprises a biotin and a fluorophore (e.g., FAM) attached at one end of a nucleic acid. The nucleic acid can be conjugated directly to the biotin molecule and then the fluorophore or directly to the fluorophore and then to the biotin. Other affinity molecules, including those described herein can be used instead of biotin. Any of the fluorophores disclosed herein can also be used in the reporter. The reporter can be suspended in solution or immobilized on the surface of the Cas chamber. Alternatively, the reporter can be immobilized on beads, such as magnetic beads, in the reaction chamber where they are held in position by a magnet placed below the chamber. When the reporter is cleaved by an activated programmable nuclease, the cleaved biotin-fluorophore accumulates at the first line, which comprises a streptavidin (or another capture molecule). Gold nanoparticles, which are on the sample pad and flown onto the strip using a chase buffer, are coated with an anti-fluorophore antibody allowing binding and accumulation of the gold nanoparticle at the first line. The nanoparticles additionally accumulate at a second line which is coated with an antibody (e.g., anti-rabbit) against the antibody coated on the gold nanoparticles (e.g., rabbit, anti-FAM). In the case of a negative result, the reporter is not cleaved and does not flow on the lateral flow strip. Thus, the nanoparticles only bind and accumulate at the second line. Multiplexing detection on the lateral flow strip can be performed by having two reporters (e.g., a biotin-FAM reporter and a biotin-DIG reporter). Anti-FAM and anti-DIG antibodies are coated onto the lateral flow strip at two different regions. Anti-biotin antibodies or streptavidin or avidin are coated on gold nanoparticles. Fluorophores are conjugated directly to the affinity molecules (e.g., biotin) by first generating a biotin-dNTP following from the nucleic acids of the reporter and then conjugating the fluorophore. In some embodiments, the lateral flow strip comprises multiple layers. In such embodiments, the multiple layers comprise a sample pad layer, a detection layer, and a wicking layer.

In some embodiments, the above lateral flow strip can be additionally interfaced with a sample preparation device.

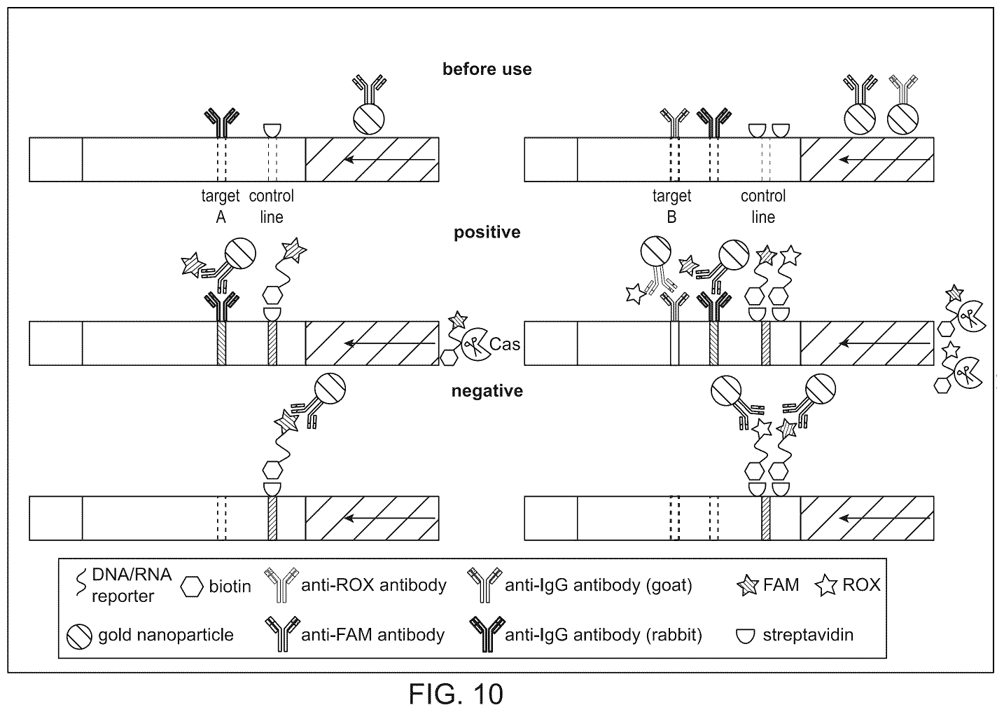

Described here are various methods for multiplexed DETECTR™ assay readout using a streamlined lateral flow strip as described herein. FIG. 10 shows both an embodiment for a single target assay format (to the left) and an embodiment for a multiplexed assay format (to the right). The top row shows the lateral flow assay strips before use. The middle row and bottom row show positive and negative results, respectively. In some embodiments, a lateral flow assay strip has more than one detection spot, line, or area. In the top row, left-hand side of FIG. 10, a lateral flow assay strip is shown with two detection lines, one for target A and one for target B.

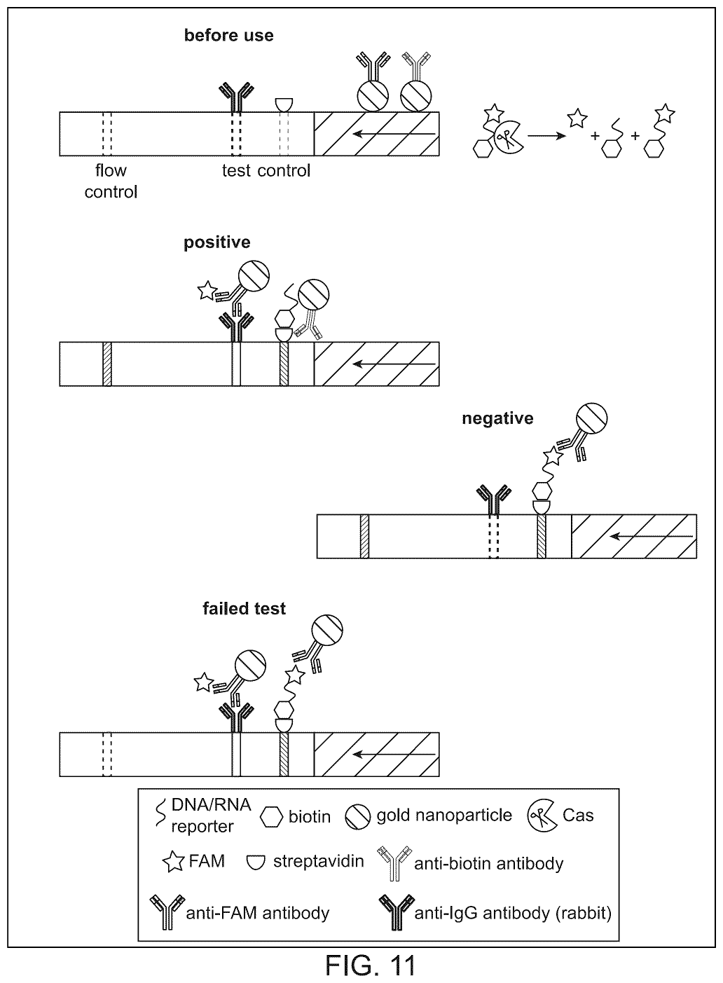

FIG. 11 shows another embodiment of a DETECTR™ assay prior to use (top), an assay with a positive result (middle left), an assay with a negative result (middle right), and a failed test (bottom). In some embodiments, the sample pad comprises anti-biotin functionalized gold nanoparticles, wherein the gold nanoparticles are labeled with anti-biotin antibody. In some embodiments, the lateral flow assay strip comprises a control line further comprising streptavidin capture probes. In some embodiments, the lateral flow assay strip comprises a test line further comprising anti-IgG antibody (rabbit). In some embodiments, a solution comprises DETECTR™ assay reagents, further comprising a Cas enzyme, wherein the Cas enzyme cleaves a reporter upon binding of a target nucleic acid (not shown) by a guide nucleic acid (not shown). In some embodiments, the reacted DETECTR™ solution containing either cleaved reporters, un-cleaved reporters, or a combination thereof is exposed to the sample pad of the lateral flow assay strip. In some embodiments, a positive result is indicated by a visible scattering signal by the anti-biotin gold nanoparticles at both the test and control line, as shown in the middle left of FIG. 11. In some embodiments, a negative result is indicated by a visible scattering signal by the anti-biotin labeled gold nanoparticles, when there is no cleavage of the reporter, when no target nucleic acid is present, as shown in the middle right of FIG. 11.

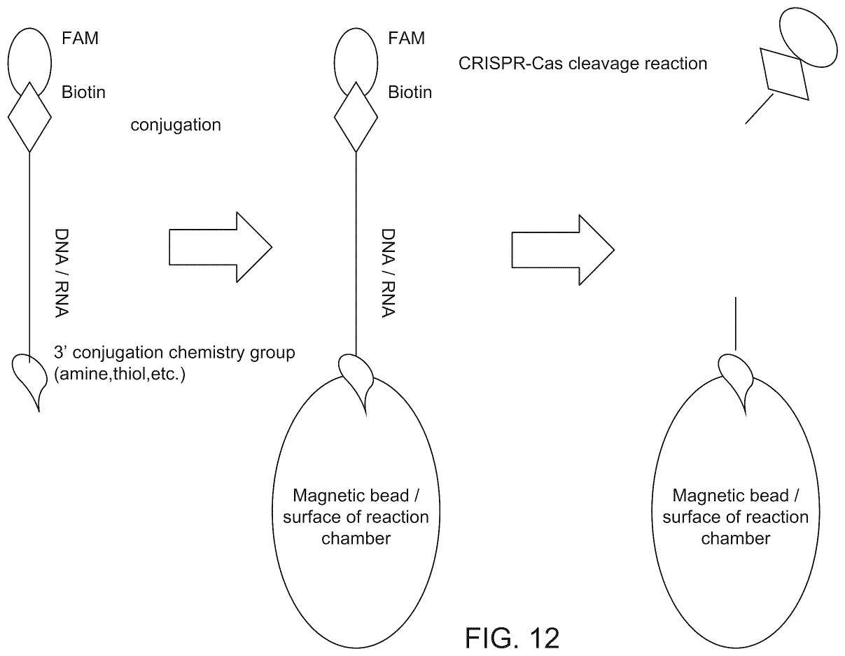

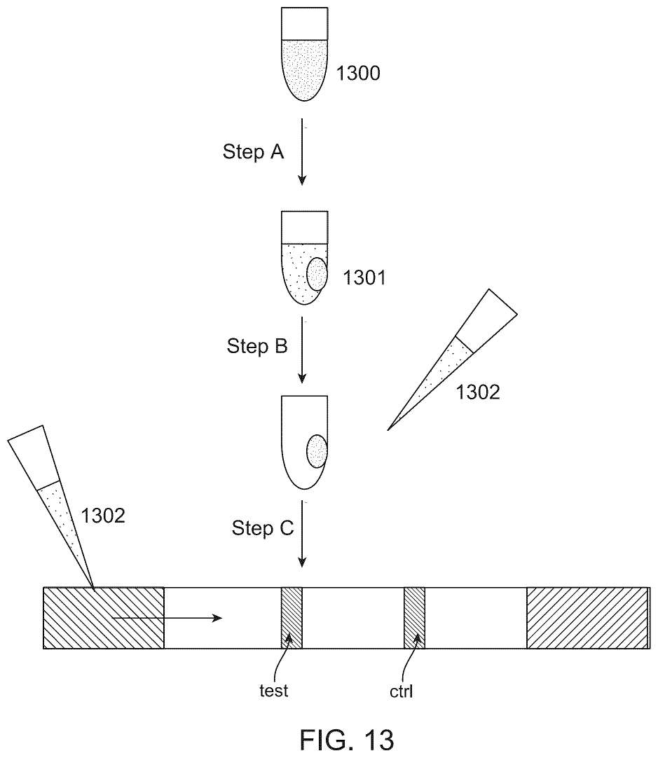

Described herein are various methods, devices, compositions, and systems for DETECTR™ assays. FIG. 12 shows one design of a tethered reporter for lateral flow applications. In some embodiments, a reporter comprising a cleavable nucleic acid sequence is labeled with both FAM and Biotin at the 5′ end and functionalized with an amine group at the 3′ end, as shown in FIG. 12 left. In some embodiments, the amine functionalized 3′ end conjugates to an amine reactive magnetic bead as shown in FIG. 12 middle. Alternatively, in some embodiments, the reporter comprising a cleavable nucleic acid sequence is labeled with both FAM and Biotin at the 5′ end and functionalized with a thiol group at the 3′ end, as shown in FIG. 12 left. In some embodiments, the thiol functionalized 3′ end conjugates to a thiol reactive surface of a reaction chamber as shown in FIG. 12 middle. In some embodiments, the cleaved reporter fragment comprising FAM and biotin is released into solution after following cleaving by an active Cas enzyme as described herein. In some embodiments, the DETECTR™ assay is carried out using magnetic beads in solution for streamlined lateral flow assays. FIG. 13 shows one embodiment of such a workflow. In some embodiments, a solution (1300) comprising the cleaved reporter fragments further comprising FAM and biotin along with the other reporter fragment immobilized to the magnetic bead as shown in FIG. 13. In some embodiments, the solution (1300) is placed in a magnetic field causing the magnetic particles to be collected into an immobilized pellet (1301) as shown in Step A of FIG. 13. In some embodiments, the supernatant solution (1302) containing the FAM and biotin labeled reporter fragments is then collected by pipet as shown in Step B of FIG. 13. In some embodiments the supernatant solution is then applied to the sample pad of the lateral flow assay strip as shown in Step C of FIG. 13. The results are then read out as described herein.

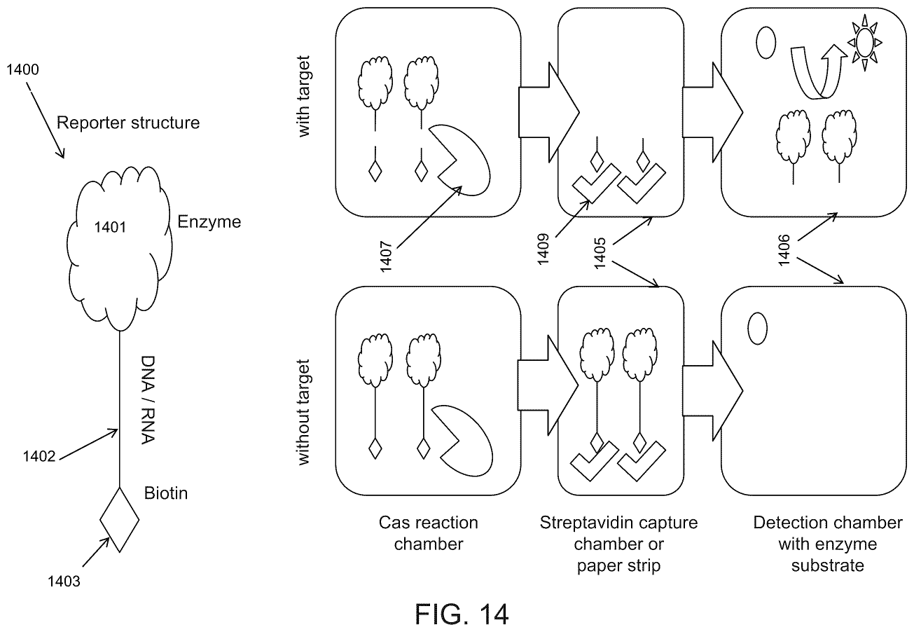

Described herein are various embodiments, wherein an enzyme modified reporter (1400) is used in a DETECTR™ assay, where streptavidin-biotin binding is used to filter out cleaved reporters before reaching the reaction chamber as shown in FIG. 14. The reporter (1400) comprises an enzyme (1401), a cleavable nucleic acid sequence (1402) and a biotin (1403). The upper row of FIG. 14 shows the DETECTR™ process when a target nucleic acid is present in solution. The lower row of FIG. 14 shows the DETECTR™ process when a target nucleic acid is not present in solution. In some embodiments, when the target nucleic acid is present in solution, the programmable nuclease (1407) is activated and cleaves the reporter (1400). In such an embodiment, the cleaved reporter fragments labeled with biotin (1408) are captured by streptavidin (1409) immobilized to a capture chamber or paper strip (1405). When the target nucleic acid is present in solution, the enzyme (1401) produces a signal emitting moiety (1410) when exposed to the detection chamber with enzyme substrate (1406). In some embodiments, the enzyme (1401) is horse radish peroxidase (HRP) as described herein.

### Reagents and Signal Detection

A number of reagents are consistent with the lateral flow devices and methods disclosed herein. These reagents include buffers (e.g., lysis buffers), programmable nucleases, reporters and guide nucleic acids. The reagents described herein for detecting a disease comprise a guide nucleic acid targeting the target nucleic acid segment indicative of the disease. The guide nucleic acid binds to the single stranded target nucleic acid comprising a portion of a nucleic acid from a virus or a bacterium or other agents responsible for a disease as described herein. The guide nucleic acid can bind to the single stranded target nucleic acid comprising a portion of a nucleic acid from a bacterium or other agents responsible for a disease as described herein and optionally further comprising a mutation, such as a single nucleotide polymorphism (SNP), that can confer resistance to a treatment, such as antibiotic treatment. The guide nucleic acid is complementary to the target nucleic acid. Often the guide nucleic acid binds specifically to the target nucleic acid. The target nucleic acid may be a RNA, DNA, or synthetic nucleic acids.

Disclosed herein are methods of assaying for a target nucleic acid as described herein. For example, a method of assaying for a target nucleic acid in a sample comprises contacting the sample to a complex comprising a) a guide nucleic acid comprising a segment that is reverse complementary to a segment of the target nucleic acid and b) a programmable nuclease that exhibits sequence independent cleavage upon forming an activated complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. As another example, a method of assaying for a target nucleic acid in a sample, for example, comprises: a) contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; b) contacting the complex to a reaction substrate; c) contacting the reaction substrate to a reagent that differentially reacts with a cleaved reaction substrate; and d) assaying for a signal indicating cleavage of the reaction substrate occurred, wherein the signal indicates a presence of the target nucleic acid in the sample and wherein absence of the signal indicates an absence of the target nucleic acid in the sample. Said method can be carried out in the streamlined, lateral flow devices disclosed herein. Often, the reaction substrate is an enzyme-nucleic acid and the reagent is an enzyme substrate corresponding to the enzyme. Sometimes, the reaction substrate is an enzyme substrate-nucleic acid and the reagent is an enzyme corresponding to the enzyme substrate.

A programmable nuclease can comprise a programmable nuclease capable of being activated when complexed with a guide nucleic acid and target nucleic acid. The programmable nuclease can become activated after binding of a guide nucleic acid with a target nucleic acid, in which the activated programmable nuclease can cleave the target nucleic acid and can have trans cleavage activity. Trans cleavage activity can be non-specific cleavage of nearby single-stranded nucleic acids by the activated programmable nuclease, such as trans cleavage of reporters with a detection moiety. Once the reporter is cleaved by the activated programmable nuclease, the detection moiety can be released from the reporter and can generate a signal. A signal can be an optical (e.g., fluorescent, colorometric, etc.), or piezo-electric signal. Often, the signal is present prior to reporter cleavage and changes upon reporter cleavage. Sometimes, the signal is absent prior to reporter cleavage and is present upon reporter cleavage. The detectable signal can be detected from a detection region or detection spot located on a support medium. In some embodiments, the detectable signal is generated by the detection moiety. In some embodiments, the detectable signal is indicative of biding of the detection moiety at the detection region or detection spot. The programmable nuclease can be a CRISPR-Cas (clustered regularly interspaced short palindromic repeats—CRISPR associated) nucleoprotein complex with trans cleavage activity, which can be activated by binding of a guide nucleic acid with a target nucleic acid. The CRISPR-Cas nucleoprotein complex can comprise a Cas protein (also referred to as a Cas nuclease) complexed with a guide nucleic acid, which can also be referred to as CRISPR enzyme. A guide nucleic acid can be a CRISPR RNA (crRNA). Sometimes, a guide nucleic acid comprises a crRNA and a trans-activating crRNA (tracrRNA).

The CRISPR/Cas system used to detect a modified target nucleic acids can comprise CRISPR RNAs (crRNAs), trans-activating crRNAs (tracrRNAs), Cas proteins, and reporters.

A. Programmable Nucleases

Described herein are reagents comprising a programmable nuclease capable of being activated when complexed with the guide nucleic acid and the target nucleic acid segment. A programmable nuclease can be capable of being activated when complexed with a guide nucleic acid and the target sequence. The programmable nuclease can be activated upon binding of the guide nucleic acid to its target nucleic acid and can non-specifically degrade a non-target nucleic acid in its environment. The programmable nuclease has trans cleavage activity once activated. A programmable nuclease can be a Cas protein (also referred to, interchangeably, as a Cas nuclease). A crRNA and Cas protein can form a CRISPR enzyme.

Several programmable nucleases are consistent with the methods and devices of the present disclosure. For example, CRISPR/Cas enzymes are programmable nucleases used in the methods and systems disclosed herein. CRISPR/Cas enzymes can include any of the known Classes and Types of CRISPR/Cas enzymes. Programmable nucleases disclosed herein include Class 1 CRISPR/Cas enzymes, such as the Type I, Type IV, or Type III CRISPR/Cas enzymes. Programmable nucleases disclosed herein also include the Class 2 CRISPR/Cas enzymes, such as the Type II, Type V, and Type VI CRISPR/Cas enzymes. Programmable nucleases included in the devices disclosed herein and methods of use thereof include a Type V or Type VI CRISPR/Cas enzyme.

In some embodiments, the Type V CRISPR/Cas enzyme is a programmable Cas12 nuclease. Type V CRISPR/Cas enzymes (e.g., Cas12 or Cas14) lack an HNH domain. In some embodiments, the CRISPR/Cas enzyme is a programmable CasPhi. A Cas12 nuclease of the present disclosure cleaves a nucleic acids via a single catalytic RuvC domain. The RuvC domain is within a nuclease, or “NUC” lobe of the protein, and the Cas12 nucleases further comprise a recognition, or “REC” lobe. The REC and NUC lobes are connected by a bridge helix and the Cas12 proteins additionally include two domains for protospacer adjacent motif (PAM)recognition termed the PAM interacting (PI) domain and the wedge (WED) domain. (Murugan et al., Mol Cell. 2017 Oct. 5; 68(1): 15-25). A programmable Cas12 nuclease can be a Cas12a (also referred to as Cpf1) protein, a Cas12b protein, Cas12c protein, Cas12d protein, or a Cas12e protein. In some cases, a suitable Cas12 protein comprises an amino acid sequence having at least 80%, at least 85%, at least 90%, at least 95%, at least 98%, at least 99%, or 100%, amino acid sequence identity to any one of SEQ ID NO: 27-SEQ ID NO: 37.

TABLE 1
Cas12 Protein Sequences

| SEQ | | |
|---|---|---|
| ID | | |
| NO | Description | Sequence |
| 27 | Lachnospiraceae | MSKLEKFTNCYSLSKTLRFKAIPVGKTQENIDNKRLLVEDEKRAEDY |
| | bacterium | KGVKKLLDRYYLSFINDVLHSIKLKNLNNYISLFRKKTRTEKENKEL |
| | ND2006 | ENLEINLRKEIAKAFKGNEGYKSLFKKDIIETILPEFLDDKDEIALV |
| | (LbCas12a) | NSFNGFTTAFTGFFDNRENMFSEEAKSTSIAFRCINENLTRYISNMD |
| | | IFEKVDAIFDKHEVQEIKEKILNSDYDVEDFFEGEFFNFVLTQEGID |
| | | VYNAIIGGFVTESGEKIKGLNEYINLYNQKTKQKLPKFKPLYKQVLS |
| | | DRESLSFYGEGYTSDEEVLEVFRNTLNKNSEIFSSIKKLEKLFKNFD |
| | | EYSSAGIFVKNGPAISTISKDIFGEWNVIRDKWNAEYDDIHLKKKAV |
| | | VTEKYEDDRRKSFKKIGSFSLEQLQEYADADLSVVEKLKEIIIQKVD |
| | | EIYKVYGSSEKLFDADFVLEKSLKKNDAVVAIMKDLLDSVKSFENYI |
| | | KAFFGEGKETNRDESFYGDFVLAYDILLKVDHIYDAIRNYVTQKPYS |
| | | KDKFKLYFQNPQFMGGWDKDKETDYRATILRYGSKYYLAIMDKKYAK |
| | | CLQKIDKDDVNGNYEKINYKLLPGPNKMLPKVFFSKKWMAYYNPSED |
| | | IQKIYKNGTFKKGDMFNLNDCHKLIDFFKDSISRYPKWSNAYDFNFS |
| | | ETEKYKDIAGFYREVEEQGYKVSFESASKKEVDKLVEEGKLYMFQIY |
| | | NKDFSDKSHGTPNLHTMYFKLLFDENNHGQIRLSGGAELFMRRASLK |
| | | KEELVVHPANSPIANKNPDNPKKTTTLSYDVYKDKRFSEDQYELHIP |
| | | IAINKCPKNIFKINTEVRVLLKHDDNPYVIGIDRGERNLLYIVVVDG |
| | | KGNIVEQYSLNEIINNFNGIRIKTDYHSLLDKKEKERFEARQNWTSI |
| | | ENIKELKAGYISQVVHKICELVEKYDAVIALEDLNSGFKNSRVKVEK |
| | | QVYQKFEKMLIDKLNYMVDKKSNPCATGGALKGYQITNKFESFKSMS |
| | | TQNGFIFYIPAWLTSKIDPSTGFVNLLKTKYTSIADSKKFISSFDRI |
| | | MYVPEEDLFEFALDYKNFSRTDADYIKKWKLYSYGNRIRIFRNPKKN |
| | | NVFDWEEVCLTSAYKELFNKYGINYQQGDIRALLCEQSDKAFYSSFM |
| | | ALMSLMLQMRNSITGRTDVDFLISPVKNSDGIFYDSRNYEAQENAIL |
| | | PKNADANGAYNIARKVLWAIGQFKKAEDEKLDKVKIAISNKEWLEYA |
| | | QTSVKH |
| 28 | Acidamino | MTQFEGFTNLYQVSKTLRFELIPQGKTLKHIQEQGFIEEDKARNDHY |
| | coccus sp. | KELKPIIDRIYKTYADQCLQLVQLDWENLSAAIDSYRKEKTEETRNA |
| | BV316 | LIEEQATYRNAIHDYFIGRTDNLTDAINKRHAEIYKGLFKAELFNGK |
| | (AsCas12a) | VLKQLGTVTTTEHENALLRSFDKFTTYFSGFYENRKNVFSAEDISTA |
| | | IPHRIVQDNFPKFKENCHIFTRLITAVPSLREHFENVKKAIGIFVST |
| | | SIEEVFSFPFYNQLLTQTQIDLYNQLLGGISREAGTEKIKGLNEVLN |
| | | LAIQKNDETAHIIASLPHRFIPLFKQILSDRNTLSFILEEFKSDEEV |
| | | IQSFCKYKTLLRNENVLETAEALFNELNSIDLTHIFISHKKLETISS |
| | | ALCDHWDTLRNALYERRISELTGKITKSAKEKVQRSLKHEDINLQEI |
| | | ISAAGKELSEAFKQKTSEILSHAHAALDQPLPTTLKKQEEKEILKSQ |
| | | LDSLLGLYHLLDWFAVDESNEVDPEFSARLTGIKLEMEPSLSFYNKA |
| | | RNYATKKPYSVEKFKLNFQMPTLASGWDVNKEKNNGAILFVKNGLYY |
| | | LGIMPKQKGRYKALSFEPTEKTSEGFDKMYYDYFPDAAKMIPKCSTQ |
| | | LKAVTAHFQTHTTPILLSNNFIEPLEITKEIYDLNNPEKEPKKFQTA |
| | | YAKKTGDQKGYREALCKWIDFTRDFLSKYTKTTSIDLSSLRPSSQYK |
| | | DLGEYYAELNPLLYHISFQRIAEKEIMDAVETGKLYLFQIYNKDFAK |
| | | GHHGKPNLHTLYWTGLFSPENLAKTSIKLNGQAELFYRPKSRMKRMA |
| | | HRLGEKMLNKKLKDQKTPIPDTLYQELYDYVNHRLSHDLSDEARALL |
| | | PNVITKEVSHEIIKDRRFTSDKFFFHVPITLNYQAANSPSKFNQRVN |
| | | AYLKEHPETPIIGIDRGERNLIYITVIDSTGKILEQRSLNTIQQFDY |
| | | QKKLDNREKERVAARQAWSVVGTIKDLKQGYLSQVIHEIVDLMIHYQ |
| | | AVVVLENLNFGFKSKRTGIAEKAVYQQFEKMLIDKLNCLVLKDYPAE |
| | | KVGGVLNPYQLTDQFTSFAKMGTQSGFLFYVPAPYTSKIDPLTGFVD |
| | | PFVWKTIKNHESRKHFLEGFDFLHYDVKTGDFILHFKMNRNLSFQRG |
| | | LPGFMPAWDIVFEKNETQFDAKGTPFIAGKRIVPVIENHRFTGRYRD |
| | | LYPANELIALLEEKGIVERDGSNILPKLLENDDSHAIDTMVALIRSV |
| | | LQMRNSNAATGEDYINSPVRDLNGVCFDSRFQNPEWPMDADANGAYH |
| | | IALKGQLLLNHLKESKDLKLQNGISNQDWLAYIQELRN |
| 29 | Francisella | MSIYQEFVNKYSLSKTLRFELIPQGKTLENIKARGLILDDEKRAKDY |
| | novicida | KKAKQIIDKYHQFFIEEILSSVCISEDLLQNYSDVYFKLKKSDDDNL |
| | U112 | QKDFKSAKDTIKKQISEYIKDSEKFKNLFNQNLIDAKKGQESDLILW |
| | (FnCas12a) | LKQSKDNGIELFKANSDITDIDEALEIIKSFKGWTTYFKGFHENRKN |
| | | VYSSNDIPTSIIYRIVDDNLPKFLENKAKYESLKDKAPEAINYEQIK |
| | | KDLAEELTFDIDYKTSEVNQRVFSLDEVFEIANFNNYLNQSGITKFN |
| | | TIIGGKFVNGENTKRKGINEYINLYSQQINDKTLKKYKMSVLFKQIL |
| | | SDTESKSFVIDKLEDDSDVVTTMQSFYEQIAAFKTVEEKSIKETLSL |
| | | LFDDLKAQKLDLSKIYFKNDKSLTDLSQQVFDDYSVIGTAVLEYITQ |
| | | QIAPKNLDNPSKKEQELIAKKTEKAKYLSLETIKLALEEFNKHRDID |
| | | KQCRFEEILANFAAIPMIFDEIAQNKDNLAQISIKYQNQGKKDLLQA |
| | | SAEDDVKAIKDLLDQTNNLLHKLKIFHISQSEDKANILDKDEHFYLV |
| | | FEECYFELANIVPLYNKIRNYITQKPYSDEKFKLNFENSTLANGWDK |
| | | NKEPDNTAILFIKDDKYYLGVMNKKNNKIFDDKAIKENKGEGYKKIV |
| | | YKLLPGANKMLPKVFFSAKSIKFYNPSEDILRIRNHSTHTKNGSPQK |
| | | GYEKFEFNIEDCRKFIDFYKQSISKHPEWKDFGFRFSDTQRYNSIDE |
| | | FYREVENQGYKLTFENISESYIDSVVNQGKLYLFQIYNKDFSAYSKG |
| | | RPNLHTLYWKALFDERNLQDVVYKLNGEAELFYRKQSIPKKITHPAK |
| | | EAIANKNKDNPKKESVFEYDLIKDKRFTEDKFFFHCPITINFKSSGA |
| | | NKFNDEINLLLKEKANDVHILSIDRGERHLAYYTLVDGKGNIIKQDT |
| | | FNIIGNDRMKTNYHDKLAAIEKDRDSARKDWKKINNIKEMKEGYLSQ |
| | | VVHEIAKLVIEYNAIVVFEDLNFGFKRGRFKVEKQVYQKLEKMLIEK |
| | | LNYLVFKDNEFDKTGGVLRAYQLTAPFETFKKMGKQTGIIYYVPAGF |
| | | TSKICPVTGFVNQLYPKYESVSKSQEFFSKFDKICYNLDKGYFEFSF |
| | | DYKNFGDKAAKGKWTIASFGSRLINFRNSDKNHNWDTREVYPTKELE |
| | | KLLKDYSIEYGHGECIKAAICGESDKKFFAKLTSVLNTILQMRNSKT |
| | | GTELDYLISPVADVNGNFFDSRQAPKNMPQDADANGAYHIGLKGLML |
| | | LGRIKNNQEGKKLNLVIKNEEYFEFVQNRNN |
| 30 | Porphyromonas | MKTQHFFEDFTSLYSLSKTIRFELKPIGKTLENIKKNGLIRRDEQRL |
| | macacae | DDYEKLKKVIDEYHEDFIANILSSFSFSEEILQSYIQNLSESEARAK |
| | (PmCas12a) | IEKTMRDTLAKAFSEDERYKSIFKKELVKKDIPVWCPAYKSLCKKFD |
| | | NFTTSLVPFHENRKNLYTSNEITASIPYRIVHVNLPKFIQNIEALCE |
| | | LQKKMGADLYLEMMENLRNVWPSFVKTPDDLCNLKTYNHLMVQSSIS |
| | | EYNRFVGGYSTEDGTKHQGINEWINIYRQRNKEMRLPGLVFLHKQIL |
| | | AKVDSSSFISDTLENDDQVFCVLRQFRKLFWNTVSSKEDDAASLKDL |
| | | FCGLSGYDPEAIYVSDAHLATISKNIFDRWNYISDAIRRKTEVLMPR |
| | | KKESVERYAEKISKQIKKRQSYSLAELDDLLAHYSEESLPAGFSLLS |
| | | YFTSLGGQKYLVSDGEVILYEEGSNIWDEVLIAFRDLQVILDKDFTE |
| | | KKLGKDEEAVSVIKKALDSALRLRKFFDLLSGTGAEIRRDSSFYALY |
| | | TDRMDKLKGLLKMYDKVRNYLTKKPYSIEKFKLHFDNPSLLSGWDKN |
| | | KELNNLSVIFRQNGYYYLGIMTPKGKNLFKTLPKLGAEEMFYEKMEY |
| | | KQIAEPMLMLPKVFFPKKTKPAFAPDQSVVDIYNKKTFKTGQKGFNK |
| | | KDLYRLIDFYKEALTVHEWKLFNFSFSPTEQYRNIGEFFDEVREQAY |
| | | KVSMVNVPASYIDEAVENGKLYLFQIYNKDFSPYSKGIPNLHTLYWK |
| | | ALFSEQNQSRVYKLCGGGELFYRKASLHMQDTTVHPKGISIHKKNLN |
| | | KKGETSLFNYDLVKDKRFTEDKFFFHVPISINYKNKKITNVNQMVRD |
| | | YIAQNDDLQIIGIDRGERNLLYISRIDTRGNLLEQFSLNVIESDKGD |
| | | LRTDYQKILGDREQERLRRRQEWKSIESIKDLKDGYMSQVVHKICNM |
| | | VVEHKAIVVLENLNLSFMKGRKKVEKSVYEKFERMLVDKLNYLVVDK |
| | | KNLSNEPGGLYAAYQLTNPLFSFEELHRYPQSGILFFVDPWNTSLTD |
| | | PSTGFVNLLGRINYTNVGDARKFFDRFNAIRYDGKGNILFDLDLSRF |
| | | DVRVETQRKLWTLTTFGSRIAKSKKSGKWMVERIENLSLCFLELFEQ |
| | | FNIGYRVEKDLKKAILSQDRKEFYVRLIYLFNLMMQIRNSDGEEDYI |
| | | LSPALNEKNLQFDSRLIEAKDLPVDADANGAYNVARKGLMVVQRIKR |
| | | GDHESIHRIGRAQWLRYVQEGIVE |
| 31 | Moraxella | MLFQDFTHLYPLSKTVRFELKPIDRTLEHIHAKNFLSQDETMADMHQ |
| | bovoculi | KVKVILDDYHRDFIADMMGEVKLTKLAEFYDVYLKFRKNPKDDELQK |
| | 237 | QLKDLQAVLRKEIVKPIGNGGKYKAGYDRLFGAKLFKDGKELGDLAK |
| | (MbCas12a) | FVIAQEGESSPKLAHLAHFEKFSTYFTGFHDNRKNMYSDEDKHTAIA |
| | | YRLIHENLPRFIDNLQILTTIKQKHSALYDQIINELTASGLDVSLAS |
| | | HLDGYHKLLTQEGITAYNTLLGGISGEAGSPKIQGINELINSHHNQH |
| | | CHKSERIAKLRPLHKQILSDGMSVSFLPSKFADDSEMCQAVNEFYRH |
| | | YADVFAKVQSLFDGFDDHQKDGIYVEHKNLNELSKQAFGDFALLGRV |
| | | LDGYYVDVVNPEFNERFAKAKTDNAKAKLTKEKDKFIKGVHSLASLE |
| | | QAIEHYTARHDDESVQAGKLGQYFKHGLAGVDNPIQKIHNNHSTIKG |
| | | FLERERPAGERALPKIKSGKNPEMTQLRQLKELLDNALNVAHFAKLL |
| | | TTKTTLDNQDGNFYGEFGVLYDELAKIPTLYNKVRDYLSQKPFSTEK |
| | | YKLNFGNPTLLNGWDLNKEKDNFGVILQKDGCYYLALLDKAHKKVFD |
| | | NAPNTGKSIYQKMIYKYLEVRKQFPKVFFSKEAIAINYHPSKELVEI |
| | | KDKGRQRSDDERLKLYRFILECLKIHPKYDKKFEGAIGDIQLFKKDK |
| | | KGREVPISEKDLFDKINGIFSSKPKLEMEDFFIGEFKRYNPSQDLVD |
| | | QYNIYKKIDSNDNRKKENFYNNHPKFKKDLVRYYYESMCKHEEWEES |
| | | FEFSKKLQDIGCYVDVNELFTEIETRRLNYKISFCNINADYIDELVE |
| | | QGQLYLFQIYNKDFSPKAHGKPNLHTLYFKALFSEDNLADPIYKLNG |
| | | EAQIFYRKASLDMNETTIHRAGEVLENKNPDNPKKRQFVYDIIKDKR |
| | | YTQDKFMLHVPITMNFGVQGMTIKEFNKKVNQSIQQYDEVNVIGIDR |
| | | GERHLLYLTVINSKGEILEQCSLNDITTASANGTQMTTPYHKILDKR |
| | | EIERLNARVGWGEIETIKELKSGYLSHVVHQISQLMLKYNAIVVLED |
| | | LNFGFKRGRFKVEKQIYQNFENALIKKLNHLVLKDKADDEIGSYKNA |
| | | LQLTNNFTDLKSIGKQTGFLFYVPAWNTSKIDPETGFVDLLKPRYEN |
| | | IAQSQAFFGKFDKICYNADKDYFEFHIDYAKFTDKAKNSRQIWTICS |
| | | HGDKRYVYDKTANQNKGAAKGINVNDELKSLFARHHINEKQPNLVMD |
| | | ICQNNDKEFHKSLMYLLKTLLALRYSNASSDEDFILSPVANDEGVFF |
| | | NSALADDTQPQNADANGAYHIALKGLWLLNELKNSDDLNKVKLAIDN |
| | | QTWLNFAQNR |
| 32 | Moraxella | MGIHGVPAALFQDFTHLYPLSKTVRFELKPIGRTLEHIHAKNFLSQD |
| | bovoculi | ETMADMYQKVKVILDDYHRDFIADMMGEVKLTKLAEFYDVYLKFRKN |
| | AAX08_00205 | PKDDGLQKQLKDLQAVLRKESVKPIGSGGKYKTGYDRLFGAKLFKDG |
| | (Mb2Cas12a) | KELGDLAKFVIAQEGESSPKLAHLAHFEKFSTYFTGFHDNRKNMYSD |
| | | EDKHTAIAYRLIHENLPRFIDNLQILTTIKQKHSALYDQIINELTAS |
| | | GLDVSLASHLDGYHKLLTQEGITAYNRIIGEVNGYTNKHNQICHKSE |
| | | RIAKLRPLHKQILSDGMGVSFLPSKFADDSEMCQAVNEFYRHYTDVF |
| | | AKVQSLFDGFDDHQKDGIYVEHKNLNELSKQAFGDFALLGRVLDGYY |
| | | VDVVNPEFNERFAKAKTDNAKAKLTKEKDKFIKGVHSLASLEQAIEH |
| | | HTARHDDESVQAGKLGQYFKHGLAGVDNPIQKIHNNHSTIKGFLERE |
| | | RPAGERALPKIKSGKNPEMTQLRQLKELLDNALNVAHFAKLLTTKTT |
| | | LDNQDGNFYGEFGVLYDELAKIPTLYNKVRDYLSQKPFSTEKYKLNF |
| | | GNPTLLNGWDLNKEKDNFGVILQKDGCYYLALLDKAHKKVFDNAPNT |
| | | GKNVYQKMVYKLLPGPNKMLPKVFFAKSNLDYYNPSAELLDKYAKGT |
| | | HKKGDNFNLKDCHALIDFFKAGINKHPEWQHFGFKFSPTSSYRDLSD |
| | | FYREVEPQGYQVKFVDINADYIDELVEQGKLYLFQIYNKDFSPKAHG |
| | | KPNLHTLYFKALFSEDNLADPIYKLNGEAQIFYRKASLDMNETTIHR |
| | | AGEVLENKNPDNPKKRQFVYDIIKDKRYTQDKFMLHVPITMNFGVQG |
| | | MTIKEFNKKVNQSIQQYDEVNVIGIDRGERHLLYLTVINSKGEILEQ |
| | | RSLNDITTASANGTQVTTPYHKILDKREIERLNARVGWGEIETIKEL |
| | | KSGYLSHVVHQINQLMLKYNAIVVLEDLNFGFKRGRFKVEKQIYQNF |
| | | ENALIKKLNHLVLKDKADDEIGSYKNALQLTNNFTDLKSIGKQTGFL |
| | | FYVPAWNTSKIDPETGFVDLLKPRYENIAQSQAFFGKFDKICYNTDK |
| | | GYFEFHIDYAKFTDKAKNSRQKWAICSHGDKRYVYDKTANQNKGAAK |
| | | GINVNDELKSLFARYHINDKQPNLVMDICQNNDKEFHKSLMCLLKTL |
| | | LALRYSNASSDEDFILSPVANDEGVFFNSALADDTQPQNADANGAYH |
| | | IALKGLWLLNELKNSDDLNKVKLAIDNQTWLNFAQNR |
| 33 | Moraxella | MGIHGVPAALFQDFTHLYPLSKTVRFELKPIGKTLEHIHAKNFLNQD |
| | bovoculi | ETMADMYQKVKAILDDYHRDFIADMMGEVKLTKLAEFYDVYLKFRKN |
| | AAX11_00205 | PKDDGLQKQLKDLQAVLRKEIVKPIGNGGKYKAGYDRLFGAKLFKDG |
| | (Mb3Cas12a) | KELGDLAKFVIAQEGESSPKLAHLAHFEKFSTYFTGFHDNRKNMYSD |
| | | EDKHTAIAYRLIHENLPRFIDNLQILATIKQKHSALYDQIINELTAS |
| | | GLDVSLASHLDGYHKLLTQEGITAYNTLLGGISGEAGSRKIQGINEL |
| | | INSHHNQHCHKSERIAKLRPLHKQILSDGMGVSFLPSKFADDSEVCQ |
| | | AVNEFYRHYADVFAKVQSLFDGFDDYQKDGIYVEYKNLNELSKQAFG |
| | | DFALLGRVLDGYYVDVVNPEFNERFAKAKTDNAKAKLTKEKDKFIKG |
| | | VHSLASLEQAIEHYTARHDDESVQAGKLGQYFKHGLAGVDNPIQKIH |
| | | NNHSTIKGFLERERPAGERALPKIKSDKSPEIRQLKELLDNALNVAH |
| | | FAKLLTTKTTLHNQDGNFYGEFGALYDELAKIATLYNKVRDYLSQKP |
| | | FSTEKYKLNFGNPTLLNGWDLNKEKDNFGVILQKDGCYYLALLDKAH |
| | | KKVFDNAPNTGKSVYQKMIYKLLPGPNKMLPKVFFAKSNLDYYNPSA |
| | | ELLDKYAQGTHKKGDNFNLKDCHALIDFFKAGINKHPEWQHFGFKFS |
| | | PTSSYQDLSDFYREVEPQGYQVKFVDINADYINELVEQGQLYLFQIY |
| | | NKDFSPKAHGKPNLHTLYFKALFSEDNLVNPIYKLNGEAEIFYRKAS |
| | | LDMNETTIHRAGEVLENKNPDNPKKRQFVYDIIKDKRYTQDKFMLHV |
| | | PITMNFGVQGMTIKEFNKKVNQSIQQYDEVNVIGIDRGERHLLYLTV |
| | | INSKGEILEQRSLNDITTASANGTQMTTPYHKILDKREIERLNARVG |
| | | WGEIETIKELKSGYLSHVVHQISQLMLKYNAIVVLEDLNFGFKRGRF |
| | | KVEKQIYQNFENALIKKLNHLVLKDKADDEIGSYKNALQLTNNFTDL |
| | | KSIGKQTGFLFYVPAWNTSKIDPETGFVDLLKPRYENIAQSQAFFGK |
| | | FDKICYNADRGYFEFHIDYAKFNDKAKNSRQIWKICSHGDKRYVYDK |
| | | TANQNKGATIGVNVNDELKSLFTRYHINDKQPNLVMDICQNNDKEFH |
| | | KSLMYLLKTLLALRYSNASSDEDFILSPVANDEGVFFNSALADDTQP |
| | | QNADANGAYHIALKGLWLLNELKNSDDLNKVKLAIDNQTWLNFAQNR |
| 34 | Thiomicrospira | MGIHGVPAATKTFDSEFFNLYSLQKTVRFELKPVGETASFVEDFKNE |
| | sp. XS5 | GLKRVVSEDERRAVDYQKVKEIIDDYHRDFIEESLNYFPEQVSKDAL |
| | (TsCas12a) | EQAFHLYQKLKAAKVEEREKALKEWEALQKKLREKVVKCFSDSNKAR |
| | | FSRIDKKELIKEDLINWLVAQNREDDIPTVETFNNFTTYFTGFHENR |
| | | KNIYSKDDHATAISFRLIHENLPKFFDNVISFNKLKEGFPELKFDKV |
| | | KEDLEVDYDLKHAFEIEYFVNFVTQAGIDQYNYLLGGKTLEDGTKKQ |
| | | GMNEQINLFKQQQTRDKARQIPKLIPLFKQILSERTESQSFIPKQFE |
| | | SDQELFDSLQKLHNNCQDKFTVLQQAILGLAEADLKKVFIKTSDLNA |
| | | LSNTIFGNYSVFSDALNLYKESLKTKKAQEAFEKLPAHSIHDLIQYL |
| | | EQFNSSLDAEKQQSTDTVLNYFIKTDELYSRFIKSTSEAFTQVQPLF |
| | | ELEALSSKRRPPESEDEGAKGQEGFEQIKRIKAYLDTLMEAVHFAKP |
| | | LYLVKGRKMIEGLDKDQSFYEAFEMAYQELESLIIPIYNKARSYLSR |
| | | KPFKADKFKINFDNNTLLSGWDANKETANASILFKKDGLYYLGIMPK |
| | | GKTFLFDYFVSSEDSEKLKQRRQKTAEEALAQDGESYFEKIRYKLLP |
| | | GASKMLPKVFFSNKNIGFYNPSDDILRIRNTASHTKNGTPQKGHSKV |
| | | EFNLNDCHKMIDFFKSSIQKHPEWGSFGFTFSDTSDFEDMSAFYREV |
| | | ENQGYVISFDKIKETYIQSQVEQGNLYLFQIYNKDFSPYSKGKPNLH |
| | | TLYWKALFEEANLNNVVAKLNGEAEIFFRRHSIKASDKVVHPANQAI |
| | | DNKNPHTEKTQSTFEYDLVKDKRYTQDKFFFHVPISLNFKAQGVSKF |
| | | NDKVNGFLKGNPDVNIIGIDRGERHLLYFTVVNQKGEILVQESLNTL |
| | | MSDKGHVNDYQQKLDKKEQERDAARKSWTTVENIKELKEGYLSHVVH |
| | | KLAHLIIKYNAIVCLEDLNFGFKRGRFKVEKQVYQKFEKALIDKLNY |
| | | LVFKEKELGEVGHYLTAYQLTAPFESFKKLGKQSGILFYVPADYTSK |
| | | IDPTTGFVNFLDLRYQSVEKAKQLLSDFNAIRFNSVQNYFEFEIDYK |
| | | KLTPKRKVGTQSKWVICTYGDVRYQNRRNQKGHWETEEVNVTEKLKA |
| | | LFASDSKTTTVIDYANDDNLIDVILEQDKASFFKELLWLLKLTMTLR |
| | | HSKIKSEDDFILSPVKNEQGEFYDSRKAGEVWPKDADANGAYHIALK |
| | | GLWNLQQINQWEKGKTLNLAIKNQDWFSFIQEKPYQE |
| 35 | Butyrivibrio | MGIHGVPAAYYQNLTKKYPVSKTIRNELIPIGKTLENIRKNNILESD |
| | sp. NC3005 | VKRKQDYEHVKGIMDEYHKQLINEALDNYMLPSLNQAAEIYLKKHVD |
| | (BsCas12a) | VEDREEFKKTQDLLRREVTGRLKEHENYTKIGKKDILDLLEKLPSIS |
| | | EEDYNALESFRNFYTYFTSYNKVRENLYSDEEKSSTVAYRLINENLP |
| | | KFLDNIKSYAFVKAAGVLADCIEEEEQDALFMVETFNMTLTQEGIDM |
| | | YNYQIGKVNSAINLYNQKNHKVEEFKKIPKMKVLYKQILSDREEVFI |
| | | GEFKDDETLLSSIGAYGNVLMTYLKSEKINIFFDALRESEGKNVYVK |
| | | NDLSKTTMSNIVFGSWSAFDELLNQEYDLANENKKKDDKYFEKRQKE |
| | | LKKNKSYTLEQMSNLSKEDISPIENYIERISEDIEKICIYNGEFEKI |
| | | VVNEHDSSRKLSKNIKAVKVIKDYLDSIKELEHDIKLINGSGQELEK |
| | | NLVVYVGQEEALEQLRPVDSLYNLTRNYLTKKPFSTEKVKLNFNKST |
| | | LLNGWDKNKETDNLGILFFKDGKYYLGIMNTTANKAFVNPPAAKTEN |
| | | VFKKVDYKLLPGSNKMLPKVFFAKSNIGYYNPSTELYSNYKKGTHKK |
| | | GPSFSIDDCHNLIDFFKESIKKHEDWSKFGFEFSDTADYRDISEFYR |
| | | EVEKQGYKLTFTDIDESYINDLIEKNELYLFQIYNKDFSEYSKGKLN |
| | | LHTLYFMMLFDQRNLDNVVYKLNGEAEVFYRPASIAENELVIHKAGE |
| | | GIKNKNPNRAKVKETSTFSYDIVKDKRYSKYKFTLHIPITMNFGVDE |
| | | VRRENDVINNALRTDDNVNVIGIDRGERNLLYVVVINSEGKILEQIS |
| | | LNSIINKEYDIETNYHALLDEREDDRNKARKDWNTIENIKELKTGYL |
| | | SQVVNVVAKLVLKYNAIICLEDLNFGFKRGRQKVEKQVYQKFEKMLI |
| | | EKLNYLVIDKSREQVSPEKMGGALNALQLTSKFKSFAELGKQSGIIY |
| | | YVPAYLTSKIDPTTGFVNLFYIKYENIEKAKQFFDGFDFIRFNKKDD |
| | | MFEFSFDYKSFTQKACGIRSKWIVYTNGERIIKYPNPEKNNLFDEKV |
| | | INVTDEIKGLFKQYRIPYENGEDIKEIIISKAEADFYKRLFRLLHQT |
| | | LQMRNSTSDGTRDYIISPVKNDRGEFFCSEFSEGTMPKDADANGAYN |
| | | IARKGLWVLEQIRQKDEGEKVNLSMTNAEWLKYAQLHLL |
| 36 | AacCas12b | MAVKSIKVKLRLDDMPEIRAGLWKLHKEVNAGVRYYTEWLSLLRQEN |
| | | LYRRSPNGDGEQECDKTAEECKAELLERLRARQVENGHRGPAGSDDE |
| | | LLQLARQLYELLVPQAIGAKGDAQQIARKFLSPLADKDAVGGLGIAK |
| | | AGNKPRWVRMREAGEPGWEEEKEKAETRKSADRTADVLRALADFGLK |
| | | PLMRVYTDSEMSSVEWKPLRKGQAVRTWDRDMFQQAIERMMSWESWN |
| | | QRVGQEYAKLVEQKNRFEQKNFVGQEHLVHLVNQLQQDMKEASPGLE |
| | | SKEQTAHYVTGRALRGSDKVFEKWGKLAPDAPFDLYDAEIKNVQRRN |
| | | TRRFGSHDLFAKLAEPEYQALWREDASFLTRYAVYNSILRKLNHAKM |
| | | FATFTLPDATAHPIWTRFDKLGGNLHQYTFLFNEFGERRHAIRFHKL |
| | | LKVENGVAREVDDVTVPISMSEQLDNLLPRDPNEPIALYFRDYGAEQ |
| | | HFTGEFGGAKIQCRRDQLAHMHRRRGARDVYLNVSVRVQSQSEARGE |
| | | RRPPYAAVFRLVGDNHRAFVHFDKLSDYLAEHPDDGKLGSEGLLSGL |
| | | RVMSVDLGLRTSASISVFRVARKDELKPNSKGRVPFFFPIKGNDNLV |
| | | AVHERSQLLKLPGETESKDLRAIREERQRTLRQLRTQLAYLRLLVRC |
| | | GSEDVGRRERSWAKLIEQPVDAANHMTPDWREAFENELQKLKSLHGI |
| | | CSDKEWMDAVYESVRRVWRHMGKQVRDWRKDVRSGERPKIRGYAKDV |
| | | VGGNSIEQIEYLERQYKFLKSWSFFGKVSGQVIRAEKGSRFAITLRE |
| | | HIDHAKEDRLKKLADRIIMEALGYVYALDERGKGKWVAKYPPCQLIL |
| | | LEELSEYQFNNDRPPSENNQLMQWSHRGVFQELINQAQVHDLLVGTM |
| | | YAAFSSRFDARTGAPGIRCRRVPARCTQEHNPEPFPWWLNKFVVEHT |
| | | LDACPLRADDLIPTGEGEIFVSPFSAEEGDFHQIHADLNAAQNLQQR |
| | | LWSDFDISQIRLRCDWGEVDGELVLIPRLTGKRTADSYSNKVFYTNT |
| | | GVTYYERERGKKRRKVFAQEKLSEEEAELLVEADEAREKSVVLMRDP |
| | | SGIINRGNWTRQKEFWSMVNQRIEGYLVKQIRSRVPLQDSACENTGD |
| | | I |
| 37 | Cas12 | MKKIDNFVGCYPVSKTLRFKAIPIGKTQENIEKKRLVEEDEVRAKDY |
| | Variant | KAVKKLIDRYHREFIEGVLDNVKLDGLEEYYMLFNKSDREESDNKKI |
| | | EIMEERFRRVISKSFKNNEEYKKIFSKKIIEEILPNYIKDEEEKELV |
| | | KGFKGFYTAFVGYAQNRENMYSDEKKSTAISYRIVNENMPRFITNIK |
| | | VFEKAKSILDVDKINEINEYILNNDYYVDDFFNIDFFNYVLNQKGID |
| | | IYNAIIGGIVTGDGRKIQGLNECINLYNQENKKIRLPQFKPLYKQIL |
| | | SESESMSFYIDEIESDDMLIDMLKESLQIDSTINNAIDDLKVLFNNI |
| | | FDYDLSGIFINNGLPITTISNDVYGQWSTISDGWNERYDVLSNAKDK |
| | | ESEKYFEKRRKEYKKVKSFSISDLQELGGKDLSICKKINEIISEMID |
| | | DYKSKIEEIQYLFDIKELEKPLVTDLNKIELIKNSLDGLKRIERYVI |
| | | PFLGTGKEQNRDEVFYGYFIKCIDAIKEIDGVYNKTRNYLTKKPYSK |
| | | DKFKLYFENPQLMGGWDRNKESDYRSTLLRKNGKYYVAIIDKSSSNC |
| | | MMNIEEDENDNYEKINYKLLPGPNKMLPKVFFSKKNREYFAPSKEIE |
| | | RIYSTGTFKKDTNFVKKDCENLITFYKDSLDRHEDWSKSFDFSFKES |
| | | SAYRDISEFYRDVEKQGYRVSFDLLSSNAVNTLVEEGKLYLFQLYNK |
| | | DFSEKSHGIPNLHTMYFRSLFDDNNKGNIRLNGGAEMFMRRASLNKQ |
| | | DVTVHKANQPIKNKNLLNPKKTTTLPYDVYKDKRFTEDQYEVHIPIT |
| | | MNKVPNNPYKINHMVREQLVKDDNPYVIGIDRGERNLIYVVVVDGQG |
| | | HIVEQLSLNEIINENNGISIRTDYHTLLDAKERERDESRKQWKQIEN |
| | | IKELKEGYISQVVHKICELVEKYDAVIALEDLNSGFKNSRVKVEKQV |
| | | YQKFEKMLITKLNYMVDKKKDYNKPGGVLNGYQLTTQFESFSKMGTQ |
| | | NGIMFYIPAWLTSKMDPTTGFVDLLKPKYKNKADAQKFFSQFDSIRY |
| | | DNQEDAFVFKVNYTKFPRTDADYNKEWEIYTNGERIRVFRNPKKNNE |
| | | YDYETVNVSERMKELFDSYDLLYDKGELKETICEMEESKFFEELIKL |
| | | FRLTLQMRNSISGRTDVDYLISPVKNSNGYFYNSNDYKKEGAKYPKD |
| | | ADANGAYNIARKVLWAIEQFKMADEDKLDKTKISIKNQEWLEYAQTH |
| | | CE |

Alternatively, or in combination (e.g., during multiplexing), the Type V CRISPR/Cas enzyme is a programmable Cas14 nuclease. A Cas14 protein of the present disclosure includes 3 partial RuvC domains (RuvC-I, RuvC-II, and RuvC-III, also referred to herein as subdomains) that are not contiguous with respect to the primary amino acid sequence of the Cas14 protein, but form a RuvC domain once the protein is produced and folds. A naturally occurring Cas14 protein functions as an endonuclease that catalyzes cleavage at a specific sequence in a target nucleic acid. A programmable Cas14 nuclease can be a Cas14a protein, a Cas14b protein, a Cas14c protein, a Cas14d protein, a Cas14e protein, a Cas14f protein, a Cas14g protein, a Cas14h protein, or a Cas14u protein. In some cases, a suitable Cas14 protein comprises an amino acid sequence having at least 80%, at least 85%, at least 90%, at least 95%, at least 98%, at least 99%, or 100%, amino acid sequence identity to any one of SEQ ID NO: 38-SEQ ID NO: 129.

TABLE 2
Cas14 Protein Sequences

| SEQ ID | |
|---|---|
| NO | Sequence |
| 38 | MEVQKTVMKTLSLRILRPLYSQEIEKEIKEEKERRKQAGGTGELDGGFYKKLEKKH |
| | SEMFSFDRLNLLLNQLQREIAKVYNHAISELYIATIAQGNKSNKHYISSIVYNRAY |
| | GYFYNAYIALGICSKVEANFRSNELLTQQSALPTAKSDNFPIVLHKQKGAEGEDGG |
| | FRISTEGSDLIFEIPIPFYEYNGENRKEPYKWVKKGGQKPVLKLILSTFRRQRNKG |
| | WAKDEGTDAEIRKVTEGKYQVSQIEINRGKKLGEHQKWFANFSIEQPIYERKPNRS |
| | IVGGLDVGIRSPLVCAINNSFSRYSVDSNDVFKFSKQVFAFRRRLLSKNSLKRKGH |
| | GAAHKLEPITEMTEKNDKFRKKIIERWAKEVTNFFVKNQVGIVQIEDLSTMKDRED |
| | HFFNQYLRGFWPYYQMQTLIENKLKEYGIEVKRVQAKYTSQLCSNPNCRYWNNYFN |
| | FEYRKVNKFPKFKCEKCNLEISADYNAARNLSTPDIEKFVAKATKGINLPEK |
| 39 | MEEAKTVSKTLSLRILRPLYSAEIEKEIKEEKERRKQGGKSGELDSGFYKKLEKKH |
| | TQMFGWDKLNLMLSQLQRQIARVENQSISELYIETVIQGKKSNKHYTSKIVYNRAY |
| | SVFYNAYLALGITSKVEANFRSTELLMQKSSLPTAKSDNFPILLHKQKGVEGEEGG |
| | FKISADGNDLIFEIPIPFYEYDSANKKEPFKWIKKGGQKPTIKLILSTFRRQRNKG |
| | WAKDEGTDAEIRKVIEGKYQVSHIEINRGKKLGDHQKWFVNFTIEQPIYERKLDKN |
| | IIGGIDVGIKSPLVCAVNNSFARYSVDSNDVLKFSKQAFAFRRRLLSKNSLKRSGH |
| | GSKNKLDPITRMTEKNDRFRKKIIERWAKEVTNFFIKNQVGTVQIEDLSTMKDRQD |
| | NFFNQYLRGFWPYYQMQNLIENKLKEYGIETKRIKARYTSQLCSNPSCRHWNSYFS |
| | FDHRKTNNFPKFKCEKCALEISADYNAARNISTPDIEKFVAKATKGINLPDKNENV |
| | ILE |
| 40 | MAKNTITKTLKLRIVRPYNSAEVEKIVADEKNNREKIALEKNKDKVKEACSKHLKV |
| | AAYCTTQVERNACLFCKARKLDDKFYQKLRGQFPDAVFWQEISEIFRQLQKQAAEI |
| | YNQSLIELYYEIFIKGKGIANASSVEHYLSDVCYTRAAELFKNAAIASGLRSKIKS |
| | NFRLKELKNMKSGLPTTKSDNFPIPLVKQKGGQYTGFEISNHNSDFIIKIPFGRWQ |
| | VKKEIDKYRPWEKFDFEQVQKSPKPISLLLSTQRRKRNKGWSKDEGTEAEIKKVMN |
| | GDYQTSYIEVKRGSKIGEKSAWMLNLSIDVPKIDKGVDPSIIGGIDVGVKSPLVCA |
| | INNAFSRYSISDNDLFHFNKKMFARRRILLKKNRHKRAGHGAKNKLKPITILTEKS |
| | ERFRKKLIERWACEIADFFIKNKVGTVQMENLESMKRKEDSYFNIRLRGFWPYAEM |
| | QNKIEFKLKQYGIEIRKVAPNNTSKTCSKCGHLNNYFNFEYRKKNKFPHFKCEKCN |
| | FKENADYNAALNISNPKLKSTKEEP |
| 41 | MERQKVPQIRKIVRVVPLRILRPKYSDVIENALKKFKEKGDDTNTNDFWRAIRDRD |
| | TEFFRKELNFSEDEINQLERDTLFRVGLDNRVLFSYFDFLQEKLMKDYNKIISKLF |
| | INRQSKSSFENDLTDEEVEELIEKDVTPFYGAYIGKGIKSVIKSNLGGKFIKSVKI |
| | DRETKKVTKLTAINIGLMGLPVAKSDTFPIKIIKTNPDYITFQKSTKENLQKIEDY |
| | ETGIEYGDLLVQITIPWFKNENKDFSLIKTKEAIEYYKLNGVGKKDLLNINLVLTT |
| | YHIRKKKSWQIDGSSQSLVREMANGELEEKWKSFFDTFIKKYGDEGKSALVKRRVN |
| | KKSRAKGEKGRELNLDERIKRLYDSIKAKSFPSEINLIPENYKWKLHFSIEIPPMV |
| | NDIDSNLYGGIDFGEQNIATLCVKNIEKDDYDFLTIYGNDLLKHAQASYARRRIMR |
| | VQDEYKARGHGKSRKTKAQEDYSERMQKLRQKITERLVKQISDFFLWRNKFHMAVC |
| | SLRYEDLNTLYKGESVKAKRMRQFINKQQLFNGIERKLKDYNSEIYVNSRYPHYTS |
| | RLCSKCGKLNLYFDFLKFRTKNIIIRKNPDGSEIKYMPFFICEFCGWKQAGDKNAS |
| | ANIADKDYQDKLNKEKEFCNIRKPKSKKEDIGEENEEERDYSRRFNRNSFIYNSLK |
| | KDNKLNQEKLFDEWKNQLKRKIDGRNKFEPKEYKDRFSYLFAYYQEIIKNESES |
| 42 | MVPTELITKTLQLRVIRPLYFEEIEKELAELKEQKEKEFEETNSLLLESKKIDAKS |
| | LKKLKRKARSSAAVEFWKIAKEKYPDILTKPEMEFIFSEMQKMMARFYNKSMTNIF |
| | IEMNNDEKVNPLSLISKASTEANQVIKCSSISSGLNRKIAGSINKTKFKQVRDGLI |
| | SLPTARTETFPISFYKSTANKDEIPISKINLPSEEEADLTITLPFPFFEIKKEKKG |
| | QKAYSYFNIIEKSGRSNNKIDLLLSTHRRQRRKGWKEEGGTSAEIRRLMEGEFDKE |
| | WEIYLGEAEKSEKAKNDLIKNMTRGKLSKDIKEQLEDIQVKYFSDNNVESWNDLSK |
| | EQKQELSKLRKKKVEELKDWKHVKEILKTRAKIGWVELKRGKRQRDRNKWFVNITI |
| | TRPPFINKELDDTKFGGIDLGVKVPFVCAVHGSPARLIIKENEILQFNKMVSARNR |
| | QITKDSEQRKGRGKKNKFIKKEIFNERNELFRKKIIERWANQIVKFFEDQKCATVQ |
| | IENLESFDRTSYK |
| 43 | MKSDTKDKKIIIHQTKTLSLRIVKPQSIPMEEFTDLVRYHQMIIFPVYNNGAIDLY |
| | KKLFKAKIQKGNEARAIKYFMNKIVYAPIANTVKNSYIALGYSTKMQSSFSGKRLW |
| | DLRFGEATPPTIKADFPLPFYNQSGFKVSSENGEFIIGIPFGQYTKKTVSDIEKKT |
| | SFAWDKFTLEDTTKKTLIELLLSTKTRKMNEGWKNNEGTEAEIKRVMDGTYQVTSL |
| | EILQRDDSWFVNFNIAYDSLKKQPDRDKIAGIHMGITRPLTAVIYNNKYRALSIYP |
| | NTVMHLTQKQLARIKEQRTNSKYATGGHGRNAKVTGTDTLSEAYRQRRKKIIEDWI |
| | ASIVKFAINNEIGTIYLEDISNTNSFFAAREQKLIYLEDISNTNSFLSTYKYPISA |
| | ISDTLQHKLEEKAIQVIRKKAYYVNQICSLCGHYNKGFTYQFRRKNKFPKMKCQGC |
| | LEATSTEFNAAANVANPDYEKLLIKHGLLQLKK |
| 44 | MSTITRQVRLSPTPEQSRLLMAHCQQYISTVNVLVAAFDSEVLTGKVSTKDFRAAL |
| | PSAVKNQALRDAQSVFKRSVELGCLPVLKKPHCQWNNQNWRVEGDQLILPICKDGK |
| | TQQERFRCAAVALEGKAGILRIKKKRGKWIADLTVTQEDAPESSGSAIMGVDLGIK |
| | VPAVAHIGGKGTRFFGNGRSQRSMRRRFYARRKTLQKAKKLRAVRKSKGKEARWMK |
| | TINHQLSRQIVNHAHALGVGTIKIEALQGIRKGTTRKSRGAAARKNNRMTNTWSFS |
| | QLTLFITYKAQRQGITVEQVDPAYTSQDCPACRARNGAQDRTYVCSECGWRGHRDT |
| | VGAINISRRAGLSGHRRGATGA |
| 45 | MIAQKTIKIKLNPTKEQIIKLNSIIEEYIKVSNFTAKKIAEIQESFTDSGLTQGTC |
| | SECGKEKTYRKYHLLKKDNKLFCITCYKRKYSQFTLQKVEFQNKTGLRNVAKLPKT |
| | YYTNAIRFASDTFSGFDEIIKKKQNRLNSIQNRLNFWKELLYNPSNRNEIKIKVVK |
| | YAPKTDTREHPHYYSEAEIKGRIKRLEKQLKKFKMPKYPEFTSETISLQRELYSWK |
| | NPDELKISSITDKNESMNYYGKEYLKRYIDLINSQTPQILLEKENNSFYLCFPITK |
| | NIEMPKIDDTFEPVGIDWGITRNIAVVSILDSKTKKPKFVKFYSAGYILGKRKHYK |
| | SLRKHFGQKKRQDKINKLGTKEDRFIDSNIHKLAFLIVKEIRNHSNKPIILMENIT |
| | DNREEAEKSMRQNILLHSVKSRLQNYIAYKALWNNIPTNLVKPEHTSQICNRCGHQ |
| | DRENRPKGSKLFKCVKCNYMSNADFNASINIARKFYIGEYEPFYKDNEKMKSGVNS |
| | ISM |
| 46 | LKLSEQENITTGVKFKLKLDKETSEGLNDYFDEYGKAINFAIKVIQKELAEDRFAG |
| | KVRLDENKKPLLNEDGKKIWDFPNEFCSCGKQVNRYVNGKSLCQECYKNKFTEYGI |
| | RKRMYSAKGRKAEQDINIKNSTNKISKTHFNYAIREAFILDKSIKKQRKERFRRLR |
| | EMKKKLQEFIEIRDGNKILCPKIEKQRVERYIHPSWINKEKKLEDFRGYSMSNVLG |
| | KIKILDRNIKREEKSLKEKGQINFKARRLMLDKSVKFLNDNKISFTISKNLPKEYE |
| | LDLPEKEKRLNWLKEKIKIIKNQKPKYAYLLRKDDNFYLQYTLETEFNLKEDYSGI |
| | VGIDRGVSHIAVYTFVHNNGKNERPLFLNSSEILRLKNLQKERDRFLRRKHNKKRK |
| | KSNMRNIEKKIQLILHNYSKQIVDFAKNKNAFIVFEKLEKPKKNRSKMSKKSQYKL |
| | SQFTFKKLSDLVDYKAKREGIKVLYISPEYTSKECSHCGEKVNTQRPFNGNSSLFK |
| | CNKCGVELNADYNASINIAKKGLNILNSTN |
| 47 | MEESIITGVKFKLRIDKETTKKLNEYFDEYGKAINFAVKIIQKELADDRFAGKAKL |
| | DQNKNPILDENGKKIYEFPDEFCSCGKQVNKYVNNKPFCQECYKIRFTENGIRKRM |
| | YSAKGRKAEHKINILNSTNKISKTHFNYAIREAFILDKSIKKQRKKRNERLRESKK |
| | RLQQFIDMRDGKREICPTIKGQKVDRFIHPSWITKDKKLEDFRGYTLSIINSKIKI |
| | LDRNIKREEKSLKEKGQIIFKAKRLMLDKSIRFVGDRKVLFTISKTLPKEYELDLP |
| | SKEKRLNWLKEKIEIIKNQKPKYAYLLRKNIESEKKPNYEYYLQYTLEIKPELKDF |
| | YDGAIGIDRGINHIAVCTFISNDGKVTPPKFFSSGEILRLKNLQKERDRFLLRKHN |
| | KNRKKGNMRVIENKINLILHRYSKQIVDMAKKLNASIVFEELGRIGKSRTKMKKSQ |
| | RYKLSLFIFKKLSDLVDYKSRREGIRVTYVPPEYTSKECSHCGEKVNTQRPFNGNY |
| | SLFKCNKCGIQLNSDYNASINIAKKGLKIPNST |
| 48 | LWTIVIGDFIEMPKQDLVTTGIKFKLDVDKETRKKLDDYFDEYGKAINFAVKIIQK |
| | NLKEDRFAGKIALGEDKKPLLDKDGKKIYNYPNESCSCGNQVRRYVNAKPFCVDCY |
| | KLKFTENGIRKRMYSARGRKADSDINIKNSTNKISKTHFNYAIREGFILDKSLKKQ |
| | RSKRIKKLLELKRKLQEFIDIRQGQMVLCPKIKNQRVDKFIHPSWLKRDKKLEEFR |
| | GYSLSVVEGKIKIFNRNILREEDSLRQRGHVNFKANRIMLDKSVRFLDGGKVNFNL |
| | NKGLPKEYLLDLPKKENKLSWLNEKISLIKLQKPKYAYLLRREGSFFIQYTIENVP |
| | KTFSDYLGAIGIDRGISHIAVCTFVSKNGVNKAPVFFSSGEILKLKSLQKQRDLFL |
| | RGKHNKIRKKSNMRNIDNKINLILHKYSRNIVNLAKSEKAFIVFEKLEKIKKSRFK |
| | MSKSLQYKLSQFTFKKLSDLVEYKAKIEGIKVDYVPPEYTSKECSHCGEKVDTQRP |
| | FNGNSSLFKCNKCRVQLNADYNASINIAKKSLNISN |
| 49 | MSKTTISVKLKIIDLSSEKKEFLDNYFNEYAKATTFCQLRIRRLLRNTHWLGKKEK |
| | SSKKWIFESGICDLCGENKELVNEDRNSGEPAKICKRCYNGRYGNQMIRKLFVSTK |
| | KREVQENMDIRRVAKLNNTHYHRIPEEAFDMIKAADTAEKRRKKNVEYDKKRQMEF |
| | IEMFNDEKKRAARPKKPNERETRYVHISKLESPSKGYTLNGIKRKIDGMGKKIERA |
| | EKGLSRKKIFGYQGNRIKLDSNWVRFDLAESEITIPSLFKEMKLRITGPTNVHSKS |
| | GQIYFAEWFERINKQPNNYCYLIRKTSSNGKYEYYLQYTYEAEVEANKEYAGCLGV |
| | DIGCSKLAAAVYYDSKNKKAQKPIEIFTNPIKKIKMRREKLIKLLSRVKVRHRRRK |
| | LMQLSKTEPIIDYTCHKTARKIVEMANTAKAFISMENLETGIKQKQQARETKKQKF |
| | YRNMFLFRKLSKLIEYKALLKGIKIVYVKPDYTSQTCSSCGADKEKTERPSQAIFR |
| | CLNPTCRYYQRDINADFNAAVNIAKKALNNTEVVTTLL |
| 50 | MARAKNQPYQKLTTTTGIKFKLDLSEEEGKRFDEYFSEYAKAVNFCAKVIYQLRKN |
| | LKFAGKKELAAKEWKFEISNCDFCNKQKEIYYKNIANGQKVCKGCHRTNFSDNAIR |
| | KKMIPVKGRKVESKFNIHNTTKKISGTHRHWAFEDAADIIESMDKQRKEKQKRLRR |
| | EKRKLSYFFELFGDPAKRYELPKVGKQRVPRYLHKIIDKDSLTKKRGYSLSYIKNK |
| | IKISERNIERDEKSLRKASPIAFGARKIKMSKLDPKRAFDLENNVFKIPGKVIKGQ |
| | YKFFGTNVANEHGKKFYKDRISKILAGKPKYFYLLRKKVAESDGNPIFEYYVQWSI |
| | DTETPAITSYDNILGIDAGITNLATTVLIPKNLSAEHCSHCGNNHVKPIFTKFFSG |
| | KELKAIKIKSRKQKYFLRGKHNKLVKIKRIRPIEQKVDGYCHVVSKQIVEMAKERN |
| | SCIALEKLEKPKKSKFRQRRREKYAVSMFVFKKLATFIKYKAAREGIEIIPVEPEG |
| | TSYTCSHCKNAQNNQRPYFKPNSKKSWTSMFKCGKCGIELNSDYNAAFNIAQKALN |
| | MTSA |
| 51 | MDEKHFFCSYCNKELKISKNLINKISKGSIREDEAVSKAISIHNKKEHSLILGIKF |
| | KLFIENKLDKKKLNEYFDNYSKAVTFAARIFDKIRSPYKFIGLKDKNTKKWTFPKA |
| | KCVFCLEEKEVAYANEKDNSKICTECYLKEFGENGIRKKIYSTRGRKVEPKYNIFN |
| | STKELSSTHYNYAIRDAFQLLDALKKQRQKKLKSIFNQKLRLKEFEDIFSDPQKRI |
| | ELSLKPHQREKRYIHLSKSGQESINRGYTLRFVRGKIKSLTRNIEREEKSLRKKTP |
| | IHFKGNRLMIFPAGIKFDFASNKVKISISKNLPNEFNFSGTNVKNEHGKSFFKSRI |
| | ELIKTQKPKYAYVLRKIKREYSKLRNYEIEKIRLENPNADLCDFYLQYTIETESRN |
| | NEEINGIIGIDRGITNLACLVLLKKGDKKPSGVKFYKGNKILGMKIAYRKHLYLLK |
| | GKRNKLRKQRQIRAIEPKINLILHQISKDIVKIAKEKNFAIALEQLEKPKKARFAQ |
| | RKKEKYKLALFTFKNLSTLIEYKSKREGIPVIYVPPEKTSQMCSHCAINGDEHVDT |
| | QRPYKKPNAQKPSYSLFKCNKCGIELNADYNAAFNIAQKGLKTLMLNHSH |
| 52 | MLQTLLVKLDPSKEQYKMLYETMERFNEACNQIAETVFAIHSANKIEVQKTVYYPI |
| | REKFGLSAQLTILAIRKVCEAYKRDKSIKPEFRLDGALVYDQRVLSWKGLDKVSLV |
| | TLQGRQIIPIKFGDYQKARMDRIRGQADLILVKGVFYLCVVVEVSEESPYDPKGVL |
| | GVDLGIKNLAVDSDGEVHSGEQTTNTRERLDSLKARLQSKGTKSAKRHLKKLSGRM |
| | AKFSKDVNHCISKKLVAKAKGTLMSIALEDLQGIRDRVTVRKAQRRNLHTWNFGLL |
| | RMFVDYKAKIAGVPLVFVDPRNTSRTCPSCGHVAKANRPTRDEFRCVSCGFAGAAD |
| | HIAAMNIAFRAEVSQPIVTRFFVQSQAPSFRVG |
| 53 | MDEEPDSAEPNLAPISVKLKLVKLDGEKLAALNDYFNEYAKAVNFCELKMQKIRKN |
| | LVNIRGTYLKEKKAWINQTGECCICKKIDELRCEDKNPDINGKICKKCYNGRYGNQ |
| | MIRKLFVSTNKRAVPKSLDIRKVARLHNTHYHRIPPEAADIIKAIETAERKRRNRI |
| | LFDERRYNELKDALENEEKRVARPKKPKEREVRYVPISKKDTPSKGYTMNALVRKV |
| | SGMAKKIERAKRNLNKRKKIEYLGRRILLDKNWVRFDFDKSEISIPTMKEFFGEMR |
| | FEITGPSNVMSPNGREYFTKWFDRIKAQPDNYCYLLRKESEDETDFYLQYTWRPDA |
| | HPKKDYTGCLGIDIGGSKLASAVYFDADKNRAKQPIQIFSNPIGKWKTKRQKVIKV |
| | LSKAAVRHKTKKLESLRNIEPRIDVHCHRIARKIVGMALAANAFISMENLEGGIRE |
| | KQKAKETKKQKFSRNMFVFRKLSKLIEYKALMEGVKVVYIVPDYTSQLCSSCGTNN |
| | TKRPKQAIFMCONTECRYFGKNINADFNAAINIAKKALNRKDIVRELS |
| 54 | MEKNNSEQTSITTGIKFKLKLDKETKEKLNNYFDEYGKAINFAVRIIQMQLNDDRL |
| | AGKYKRDEKGKPILGEDGKKILEIPNDFCSCGNQVNHYVNGVSFCQECYKKRFSEN |
| | GIRKRMYSAKGRKAEQDINIKNSTNKISKTHFNYAIREAFNLDKSIKKQREKRFKK |
| | LKDMKRKLQEFLEIRDGKRVICPKIEKQKVERYIHPSWINKEKKLEEFRGYSLSIV |
| | NSKIKSFDRNIQREEKSLKEKGQINFKAQRLMLDKSVKFLKDNKVSFTISKELPKT |
| | FELDLPKKEKKLNWLNEKLEIIKNQKPKYAYLLRKENNIFLQYTLDSIPEIHSEYS |
| | GAVGIDRGVSHIAVYTFLDKDGKNERPFFLSSSGILRLKNLQKERDKFLRKKHNKI |
| | RKKGNMRNIEQKINLILHEYSKQIVNFAKDKNAFIVFELLEKPKKSRERMSKKIQY |
| | KLSQFTFKKLSDLVDYKAKREGIKVIYVEPAYTSKDCSHCGERVNTQRPFNGNFSL |
| | FKCNKCGIVLNSDYNASLNIARKGLNISAN |
| 55 | MAEEKFFFCEKCNKDIKIPKNYINKQGAEEKARAKHEHRVHALILGIKFKIYPKKE |
| | DISKLNDYFDEYAKAVTFTAKIVDKLKAPFLFAGKRDKDTSKKKWVFPVDKCSFCK |
| | EKTEINYRTKQGKNICNSCYLTEFGEQGLLEKIYATKGRKVSSSFNLFNSTKKLTG |
| | THNNYVVKESLQLLDALKKQRSKRLKKLSNTRRKLKQFEEMFEKEDKRFQLPLKEK |
| | QRELRFIHVSQKDRATEFKGYTMNKIKSKIKVLRRNIEREQRSLNRKSPVFFRGTR |
| | IRLSPSVQFDDKDNKIKLTLSKELPKEYSFSGLNVANEHGRKFFAEKLKLIKENKS |
| | KYAYLLRRQVNKNNKKPIYDYYLQYTVEFLPNIITNYNGILGIDRGINTLACIVLL |
| | ENKKEKPSFVKFFSGKGILNLKNKRRKQLYFLKGVHNKYRKQQKIRPIEPRIDQIL |
| | HDISKQIIDLAKEKRVAISLEQLEKPQKPKFRQSRKAKYKLSQFNFKTLSNYIDYK |
| | AKKEGIRVIYIAPEMTSQNCSRCAMKNDLHVNTQRPYKNTSSLFKCNKCGVELNAD |
| | YNAAFNIAQKGLKILNS |
| 56 | MISLKLKLLPDEEQKKLLDEMFWKWASICTRVGFGRADKEDLKPPKDAEGVWFSLT |
| | QLNQANTDINDLREAMKHQKHRLEYEKNRLEAQRDDTQDALKNPDRREISTKRKDL |
| | FRPKASVEKGFLKLKYHQERYWVRRLKEINKLIERKTKTLIKIEKGRIKFKATRIT |
| | LHQGSFKIRFGDKPAFLIKALSGKNQIDAPFVVVPEQPICGSVVNSKKYLDEITTN |
| | FLAYSVNAMLFGLSRSEEMLLKAKRPEKIKKKEEKLAKKQSAFENKKKELQKLLGR |
| | ELTQQEEAIIEETRNQFFQDFEVKITKQYSELLSKIANELKQKNDFLKVNKYPILL |
| | RKPLKKAKSKKINNLSPSEWKYYLQFGVKPLLKQKSRRKSRNVLGIDRGLKHLLAV |
| | TVLEPDKKTFVWNKLYPNPITGWKWRRRKLLRSLKRLKRRIKSQKHETIHENQTRK |
| | KLKSLQGRIDDLLHNISRKIVETAKEYDAVIVVEDLQSMRQHGRSKGNRLKTLNYA |
| | LSLFDYANVMQLIKYKAGIEGIQIYDVKPAGTSQNCAYCLLAQRDSHEYKRSQENS |
| | KIGVCLNPNCQNHKKQIDADLNAARVIASCYALKINDSQPFGTRKRFKKRTTN |
| 57 | METLSLKLKLNPSKEQLLVLDKMFWKWASICTRLGLKKAEMSDLEPPKDAEGVWFS |
| | KTQLNQANTDVNDLRKAMQHQGKRIEYELDKVENRRNEIQEMLEKPDRRDISPNRK |
| | DLFRPKAAVEKGYLKLKYHKLGYWSKELKTANKLIERKRKTLAKIDAGKMKFKPTR |
| | ISLHTNSFRIKFGEEPKIALSTTSKHEKIELPLITSLQRPLKTSCAKKSKTYLDAA |
| | ILNFLAYSTNAALFGLSRSEEMLLKAKKPEKIEKRDRKLATKRESFDKKLKTLEKL |
| | LERKLSEKEKSVFKRKQTEFFDKFCITLDETYVEALHRIAEELVSKNKYLEIKKYP |
| | VLLRKPESRLRSKKLKNLKPEDWTYYIQFGFQPLLDTPKPIKTKTVLGIDRGVRHL |
| | LAVSIFDPRTKTFTFNRLYSNPIVDWKWRRRKLLRSIKRLKRRLKSEKHVHLHENQ |
| | FKAKLRSLEGRIEDHFHNLSKEIVDLAKENNSVIVVENLGGMRQHGRGRGKWLKAL |
| | NYALSHFDYAKVMQLIKYKAELAGVFVYDVAPAGTSINCAYCLLNDKDASNYTRGK |
| | VINGKKNTKIGECKTCKKEFDADLNAARVIALCYEKRLNDPQPFGTRKQFKPKKP |
| 58 | MKALKLQLIPTRKQYKILDEMFWKWASLANRVSQKGESKETLAPKKDIQKIQFNAT |
| | QLNQIEKDIKDLRGAMKEQQKQKERLLLQIQERRSTISEMLNDDNNKERDPHRPLN |
| | FRPKGWRKFHTSKHWVGELSKILRQEDRVKKTIERIVAGKISFKPKRIGIWSSNYK |
| | INFFKRKISINPLNSKGFELTLMTEPTQDLIGKNGGKSVLNNKRYLDDSIKSLLMF |
| | ALHSRFFGLNNTDTYLLGGKINPSLVKYYKKNQDMGEFGREIVEKFERKLKQEINE |
| | QQKKIIMSQIKEQYSNRDSAFNKDYLGLINEFSEVENQRKSERAEYLLDSFEDKIK |
| | QIKQEIGESLNISDWDFLIDEAKKAYGYEEGFTEYVYSKRYLEILNKIVKAVLITD |
| | IYFDLRKYPILLRKPLDKIKKISNLKPDEWSYYIQFGYDSINPVQLMSTDKFLGID |
| | RGLTHLLAYSVFDKEKKEFIINQLEPNPIMGWKWKLRKVKRSLQHLERRIRAQKMV |
| | KLPENQMKKKLKSIEPKIEVHYHNISRKIVNLAKDYNASIVVESLEGGGLKQHGRK |
| | KNARNRSLNYALSLFDYGKIASLIKYKADLEGVPMYEVLPAYTSQQCAKCVLEKGS |
| | FVDPEIIGYVEDIGIKGSLLDSLFEGTELSSIQVLKKIKNKIELSARDNHNKEINL |
| | ILKYNFKGLVIVRGQDKEEIAEHPIKEINGKFAILDFVYKRGKEKVGKKGNQKVRY |
| | TGNKKVGYCSKHGQVDADLNASRVIALCKYLDINDPILFGEQRKSFK |
| 59 | MVTRAIKLKLDPTKNQYKLLNEMFWKWASLANRFSQKGASKETLAPKDGTQKIQFN |
| | ATQLNQIKKDVDDLRGAMEKQGKQKERLLIQIQERLLTISEILRDDSKKEKDPHRP |
| | QNFRPFGWRRFHTSAYWSSEASKLTRQVDRVRRTIERIKAGKINFKPKRIGLWSST |
| | YKINFLKKKINISPLKSKSFELDLITEPQQKIIGKEGGKSVANSKKYLDDSIKSLL |
| | IFAIKSRLFGLNNKDKPLFENIITPNLVRYHKKGQEQENFKKEVIKKFENKLKKEI |
| | SQKQKEIIFSQIERQYENRDATFSEDYLRAISEFSEIFNQRKKERAKELLNSFNEK |
| | IRQLKKEVNGNISEEDLKILEVEAEKAYNYENGFIEWEYSEQFLGVLEKIARAVLI |
| | SDNYFDLKKYPILIRKPTNKSKKITNLKPEEWDYYIQFGYGLINSPMKIETKNFMG |
| | IDRGLTHLLAYSIFDRDSEKFTINQLELNPIKGWKWKLRKVKRSLQHLERRMRAQK |
| | GVKLPENQMKKRLKSIEPKIESYYHNLSRKIVNLAKANNASIVVESLEGGGLKQHG |
| | RKKNSRHRALNYALSLFDYGKIASLIKYKSDLEGVPMYEVLPAYTSQQCAKCVLKK |
| | GSFVEPEIIGYIEEIGFKENLLTLLFEDTGLSSVQVLKKSKNKMTLSARDKEGKMV |
| | DLVLKYNFKGLVISQEKKKEEIVEFPIKEIDGKFAVLDSAYKRGKERISKKGNQKL |
| | VYTGNKKVGYCSVHGQVDADLNASRVIALCKYLGINEPIVFGEQRKSFK |
| 60 | LDLITEPIQPHKSSSLRSKEFLEYQISDFLNFSLHSLFFGLASNEGPLVDFKIYDK |
| | IVIPKPEERFPKKESEEGKKLDSFDKRVEEYYSDKLEKKIERKLNTEEKNVIDREK |
| | TRIWGEVNKLEEIRSIIDEINEIKKQKHISEKSKLLGEKWKKVNNIQETLLSQEYV |
| | SLISNLSDELTNKKKELLAKKYSKFDDKIKKIKEDYGLEFDENTIKKEGEKAFLNP |
| | DKFSKYQFSSSYLKLIGEIARSLITYKGFLDLNKYPIIFRKPINKVKKIHNLEPDE |
| | WKYYIQFGYEQINNPKLETENILGIDRGLTHILAYSVFEPRSSKFILNKLEPNPIE |
| | GWKWKLRKLRRSIQNLERRWRAQDNVKLPENQMKKNLRSIEDKVENLYHNLSRKIV |
| | DLAKEKNACIVFEKLEGQGMKQHGRKKSDRLRGLNYKLSLFDYGKIAKLIKYKAEI |
| | EGIPIYRIDSAYTSQNCAKCVLESRRFAQPEEISCLDDFKEGDNLDKRILEGTGLV |
| | EAKIYKKLLKEKKEDFEIEEDIAMFDTKKVIKENKEKTVILDYVYTRRKEIIGTNH |
| | KKNIKGIAKYTGNTKIGYCMKHGQVDADLNASRTIALCKNFDINNPEIWK |
| 61 | MSDESLVSSEDKLAIKIKIVPNAEQAKMLDEMFKKWSSICNRISRGKEDIETLRPD |
| | EGKELQFNSTQLNSATMDVSDLKKAMARQGERLEAEVSKLRGRYETIDASLRDPSR |
| | RHTNPQKPSSFYPSDWDISGRLTPRFHTARHYSTELRKLKAKEDKMLKTINKIKNG |
| | KIVFKPKRITLWPSSVNMAFKGSRLLLKPFANGFEMELPIVISPQKTADGKSQKAS |
| | AEYMRNALLGLAGYSINQLLFGMNRSQKMLANAKKPEKVEKFLEQMKNKDANFDKK |
| | IKALEGKWLLDRKLKESEKSSIAVVRTKFFKSGKVELNEDYLKLLKHMANEILERD |
| | GFVNLNKYPILSRKPMKRYKQKNIDNLKPNMWKYYIQFGYEPIFERKASGKPKNIM |
| | GIDRGLTHLLAVAVFSPDQQKFLFNHLESNPIMHWKWKLRKIRRSIQHMERRIRAE |
| | KNKHIHEAQLKKRLGSIEEKTEQHYHIVSSKIINWAIEYEAAIVLESLSHMKQRGG |
| | KKSVRTRALNYALSLFDYEKVARLITYKARIRGIPVYDVLPGMTSKTCATCLLNGS |
| | QGAYVRGLETTKAAGKATKRKNMKIGKCMVCNSSENSMIDADLNAARVIAICKYKN |
| | LNDPQPAGSRKVFKRF |
| 62 | MLALKLKIMPTEKQAEILDAMFWKWASICSRIAKMKKKVSVKENKKELSKKIPSNS |
| | DIWFSKTQLCQAEVDVGDHKKALKNFEKRQESLLDELKYKVKAINEVINDESKREI |
| | DPNNPSKFRIKDSTKKGNLNSPKFFTLKKWQKILQENEKRIKKKESTIEKLKRGNI |
| | FFNPTKISLHEEEYSINFGSSKLLLNCFYKYNKKSGINSDQLENKFNEFQNGLNII |
| | CSPLQPIRGSSKRSFEFIRNSIINFLMYSLYAKLFGIPRSVKALMKSNKDENKLKL |
| | EEKLKKKKSSFNKTVKEFEKMIGRKLSDNESKILNDESKKFFEIIKSNNKYIPSEE |
| | YLKLLKDISEEIYNSNIDFKPYKYSILIRKPLSKFKSKKLYNLKPTDYKYYLQLSY |
| | EPFSKQLIATKTILGIDRGLKHLLAVSVFDPSQNKFVYNKLIKNPVFKWKKRYHDL |
| | KRSIRNRERRIRALTGVHIHENQLIKKLKSMKNKINVLYHNVSKNIVDLAKKYEST |
| | IVLERLENLKQHGRSKGKRYKKLNYVLSNFDYKKIESLISYKAKKEGVPVSNINPK |
| | YTSKTCAKCLLEVNQLSELKNEYNRDSKNSKIGICNIHGQIDADLNAARVIALCYS |
| | KNLNEPHFK |
| 63 | VINLFGYKFALYPNKTQEELLNKHLGECGWLYNKAIEQNEYYKADSNIEEAQKKFE |
| | LLPDKNSDEAKVLRGNISKDNYVYRTLVKKKKSEINVQIRKAVVLRPAETIRNLAK |
| | VKKKGLSVGRLKFIPIREWDVLPFKQSDQIRLEENYLILEPYGRLKFKMHRPLLGK |
| | PKTFCIKRTATDRWTISFSTEYDDSNMRKNDGGQVGIDVGLKTHLRLSNENPDEDP |
| | RYPNPKIWKRYDRRLTILQRRISKSKKLGKNRTRLRLRLSRLWEKIRNSRADLIQN |
| | ETYEILSENKLIAIEDLNVKGMQEKKDKKGRKGRTRAQEKGLHRSISDAAFSEFRR |
| | VLEYKAKRFGSEVKPVSAIDSSKECHNCGNKKGMPLESRIYECPKCGLKIDRDLNS |
| | AKVILARATGVRPGSNARADTKISATAGASVQTEGTVSEDFRQQMETSDQKPMQGE |
| | GSKEPPMNPEHKSSGRGSKHVNIGCKNKVGLYNEDENSRSTEKQIMDENRSTTEDM |
| | VEIGALHSPVLTT |
| 64 | MIASIDYEAVSQALIVFEFKAKGKDSQYQAIDEAIRSYRFIRNSCLRYWMDNKKVG |
| | KYDLNKYCKVLAKQYPFANKLNSQARQSAAECSWSAISRFYDNCKRKVSGKKGFPK |
| | FKKHARSVEYKTSGWKLSENRKAITFTDKNGIGKLKLKGTYDLHFSQLEDMKRVRL |
| | VRRADGYYVQFCISVDVKVETEPTGKAIGLDVGIKYFLADSSGNTIENPQFYRKAE |
| | KKLNRANRRKSKKYIRGVKPQSKNYHKARCRYARKHLRVSRQRKEYCKRVAYCVIH |
| | SNDVVAYEDLNVKGMVKNRHLAKSISDVAWSTFRHWLEYFAIKYGKLTIPVAPHNT |
| | SQNCSNCDKKVPKSLSTRTHICHHCGYSEDRDVNAAKNILKKALSTVGQTGSLKLG |
| | EIEPLLVLEQSCTRKFDL |
| 65 | LAEENTLHLTLAMSLPLNDLPENRTRSELWRRQWLPQKKLSLLLGVNQSVRKAAAD |
| | CLRWFEPYQELLWWEPTDPDGKKLLDKEGRPIKRTAGHMRVLRKLEEIAPFRGYQL |
| | GSAVKNGLRHKVADLLLSYAKRKLDPQFTDKTSYPSIGDQFPIVWTGAFVCYEQSI |
| | TGQLYLYLPLFPRGSHQEDITNNYDPDRGPALQVFGEKEIARLSRSTSGLLLPLQF |
| | DKWGEATFIRGENNPPTWKATHRRSDKKWLSEVLLREKDFQPKRVELLVRNGRIFV |
| | NVACEIPTKPLLEVENFMGVSFGLEHLVTVVVINRDGNVVHQRQEPARRYEKTYFA |
| | RLERLRRRGGPFSQELETFHYRQVAQIVEEALRFKSVPAVEQVGNIPKGRYNPRLN |
| | LRLSYWPFGKLADLTSYKAVKEGLPKPYSVYSATAKMLCSTCGAANKEGDQPISLK |
| | GPTVYCGNCGTRHNTGENTALNLARRAQELFVKGVVAR |
| 66 | MSQSLLKWHDMAGRDKDASRSLQKSAVEGVLLHLTASHRVALEMLEKSVSQTVAVT |
| | MEAAQQRLVIVLEDDPTKATSRKRVISADLQFTREEFGSLPNWAQKLASTCPEIAT |
| | KYADKHINSIRIAWGVAKESTNGDAVEQKLQWQIRLLDVTMFLQQLVLQLADKALL |
| | EQIPSSIRGGIGQEVAQQVTSHIQLLDSGTVLKAELPTISDRNSELARKQWEDAIQ |
| | TVCTYALPFSRERARILDPGKYAAEDPRGDRLINIDPMWARVLKGPTVKSLPLLFV |
| | SGSSIRIVKLTLPRKHAAGHKHTFTATYLVLPVSREWINSLPGTVQEKVQWWKKPD |
| | VLATQELLVGKGALKKSANTLVIPISAGKKRFFNHILPALQRGFPLQWQRIVGRSY |
| | RRPATHRKWFAQLTIGYTNPSSLPEMALGIHFGMKDILWWALADKQGNILKDGSIP |
| | GNSILDFSLQEKGKIERQQKAGKNVAGKKYGKSLLNATYRVVNGVLEFSKGISAEH |
| | ASQPIGLGLETIRFVDKASGSSPVNARHSNWNYGQLSGIFANKAGPAGFSVTEITL |
| | KKAQRDLSDAEQARVLAIEATKRFASRIKRLATKRKDDTLFV |
| 67 | VEPVEKERFYYRTYTFRLDGQPRTQNLTTQSGWGLLTKAVLDNTKHYWEIVHHARI |
| | ANQPIVFENPVIDEQGNPKLNKLGQPRFWKRPISDIVNQLRALFENQNPYQLGSSL |
| | IQGTYWDVAENLASWYALNKEYLAGTATWGEPSFPEPHPLTEINQWMPLTFSSGKV |
| | VRLLKNASGRYFIGLPILGENNPCYRMRTIEKLIPCDGKGRVTSGSLILFPLVGIY |
| | AQQHRRMTDICESIRTEKGKLAWAQVSIDYVREVDKRRRMRRTRKSQGWIQGPWQE |
| | VFILRLVLAHKAPKLYKPRCFAGISLGPKTLASCVILDQDERVVEKQQWSGSELLS |
| | LIHQGEERLRSLREQSKPTWNAAYRKQLKSLINTQVFTIVTFLRERGAAVRLESIA |
| | RVRKSTPAPPVNFLLSHWAYRQITERLKDLAIRNGMPLTHSNGSYGVRFTCSQCGA |
| | TNQGIKDPTKYKVDIESETFLCSICSHREIAAVNTATNLAKQLLDE |
| 68 | MNDTETSETLTSHRTVCAHLHVVGETGSLPRLVEAALAELITLNGRATQALLSLAK |
| | NGLVLRRDKEENLIAAELTLPCRKNKYADVAAKAGEPILATRINNKGKLVTKKWYG |
| | EGNSYHIVRFTPETGMFTVRVFDRYAFDEELLHLHSEVVFGSDLPKGIKAKTDSLP |
| | ANFLQAVFTSFLELPFQGFPDIVVKPAMKQAAEQLLSYVQLEAGENQQAEYPDTNE |
| | RDPELRLVEWQKSLHELSVRTEPFEFVRARDIDYYAETDRRGNRFVNITPEWTKFA |
| | ESPFARRLPLKIPPEFCILLRRKTEGHAKIPNRIYLGLQIFDGVTPDSTLGVLATA |
| | EDGKLFWWHDHLDEFSNLEGKPEPKLKNKPQLLMVSLEYDREQRFEESVGGDRKIC |
| | LVTLKETRNFRRGWNGRILGIHFQHNPVITWALMDHDAEVLEKGFIEGNAFLGKAL |
| | DKQALNEYLQKGGKWVGDRSFGNKLKGITHTLASLIVRLAREKDAWIALEEISWVQ |
| | KQSADSVANHEIVEQPHHSLTR |
| 69 | MNDTETSETLTSHRTVCAHLHVVGETGSLPRLVEAALAELITLNGRATQALLSLAK |
| | NGLVLRRDKEENLIAAELTLPCRKNKYADVAAKAGEPILATRINNKGKLVTKKWYG |
| | EGNSYHIVRFTPETGMFTVRVFDRYAFDEELLHLHSEVVFGSDLPKGIKAKTDSLP |
| | ANFLQAVFTSFLELPFQGFPDIVVKPAMKQAAEQLLSYVQLEAGENQQAEYPDTNE |
| | RDPELRLVEWQKSLHELSVRTEPFEFVRARDIDYYAETDRRGNRFVNITPEWTKFA |
| | ESPFARRLPLKIPPEFCILLRRKTEGHAKIPNRIYLGLQIFDGVTPDSTLGVLATA |
| | EDGKLFWWHDHLDEFSNLEGKPEPKLKNKPQLLMVSLEYDREQRFEESVGGDRKIC |
| | LVILKETRNFRRGRHGHTRTDRLPAGNTLWRADFATSAEVAAPKWNGRILGIHFQH |
| | NPVITWALMDHDAEVLEKGFIEGNAFLGKALDKQALNEYLQKGGKWVGDRSFGNKL |
| | KGITHTLASLIVRLAREKDAWIALEEISWVQKQSADSVANRRFSMWNYSRLATLIE |
| | WLGTDIATRDCGTAAPLAHKVSDYLTHFTCPECGACRKAGQKKEIADTVRAGDILT |
| | CRKCGFSGPIPDNFIAEFVAKKALERMLKKKPV |
| 70 | MAKRNFGEKSEALYRAVRFEVRPSKEELSILLAVSEVLRMLFNSALAERQQVFTEF |
| | IASLYAELKSASVPEEISEIRKKLREAYKEHSISLFDQINALTARRVEDEAFASVT |
| | RNWQEETLDALDGAYKSFLSLRRKGDYDAHSPRSRDSGFFQKIPGRSGFKIGEGRI |
| | ALSCGAGRKLSFPIPDYQQGRLAETTKLKKFELYRDQPNLAKSGRFWISVVYELPK |
| | PEATTCQSEQVAFVALGASSIGVVSQRGEEVIALWRSDKHWVPKIEAVEERMKRRV |
| | KGSRGWLRLLNSGKRRMHMISSRQHVQDEREIVDYLVRNHGSHFVVTELVVRSKEG |
| | KLADSSKPERGGSLGLNWAAQNTGSLSRLVRQLEEKVKEHGGSVRKHKLTLTEAPP |
| | ARGAENKLWMARKLRESFLKEV |
| 71 | LAKNDEKELLYQSVKFEIYPDESKIRVLTRVSNILVLVWNSALGERRARFELYIAP |
| | LYEELKKFPRKSAESNALRQKIREGYKEHIPTFFDQLKKLLTPMRKEDPALLGSVP |
| | RAYQEETLNTLNGSFVSFMTLRRNNDMDAKPPKGRAEDRFHEISGRSGFKIDGSEF |
| | VLSTKEQKLRFPIPNYQLEKLKEAKQIKKFTLYQSRDRRFWISIAYEIELPDQRPF |
| | NPEEVIYIAFGASSIGVISPEGEKVIDFWRPDKHWKPKIKEVENRMRSCKKGSRAW |
| | KKRAAARRKMYAMTQRQQKLNHREIVASLLRLGFHFVVTEYTVRSKPGKLADGSNP |
| | KRGGAPQGFNWSAQNTGSFGEFILWLKQKVKEQGGTVQTFRLVLGQSERPEKRGRD |
| | NKIEMVRLLREKYLESQTIVV |
| 72 | MAKGKKKEGKPLYRAVRFEIFPTSDQITLFLRVSKNLQQVWNEAWQERQSCYEQFF |
| | GSIYERIGQAKKRAQEAGFSEVWENEAKKGLNKKLRQQEISMQLVSEKESLLQELS |
| | IAFQEHGVTLYDQINGLTARRIIGEFALIPRNWQEETLDSLDGSFKSFLALRKNGD |
| | PDAKPPRQRVSENSFYKIPGRSGFKVSNGQIYLSFGKIGQTLTSVIPEFQLKRLET |
| | AIKLKKFELCRDERDMAKPGRFWISVAYEIPKPEKVPVVSKQITYLAIGASRLGVV |
| | SPKGEFCLNLPRSDYHWKPQINALQERLEGVVKGSRKWKKRMAACTRMFAKLGHQQ |
| | KQHGQYEVVKKLLRHGVHFVVTELKVRSKPGALADASKSDRKGSPTGPNWSAQNTG |
| | NIARLIQKLTDKASEHGGTVIKRNPPLLSLEERQLPDAQRKIFIAKKLREEFLADQ |
| | K |
| 73 | MAKREKKDDVVLRGTKMRIYPTDRQVTLMDMWRRRCISLWNLLLNLETAAYGAKNT |
| | RSKLGWRSIWARVVEENHAKALIVYQHGKCKKDGSFVLKRDGTVKHPPRERFPGDR |
| | KILLGLFDALRHTLDKGAKCKCNVNQPYALTRAWLDETGHGARTADIIAWLKDFKG |
| | ECDCTAISTAAKYCPAPPTAELLTKIKRAAPADDLPVDQAILLDLFGALRGGLKQK |
| | ECDHTHARTVAYFEKHELAGRAEDILAWLIAHGGTCDCKIVEEAANHCPGPRLFIW |
| | EHELAMIMARLKAEPRTEWIGDLPSHAAQTVVKDLVKALQTMLKERAKAAAGDESA |
| | RKTGFPKFKKQAYAAGSVYFPNTTMFFDVAAGRVQLPNGCGSMRCEIPRQLVAELL |
| | ERNLKPGLVIGAQLGLLGGRIWRQGDRWYLSCQWERPQPTLLPKTGRTAGVKIAAS |
| | IVFTTYDNRGQTKEYPMPPADKKLTAVHLVAGKQNSRALEAQKEKEKKLKARKERL |
| | RLGKLEKGHDPNALKPLKRPRVRRSKLFYKSAARLAACEAIERDRRDGFLHRVTNE |
| | IVHKFDAVSVQKMSVAPMMRRQKQKEKQIESKKNEAKKEDNGAAKKPRNLKPVRKL |
| | LRHVAMARGRQFLEYKYNDLRGPGSVLIADRLEPEVQECSRCGTKNPQMKDGRRLL |
| | RCIGVLPDGTDCDAVLPRNRNAARNAEKRLRKHREAHNA |
| 74 | MNEVLPIPAVGEDAADTIMRGSKMRIYPSVRQAATMDLWRRRCIQLWNLLLELEQA |
| | AYSGENRRTQIGWRSIWATVVEDSHAEAVRVAREGKKRKDGTFRKAPSGKEIPPLD |
| | PAMLAKIQRQMNGAVDVDPKTGEVTPAQPRLFMWEHELQKIMARLKQAPRTHWIDD |
| | LPSHAAQSVVKDLIKALQAMLRERKKRASGIGGRDTGFPKFKKNRYAAGSVYFANT |
| | QLRFEAKRGKAGDPDAVRGEFARVKLPNGVGWMECRMPRHINAAHAYAQATLMGGR |
| | IWRQGENWYLSCQWKMPKPAPLPRAGRTAAIKIAAAIPITTVDNRGQTREYAMPPI |
| | DRERIAAHAAAGRAQSRALEARKRRAKKREAYAKKRHAKKLERGIAAKPPGRARIK |
| | LSPGFYAAAAKLAKLEAEDANAREAWLHEITTQIVRNFDVIAVPRMEVAKLMKKPE |
| | PPEEKEEQVKAPWQGKRRSLKAARVMMRRTAMALIQTTLKYKAVDLRGPQAYEEIA |
| | PLDVTAAACSGCGVLKPEWKMARAKGREIMRCQEPLPGGKTCNTVLTYTRNSARVI |
| | GRELAVRLAERQKA |
| 75 | MTTQKTYNFCFYDQRFFELSKEAGEVYSRSLEEFWKIYDETGVWLSKFDLQKHMRN |
| | KLERKLLHSDSFLGAMQQVHANLASWKQAKKVVPDACPPRKPKFLQAILFKKSQIK |
| | YKNGFLRLTLGTEKEFLYLKWDINIPLPIYGSVTYSKTRGWKINLCLETEVEQKNL |
| | SENKYLSIDLGVKRVATIFDGENTITLSGKKFMGLMHYRNKLNGKTQSRLSHKKKG |
| | SNNYKKIQRAKRKTTDRLLNIQKEMLHKYSSFIVNYAIRNDIGNIIIGDNSSTHDS |
| | PNMRGKTNQKISQNPEQKLKNYIKYKFESISGRVDIVPEPYTSRKCPHCKNIKKSS |
| | PKGRTYKCKKCGFIFDRDGVGAINIYNENVSFGQIISPGRIRSLTEPIGMKFHNEI |
| | YFKSYVAA |
| 76 | MSVRSFQARVECDKQTMEHLWRTHKVFNERLPEIIKILFKMKRGECGQNDKQKSLY |
| | KSISQSILEANAQNADYLLNSVSIKGWKPGTAKKYRNASFTWADDAAKLSSQGIHV |
| | YDKKQVLGDLPGMMSQMVCRQSVEAISGHIELTKKWEKEHNEWLKEKEKWESEDEH |
| | KKYLDLREKFEQFEQSIGGKITKRRGRWHLYLKWLSDNPDFAAWRGNKAVINPLSE |
| | KAQIRINKAKPNKKNSVERDEFFKANPEMKALDNLHGYYERNFVRRRKTKKNPDGF |
| | DHKPTFTLPHPTIHPRWFVFNKPKTNPEGYRKLILPKKAGDLGSLEMRLLTGEKNK |
| | GNYPDDWISVKFKADPRLSLIRPVKGRRVVRKGKEQGQTKETDSYEFFDKHLKKWR |
| | PAKLSGVKLIFPDKTPKAAYLYFTCDIPDEPLTETAKKIQWLETGDVTKKGKKRKK |
| | KVLPHGLVSCAVDLSMRRGTTGFATLCRYENGKIHILRSRNLWVGYKEGKGCHPYR |
| | WTEGPDLGHIAKHKREIRILRSKRGKPVKGEESHIDLQKHIDYMGEDRFKKAARTI |
| | VNFALNTENAASKNGFYPRADVLLLENLEGLIPDAEKERGINRALAGWNRRHLVER |
| | VIEMAKDAGFKRRVFEIPPYGTSQVCSKCGALGRRYSIIRENNRREIRFGYVEKLF |
| | ACPNCGYCANADHNASVNLNRRFLIEDSFKSYYDWKRLSEKKQKEEIETIESKLMD |
| | KLCAMHKISRGSISK |
| 77 | MHLWRTHCVFNQRLPALLKRLFAMRRGEVGGNEAQRQVYQRVAQFVLARDAKDSVD |
| | LLNAVSLRKRSANSAFKKKATISCNGQAREVTGEEVFAEAVALASKGVFAYDKDDM |
| | RAGLPDSLFQPLTRDAVACMRSHEELVATWKKEYREWRDRKSEWEAEPEHALYLNL |
| | RPKFEEGEAARGGRFRKRAERDHAYLDWLEANPQLAAWRRKAPPAVVPIDEAGKRR |
| | IARAKAWKQASVRAEEFWKRNPELHALHKIHVQYLREFVRPRRTRRNKRREGFKQR |
| | PTFTMPDPVRHPRWCLFNAPQTSPQGYRLLRLPQSRRTVGSVELRLLTGPSDGAGF |
| | PDAWVNVRFKADPRLAQLRPVKVPRTVTRGKNKGAKVEADGFRYYDDQLLIERDAQ |
| | VSGVKLLFRDIRMAPFADKPIEDRLLSATPYLVFAVEIKDEARTERAKAIRFDETS |
| | ELTKSGKKRKTLPAGLVSVAVDLDTRGVGFLTRAVIGVPEIQQTHHGVRLLQSRYV |
| | AVGQVEARASGEAEWSPGPDLAHIARHKREIRRLRQLRGKPVKGERSHVRLQAHID |
| | RMGEDRFKKAARKIVNEALRGSNPAAGDPYTRADVLLYESLETLLPDAERERGINR |
| | ALLRWNRAKLIEHLKRMCDDAGIRHFPVSPFGTSQVCSKCGALGRRYSLARENGRA |
| | VIRFGWVERLFACPNPECPGRRPDRPDRPFTCNSDHNASVNLHRVFALGDQAVAAF |
| | RALAPRDSPARTLAVKRVEDTLRPQLMRVHKLADAGVDSPF |
| 78 | MATLVYRYGVRAHGSARQQDAVVSDPAMLEQLRLGHELRNALVGVQHRYEDGKRAV |
| | WSGFASVAAADHRVTTGETAVAELEKQARAEHSADRTAATRQGTAESLKAARAAVK |
| | QARADRKAAMAAVAEQAKPKIQALGDDRDAEIKDLYRRFCQDGVLLPRCGRCAGDL |
| | RSDGDCTDCGAAHEPRKLYWATYNAIREDHQTAVKLVEAKRKAGQPARLRFRRWTG |
| | DGTLTVQLQRMHGPACRCVTCAEKLTRRARKTDPQAPAVAADPAYPPTDPPRDPAL |
| | LASGQGKWRNVLQLGTWIPPGEWSAMSRAERRRVGRSHIGWQLGGGRQLTLPVQLH |
| | RQMPADADVAMAQLTRVRVGGRHRMSVALTAKLPDPPQVQGLPPVALHLGWRQRPD |
| | GSLRVATWACPQPLDLPPAVADVVVSHGGRWGEVIMPARWLADAEVPPRLLGRRDK |
| | AMEPVLEALADWLEAHTEACTARMTPALVRRWRSQGRLAGLTNRWRGQPPTGSAEI |
| | LTYLEAWRIQDKLLWERESHLRRRLAARRDDAWRRVASWLARHAGVLVVDDADIAE |
| | LRRRDDPADTDPTMPASAAQAARARAALAAPGRLRHLATITATRDGLGVHTVASAG |
| | LTRLHRKCGHQAQPDPRYAASAVVTCPGCGNGYDQDYNAAMLMLDRQQQP |
| 79 | MSRVELHRAYKFRLYPTPAQVAELAEWERQLRRLYNLAHSQRLAAMQRHVRPKSPG |
| | VLKSECLSCGAVAVAEIGTDGKAKKTVKHAVGCSVLECRSCGGSPDAEGRTAHTAA |
| | CSFVDYYRQGREMTQLLEEDDQLARVVCSARQETLRDLEKAWQRWHKMPGFGKPHF |
| | KKRIDSCRIYFSTPKSWAVDLGYLSFTGVASSVGRIKIRQDRVWPGDAKFSSCHVV |
| | RDVDEWYAVFPLTFTKEIEKPKGGAVGINRGAVHAIADSTGRVVDSPKFYARSLGV |
| | IRHRARLLDRKVPFGRAVKPSPTKYHGLPKADIDAAAARVNASPGRLVYEARARGS |
| | IAAAEAHLAALVLPAPRQTSQLPSEGRNRERARRFLALAHQRVRRQREWFLHNESA |
| | HYAQSYTKIAIEDWSTKEMTSSEPRDAEEMKRVTRARNRSILDVGWYELGRQIAYK |
| | SEATGAEFAKVDPGLRETETHVPEAIVRERDVDVSGMLRGEAGISGTCSRCGGLLR |
| | ASASGHADAECEVCLHVEVGDVNAAVNVLKRAMFPGAAPPSKEKAKVTIGIKGRKK |
| | KRAA |
| 80 | MSRVELHRAYKFRLYPTPVQVAELSEWERQLRRLYNLGHEQRLLTLTRHLRPKSPG |
| | VLKGECLSCDSTQVQEVGADGRPKTTVRHAEQCPTLACRSCGALRDAEGRTAHTVA |
| | CAFVDYYRQGREMTELLAADDQLARVVCSARQEVLRDLDKAWQRWRKMPGFGKPRF |
| | KRRTDSCRIYFSTPKAWKLEGGHLSFTGAATTVGAIKMRQDRNWPASVQFSSCHVV |
| | RDVDEWYAVFPLTFVAEVARPKGGAVGINRGAVHAIADSTGRVVDSPRYYARALGV |
| | IRHRARLFDRKVPSGHAVKPSPTKYRGLSAIEVDRVARATGFTPGRVVTEALNRGG |
| | VAYAECALAAIAVLGHGPERPLTSDGRNREKARKFLALAHQRVRRQREWFLHNESA |
| | HYARTYSKIAIEDWSTKEMTASEPQGEETRRVTRSRNRSILDVGWYELGRQLAYKT |
| | EATGAEFAQVDPGLKETETNVPKAIADARDVDVSGMLRGEAGISGTCSKCGGLLRA |
| | PASGHADAECEICLNVEVGDVNAAVNVLKRAMFPGDAPPASGEKPKVSIGIKGRQK |
| | KKKAA |
| 81 | MEAIATGMSPERRVELGILPGSVELKRAYKFRLYPMKVQQAELSEWERQLRRLYNL |
| | AHEQRLAALLRYRDWDFQKGACPSCRVAVPGVHTAACDHVDYFRQAREMTQLLEVD |
| | AQLSRVICCARQEVLRDLDKAWQRWRKKLGGRPRFKRRTDSCRIYLSTPKHWEIAG |
| | RYLRLSGLASSVGEIRIEQDRAFPEGALLSSCSIVRDVDEWYACLPLTFTQPIERA |
| | PHRSVGLNRGVVHALADSDGRVVDSPKFFERALATVQKRSRDLARKVSGSRNAHKA |
| | RIKLAKAHQRVRRQRAAFLHQESAYYSKGFDLVALEDMSVRKMTATAGEAPEMGRG |
| | AQRDLNRGILDVGWYELARQIDYKRLAHGGELLRVDPGQTTPLACVTEEQPARGIS |
| | SACAVCGIPLARPASGNARMRCTACGSSQVGDVNAAENVLTRALSSAPSGPKSPKA |
| | SIKIKGRQKRLGTPANRAGEASGGDPPVRGPVEGGTLAYVVEPVSESQSDT |
| 82 | MTVRTYKYRAYPTPEQAEALTSWLRFASQLYNAALEHRKNAWGRHDAHGRGFRFWD |
| | GDAAPRKKSDPPGRWVYRGGGGAHISKNDQGKLLTEFRREHAELLPPGMPALVQHE |
| | VLARLERSMAAFFQRATKGQKAGYPRWRSEHRYDSLTFGLTSPSKERFDPETGESL |
| | GRGKTVGAGTYHNGDLRLTGLGELRILEHRRIPMGAIPKSVIVRRSGKRWFVSIAM |
| | EMPSVEPAASGRPAVGLDMGVVTWGTAFTADTSAAAALVADLRRMATDPSDCRRLE |
| | ELEREAAQLSEVLAHCRARGLDPARPRRCPKELTKLYRRSLHRLGELDRACARIRR |
| | RLQAAHDIAEPVPDEAGSAVLIEGSNAGMRHARRVARTQRRVARRTRAGHAHSNRR |
| | KKAVQAYARAKERERSARGDHRHKVSRALVRQFEEISVEALDIKQLTVAPEHNPDP |
| | QPDLPAHVQRRRNRGELDAAWGAFFAALDYKAADAGGRVARKPAPHTTQECARCGT |
| | LVPKPISLRVHRCPACGYTAPRTVNSARNVLQRPLEEPGRAGPSGANGRGVPHAVA |
| 83 | MNCRYRYRIYPTPGQRQSLARLFGCVRVVWNDALFLCRQSEKLPKNSELQKLCITQ |
| | AKKTEARGWLGQVSAIPLQQSVADLGVAFKNFFQSRSGKRKGKKVNPPRVKRRNNR |
| | QGARFTRGGFKVKTSKVYLARIGDIKIKWSRPLPSEPSSVTVIKDCAGQYFLSFVV |
| | EVKPEIKPPKNPSIGIDLGLKTFASCSNGEKIDSPDYSRLYRKLKRCQRRLAKRQR |
| | GSKRRERMRVKVAKLNAQIRDKRKDFLHKLSTKVVNENQVIALEDLNVGGMLKNRK |
| | LSRAISQAGWYEFRSLCEGKAEKHNRDFRVISRWEPTSQVCSECGYRWGKIDLSVR |
| | SIVCINCGVEHDRDDNASVNIEQAGLKVGVGHTHDSKRTGSACKTSNGAVCVEPST |
| | HREYVQLTLFDW |
| 84 | MKSRWTFRCYPTPEQEQHLARTFGCVRFVWNWALRARTDAFRAGERIGYPATDKAL |
| | TLLKQQPETVWLNEVSSVCLQQALRDLQVAFSNFFDKRAAHPSFKRKEARQSANYT |
| | ERGFSFDHERRILKLAKIGAIKVKWSRKAIPHPSSIRLIRTASGKYFVSLVVETQP |
| | APMPETGESVGVDFGVARLATLSNGERISNPKHGAKWQRRLAFYQKRLARATKGSK |
| | RRMRIKRHVARIHEKIGNSRSDTLHKLSTDLVTRFDLICVEDLNLRGMVKNHSLAR |
| | SLHDASIGSAIRMIEEKAERYGKNVVKIDRWFPSSKTCSDCGHIVEQLPLNVREWT |
| | CPECGTTHDRDANAAANILAVGQTVSAHGGTVRRSRAKASERKSQRSANRQGVNRA |
| 85 | KEPLNIGKTAKAVFKEIDPTSLNRAANYDASIELNCKECKFKPFKNVKRYEFNFYN |
| | NWYRCNPNSCLQSTYKAQVRKVEIGYEKLKNEILTQMQYYPWFGRLYQNFFHDERD |
| | KMTSLDEIQVIGVQNKVFFNTVEKAWREIIKKRFKDNKETMETIPELKHAAGHGKR |
| | KLSNKSLLRRRFAFVQKSFKFVDNSDVSYRSFSNNIACVLPSRIGVDLGGVISRNP |
| | KREYIPQEISFNAFWKQHEGLKKGRNIEIQSVQYKGETVKRIEADTGEDKAWGKNR |
| | QRRFTSLILKLVPKQGGKKVWKYPEKRNEGNYEYFPIPIEFILDSGETSIRFGGDE |
| | GEAGKQKHLVIPFNDSKATPLASQQTLLENSRFNAEVKSCIGLAIYANYFYGYARN |
| | YVISSIYHKNSKNGQAITAIYLESIAHNYVKAIERQLQNLLLNLRDFSFMESHKKE |
| | LKKYFGGDLEGTGGAQKRREKEEKIEKEIEQSYLPRLIRLSLTKMVTKQVEM |
| 86 | ELIVNENKDPLNIGKTAKAVFKEIDPTSINRAANYDASIELACKECKFKPFNNTKR |
| | HDFSFYSNWHRCSPNSCLQSTYRAKIRKTEIGYEKLKNEILNQMQYYPWFGRLYQN |
| | FFNDQRDKMTSLDEIQVTGVQNKIFFNTVEKAWREIIKKRFRDNKETMRTIPDLKN |
| | KSGHGSRKLSNKSLLRRRFAFAQKSFKLVDNSDVSYRAFSNNVACVLPSKIGVDIG |
| | GIINKDLKREYIPQEITFNVFWKQHDGLKKGRNIEIHSVQYKGEIVKRIEADTGED |
| | KAWGKNRQRRFTSLILKITPKQGGKKIWKFPEKKNASDYEYFPIPIEFILDNGDAS |
| | IKFGGEEGEVGKQKHLLIPFNDSKATPLSSKQMLLETSRFNAEVKSTIGLALYANY |
| | FVSYARNYVIKSTYHKNSKKGQIVTEIYLESISQNFVRAIQRQLQSLMLNLKDWGF |
| | MQTHKKELKKYFGSDLEGSKGGQKRREKEEKIEKEIEASYLPRLIRLSLTKSVTKA |
| | EEM |
| 87 | PEEKTSKLKPNSINLAANYDANEKFNCKECKFHPFKNKKRYEFNFYNNLHGCKSCT |
| | KSTNNPAVKRIEIGYQKLKFEIKNQMEAYPWFGRLRINFYSDEKRKMSELNEMQVT |
| | GVKNKIFFDAIECAWREILKKRFRESKETLITIPKLKNKAGHGARKHRNKKLLIRR |
| | RAFMKKNFHFLDNDSISYRSFANNIACVLPSKVGVDIGGIISPDVGKDIKPVDISL |
| | NLMWASKEGIKSGRKVEIYSTQYDGNMVKKIEAETGEDKSWGKNRKRRQTSLLLSI |
| | PKPSKQVQEFDFKEWPRYKDIEKKVQWRGFPIKIIFDSNHNSIEFGTYQGGKQKVL |
| | PIPFNDSKTTPLGSKMNKLEKLRFNSKIKSRLGSAIAANKFLEAARTYCVDSLYHE |
| | VSSANAIGKGKIFIEYYLEILSQNYIEAAQKQLQRFIESIEQWFVADPFQGRLKQY |
| | FKDDLKRAKCFLCANREVQTTCYAAVKLHKSCAEKVKDKNKELAIKERNNKEDAVI |
| | KEVEASNYPRVIRLKLTKTITNKAM |
| 88 | SESENKIIEQYYAFLYSFRDKYEKPEFKNRGDIKRKLQNKWEDFLKEQNLKNDKKL |
| | SNYIFSNRNFRRSYDREEENEEGIDEKKSKPKRINCFEKEKNLKDQYDKDAINASA |
| | NKDGAQKWGCFECIFFPMYKIESGDPNKRIIINKTRFKLFDFYLNLKGCKSCLRST |
| | YHPYRSNVYIESNYDKLKREIGNFLQQKNIFQRMRKAKVSEGKYLTNLDEYRLSCV |
| | AMHFKNRWLFFDSIQKVLRETIKQRLKQMRESYDEQAKTKRSKGHGRAKYEDQVRM |
| | IRRRAYSAQAHKLLDNGYITLFDYDDKEINKVCLTAINQEGFDIGGYLNSDIDNVM |
| | PPIEISFHLKWKYNEPILNIESPFSKAKISDYLRKIREDLNLERGKEGKARSKKNV |
| | RRKVLASKGEDGYKKIFTDFFSKWKEELEGNAMERVLSQSSGDIQWSKKKRIHYTT |
| | LVLNINLLDKKGVGNLKYYEIAEKTKILSFDKNENKFWPITIQVLLDGYEIGTEYD |
| | EIKQLNEKTSKQFTIYDPNTKIIKIPFTDSKAVPLGMLGINIATLKTVKKTERDIK |
| | VSKIFKGGLNSKIVSKIGKGIYAGYFPTVDKEILEEVEEDTLDNEFSSKSQRNIFL |
| | KSIIKNYDKMLKEQLFDFYSFLVRNDLGVRFLTDRELQNIEDESFNLEKRFFETDR |
| | DRIARWFDNTNTDDGKEKFKKLANEIVDSYKPRLIRLPVVRVIKRIQPVKQREM |
| 89 | KYSTRDFSELNEIQVTACKQDEFFKVIQNAWREIIKKRFLENRENFIEKKIFKNKK |
| | GRGKRQESDKTIQRNRASVMKNFQLIENEKIILRAPSGHVACVFPVKVGLDIGGFK |
| | TDDLEKNIFPPRTITINVFWKNRDRQRKGRKLEVWGIKARTKLIEKVHKWDKLEEV |
| | KKKRLKSLEQKQEKSLDNWSEVNNDSFYKVQIDELQEKIDKSLKGRTMNKILDNKA |
| | KESKEAEGLYIEWEKDFEGEMLRRIEASTGGEEKWGKRRQRRHTSLLLDIKNNSRG |
| | SKEIINFYSYAKQGKKEKKIEFFPFPLTITLDAEEESPLNIKSIPIEDKNATSKYF |
| | SIPFTETRATPLSILGDRVQKFKTKNISGAIKRNLGSSISSCKIVQNAETSAKSIL |
| | SLPNVKEDNNMEIFINTMSKNYFRAMMKQMESFIFEMEPKTLIDPYKEKAIKWFEV |
| | AASSRAKRKLKKLSKADIKKSELLLSNTEEFEKEKQEKLEALEKEIEEFYLPRIVR |
| | LQLTKTILETPVM |
| 90 | KKLQLLGHKILLKEYDPNAVNAAANFETSTAELCGQCKMKPFKNKRRFQYTFGKNY |
| | HGCLSCIQNVYYAKKRIVQIAKEELKHQLTDSIASIPYKYTSLFSNTNSIDELYIL |
| | KQERAAFFSNTNSIDELYITGIENNIAFKVISAIWDEIIKKRRQRYAESLTDTGTV |
| | KANRGHGGTAYKSNTRQEKIRALQKQTLHMVTNPYISLARYKNNYIVATLPRTIGM |
| | HIGAIKDRDPQKKLSDYAINFNVFWSDDRQLIELSTVQYTGDMVRKIEAETGENNK |
| | WGENMKRTKTSLLLEILTKKTTDELTFKDWAFSTKKEIDSVTKKTYQGFPIGIIFE |
| | GNESSVKFGSQNYFPLPFDAKITPPTAEGFRLDWLRKGSFSSQMKTSYGLAIYSNK |
| | VTNAIPAYVIKNMFYKIARAENGKQIKAKFLKKYLDIAGNNYVPFIIMQHYRVLDT |
| | FEEMPISQPKVIRLSLTKTQHIIIKKDKTDSKM |
| 91 | NTSNLINLGKKAINISANYDANLEVGCKNCKFLSSNGNFPRQTNVKEGCHSCEKST |
| | YEPSIYLVKIGERKAKYDVLDSLKKFTFQSLKYQSKKSMKSRNKKPKELKEFVIFA |
| | NKNKAFDVIQKSYNHLILQIKKEINRMNSKKRKKNHKRRLFRDREKQLNKLRLIES |
| | SNLFLPRENKGNNHVFTYVAIHSVGRDIGVIGSYDEKLNFETELTYQLYFNDDKRL |
| | LYAYKPKQNKIIKIKEKLWNLRKEKEPLDLEYEKPLNKSITFSIKNDNLFKVSKDL |
| | MLRRAKFNIQGKEKLSKEERKINRDLIKIKGLVNSMSYGRFDELKKEKNIWSPHIY |
| | REVRQKEIKPCLIKNGDRIEIFEQLKKKMERLRRFREKRQKKISKDLIFAERIAYN |
| | FHTKSIKNTSNKINIDQEAKRGKASYMRKRIGYETFKNKYCEQCLSKGNVYRNVQK |
| | GCSCFENPFDWIKKGDENLLPKKNEDLRVKGAFRDEALEKQIVKIAFNIAKGYEDF |
| | YDNLGESTEKDLKLKFKVGTTINEQESLKL |
| 92 | TSNPIKLGKKAINISANYDSNLQIGCKNCKFLSYNGNFPRQTNVKEGCHSCEKSTY |
| | EPPVYTVRIGERRSKYDVLDSLKKFIFLSLKYRQSKKMKTRSKGIRGLEEFVISAN |
| | LKKAMDVIQKSYRHLILNIKNEIVRMNGKKRNKNHKRLLFRDREKQLNKLRLIEGS |
| | SFFKPPTVKGDNSIFTCVAIHNIGRDIGIAGDYFDKLEPKIELTYQLYYEYNPKKE |
| | SEINKRLLYAYKPKQNKIIEIKEKLWNLRKEKSPLDLEYEKPLTKSITFLVKRDGV |
| | FRISKDLMLRKAKFIIQGKEKLSKEERKINRDLIKIKSNIISLTYGRFDELKKDKT |
| | IWSPHIFRDVKQGKITPCIERKGDRMDIFQQLRKKSERLRENRKKRQKKISKDLIF |
| | AERIAYNFHTKSIKNTSNLINIKHEAKRGKASYMRKRIGNETFRIKYCEQCFPKNN |
| | VYKNVQKGCSCFEDPFEYIKKGNEDLIPNKNQDLKAKGAFRDDALEKQIIKVAFNI |
| | AKGYEDFYENLKKTTEKDIRLKFKVGTIISEEM |
| 93 | NNSINLSKKAINISANYDANLQVRCKNCKFLSSNGNFPRQTDVKEGCHSCEKSTYE |
| | PPVYDVKIGEIKAKYEVLDSLKKFTFQSLKYQLSKSMKFRSKKIKELKEFVIFAKE |
| | SKALNVINRSYKHLILNIKNDINRMNSKKRIKNHKGRLFLDRQKQLSKLKLIEGSS |
| | FFVPAKNVGNKSVFTCVAIHSIGRDIGIAGLYDSFTKPVNEITYQIFFSGERRLLY |
| | AYKPKQLKILSIKENLWSLKNEKKPLDLLYEKPLGKNLNFNVKGGDLFRVSKDLMI |
| | RNAKFNVHGRQRLSDEERLINRNFIKIKGEVVSLSYGRFEELKKDRKLWSPHIFKD |
| | VRQNKIKPCLVMQGQRIDIFEQLKRKLELLKKIRKSRQKKLSKDLIFGERIAYNFH |
| | TKSIKNTSNKINIDSDAKRGRASYMRKRIGNETFKLKYCDVCFPKANVYRRVQNGC |
| | SCSENPYNYIKKGDKDLLPKKDEGLAIKGAFRDEKLNKQIIKVAFNIAKGYEDFYD |
| | DLKKRTEKDVDLKFKIGTTVLDQKPMEIFDGIVITWL |
| 94 | LLTTVVETNNLAKKAINVAANFDANIDRQYYRCTPNLCRFIAQSPRETKEKDAGCS |
| | SCTQSTYDPKVYVIKIGKLLAKYEILKSLKRFLFMNRYFKQKKTERAQQKQKIGTE |
| | LNEMSIFAKATNAMEVIKRATKHCTYDIIPETKSLQMLKRRRHRVKVRSLLKILKE |
| | RRMKIKKIPNTFIEIPKQAKKNKSDYYVAAALKSCGIDVGLCGAYEKNAEVEAEYT |
| | YQLYYEYKGNSSTKRILYCYNNPQKNIREFWEAFYIQGSKSHVNTPGTIRLKMEKF |
| | LSPITIESEALDFRVWNSDLKIRNGQYGFIKKRSLGKEAREIKKGMGDIKRKIGNL |
| | TYGKSPSELKSIHVYRTERENPKKPRAARKKEDNFMEIFEMQRKKDYEVNKKRRKE |
| | ATDAAKIMDFAEEPIRHYHTNNLKAVRRIDMNEQVERKKTSVFLKRIMQNGYRGNY |
| | CRKCIKAPEGSNRDENVLEKNEGCLDCIGSEFIWKKSSKEKKGLWHTNRLLRRIRL |
| | QCFTTAKAYENFYNDLFEKKESSLDIIKLKVSITTKSM |
| 95 | ASTMNLAKQAINFAANYDSNLEIGCKGCKFMSTWSKKSNPKFYPRQNNQANKCHSC |
| | TYSTGEPEVPIIEIGERAAKYKIFTALKKFVFMSVAYKERRRQRFKSKKPKELKEL |
| | AICSNREKAMEVIQKSVVHCYGDVKQEIPRIRKIKVLKNHKGRLFYKQKRSKIKIA |
| | KLEKGSFFKTFIPKVHNNGCHSCHEASLNKPILVTTALNTIGADIGLINDYSTIAP |
| | TETDISWQVYYEFIPNGDSEAVKKRLLYFYKPKGALIKSIRDKYFKKGHENAVNTG |
| | FFKYQGKIVKGPIKFVNNELDFARKPDLKSMKIKRAGFAIPSAKRLSKEDREINRE |
| | SIKIKNKIYSLSYGRKKTLSDKDIIKHLYRPVRQKGVKPLEYRKAPDGFLEFFYSL |
| | KRKERRLRKQKEKRQKDMSEIIDAADEFAWHRHTGSIKKTTNHINFKSEVKRGKVP |
| | IMKKRIANDSFNTRHCGKCVKQGNAINKYYIEKQKNCFDCNSIEFKWEKAALEKKG |
| | AFKLNKRLQYIVKACFNVAKAYESFYEDFRKGEEESLDLKFKIGTTTTLKQYPQNK |
| | ARAM |
| 96 | HSHNLMLTKLGKQAINFAANYDANLEIGCKNCKFLSYSPKQANPKKYPRQTDVHED |
| | GNIACHSCMQSTKEPPVYIVPIGERKSKYEILTSLNKFTFLALKYKEKKRQAFRAK |
| | KPKELQELAIAFNKEKAIKVIDKSIQHLILNIKPEIARIQRQKRLKNRKGKLLYLH |
| | KRYAIKMGLIKNGKYFKVGSPKKDGKKLLVLCALNTIGRDIGIIGNIEENNRSETE |
| | ITYQLYFDCLDANPNELRIKEIEYNRLKSYERKIKRLVYAYKPKQTKILEIRSKFF |
| | SKGHENKVNTGSFNFENPLNKSISIKVKNSAFDFKIGAPFIMLRNGKFHIPTKKRL |
| | SKEEREINRTLSKIKGRVFRLTYGRNISEQGSKSLHIYRKERQHPKLSLEIRKQPD |
| | SFIDEFEKLRLKQNFISKLKKQRQKKLADLLQFADRIAYNYHTSSLEKTSNFINYK |
| | PEVKRGRTSYIKKRIGNEGFEKLYCETCIKSNDKENAYAVEKEELCFVCKAKPFTW |
| | KKTNKDKLGIFKYPSRIKDFIRAAFTVAKSYNDFYENLKKKDLKNEIFLKFKIGLI |
| | LSHEKKNHISIAKSVAEDERISGKSIKNILNKSIKLEKNCYSCFFHKEDM |
| 97 | SLERVIDKRNLAKKAINIAANFDANINKGFYRCETNQCMFIAQKPRKTNNTGCSSC |
| | LQSTYDPVIYVVKVGEMLAKYEILKSLKRFVFMNRSFKQKKTEKAKQKERIGGELN |
| | EMSIFANAALAMGVIKRAIRHCHVDIRPEINRLSELKKTKHRVAAKSLVKIVKQRK |
| | TKWKGIPNSFIQIPQKARNKDADFYVASALKSGGIDIGLCGTYDKKPHADPRWTYQ |
| | LYFDTEDESEKRLLYCYNDPQAKIRDFWKTFYERGNPSMVNSPGTIEFRMEGFFEK |
| | MTPISIESKDFDFRVWNKDLLIRRGLYEIKKRKNLNRKAREIKKAMGSVKRVLANM |
| | TYGKSPTDKKSIPVYRVEREKPKKPRAVRKEENELADKLENYRREDFLIRNRRKRE |
| | ATEIAKIIDAAEPPIRHYHTNHLRAVKRIDLSKPVARKNTSVFLKRIMQNGYRGNY |
| | CKKCIKGNIDPNKDECRLEDIKKCICCEGTQNIWAKKEKLYTGRINVLNKRIKQMK |
| | LECFNVAKAYENFYDNLAALKEGDLKVLKLKVSIPALNPEASDPEEDM |
| 98 | NASINLGKRAINLSANYDSNLVIGCKNCKFLSFNGNFPRQTNVREGCHSCDKSTYA |
| | PEVYIVKIGERKAKYDVLDSLKKFTFQSLKYQIKKSMRERSKKPKELLEFVIFANK |
| | DKAFNVIQKSYEHLILNIKQEINRMNGKKRIKNHKKRLFKDREKQLNKLRLIGSSS |
| | LFFPRENKGDKDLFTYVAIHSVGRDIGVAGSYESHIEPISDLTYQLFINNEKRLLY |
| | AYKPKQNKIIELKENLWNLKKEKKPLDLEFTKPLEKSITFSVKNDKLFKVSKDLML |
| | RQAKFNIQGKEKLSKEERQINRDFSKIKSNVISLSYGRFEELKKEKNIWSPHIYRE |
| | VKQKEIKPCIVRKGDRIELFEQLKRKMDKLKKFRKERQKKISKDLNFAERIAYNFH |
| | TKSIKNTSNKINIDQEAKRGKASYMRKRIGNESFRKKYCEQCFSVGNVYHNVQNGC |
| | SCFDNPIELIKKGDEGLIPKGKEDRKYKGALRDDNLQMQIIRVAFNIAKGYEDFYN |
| | NLKEKTEKDLKLKFKIGTTISTQESNNKEM |
| 99 | SNLIKLGKQAINFAANYDANLEVGCKNCKFLSSTNKYPRQTNVHLDNKMACRSCNQ |
| | STMEPAIYIVRIGEKKAKYDIYNSLTKFNFQSLKYKAKRSQRFKPKQPKELQELSI |
| | AVRKEKALDIIQKSIDHLIQDIRPEIPRIKQQKRYKNHVGKLFYLQKRRKNKLNLI |
| | GKGSFFKVFSPKEKKNELLVICALTNIGRDIGLIGNYNTIINPLFEVTYQLYYDYI |
| | PKKNNKNVQRRLLYAYKSKNEKILKLKEAFFKRGHENAVNLGSFSYEKPLEKSLTL |
| | KIKNDKDDFQVSPSLRIRTGRFFVPSKRNLSRQEREINRRLVKIKSKIKNMTYGKF |
| | ETARDKQSVHIFRLERQKEKLPLQFRKDEKEFMEEFQKLKRRTNSLKKLRKSRQKK |
| | LADLLQLSEKVVYNNHTGTLKKTSNFLNFSSSVKRGKTAYIKELLGQEGFETLYCS |
| | NCINKGQKTRYNIETKEKCFSCKDVPFVWKKKSTDKDRKGAFLFPAKLKDVIKATF |
| | TVAKAYEDFYDNLKSIDEKKPYIKFKIGLILAHVRHEHKARAKEEAGQKNIYNKPI |
| | KIDKNCKECFFFKEEAM |
| 100 | NTTRKKFRKRTGFPQSDNIKLAYCSAIVRAANLDADIQKKHNQCNPNLCVGIKSNE |
| | QSRKYEHSDRQALLCYACNQSTGAPKVDYIQIGEIGAKYKILQMVNAYDFLSLAYN |
| | LTKLRNGKSRGHQRMSQLDEVVIVADYEKATEVIKRSINHLLDDIRGQLSKLKKRT |
| | QNEHITEHKQSKIRRKLRKLSRLLKRRRWKWGTIPNPYLKNWVFTKKDPELVTVAL |
| | LHKLGRDIGLVNRSKRRSKQKLLPKVGFQLYYKWESPSLNNIKKSKAKKLPKRLLI |
| | PYKNVKLFDNKQKLENAIKSLLESYQKTIKVEFDQFFQNRTEEIIAEEQQTLERGL |
| | LKQLEKKKNEFASQKKALKEEKKKIKEPRKAKLLMEESRSLGFLMANVSYALFNTT |
| | IEDLYKKSNVVSGCIPQEPVVVFPADIQNKGSLAKILFAPKDGFRIKFSGQHLTIR |
| | TAKFKIRGKEIKILTKTKREILKNIEKLRRVWYREQHYKLKLFGKEVSAKPRFLDK |
| | RKTSIERRDPNKLADQTDDRQAELRNKEYELRHKQHKMAERLDNIDTNAQNLQTLS |
| | FWVGEADKPPKLDEKDARGFGVRTCISAWKWFMEDLLKKQEEDPLLKLKLSIM |
| 101 | PKKPKFQKRTGFPQPDNLRKEYCLAIVRAANLDADFEKKCTKCEGIKTNKKGNIVK |
| | GRTYNSADKDNLLCYACNISTGAPAVDYVFVGALEAKYKILQMVKAYDFHSLAYNL |
| | AKLWKGRGRGHQRMGGLNEVVIVSNNEKALDVIEKSLNHFHDEIRGELSRLKAKFQ |
| | NEHLHVHKESKLRRKLRKISRLLKRRRWKWDVIPNSYLRNFTFTKTRPDFISVALL |
| | HRVGRDIGLVTKTKIPKPTDLLPQFGFQIYYTWDEPKLNKLKKSRLRSEPKRLLVP |
| | YKKIELYKNKSVLEEAIRHLAEVYTEDLTICFKDFFETQKRKFVSKEKESLKRELL |
| | KELTKLKKDFSERKTALKRDRKEIKEPKKAKLLMEESRSLGFLAANTSYALFNLIA |
| | ADLYTKSKKACSTKLPRQLSTILPLEIKEHKSTTSLAIKPEEGFKIRFSNTHLSIR |
| | TPKFKMKGADIKALTKRKREILKNATKLEKSWYGLKHYKLKLYGKEVAAKPRFLDK |
| | RNPSIDRRDPKELMEQIENRRNEVKDLEYEIRKGQHQMAKRLDNVDTNAQNLQTKS |
| | FWVGEADKPPELDSMEAKKLGLRTCISAWKWFMKDLVLLQEKSPNLKLKLSLTEM |
| 102 | KFSKRQEGFLIPDNIDLYKCLAIVRSANLDADVQGHKSCYGVKKNGTYRVKQNGKK |
| | GVKEKGRKYVFDLIAFKGNIEKIPHEAIEEKDQGRVIVLGKFNYKLILNIEKNHND |
| | RASLEIKNKIKKLVQISSLETGEFLSDLLSGKIGIDEVYGIIEPDVFSGKELVCKA |
| | CQQSTYAPLVEYMPVGELDAKYKILSAIKGYDFLSLAYNLSRNRANKKRGHQKLGG |
| | GELSEVVISANYDKALNVIKRSINHYHVEIKPEISKLKKKMQNEPLKVMKQARIRR |
| | ELHQLSRKVKRLKWKWGMIPNPELQNIIFEKKEKDFVSYALLHTLGRDIGLFKDTS |
| | MLQVPNISDYGFQIYYSWEDPKLNSIKKIKDLPKRLLIPYKRLDFYIDTILVAKVI |
| | KNLIELYRKSYVYETFGEEYGYAKKAEDILFDWDSINLSEGIEQKIQKIKDEFSDL |
| | LYEARESKRQNFVESFENILGLYDKNFASDRNSYQEKIQSMIIKKQQENIEQKLKR |
| | EFKEVIERGFEGMDQNKKYYKVLSPNIKGGLLYTDTNNLGFFRSHLAFMLLSKISD |
| | DLYRKNNLVSKGGNKGILDQTPETMLTLEFGKSNLPNISIKRKFFNIKYNSSWIGI |
| | RKPKFSIKGAVIREITKKVRDEQRLIKSLEGVWHKSTHFKRWGKPRFNLPRHPDRE |
| | KNNDDNLMESITSRREQIQLLLREKQKQQEKMAGRLDKIDKEIQNLQTANFQIKQI |
| | DKKPALTEKSEGKQSVRNALSAWKWFMEDLIKYQKRTPILQLKLAKM |
| 103 | KFSKRQEGFVIPENIGLYKCLAIVRSANLDADVQGHVSCYGVKKNGTYVLKQNGKK |
| | SIREKGRKYASDLVAFKGDIEKIPFEVIEEKKKEQSIVLGKFNYKLVLDVMKGEKD |
| | RASLTMKNKSKKLVQVSSLGTDEFLLTLLNEKFGIEEIYGIIEPEVFSGKKLVCKA |
| | CQQSTYAPLVEYMPVGELDSKYKILSAIKGYDFLSLAYNLARHRSNKKRGHQKLGG |
| | GELSEVVISANNAKALNVIKRSLNHYYSEIKPEISKLRKKMQNEPLKVGKQARMRR |
| | ELHQLSRKVKRLKWKWGKIPNLELQNITFKESDRDFISYALLHTLGRDIGMFNKTE |
| | IKMPSNILGYGFQIYYDWEEPKLNTIKKSKNTPKRILIPYKKLDFYNDSILVARAI |
| | KELVGLFQESYEWEIFGNEYNYAKEAEVELIKLDEESINGNVEKKLQRIKENFSNL |
| | LEKAREKKRQNFIESFESIARLYDESFTADRNEYQREIQSFIIEKQKQSIEKKLKN |
| | EFKKIVEKKFNEQEQGKKHYRVLNPTIINEFLPKDKNNLGFLRSKIAFILLSKISD |
| | DLYKKSNAVSKGGEKGIIKQQPETILDLEFSKSKLPSINIKKKLFNIKYTSSWLGI |
| | RKPKFNIKGAKIREITRRVRDVQRTLKSAESSWYASTHFRRWGFPRFNQPRHPDKE |
| | KKSDDRLIESITLLREQIQILLREKQKGQKEMAGRLDDVDKKIQNLQTANFQIKQT |
| | GDKPALTEKSAGKQSFRNALSAWKWFMENLLKYQNKTPDLKLKIARTVM |
| 104 | KWIEPNNIDFNKCLAITRSANLDADVQGHKMCYGIKTNGTYKAIGKINKKHNTGII |
| | EKRRTYVYDLIVTKEKNEKIVKKTDFMAIDEEIEFDEKKEKLLKKYIKAEVLGTGE |
| | LIRKDLNDGEKFDDLCSIEEPQAFRRSELVCKACNQSTYASDIRYIPIGEIEAKYK |
| | ILKAIKGYDFLSLKYNLGRLRDSKKRGHQKMGQGELKEFVICANKEKALDVIKRSL |
| | NHYLNEVKDEISRLNKKMQNEPLKVNDQARWRRELNQISRRLKRLKWKWGEIPNPE |
| | LKNLIFKSSRPEFVSYALIHTLGRDIGLINETELKPNNIQEYGFQIYYKWEDPELN |
| | HIKKVKNIPKRFIIPYKNLDLFGKYTILSRAIEGILKLYSSSFQYKSFKDPNLFAK |
| | EGEKKITNEDFELGYDEKIKKIKDDFKSYKKALLEKKKNTLEDSLNSILSVYEQSL |
| | LTEQINNVKKWKEGLLKSKESIHKQKKIENIEDIISRIEELKNVEGWIRTKERDIV |
| | NKEETNLKREIKKELKDSYYEEVRKDFSDLKKGEESEKKPFREEPKPIVIKDYIKF |
| | DVLPGENSALGFFLSHLSFNLFDSIQYELFEKSRLSSSKHPQIPETILDL |
| 105 | FRKFVKRSGAPQPDNLNKYKCIAIVRAANLDADIMSNESSNCVMCKGIKMNKRKTA |
| | KGAAKTTELGRVYAGQSGNLLCTACTKSTMGPLVDYVPIGRIRAKYTILRAVKEYD |
| | FLSLAYNLARTRVSKKGGRQKMHSLSELVIAAEYEIAWNIIKSSVIHYHQETKEEI |
| | SGLRKKLQAEHIHKNKEARIRREMHQISRRIKRLKWKWHMIPNSELHNFLFKQQDP |
| | SFVAVALLHTLGRDIGMINKPKGSAKREFIPEYGFQIYYKWMNPKLNDINKQKYRK |
| | MPKRSLIPYKNLNVFGDRELIENAMHKLLKLYDENLEVKGSKFFKTRVVAISSKES |
| | EKLKRDLLWKGELAKIKKDFNADKNKMQELFKEVKEPKKANALMKQSRNMGFLLQN |
| | ISYGALGLLANRMYEASAKQSKGDATKQPSIVIPLEMEFGNAFPKLLLRSGKFAMN |
| | VSSPWLTIRKPKFVIKGNKIKNITKLMKDEKAKLKRLETSYHRATHFRPTLRGSID |
| | WDSPYFSSPKQPNTHRRSPDRLSADITEYRGRLKSVEAELREGQRAMAKKLDSVDM |
| | TASNLQTSNFQLEKGEDPRLTEIDEKGRSIRNCISSWKKFMEDLMKAQEANPVIKI |
| | KIALKDESSVLSEDSM |
| 106 | KFHPENLNKSYCLAIVRAANLDADIQGHINCIGIKSNKSDRNYENKLESLQNVELL |
| | CKACTKSTYKPNINSVPVGEKKAKYSILSEIKKYDFNSLVYNLKKYRKGKSRGHQK |
| | LNELRELVITSEYKKALDVINKSVNHYLVNIKNKMSKLKKILQNEHIHVGTLARIR |
| | RERNRISRKLDHYRKKWKFVPNKILKNYVFKNQSPDFVSVALLHKLGRDIGLITKT |
| | AILQKSFPEYSLQLYYKYDTPKLNYLKKSKFKSLPKRILISYKYPKFDINSNYIEE |
| | SIDKLLKLYEESPIYKNNSKIIEFFKKSEDNLIKSENDSLKRGIMKEFEKVTKNFS |
| | SKKKKLKEELKLKNEDKNSKMLAKVSRPIGFLKAYLSYMLFNIISNRIFEFSRKSS |
| | GRIPQLPSCIINLGNQFENFKNELQDSNIGSKKNYKYFCNLLLKSSGFNISYEEEH |
| | LSIKTPNFFINGRKLKEITSEKKKIRKENEQLIKQWKKLTFFKPSNLNGKKTSDKI |
| | RFKSPNNPDIERKSEDNIVENIAKVKYKLEDLLSEQRKEFNKLAKKHDGVDVEAQC |
| | LQTKSFWIDSNSPIKKSLEKKNEKVSVKKKMKAIRSCISAWKWFMADLIEAQKETP |
| | MIKLKLALM |
| 107 | TTLVPSHLAGIEVMDETTSRNEDMIQKETSRSNEDENYLGVKNKCGINVHKSGRGS |
| | SKHEPNMPPEKSGEGQMPKQDSTEMQQRFDESVTGETQVSAGATASIKTDARANSG |
| | PRVGTARALIVKASNLDRDIKLGCKPCEYIRSELPMGKKNGCNHCEKSSDIASVPK |
| | VESGFRKAKYELVRRFESFAADSISRHLGKEQARTRGKRGKKDKKEQMGKVNLDEI |
| | AILKNESLIEYTENQILDARSNRIKEWLRSLRLRLRTRNKGLKKSKSIRRQLITLR |
| | RDYRKWIKPNPYRPDEDPNENSLRLHTKLGVDIGVQGGDNKRMNSDDYETSFSITW |
| | RDTATRKICFTKPKGLLPRHMKFKLRGYPELILYNEELRIQDSQKFPLVDWERIPI |
| | FKLRGVSLGKKKVKALNRITEAPRLVVAKRIQVNIESKKKKVLTRYVYNDKSINGR |
| | LVKAEDSNKDPLLEFKKQAEEINSDAKYYENQEIAKNYLWGCEGLHKNLLEEQTKN |
| | PYLAFKYGFLNIV |
| 108 | LDFKRTCSQELVLLPEIEGLKLSGTQGVTSLAKKLINKAANVDRDESYGCHHCIHT |
| | RTSLSKPVKKDCNSCNQSTNHPAVPITLKGYKIAFYELWHRFTSWAVDSISKALHR |
| | NKVMGKVNLDEYAVVDNSHIVCYAVRKCYEKRQRSVRLHKRAYRCRAKHYNKSQPK |
| | VGRIYKKSKRRNARNLKKEAKRYFQPNEITNGSSDALFYKIGVDLGIAKGTPETEV |
| | KVDVSICFQVYYGDARRVLRVRKMDELQSFHLDYTGKLKLKGIGNKDTFTIAKRNE |
| | SLKWGSTKYEVSRAHKKFKPFGKKGSVKRKCNDYFRSIASWSCEAASQRAQSNLKN |
| | AFPYQKALVKCYKNLDYKGVKKNDMWYRLCSNRIFRYSRIAEDIAQYQSDKGKAKF |
| | EFVILAQSVAEYDISAIM |
| 109 | VFLTDDKRKTALRKIRSAFRKTAEIALVRAQEADSLDRQAKKLTIETVSFGAPGAK |
| | NAFIGSLQGYNWNSHRANVPSSGSAKDVFRITELGLGIPQSAHEASIGKSFELVGN |
| | VVRYTANLLSKGYKKGAVNKGAKQQREIKGKEQLSFDLISNGPISGDKLINGQKDA |
| | LAWWLIDKMGFHIGLAMEPLSSPNTYGITLQAFWKRHTAPRRYSRGVIRQWQLPFG |
| | RQLAPLIHNFFRKKGASIPIVLTNASKKLAGKGVLLEQTALVDPKKWWQVKEQVTG |
| | PLSNIWERSVPLVLYTATFTHKHGAAHKRPLTLKVIRISSGSVFLLPLSKVTPGKL |
| | VRAWMPDINILRDGRPDEAAYKGPDLIRARERSFPLAYTCVTQIADEWQKRALESN |
| | RDSITPLEAKLVTGSDLLQIHSTVQQAVEQGIGGRISSPIQELLAKDALQLVLQQL |
| | FMTVDLLRIQWQLKQEVADGNTSEKAVGWAIRISNIHKDAYKTAIEPCTSALKQAW |
| | NPLSGFEERTFQLDASIVRKRSTAKTPDDELVIVLRQQAAEMTVAVTQSVSKELME |
| | LAVRHSATLHLLVGEVASKQLSRSADKDRGAMDHWKLLSQSM |
| 110 | EDLLQKALNTATNVAAIERHSCISCLFTESEIDVKYKTPDKIGQNTAGCQSCTFRV |
| | GYSGNSHTLPMGNRIALDKLRETIQRYAWHSLLFNVPPAPTSKRVRAISELRVAAG |
| | RERLFTVITFVQTNILSKLQKRYAANWTPKSQERLSRLREEGQHILSLLESGSWQQ |
| | KEVVREDQDLIVCSALTKPGLSIGAFCRPKYLKPAKHALVLRLIFVEQWPGQIWGQ |
| | SKRTRRMRRRKDVERVYDISVQAWALKGKETRISECIDTMRRHQQAYIGVLPFLIL |
| | SGSTVRGKGDCPILKEITRMRYCPNNEGLIPLGIFYRGSANKLLRVVKGSSFTLPM |
| | WQNIETLPHPEPFSPEGWTATGALYEKNLAYWSALNEAVDWYTGQILSSGLQYPNQ |
| | NEFLARLQNVIDSIPRKWFRPQGLKNLKPNGQEDIVPNEFVIPQNAIRAHHVIEWY |
| | HKTNDLVAKTLLGWGSQTTLNQTRPQGDLRFTYTRYYFREKEVPEV |
| 111 | VPKKKLMRELAKKAVFEAIFNDPIPGSFGCKRCTLIDGARVTDAIEKKQGAKRCAG |
| | CEPCTFHTLYDSVKHALPAATGCDRTAIDTGLWEILTALRSYNWMSFRRNAVSDAS |
| | QKQVWSIEELAIWADKERALRVILSALTHTIGKLKNGFSRDGVWKGGKQLYENLAQ |
| | KDLAKGLFANGEIFGKELVEADHDMLAWTIVPNHQFHIGLIRGNWKPAAVEASTAF |
| | DARWLTNGAPLRDTRTHGHRGRRFNRTEKLTVLCIKRDGGVSEEFRQERDYELSVM |
| | LLQPKNKLKPEPKGELNSFEDLHDHWWFLKGDEATALVGLTSDPTVGDFIQLGLYI |
| | RNPIKAHGETKRRLLICFEPPIKLPLRRAFPSEAFKTWEPTINVFRNGRRDTEAYY |
| | DIDRARVFEFPETRVSLEHLSKQWEVLRLEPDRENTDPYEAQQNEGAELQVYSLLQ |
| | EAAQKMAPKVVIDPFGQFPLELFSTFVAQLFNAPLSDTKAKIGKPLDSGFVVESHL |
| | HLLEEDFAYRDFVRVTFMGTEPTFRVIHYSNGEGYWKKTVLKGKNNIRTALIPEGA |
| | KAAVDAYKNKRCPLTLEAAILNEEKDRRLVLGNKALSLLAQTARGNLTILEALAAE |
| | VLRPLSGTEGVVHLHACVTRHSTLTESTETDNM |
| 112 | VEKLFSERLKRAMWLKNEAGRAPPAETLTLKHKRVSGGHEKVKEELQRVLRSLSGT |
| | NQAAWNLGLSGGREPKSSDALKGEKSRVVLETVVFHSGHNRVLYDVIEREDQVHQR |
| | SSIMHMRRKGSNLLRLWGRSGKVRRKMREEVAEIKPVWHKDSRWLAIVEEGRQSVV |
| | GISSAGLAVFAVQESQCTTAEPKPLEYVVSIWFRGSKALNPQDRYLEFKKLKTTEA |
| | LRGQQYDPIPFSLKRGAGCSLAIRGEGIKFGSRGPIKQFFGSDRSRPSHADYDGKR |
| | RLSLFSKYAGDLADLTEEQWNRTVSAFAEDEVRRATLANIQDFLSISHEKYAERLK |
| | KRIESIEEPVSASKLEAYLSAIFETFVQQREALASNFLMRLVESVALLISLEEKSP |
| | RVEFRVARYLAESKEGFNRKAM |
| 113 | VVITQSELYKERLLRVMEIKNDRGRKEPRESQGLVLRFTQVTGGQEKVKQKLWLIF |
| | EGFSGTNQASWNFGQPAGGRKPNSGDALKGPKSRVTYETVVFHFGLRLLSAVIERH |
| | NLKQQRQTMAYMKRRAAARKKWARSGKKCSRMRNEVEKIKPKWHKDPRWFDIVKEG |
| | EPSIVGISSAGFAIYIVEEPNFPRQDPLEIEYAISIWFRRDRSQYLTFKKIQKAEK |
| | LKELQYNPIPFRLKQEKTSLVFESGDIKFGSRGSIEHFRDEARGKPPKADMDNNRR |
| | LTMFSVFSGNLTNLTEEQYARPVSGLLAPDEKRMPTLLKKLQDFFTPIHEKYGERI |
| | KQRLANSEASKRPFKKLEEYLPAIYLEFRARREGLASNWVLVLINSVRTLVRIKSE |
| | DPYIEFKVSQYLLEKEDNKAL |
| 114 | KQDALFEERLKKAIFIKRQADPLQREELSLLPPNRKIVTGGHESAKDTLKQILRAI |
| | NGTNQASWNPGTPSGKRDSKSADALAGPKSRVKLETVVFHVGHRLLKKVVEYQGHQ |
| | KQQHGLKAFMRTCAAMRKKWKRSGKVVGELREQLANIQPKWHYDSRPLNLCFEGKP |
| | SVVGLRSAGIALYTIQKSVVPVKEPKPIEYAVSIWFRGPKAMDREDRCLEFKKLKI |
| | ATELRKLQFEPIVSTLTQGIKGFSLYIQGNSVKFGSRGPIKYFSNESVRQRPPKAD |
| | PDGNKRLALFSKFSGDLSDLTEEQWNRPILAFEGIIRRATLGNIQDYLTVGHEQFA |
| | ISLEQLLSEKESVLQMSIEQQRLKKNLGKKAENEWVESFGAEQARKKAQGIREYIS |
| | GFFQEYCSQREQWAENWVQQLNKSVRLFLTIQDSTPFIEFRVARYLPKGEKKKGKA |
| | M |
| 115 | ANHAERHKRLRKEANRAANRNRPLVADCDTGDPLVGICRLLRRGDKMQPNKTGCRS |
| | CEQVEPELRDAILVSGPGRLDNYKYELFQRGRAMAVHRLLKRVPKLNRPKKAAGND |
| | EKKAENKKSEIQKEKQKQRRMMPAVSMKQVSVADFKHVIENTVRHLFGDRRDREIA |
| | ECAALRAASKYFLKSRRVRPRKLPKLANPDHGKELKGLRLREKRAKLKKEKEKQAE |
| | LARSNQKGAVLHVATLKKDAPPMPYEKTQGRNDYTTFVISAAIKVGATRGTKPLLT |
| | PQPREWQCSLYWRDGQRWIRGGLLGLQAGIVLGPKLNRELLEAVLQRPIECRMSGC |
| | GNPLQVRGAAVDFFMTTNPFYVSGAAYAQKKFKPFGTKRASEDGAAAKAREKLMTQ |
| | LAKVLDKVVTQAAHSPLDGIWETRPEAKLRAMIMALEHEWIFLRPGPCHNAAEEVI |
| | KCDCTGGHAILWALIDEARGALEHKEFYAVTRAHTHDCEKQKLGGRLAGFLDLLIA |
| | QDVPLDDAPAARKIKTLLEATPPAPCYKAATSIATCDCEGKFDKLWAIIDATRAGH |
| | GTEDLWARTLAYPQNVNCKCKAGKDLTHRLADFLGLLIKRDGPFRERPPHKVTGDR |
| | KLVFSGDKKCKGHQYVILAKAHNEEVVRAWISRWGLKSRTNKAGYAATELNLLLNW |
| | LSICRRRWMDMLTVQRDTPYIRMKTGRLVVDDKKERKAM |
| 116 | AKQREALRVALERGIVRASNRTYTLVTNCTKGGPLPEQCRMIERGKARAMKWEPKL |
| | VGCGSCAAATVDLPAIEEYAQPGRLDVAKYKLTTQILAMATRRMMVRAAKLSRRKG |
| | QWPAKVQEEKEEPPEPKKMLKAVEMRPVAIVDFNRVIQTTIEHLWAERANADEAEL |
| | KALKAAAAYFGPSLKIRARGPPKAAIGRELKKAHRKKAYAERKKARRKRAELARSQ |
| | ARGAAAHAAIRERDIPPMAYERTQGRNDVTTIPIAAAIKIAATRGARPLPAPKPMK |
| | WQCSLYWNEGQRWIRGGMLTAQAYAHAANIHRPMRCEMWGVGNPLKVRAFEGRVAD |
| | PDGAKGRKAEFRLQTNAFYVSGAAYRNKKFKPFGTDRGGIGSARKKRERLMAQLAK |
| | ILDKVVSQAAHSPLDDIWHTRPAQKLRAMIKQLEHEWMFLRPQAPTVEGTKPDVDV |
| | AGNMQRQIKALMAPDLPPIEKGSPAKRFTGDKRKKGERAVRVAEAHSDEVVTAWIS |
| | RWGIQTRRNEGSYAAQELELLLNWLQICRRRWLDMTAAQRVSPYIRMKSGRMITDA |
| | ADEGVAPIPLVENM |
| 117 | KSISGRSIKHMACLKDMLKSEITEIEEKQKKESLRKWDYYSKFSDEILFRRNLNVS |
| | ANHDANACYGCNPCAFLKEVYGFRIERRNNERIISYRRGLAGCKSCVQSTGYPPIE |
| | FVRRKFGADKAMEIVREVLHRRNWGALARNIGREKEADPILGELNELLLVDARPYF |
| | GNKSAANETNLAFNVITRAAKKFRDEGMYDIHKQLDIHSEEGKVPKGRKSRLIRIE |
| | RKHKAIHGLDPGETWRYPHCGKGEKYGVWLNRSRLIHIKGNEYRCLTAFGTTGRRM |
| | SLDVACSVLGHPLVKKKRKKGKKTVDGTELWQIKKATETLPEDPIDCTFYLYAAKP |
| | TKDPFILKVGSLKAPRWKKLHKDFFEYSDTEKTQGQEKGKRVVRRGKVPRILSLRP |
| | DAKFKVSIWDDPYNGKNKEGTLLRMELSGLDGAKKPLILKRYGEPNTKPKNFVFWR |
| | PHITPHPLTFTPKHDFGDPNKKTKRRRVFNREYYGHLNDLAKMEPNAKFFEDREVS |
| | NKKNPKAKNIRIQAKESLPNIVAKNGRWAAFDPNDSLWKLYLHWRGRRKTIKGGIS |
| | QEFQEFKERLDLYKKHEDESEWKEKEKLWENHEKEWKKTLEIHGSIAEVSQRCVMQ |
| | SMMGPLDGLVQKKDYVHIGQSSLKAADDAWTFSANRYKKATGPKWGKISVSNLLYD |
| | ANQANAELISQSISKYLSKQKDNQGCEGRKMKFLIKIIEPLRENFVKHTRWLHEMT |
| | QKDCEVRAQFSRVSM |
| 118 | FPSDVGADALKHVRMLQPRLTDEVRKVALTRAPSDRPALARFAAVAQDGLAFVRHL |
| | NVSANHDSNCTFPRDPRDPRRGPCEPNPCAFLREVWGFRIVARGNERALSYRRGLA |
| | GCKSCVQSTGFPSVPFHRIGADDCMRKLHEILKARNWRLLARNIGREREADPLLTE |
| | LSEYLLVDARTYPDGAAPNSGRLAENVIKRAAKKFRDEGMRDIHAQLRVHSREGKV |
| | PKGRLQRLRRIERKHRAIHALDPGPSWEAEGSARAEVQGVAVYRSQLLRVGHHTQQ |
| | IEPVGIVARTLFGVGRTDLDVAVSVLGAPLTKRKKGSKTLESTEDFRIAKARETRA |
| | EDKIEVAFVLYPTASLLRDEIPKDAFPAMRIDRFLLKVGSVQADREILLQDDYYRF |
| | GDAEVKAGKNKGRTVTRPVKVPRLQALRPDAKFRVNVWADPFGAGDSPGTLLRLEV |
| | SGVTRRSQPLRLLRYGQPSTQPANFLCWRPHRVPDPMTFTPRQKFGERRKNRRTRR |
| | PRVFERLYQVHIKHLAHLEPNRKWFEEARVSAQKWAKARAIRRKGAEDIPVVAPPA |
| | KRRWAALQPNAELWDLYAHDREARKRFRGGRAAEGEEFKPRLNLYLAHEPEAEWES |
| | KRDRWERYEKKWTAVLEEHSRMCAVADRTLPQFLSDPLGARMDDKDYAFVGKSALA |
| | VAEAFVEEGTVERAQGNCSITAKKKFASNASRKRLSVANLLDVSDKADRALVFQAV |
| | RQYVQRQAENGGVEGRRMAFLRKLLAPLRQNFVCHTRWLHM |
| 119 | AARKKKRGKIGITVKAKEKSPPAAGPFMARKLVNVAANVDGVEVHLCVECEADAHG |
| | SASARLLGGCRSCTGSIGAEGRLMGSVDVDRERVIAEPVHTETERLGPDVKAFEAG |
| | TAESKYAIQRGLEYWGVDLISRNRARTVRKMEEADRPESSTMEKTSWDEIAIKTYS |
| | QAYHASENHLFWERQRRVRQHALALFRRARERNRGESPLQSTQRPAPLVLAALHAE |
| | AAAISGRARAEYVLRGPSANVRAAAADIDAKPLGHYKTPSPKVARGFPVKRDLLRA |
| | RHRIVGLSRAYFKPSDVVRGTSDAIAHVAGRNIGVAGGKPKEIEKTFTLPFVAYWE |
| | DVDRVVHCSSFKADGPWVRDQRIKIRGVSSAVGTFSLYGLDVAWSKPTSFYIRCSD |
| | IRKKFHPKGFGPMKHWRQWAKELDRLTEQRASCVVRALQDDEELLQTMERGQRYYD |
| | VFSCAATHATRGEADPSGGCSRCELVSCGVAHKVTKKAKGDTGIEAVAVAGCSLCE |
| | SKLVGPSKPRVHRQMAALRQSHALNYLRRLQREWEALEAVQAPTPYLRFKYARHLE |
| | VRSM |
| 120 | AAKKKKQRGKIGISVKPKEGSAPPADGPFMARKLVNVAANVDGVEVNLCIECEADA |
| | HGSAPARLLGGCKSCTGSIGAEGRLMGSVDVDRADAIAKPVNTETEKLGPDVQAFE |
| | AGTAETKYALQRGLEYWGVDLISRNRSRTVRRTEEGQPESATMEKTSWDEIAIKSY |
| | TRAYHASENHLFWERQRRVRQHALALFKRAKERNRGDSTLPREPGHGLVAIAALAC |
| | EAYAVGGRNLAETVVRGPTFGTARAVRDVEIASLGRYKTPSPKVAHGSPVKRDFLR |
| | ARHRIVGLARAYYRPSDVVRGTSDAIAHVAGRNIGVAGGKPRAVEAVFTLPFVAYW |
| | EDVDRVVHCSSFQVSAPWNRDQRMKIAGVTTAAGTFSLHGGELKWAKPTSFYIRCS |
| | DTRRKFRPKGFGPMKRWRQWAKDLDRLVEQRASCVVRALQDDAALLETMERGQRYY |
| | DVFACAVTHATRGEADRLAGCSRCALTPCQEAHRVTTKPRGDAGVEQVQTSDCSLC |
| | EGKLVGPSKPRLHRTLTLLRQEHGLNYLRRLQREWESLEAVQVPTPYLRFKYARHL |
| | EVRSM |
| 121 | TDSQSESVPEVVYALTGGEVPGRVPPDGGSAEGARNAPTGLRKQRGKIKISAKPSK |
| | PGSPASSLARTLVNEAANVDGVQSSGCATCRMRANGSAPRALPIGCVACASSIGRA |
| | PQEETVCALPTTQGPDVRLLEGGHALRKYDIQRALEYWGVDLIGRNLDRQAGRGME |
| | PAEGATATMKRVSMDELAVLDFGKSYYASEQHLFAARQRRVRQHAKALKIRAKHAN |
| | RSGSVKRALDRSRKQVTALAREFFKPSDVVRGDSDALAHVVGRNLGVSRHPAREIP |
| | QTFTLPLCAYWEDVDRVISCSSLLAGEPFARDQEIRIEGVSSALGSLRLYRGAIEW |
| | HKPTSLYIRCSDTRRKFRPRGGLKKRWRQWAKDLDRLVEQRACCIVRSLQADVELL |
| | QTMERAQRFYDVHDCAATHVGPVAVRCSPCAGKQFDWDRYRLLAALRQEHALNYLR |
| | RLQREWESLEAQQVKMPYLRFKYARKLEVSGPLIGLEVRREPSMGTAIAEM |
| 122 | AGTAGRRHGSLGARRSINIAGVTDRHGRWGCESCVYTRDQAGNRARCAPCDQSTYA |
| | PDVQEVTIGQRQAKYTIFLTLQSFSWTNTMRNNKRAAAGRSKRTTGKRIGQLAEIK |
| | ITGVGLAHAHNVIQRSLQHNITKMWRAEKGKSKRVARLKKAKQLTKRRAYFRRRMS |
| | RQSRGNGFFRTGKGGIHAVAPVKIGLDVGMIASGSSEPADEQTVTLDAIWKGRKKK |
| | IRLIGAKGELAVAACRFREQQTKGDKCIPLILQDGEVRWNQNNWQCHPKKLVPLCG |
| | LEVSRKFVSQADRLAQNKVASPLAARFDKTSVKGTLVESDFAAVLVNVTSIYQQCH |
| | AMLLRSQEPTPSLRVQRTITSM |
| 123 | GVRFSPAQSQVFFRTVIPQSVEARFAINMAAIHDAAGAFGCSVCRFEDRTPRNAKA |
| | VHGCSPCTRSTNRPDVFVLPVGAIKAKYDVFMRLLGFNWTHLNRRQAKRVTVRDRI |
| | GQLDELAISMLTGKAKAVLKKSICHNVDKSFKAMRGSLKKLHRKASKTGKSQLRAK |
| | LSDLRERTNTTQEGSHVEGDSDVALNKIGLDVGLVGKPDYPSEESVEVVVCLYFVG |
| | KVLILDAQGRIRDMRAKQYDGFKIPIIQRGQLTVLSVKDLGKWSLVRQDYVLAGDL |
| | RFEPKISKDRKYAECVKRIALITLQASLGFKERIPYYVTKQVEIKNASHIAFVTEA |
| | IQNCAENFREMTEYLMKYQEKSPDLKVLLTQLM |
| 124 | RAVVGKVFLEQARRALNLATNFGTNHRTGCNGCYVTPGKLSIPQDGEKNAAGCTSC |
| | LMKATASYVSYPKPLGEKVAKYSTLDALKGFPWYSLRLNLRPNYRGKPINGVQEVA |
| | PVSKFRLAEEVIQAVQRYHFTELEQSFPGGRRRLRELRAFYTKEYRRAPEQRQHVV |
| | NGDRNIVVVTVLHELGFSVGMFNEVELLPKTPIECAVNVFIRGNRVLLEVRKPQFD |
| | KERLLVESLWKKDSRRHTAKWTPPNNEGRIFTAEGWKDFQLPLLLGSTSRSLRAIE |
| | KEGFVQLAPGRDPDYNNTIDEQHSGRPFLPLYLYLQGTISQEYCVFAGTWVIPFQD |
| | GISPYSTKDTFQPDLKRKAYSLLLDAVKHRLGNKVASGLQYGRFPAIEELKRLVRM |
| | HGATRKIPRGEKDLLKKGDPDTPEWWLLEQYPEFWRLCDAAAKRVSQNVGLLLSLK |
| | KQPLWQRRWLESRTRNEPLDNLPLSMALTLHLTNEEAL |
| 125 | AAVYSKFYIENHFKMGIPETLSRIRGPSIIQGFSVNENYINIAGVGDRDFIFGCKK |
| | CKYTRGKPSSKKINKCHPCKRSTYPEPVIDVRGSISEFKYKIYNKLKQEPNQSIKQ |
| | NTKGRMNPSDHTSSNDGIIINGIDNRIAYNVIFSSYKHLMEKQINLLRDTTKRKAR |
| | QIKKYNNSGKKKHSLRSQTKGNLKNRYHMLGMFKKGSLTITNEGDFITAVRKVGLD |
| | ISLYKNESLNKQEVETELCLNIKWGRTKSYTVSGYIPLPINIDWKLYLFEKETGLT |
| | LRLFGNKYKIQSKKFLIAQLFKPKRPPCADPVVKKAQKWSALNAHVQQMAGLFSDS |
| | HLLKRELKNRMHKQLDFKSLWVGTEDYIKWFEELSRSYVEGAEKSLEFFRQDYFCF |
| | NYTKQTTM |
| 126 | PQQQRDLMLMAANYDQDYGNGCGPCTVVASAAYRPDPQAQHGCKRHLRTLGASAVT |
| | HVGLGDRTATITALHRLRGPAALAARARAAQAASAPMTPDTDAPDDRRRLEAIDAD |
| | DVVLVGAHRALWSAVRRWADDRRAALRRRLHSEREWLLKDQIRWAELYTLIEASGT |
| | PPQGRWRNTLGALRGQSRWRRVLAPTMRATCAETHAELWDALAELVPEMAKDRRGL |
| | LRPPVEADALWRAPMIVEGWRGGHSVVVDAVAPPLDLPQPCAWTAVRLSGDPRQRW |
| | GLHLAVPPLGQVQPPDPLKATLAVSMRHRGGVRVRTLQAMAVDADAPMQRHLQVPL |
| | TLQRGGGLQWGIHSRGVRRREARSMASWEGPPIWTGLQLVNRWKGQGSALLAPDRP |
| | PDTPPYAPDAAVAPAQPDTKRARRTLKEACTVCRCAPGHMRQLQVTLTGDGTWRRF |
| | RLRAPQGAKRKAEVLKVATQHDERIANYTAWYLKRPEHAAGCDTCDGDSRLDGACR |
| | GCRPLLVGDQCFRRYLDKIEADRDDGLAQIKPKAQEAVAAMAAKRDARAQKVAARA |
| | AKLSEATGQRTAATRDASHEARAQKELEAVATEGTTVRHDAAAVSAFGSWVARKGD |
| | EYRHQVGVLANRLEHGLRLQELMAPDSVVADQQRASGHARVGYRYVLTAM |
| 127 | AVAHPVGRGNAGSPGARGPEELPRQLVNRASNVTRPATYGCAPCRHVRLSIPKPVL |
| | TGCRACEQTTHPAPKRAVRGGADAAKYDLAAFFAGWAADLEGRNRRRQVHAPLDPQ |
| | PDPNHEPAVTLQKIDLAEVSIEEFQRVLARSVKHRHDGRASREREKARAYAQVAKK |
| | RRNSHAHGARTRRAVRRQTRAVRRAHRMGANSGEILVASGAEDPVPEAIDHAAQLR |
| | RRIRACARDLEGLRHLSRRYLKTLEKPCRRPRAPDLGRARCHALVESLQAAERELE |
| | ELRRCDSPDTAMRRLDAVLAAAASTDATFATGWTVVGMDLGVAPRGSAAPEVSPME |
| | MAISVFWRKGSRRVIVSKPIAGMPIRRHELIRLEGLGTLRLDGNHYTGAGVTKGRG |
| | LSEGTEPDFREKSPSTLGFTLSDYRHESRWRPYGAKQGKTARQFFAAMSRELRALV |
| | EHQVLAPMGPPLLEAHERRFETLLKGQDNKSIHAGGGGRYVWRGPPDSKKRPAADG |
| | DWFRFGRGHADHRGWANKRHELAANYLQSAFRLWSTLAEAQEPTPYARYKYTRVTM |
| 128 | WDFLTLQVYERHTSPEVCVAGNSTKCASGTRKSDHTHGVGVKLGAQEINVSANDDR |
| | DHEVGCNICVISRVSLDIKGWRYGCESCVQSTPEWRSIVRFDRNHKEAKGECLSRF |
| | EYWGAQSIARSLKRNKLMGGVNLDELAIVQNENVVKTSLKHLFDKRKDRIQANLKA |
| | VKVRMRERRKSGRQRKALRRQCRKLKRYLRSYDPSDIKEGNSCSAFTKLGLDIGIS |
| | PNKPPKIEPKVEVVFSLFYQGACDKIVTVSSPESPLPRSWKIKIDGIRALYVKSTK |
| | VKFGGRTFRAGQRNNRRKVRPPNVKKGKRKGSRSQFFNKFAVGLDAVSQQLPIASV |
| | QGLWGRAETKKAQTICLKQLESNKPLKESQRCLFLADNWVVRVCGFLRALSQRQGP |
| | TPYIRYRYRCNM |
| 129 | ARNVGQRNASRQSKRESAKARSRRVTGGHASVTQGVALINAAANADRDHTTGCEPC |
| | TWERVNLPLQEVIHGCDSCTKSSPFWRDIKVVNKGYREAKEEIMRIASGISADHLS |
| | RALSHNKVMGRLNLDEVCILDFRTVLDTSLKHLTDSRSNGIKEHIRAVHRKIRMRR |
| | KSGKTARALRKQYFALRRQWKAGHKPNSIREGNSLTALRAVGFDVGVSEGTEPMPA |
| | PQTEVVLSVFYKGSATRILRISSPHPIAKRSWKVKIAGIKALKLIRREHDFSFGRE |
| | TYNASQRAEKRKFSPHAARKDFFNSFAVQLDRLAQQLCVSSVENLWVTEPQQKLLT |
| | LAKDTAPYGIREGARFADTRARLAWNWVFRVCGFTRALHQEQEPTPYCRFTWRSKM |

In some embodiments, the Type VI CRISPR/Cas enzyme is a programmable Cas13 nuclease. The general architecture of a Cas13 protein includes an N-terminal domain and two HEPN (higher eukaryotes and prokaryotes nucleotide-binding) domains separated by two helical domains (Liu et al., Cell 2017 Jan. 12; 168(1-2):121-134.e12). The HEPN domains each comprise aR-X₄—H motif. Shared features across Cas13 proteins include that upon binding of the crRNA of the guide nucleic acid to a target nucleic acid, the protein undergoes a conformational change to bring together the HEPN domains and form a catalytically active RNase. (Tambe et al., Cell Rep. 2018 Jul. 24; 24(4): 1025-1036). Thus, two activatable HEPN domains are characteristic of a programmable Cas13 nuclease of the present disclosure. However, programmable Cas13 nucleases also consistent with the present disclosure include Cas13 nucleases comprising mutations in the HEPN domain that enhance the Cas13 proteins cleavage efficiency or mutations that catalytically inactivate the HEPN domains. Programmable Cas13 nucleases consistent with the present disclosure also Cas13 nucleases comprising catalytic components.

A programmable Cas13 nuclease can be a Cas13a protein (also referred to as “c2c2”), a Cas13b protein, a Cas13c protein, a Cas13d protein, or a Cas13e protein. Example C2c2 proteins are set forth as SEQ ID NO: 130-SEQ ID NO: 137. In some cases, a subject C2c2 protein includes an amino acid sequence having 80% or more (e.g., 85% or more, 90% or more, 95% or more, 98% or more, 99% or more, 99.5% or more, or 100%) amino acid sequence identity with the amino acid sequence set forth in any one of SEQ ID NOs: 130-SEQ ID NO: 137. In some cases, a suitable C2c2 polypeptide comprises an amino acid sequence having at least 80%, at least 85%, at least 90%, at least 95%, at least 98%, at least 99%, or 100%, amino acid sequence identity to the Listeria seeligeri C2c2 amino acid sequence set forth in SEQ ID NO: 130. In some cases, a suitable C2c2 polypeptide comprises an amino acid sequence having at least 80%, at least 85%, at least 90%, at least 95%, at least 98%, at least 99%, or 100%, amino acid sequence identity to the Leptotrichia buccalis C2c2 amino acid sequence set forth in SEQ ID NO: 131. In some cases, a suitable C2c2 polypeptide comprises an amino acid sequence having at least 80%, at least 85%, at least 90%, at least 95%, at least 98%, at least 99%, or 100%, amino acid sequence identity to the Rhodobacter capsulatus C2c2 amino acid sequence set forth in SEQ ID NO: 133. In some cases, a suitable C2c2 polypeptide comprises an amino acid sequence having at least 80%, at least 85%, at least 90%, at least 95%, at least 98%, at least 99%, or 100%, amino acid sequence identity to the Carnobacterium gallinarum C2c2 amino acid sequence set forth in SEQ ID NO: 134. In some cases, a suitable C2c2 polypeptide comprises an amino acid sequence having at least 80%, at least 85%, at least 90%, at least 95%, at least 98%, at least 99%, or 100%, amino acid sequence identity to the Herbinix hemicellulosilytica C2c2 amino acid sequence set forth in SEQ ID NO: 135. In some cases, the C2c2 protein includes an amino acid sequence having 80% or more amino acid sequence identity with the Leptotrichia buccalis (Lbu) C2c2 amino acid sequence set forth in SEQ ID NO: 131. In some cases, the C2c2 protein is a Leptotrichia buccalis (Lbu) C2c2 protein (e.g., see SEQ ID NO: 131). In some cases, the C2c2 protein includes the amino acid sequence set forth in any one of SEQ ID NOs: 130-131 and SEQ ID NOs: 133-137. In some cases, a C2c2 protein used in a method of the present disclosure is not a Leptotrichia shahii (Lsh) C2c2 protein. In some cases, a C2c2 protein used in a method of the present disclosure is not a C2c2 polypeptide having at least 80%, at least 85%, at least 90%, at least 95%, at least 98%, at least 99%, or 100%, amino acid sequence identity to the Lsh C2c2 polypeptide set forth in SEQ ID NO: 132. Other Cas13 protein sequences are set forth in SEQ ID NO: 130-SEQ ID NO: 147.

TABLE 3
Cas13 Protein Sequences

| SEQ | | |
|---|---|---|
| ID | | |
| NO | Description | Sequence |
| 130 | Listeria | MWISIKTLIHHLGVLFFCDYMYNRREKKIIEVKTMRITKVEVDR |
| | seeligeri | KKVLISRDKNGGKLVYENEMQDNTEQIMHHKKSSFYKSVVNKT |
| | C2c2 amino | ICRPEQKQMKKLVHGLLQENSQEKIKVSDVTKLNISNFLNHRFK |
| | acid | KSLYYFPENSPDKSEEYRIEINLSQLLEDSLKKQQGTFICWESFS |
| | sequence | KDMELYINWAENYISSKTKLIKKSIRNNRIQSTESRSGQLMDRY |
| | | MKDILNKNKPFDIQSVSEKYQLEKLTSALKATFKEAKKNDKEIN |
| | | YKLKSTLQNHERQIIEELKENSELNQFNIEIRKHLETYFPIKKTN |
| | | RKVGDIRNLEIGEIQKIVNHRLKNKIVQRILQEGKLASYEIESTV |
| | | NSNSLQKIKIEEAFALKFINACLFASNNLRNMVYPVCKKDILMIG |
| | | EFKNSFKEIKHKKFIRQWSQFFSQEITVDDIELASWGLRGAIAPI |
| | | RNEIIHLKKHSWKKFFNNPTFKVKKSKIINGKTKDVTSEFLYKE |
| | | TLFKDYFYSELDSVPELIINKMESSKILDYYSSDQLNQVFTIPNFE |
| | | LSLLTSAVPFAPSFKRVYLKGFDYQNQDEAQPDYNLKLNIYNEK |
| | | AFNSEAFQAQYSLFKMVYYQVFLPQFTTNNDLFKSSVDFILTLN |
| | | KERKGYAKAFQDIRKMNKDEKPSEYMSYIQSQLMLYQKKQEE |
| | | KEKINHFEKFINQVFIKGFNSFIEKNRLTYICHPTKNTVPENDNIE |
| | | IPFHTDMDDSNIAFWLMCKLLDAKQLSELRNEMIKFSCSLQSTE |
| | | EISTFTKAREVIGLALLNGEKGCNDWKELFDDKEAWKKNMSLY |
| | | VSEELLQSLPYTQEDGQTPVINRSIDLVKKYGTETILEKLFSSSD |
| | | DYKVSAKDIAKLHEYDVTEKIAQQESLHKQWIEKPGLARDSAW |
| | | TKKYQNVINDISNYQWAKTKVELTQVRHLHQLTIDLLSRLAGY |
| | | MSIADRDFQFSSNYILERENSEYRVTSWILLSENKNKNKYNDYEL |
| | | YNLKNASIKVSSKNDPQLKVDLKQLRLTLEYLELFDNRLKEKR |
| | | NNISHFNYLNGQLGNSILELFDDARDVLSYDRKLKNAVSKSLKEI |
| | | LSSHGMEVTFKPLYQTNHHLKIDKLQPKKIHHLGEKSTVSSNQ |
| | | VSNEYCQLVRTLLTMK |
| 131 | Leptotrichia | MKVTKVGGISHKKYTSEGRLVKSESEENRTDERLSALLNMRLD |
| | buccalis (Lbu) | MYIKNPSSTETKENQKRIGKLKKFFSNKMVYLKDNTLSLKNGK |
| | C2c2 amino | KENIDREYSETDILESDVRDKKNFAVLKKIYLNENVNSEELEVFR |
| | acid sequence | NDIKKKLNKINSLKYSFEKNKANYQKINENNIEKVEGKSKRNIIY |
| | | DYYRESAKRDAYVSNVKEAFDKLYKEEDIAKLVLEIENLTKLEK |
| | | YKIREFYHEIIGRKNDKENFAKIIYEEIQNVNNMKELIEKVPDMS |
| | | ELKKSQVFYKYYLDKEELNDKNIKYAFCHFVEIEMSQLLKNYV |
| | | YKRLSNISNDKIKRIFEYQNLKKLIENKLLNKLDTYVRNCGKYN |
| | | YYLQDGEIATSDFIARNRQNEAFLRNIIGVSSVAYFSLRNILETEN |
| | | ENDITGRMRGKTVKNNKGEEKYVSGEVDKIYNENKKNEVKEN |
| | | LKMFYSYDFNMDNKNEIEDFFANIDEAISSIRHGIVHFNLELEGK |
| | | DIFAFKNIAPSEISKKMFQNEINEKKLKLKIFRQLNSANVFRYLE |
| | | KYKILNYLKRTRFEFVNKNIPFVPSFTKLYSRIDDLKNSLGIYWK |
| | | TPKTNDDNKTKEIIDAQIYLLKNIYYGEFLNYFMSNNGNFFEISK |
| | | EIIELNKNDKRNLKTGFYKLQKFEDIQEKIPKEYLANIQSLYMIN |
| | | AGNQDEEEKDTYIDFIQKIFLKGFMTYLANNGRLSLIYIGSDEET |
| | | NTSLAEKKQEFDKFLKKYEQNNNIKIPYEINEFLREIKLGNILKY |
| | | TERLNMFYLILKLLNHKELTNLKGSLEKYQSANKEEAFSDQLEL |
| | | INLLNLDNNRVTEDFELEADEIGKFLDFNGNKVKDNKELKKFDT |
| | | NKIYFDGENIIKHRAFYNIKKYGMLNLLEKIADKAGYKISIEELK |
| | | KYSNKKNEIEKNHKMQENLHRKYARPRKDEKFTDEDYESYKQ |
| | | AIENIEEYTHLKNKVEFNELNLLQGLLLRILHRLVGYTSIWERD |
| | | LRFRLKGEFPENQYIEEIFNFENKKNVKYKGGQIVEKYIKFYKE |
| | | LHQNDEVKINKYSSANIKVLKQEKKDLYIRNYIAHFNYIPHAEIS |
| | | LLEVLENLRKLLSYDRKLKNAVMKSVVDILKEYGFVATFKIGA |
| | | DKKIGIQTLESEKIVHLKNLKKKKLMTDRNSEELCKLVKIMFEY |
| | | KMEEKKSEN |
| 132 | Leptotrichia | MGNLFGHKRWYEVRDKKDFKIKRKVKVKRNYDGNKYILNINE |
| | shahii (Lsh) | NNNKEKIDNNKFIRKYINYKKNDNILKEFTRKFHAGNILFKLKG |
| | C2c2 protein | KEGIIRIENNDDFLETEEVVLYIEAYGKSEKLKALGITKKKIIDEA |
| | | IRQGITKDDKKIEIKRQENEEEIEIDIRDEYTNKTLNDCSIILRIIE |
| | | NDELETKKSIYEIFKNINMSLYKIIEKIIENETEKVFENRYYEEHL |
| | | REKLLKDDKIDVILTNFMEIREKIKSNLEILGFVKFYLNVGGDK |
| | | KKSKNKKML VEKILNINVDLTVEDIADFVIKELEFWNITKRIEKV |
| | | KKVNNEFLEKRRNRTYIKSYVLLDKHEKFKIERENKKDKIVKFF |
| | | VENIKNNSIKEKIEKILAEFKIDELIKKLEKELKKGNCDTEIFGIF |
| | | KKHYKVNFDSKKFSKKSDEEKELYKIIYRYLKGRIEKILVNEQK |
| | | VRLKKMEKIEIEKILNESILSEKILKRVKQYTLEHIMYLGKLRH |
| | | NDIDMTTVNTDDFSRLHAKEELDLELITFFASTNMELNKIFSREN |
| | | INNDENIDFFGGDREKNYVLDKKILNSKIKIIRDLDFIDNKNNITN |
| | | NFIRKFTKIGTNERNRILHAISKERDLQGTQDDYNKVINIIQNLKI |
| | | SDEEVSKALNLDVVFKDKKNIITKINDIKISEENNNDIKYLPSFSK |
| | | VLPEILNLYRNNPKNEPFDTIETEKIVLNALIYVNKELYKKLILE |
| | | DDLEENESKNIFLQELKKTLGNIDEIDENIIENYYKNAQISASKGN |
| | | NKAIKKYQKKVIECYIGYLRKNYEELFDFSDFKMNIQEIKKQIK |
| | | DINDNKTYERITVKTSDKTIVINDDFEYIISIFALLNSNAVINKIRN |
| | | RFFATSVWLNTSEYQNIIDILDEIMQLNTLRNECITENWNLNLEE |
| | | FIQKMKEIEKDFDDFKIQTKKEIFNNYYEDIKNNILTEFKDDING |
| | | CDVLEKKLEKIVIFDDETKFEIDKKSNILQDEQRKLSNINKKDLK |
| | | KKVDQYIKDKDQEIKSKILCRIIFNSDFLKKYKKEIDNLIEDMES |
| | | ENENKFQEIYYPKERKNELYIYKKNLFLNIGNPNFDKIYGLISNDI |
| | | KMADAKFLFNIDGKNIRKNKISEIDAILKNLNDKLNGYSKEYKE |
| | | KYIKKLKENDDFFAKNIQNKNYKSFEKDYNRVSEYKKIRDLVEF |
| | | NYLNKIESYLIDINWKLAIQMARFERDMHYIVNGLRELGIIKLSG |
| | | YNTGISRAYPKRNGSDGFYTTTAYYKFFDEESYKKFEKICYGFGI |
| | | DLSENSEINKPENESIRNYISHFYIVRNPFADYSIAEQIDRVSNLLS |
| | | YSTRYNNSTYASVFEVFKKDVNLDYDELKKKFKLIGNNDILERL |
| | | MKPKKVSVLELESYNSDYIKNLIIELLTKIENTNDTL |
| 133 | Rhodobacter | MQIGKVQGRTISEFGDPAGGLKRKISTDGKNRKELPAHLSSDPK |
| | capsulatus | ALIGQWISGIDKIYRKPDSRKSDGKAIHSPTPSKMQFDARDDLGE |
| | C2c2 amino | AFWKLVSEAGLAQDSDYDQFKRRLHPYGDKFQPADSGAKLKFE |
| | acid sequence | ADPPEPQAFHGRWYGAMSKRGNDAKELAAALYEHLHVDEKRI |
| | | DGQPKRNPKTDKFAPGLVVARALGIESSVLPRGMARLARNWGE |
| | | EEIQTYFVVDVAASVKEVAKAAVSAAQAFDPPRQVSGRSLSPKV |
| | | GFALAEHLERVTGSKRCSFDPAAGPSVLALHDEVKKTYKRLCA |
| | | RGKNAARAFPADKTELLALMRHTHENRVRNQMVRMGRVSEY |
| | | RGQQAGDLAQSHYWTSAGQTEIKESEIFVRLWVGAFALAGRSM |
| | | KAWIDPMGKIVNTEKNDRDLTAAVNIRQVISNKEMVAEAMARR |
| | | GIYFGETPELDRLGAEGNEGFVFALLRYLRGCRNQTFHLGARA |
| | | GFLKEIRKELEKTRWGKAKEAEHVVLTDKTVAAIRAIIDNDAK |
| | | ALGARLLADLSGAFVAHYASKEHFSTLYSEIVKAVKDAPEVSSG |
| | | LPRLKLLLKRADGVRGYVHGLRDTRKHAFATKLPPPPAPRELD |
| | | DPATKARYIALLRLYDGPFRAYASGITGTALAGPAARAKEAATA |
| | | LAQSVNVTKAYSDVMEGRSSRLRPPNDGETLREYLSALTGETAT |
| | | EFRVQIGYESDSENARKQAEFIENYRRDMLAFMFEDYIRAKGFD |
| | | WILKIEPGATAMTRAPVLPEPIDTRGQYEHWQAALYLVMHFVP |
| | | ASDVSNLLHQLRKWEALQGKYELVQDGDATDQADARREALDL |
| | | VKRFRDVLVLFLKTGEARFEGRAAPFDLKPFRALFANPATFDRL |
| | | FMATPTTARPAEDDPEGDGASEPELRVARTLRGLRQIARYNHM |
| | | AVLSDLFAKHKVRDEEVARLAEIEDETQEKSQIVAAQELRTDLH |
| | | DKVMKCHPKTISPEERQSYAAAIKTIEEHRFLVGRVYLGDHLRL |
| | | HRLMMDVIGRLIDYAGAYERDTGTFLINASKQLGAGADWAVTI |
| | | AGAANTDARTQTRKDLAHFNVLDRADGTPDLTALVNRAREMM |
| | | AYDRKRKNAVPRSILDMLARLGLTLKWQMKDHLLQDATITQA |
| | | AIKHLDKVRLTVGGPAAVTEARFSQDYLQMVAAVENGSVQNPK |
| | | PRRRDDGDAWHKPPKPATAQSQPDQKPPNKAPSAGSRLPPPQV |
| | | GEVYEGVVVKVIDTGSLGFLAVEGVAGNIGLHISRLRRIREDAII |
| | | VGRRYRFRVEIYVPPKSNTSKLNAADLVRID |
| 134 | Carno- | MRITKVKIKLDNKLYQVTMQKEEKYGTLKLNEESRKSTAEILR |
| | bacterium | LKKASFNKSFHSKTINSQKENKNATIKKNGDYISQIFEKLVGVDT |
| | gallinarum | NKNIRKPKMSLTDLKDLPKKDLALFIKRKFKNDDIVEIKNLDLIS |
| | C2c2 amino | LFYNALQKVPGEHFTDESWADFCQEMMPYREYKNKFIERKIIL |
| | acid sequence | LANSIEQNKGFSINPETFSKRKRVLHQWAIEVQERGDFSILDEKL |
| | | SKLAEIYNFKKMCKRVQDELNDLEKSMKKGKNPEKEKEAYKK |
| | | QKNFKIKTIWKDYPYKTHIGLIEKIKENEELNQFNIEIGKYFEHY |
| | | FPIKKERCTEDEPYYLNSETIATTVNYQLKNALISYLMQIGKYK |
| | | QFGLENQVLDSKKLQEIGIYEGFQTKFMDACVFATSSLKNIIEP |
| | | MRSGDILGKREFKEAIATSSFVNYHHFFPYFPFELKGMKDRESE |
| | | LIPFGEQTEAKQMQNIWALRGSVQQIRNEIFHSFDKNQKFNLPQ |
| | | LDKSNFEFDASENSTGKSQSYIETDYKFLFEAEKNQLEQFFIERI |
| | | KSSGALEYYPLKSLEKLFAKKEMKFSLGSQVVAFAPSYKKLVK |
| | | KGHSYQTATEGTANYLGLSYYNRYELKEESFQAQYYLLKLIYQ |
| | | YVFLPNFSQGNSPAFRETVKAILRINKDEARKKMKKNKKFLRK |
| | | YAFEQVREMEFKETPDQYMSYLQSEMREEKVRKAEKNDKGFE |
| | | KNITMNFEKLLMQIFVKGFDVFLTTFAGKELLLSSEEKVIKETEI |
| | | SLSKKINEREKTLKASIQVEHQLVATNSAISYWLFCKLLDSRHL |
| | | NELRNEMIKFKQSRIKFNHTQHAELIQNLLPIVELTILSNDYDEK |
| | | NDSQNVDVSAYFEDKSLYETAPYVQTDDRTRVSFRPILKLEKYH |
| | | TKSLIEALLKDNPQFRVAATDIQEWMHKREEIGELVEKRKNLH |
| | | TEWAEGQQTLGAEKREEYRDYCKKIDRFNWKANKVTLTYLSQ |
| | | LHYLITDLLGRMVGFSALFERDLVYFSRSFSELGGETYHISDYK |
| | | NLSGVLRLNAEVKPIKIKNIKVIDNEENPYKGNEPEVKPFLDRLH |
| | | AYLENVIGIKAVHGKIRNQTAHLSVLQLELSMIESMNNLRDLMA |
| | | YDRKLKNAVTKSMIKILDKHGMILKLKIDENHKNFEIESLIPKEI |
| | | IHLKDKAIKTNQVSEEYCQLVLALLTTNPGNQLN |
| 135 | Herbinix | MKLTRRRISGNSVDQKITAAFYRDMSQGLLYYDSEDNDCTDKVI |
| | hemicellu- | ESMDFERSWRGRILKNGEDDKNPFYMFVKGLVGSNDKIVCEPI |
| | losilytica | DVDSDPDNLDILINKNLTGFGRNLKAPDSNDTLENLIRKIQAGIP |
| | C2c2 | EEEVLPELKKIKEMIQKDIVNRKEQLLKSIKNNRIPFSLEGSKLV |
| | amino acid | PSTKKMKWLFKLIDVPNKTFNEKMLEKYWEIYDYDKLKANITN |
| | sequence | RLDKTDKKARSISRAVSEELREYHKNLRTNYNRFVSGDRPAAGL |
| | | DNGGSAKYNPDKEEFLLFLKEVEQYFKKYFPVKSKHSNKSKDK |
| | | SLVDKYKNYCSYKVVKKEVNRSIINQLVAGLIQQGKLLYYFYYN |
| | | DTWQEDFLNSYGLSYIQVEEAFKKSVMTSLSWGINRLTSFFIDDS |
| | | NTVKFDDITTKKAKEAIESNYFNKLRTCSRMQDHFKEKLAFFYP |
| | | VYVKDKKDRPDDDIENLIVLVKNAIESVSYLRNRTFHFKESSLLE |
| | | LLKELDDKNSGQNKIDYSVAAEFIKRDIENLYDVFREQIRSLGIA |
| | | EYYKADMISDCFKTCGLEFALYSPKNSLMPAFKNVYKRGANLN |
| | | KAYIRDKGPKETGDQGQNSYKALEEYRELTWYIEVKNNDQSYN |
| | | AYKNLLQLIYYHAFLPEVRENEALITDFINRTKEWNRKETEERL |
| | | NTKNNKKHKNFDENDDITVNTYRYESIPDYQGESLDDYLKVLQ |
| | | RKQMARAKEVNEKEEGNNNYIQFIRDVVVWAFGAYLENKLKN |
| | | YKNELQPPLSKENIGLNDTLKELFPEEKVKSPFNIKCRFSISTFID |
| | | NKGKSTDNTSAEAVKTDGKEDEKDKKNIKRKDLLCFYLFLRLL |
| | | DENEICKLQHQFIKYRCSLKERRFPGNRTKLEKETELLAELEEL |
| | | MELVRFTMPSIPEISAKAESGYDTMIKKYFKDFIEKKVFKNPKTS |
| | | NLYYHSDSKTPVTRKYMALLMRSAPLHLYKDIFKGYYLITKKE |
| | | CLEYIKLSNIIKDYQNSLNELHEQLERIKLKSEKQNGKDSLYLDK |
| | | KDFYKVKEYVENLEQVARYKHLQHKINFESLYRIFRIHVDIAAR |
| | | MVGYTQDWERDMHFLFKALVYNGVLEERRFEAIFNNNDDNND |
| | | GRIVKKIQNNLNNKNRELVSMLCWNKKLNKNEFGAIIWKRNPI |
| | | AHLNHFTQTEQNSKSSLESLINSLRILLAYDRKRQNAVTKTINDL |
| | | LLNDYHIRIKWEGRVDEGQIYFNIKEKEDIENEPIIHLKHLHKKD |
| | | CYIYKNSYMFDKQKEWICNGIKEEVYDKSILKCIGNLFKFDYED |
| | | KNKSSANPKHT |
| 136 | Paludibacter | MRVSKVKVKDGGKDKMVLVHRKTTGAQLVYSGQPVSNETSNI |
| | propioni- | LPEKKRQSFDLSTLNKTIIKFDTAKKQKLNVDQYKIVEKIFKYP |
| | cigenes | KQELPKQIKAEEILPFLNHKFQEPVKYWKNGKEESFNLTLLIVE |
| | C2c2 | AVQAQDKRKLQPYYDWKTWYIQTKSDLLKKSIENNRIDLTENL |
| | amino acid | SKRKKALLAWETEFTASGSIDLTHYHKVYMTDVLCKMLQDVK |
| | sequence | PLTDDKGKINTNAYHRGLKKALQNHQPAIFGTREVPNEANRAD |
| | | NQLSIYHLEVVKYLEHYFPIKTSKRRNTADDIAHYLKAQTLKTT |
| | | IEKQLVNAIRANIIQQGKTNHHELKADTTSNDLIRIKTNEAFVLN |
| | | LTGTCAFAANNIRNMVDNEQTNDILGKGDFIKSLLKDNTNSQLY |
| | | SFFFGEGLSTNKAEKETQLWGIRGAVQQIRNNVNHYKKDALKT |
| | | VFNISNFENPTITDPKQQTNYADTIYKARFINELEKIPEAFAQQLK |
| | | TGGAVSYYTIENLKSLLTTFQFSLCRSTIPFAPGFKKVENGGINY |
| | | QNAKQDESFYELMLEQYLRKENFAEESYNARYFMLKLIYNNLF |
| | | LPGFTTDRKAFADSVGFVQMQNKKQAEKVNPRKKEAYAFEAV |
| | | RPMTAADSIADYMAYVQSELMQEQNKKEEKVAEETRINFEKFV |
| | | LQVFIKGFDSFLRAKEFDFVQMPQPQLTATASNQQKADKLNQL |
| | | EASITADCKLTPQYAKADDATHIAFYVFCKLLDAAHLSNLRNEL |
| | | IKFRESVNEFKFHHLLEIIEICLLSADVVPTDYRDLYSSEADCLAR |
| | | LRPFIEQGADITNWSDLFVQSDKHSPVIHANIELSVKYGTTKLLE |
| | | QIINKDTQFKTTEANFTAWNTAQKSIEQLIKQREDHHEQWVKA |
| | | KNADDKEKQERKREKSNFAQKFIEKHGDDYLDICDYINTYNWL |
| | | DNKMHFVHLNRLHGLTIELLGRMAGFVALFDRDFQFFDEQQIA |
| | | DEFKLHGFVNLHSIDKKLNEVPTKKIKEIYDIRNKIIQINGNKINE |
| | | SVRANLIQFISSKRNYYNNAFLHVSNDEIKEKQMYDIRNHIAHFN |
| | | YLTKDAADFSLIDLINELRELLHYDRKLKNAVSKAFIDLFDKHG |
| | | MILKLKLNADHKLKVESLEPKKIYHLGSSAKDKPEYQYCTNQV |
| | | MMAYCNMCRSLLEMKK |
| 137 | Leptotrichia | MYMKITKIDGVSHYKKQDKGILKKKWKDLDERKQREKIEARY |
| | wadei (Lwa) | NKQIESKIYKEFFRLKNKKRIEKEEDQNIKSLYFFIKELYLNEKN |
| | C2c2 amino | EEWELKNINLEILDDKERVIKGYKFKEDVYFFKEGYKEYYLRIL |
| | acid sequence | FNNLIEKVQNENREKVRKNKEFLDLKEIFKKYKNRKIDLLLKSI |
| | | NNNKINLEYKKENVNEEIYGINPTNDREMTFYELLKEIIEKKDEQ |
| | | KSILEEKLDNFDITNFLENIEKIFNEETEINIIKGKVLNELREYIKE |
| | | KEENNSDNKLKQIYNLELKKYIENNFSYKKQKSKSKNGKNDYL |
| | | YLNFLKKIMFIEEVDEKKEINKEKFKNKINSNFKNLFVQHILDYG |
| | | KLLYYKENDEYIKNTGQLETKDLEYIKTKETLIRKMAVLVSFAA |
| | | NSYYNLFGRVSGDILGTEVVKSSKTNVIKVGSHIFKEKMLNYFF |
| | | DFEIFDANKIVEILESISYSIYNVRNGVGHFNKLILGKYKKKDINT |
| | | NKRIEEDLNNNEEIKGYFIKKRGEIERKVKEKFLSNNLQYYYSK |
| | | EKIENYFEVYEFEILKRKIPFAPNFKRIIKKGEDLFNNKNNKKYE |
| | | YFKNFDKNSAEEKKEFLKTRNFLLKELYYNNFYKEFLSKKEEFE |
| | | KIVLEVKEEKKSRGNINNKKSGVSFQSIDDYDTKINISDYIASIHK |
| | | KEMERVEKYNEEKQKDTAKYIRDFVEEIFLTGFINYLEKDKRLH |
| | | FLKEEFSILCNNNNNVVDFNININEEKIKEFLKENDSKTLNLYLFF |
| | | NMIDSKRISEFRNELVKYKQFTKKRLDEEKEFLGIKIELYETLIE |
| | | FVILTREKLDTKKSEEIDAWLVDKLYVKDSNEYKEYEEILKLFV |
| | | DEKILSSKEAPYYATDNKTPILLSNFEKTRKYGTQSFLSEIQSNY |
| | | KYSKVEKENIEDYNKKEEIEQKKKSNIEKLQDLKVELHKKWEQ |
| | | NKITEKEIEKYNNTTRKINEYNYLKNKEELQNVYLLHEMLSDLL |
| | | ARNVAFFNKWERDFKFIVIAIKQFLRENDKEKVNEFLNPPDNSK |
| | | GKKVYFSVSKYKNTVENIDGIHKNFMNLIFLNNKFMNRKIDKM |
| | | NCAIWVYFRNYIAHFLHLHTKNEKISLISQMNLLIKLFSYDKKV |
| | | QNHILKSTKTLLEKYNIQINFEISNDKNEVFKYKIKNRLYSKKGK |
| | | MLGKNNKFEILENEFLENVKAMLEYSE |
| 138 | Bergeyella | MENKTSLGNNIYYNPFKPQDKSYFAGYFNAAMENTDSVFRELG |
| | zoohelcum | KRLKGKEYTSENFFDAIFKENISLVEYERYVKLLSDYFPMARLL |
| | Cas13b | DKKEVPIKERKENFKKNFKGIIKAVRDLRNFYTHKEHGEVEITD |
| | | EIFGVLDEMLKSTVLTVKKKKVKTDKTKEILKKSIEKQLDILCQ |
| | | KKLEYLRDTARKIEEKRRNQRERGEKELVAPFKYSDKRDDLIA |
| | | AIYNDAFDVYIDKKKDSLKESSKAKYNTKSDPQQEEGDLKIPISK |
| | | NGVVFLLSLFLTKQEIHAFKSKIAGFKATVIDEATVSEATVSHGK |
| | | NSICFMATHEIFSHLAYKKLKRKVRTAEINYGEAENAEQLSVYA |
| | | KETLMMQMLDELSKVPDVVYQNLSEDVQKTFIEDWNEYLKEN |
| | | NGDVGTMEEEQVIHPVIRKRYEDKFNYFAIRFLDEFAQFPTLRF |
| | | QVHLGNYLHDSRPKENLISDRRIKEKITVFGRLSELEHKKALFIK |
| | | NTETNEDREHYWEIFPNPNYDFPKENISVNDKDFPIAGSILDREK |
| | | QPVAGKIGIKVKLLNQQYVSEVDKAVKAHQLKQRKASKPSIQNI |
| | | IEEIVPINESNPKEAIVFGGQPTAYLSMNDIHSILYEFFDKWEKK |
| | | KEKLEKKGEKELRKEIGKELEKKIVGKIQAQIQQIIDKDTNAKI |
| | | LKPYQDGNSTAIDKEKLIKDLKQEQNILQKLKDEQTVREKEYN |
| | | DFIAYQDKNREINKVRDRNHKQYLKDNLKRKYPEAPARKEVLY |
| | | YREKGKVAVWLANDIKRFMPTDFKNEWKGEQHSLLQKSLAYY |
| | | EQCKEELKNLLPEKVFQHLPFKLGGYFQQKYLYQFYTCYLDKR |
| | | LEYISGLVQQAENFKSENKVFKKVENECFKFLKKQNYTHKELD |
| | | ARVQSILGYPIFLERGFMDEKPTIIKGKTFKGNEALFADWFRYY |
| | | KEYQNFQTFYDTENYPLVELEKKQADRKRKTKIYQQKKNDVFT |
| | | LLMAKHIFKSVFKQDSIDQFSLEDLYQSREERLGNQERARQTGE |
| | | RNTNYIWNKTVDLKLCDGKITVENVKLKNVGDFIKYEYDQRVQ |
| | | AFLKYEENIEWQAFLIKESKEEENYPYVVEREIEQYEKVRREEL |
| | | LKEVHLIEEYILEKVKDKEILKKGDNQNFKYYILNGLLKQLKNE |
| | | DVESYKVFNLNTEPEDVNINQLKQEATDLEQKAFVLTYIRNKFA |
| | | HNQLPKKEFWDYCQEKYGKIEKEKTYAEYFAEVFKKEKEALIK |
| 139 | Prevotella | MEDDKKTTDSIRYELKDKHFWAAFLNLARHNVYITVNHINKILE |
| | intermedia | EGEINRDGYETTLKNTWNEIKDINKKDRLSKLIIKHFPFLEAATY |
| | Cas13b | RLNPTDTTKQKEEKQAEAQSLESLRKSFFVFIYKLRDLRNHYSH |
| | | YKHSKSLERPKFEEGLLEKMYNIFNASIRLVKEDYQYNKDINPD |
| | | EDFKHLDRTEEEFNYYFTKDNEGNITESGLLFFVSLFLEKKDAI |
| | | WMQQKLRGFKDNRENKKKMTNEVFCRSRMLLPKLRLQSTQT |
| | | QDWILLDMLNELIRCPKSLYERLREEDREKFRVPIEIADEDYDA |
| | | EQEPFKNTLVRHQDRFPYFALRYFDYNEIFTNLRFQIDLGTYHFS |
| | | IYKKQIGDYKESHHLTHKLYGFERIQEFTKQNRPDEWRKFVKT |
| | | FNSFETSKEPYIPETTPHYHLENQKIGIRFRNDNDKIWPSLKTNS |
| | | EKNEKSKYKLDKSFQAEAFLSVHELLPMMFYYLLLKTENTDND |
| | | NEIETKKKENKNDKQEKHKIEEIIENKITEIYALYDTFANGEIKSI |
| | | DELEEYCKGKDIEIGHLPKQMIAILKDEHKVMATEAERKQEEM |
| | | LVDVQKSLESLDNQINEEIENVERKNSSLKSGKIASWLVNDMMR |
| | | FQPVQKDNEGKPLNNSKANSTEYQLLQRTLAFFGSEHERLAPYF |
| | | KQTKLIESSNPHPFLKDTEWEKCNNILSFYRSYLEAKKNFLESL |
| | | KPEDWEKNQYFLKLKEPKTKPKTLVQGWKNGFNLPRGIFTEPI |
| | | RKWFMKHRENITVAELKRVGLVAKVIPLFFSEEYKDSVQPFYN |
| | | YHFNVGNINKPDEKNFLNCEERRELLRKKKDEFKKMTDKEKEE |
| | | NPSYLEFKSWNKFERELRLVRNQDIVTWLLCMELFNKKKIKEL |
| | | NVEKIYLKNINTNTTKKEKNTEEKNGEEKNIKEKNNILNRIMPM |
| | | RLPIKVYGRENFSKNKKKKIRRNTFFTVYIEEKGTKLLKQGNFK |
| | | ALERDRRLGGLFSFVKTPSKAESKSNTISKLRVEYELGEYQKAR |
| | | IEIIKDMLALEKTLIDKYNSLDTDNFNKMLTDWLELKGEPDKAS |
| | | FQNDVDLLIAVRNAFSHNQYPMRNRIAFANINPFSLSSANTSEEK |
| | | GLGIANQLKDKTHKTIEKIIEIEKPIETKE |
| 140 | Prevotella | MQKQDKLFVDRKKNAIFAFPKYITIMENKEKPEPIYYELTDKHF |
| | buccae | WAAFLNLARHNVYTTINHINRRLEIAELKDDGYMMGIKGSWNE |
| | Cas13b | QAKKLDKKVRLRDLIMKHFPFLEAAAYEMTNSKSPNNKEQREK |
| | | EQSEALSLNNLKNVLFIFLEKLQVLRNYYSHYKYSEESPKPIFET |
| | | SLLKNMYKVFDANVRLVKRDYMHHENIDMQRDFTHLNRKKQV |
| | | GRTKNIIDSPNFHYHFADKEGNMTIAGLLFFVSLFLDKKDAIWM |
| | | QKKLKGFKDGRNLREQMTNEVFCRSRISLPKLKLENVQTKDW |
| | | MQLDMLNELVRCPKSLYERLREKDRESFKVPFDIFSDDYNAEEE |
| | | PFKNTLVRHQDRFPYFVLRYFDLNEIFEQLRFQIDLGTYHFSIYN |
| | | KRIGDEDEVRHLTHHLYGFARIQDFAPQNQPEEWRKLVKDLDH |
| | | FETSQEPYISKTAPHYHLENEKIGIKFCSAHNNLFPSLQTDKTCN |
| | | GRSKFNLGTQFTAEAFLSVHELLPMMFYYLLLTKDYSRKESAD |
| | | KVEGIIRKEISNIYAIYDAFANNEINSIADLTRRLQNTNILQGHLP |
| | | KQMISILKGRQKDMGKEAERKIGEMIDDTQRRLDLLCKQTNQ |
| | | KIRIGKRNAGLLKSGKIADWLVNDMMRFQPVQKDQNNIPINNS |
| | | KANSTEYRMLQRALALFGSENFRLKAYFNQMNLVGNDNPHPFL |
| | | AETQWEHQTNILSFYRNYLEARKKYLKGLKPQNWKQYQHFLI |
| | | LKVQKTNRNTLVTGWKNSFNLPRGIFTQPIREWFEKHNNSKRIY |
| | | DQILSFDRVGFVAKAIPLYFAEEYKDNVQPFYDYPFNIGNRLKPK |
| | | KRQFLDKKERVELWQKNKELFKNYPSEKKKTDLAYLDFLSWK |
| | | KFERELRLIKNQDIVTWLMFKELFNMATVEGLKIGEIHLRDIDT |
| | | NTANEESNNILNRIMPMKLPVKTYETDNKGNILKERPLATFYIEE |
| | | TETKVLKQGNFKALVKDRRLNGLFSFAETTDLNLEEHPISKLSV |
| | | DLELIKYQTTRISIFEMTLGLEKKLIDKYSTLPTDSFRNMLERWL |
| | | QCKANRPELKNYVNSLIAVRNAFSHNQYPMYDATLFAEVKKFT |
| | | LFPSVDTKKIELNIAPQLLEIVGKAIKEIEKSENKN |
| 141 | Porphyromonas | MNTVPASENKGQSRTVEDDPQYFGLYLNLARENLIEVESHVRIK |
| | gingivalis | FGKKKLNEESLKQSLLCDHLLSVDRWTKVYGHSRRYLPFLHYF |
| | Cas13b | DPDSQIEKDHDSKTGVDPDSAQRLIRELYSLLDFLRNDFSHNRLD |
| | | GTTFEHLEVSPDISSFITGTYSLACGRAQSRFAVFFKPDDFVLAK |
| | | NRKEQLISVADGKECLTVSGFAFFICLFLDREQASGMLSRIRGFK |
| | | RTDENWARAVHETFCDLCIRHPHDRLESSNTKEALLLDMLNEL |
| | | NRCPRILYDMLPEEERAQFLPALDENSMNNLSENSLDEESRLLW |
| | | DGSSDWAEALTKRIRHQDRFPYLMLRFIEEMDLLKGIRFRVDL |
| | | GEIELDSYSKKVGRNGEYDRTITDHALAFGKLSDFQNEEEVSRM |
| | | ISGEASYPVRFSLFAPRYAIYDNKIGYCHTSDPVYPKSKTGEKRA |
| | | LSNPQSMGFISVHDLRKLLLMELLCEGSFSRMQSDFLRKANRIL |
| | | DETAEGKLQFSALFPEMRHRFIPPQNPKSKDRREKAETTLEKYK |
| | | QEIKGRKDKLNSQLLSAFDMDQRQLPSRLLDEWMNIRPASHSV |
| | | KLRTYVKQLNEDCRLRLRKFRKDGDGKARAIPLVGEMATFLSQ |
| | | DIVRMIISEETKKLITSAYYNEMQRSLAQYAGEENRRQFRAIVAE |
| | | LRLLDPSSGHPFLSATMETAHRYTEGFYKCYLEKKREWLAKIF |
| | | YRPEQDENTKRRISVFFVPDGEARKLLPLLIRRRMKEQNDLQD |
| | | WIRNKQAHPIDLPSHLFDSKVMELLKVKDGKKKWNEAFKDW |
| | | WSTKYPDGMQPFYGLRRELNIHGKSVSYIPSDGKKFADCYTHL |
| | | MEKTVRDKKRELRTAGKPVPPDLAADIKRSFHRAVNEREFMLR |
| | | LVQEDDRLMLMAINKMMTDREEDILPGLKNIDSILDEENQFSLA |
| | | VHAKVLEKEGEGGDNSLSLVPATIEIKSKRKDWSKYIRYRYDRR |
| | | VPGLMSHFPEHKATLDEVKTLLGEYDRCRIKIFDWAFALEGAI |
| | | MSDRDLKPYLHESSSREGKSGEHSTLVKMLVEKKGCLTPDESQ |
| | | YLILIRNKAAHNQFPCAAEMPLIYRDVSAKVGSIEGSSAKDLPEG |
| | | SSLVDSLWKKYEMIIRKILPILDPENRFFGKLLNNMSQPINDL |
| 142 | Bacteroides | MESIKNSQKSTGKTLQKDPPYFGLYLNMALLNVRKVENHIRKW |
| | pyogenes | LGDVALLPEKSGFHSLLTTDNLSSAKWTRFYYKSRKFLPFLEMF |
| | Cas13b | DSDKKSYENRRETAECLDTIDRQKISSLLKEVYGKLQDIRNAFS |
| | | HYHIDDQSVKHTALIISSEMHRFIENAYSFALQKTRARFTGVFVE |
| | | TDFLQAEEKGDNKKFFAIGGNEGIKLKDNALIFLICLFLDREEAF |
| | | KFLSRATGFKSTKEKGFLAVRETFCALCCRQPHERLLSVNPREA |
| | | LLMDMLNELNRCPDILFEMLDEKDQKSFLPLLGEEEQAHILENS |
| | | LNDELCEAIDDPFEMIASLSKRVRYKNRFPYLMLRYIEEKNLLPF |
| | | IRFRIDLGCLELASYPKKMGEENNYERSVTDHAMAFGRLTDFH |
| | | NEDAVLQQITKGITDEVRFSLYAPRYAIYNNKIGFVRTSGSDKISF |
| | | PTLKKKGGEGHCVAYTLQNTKSFGFISIYDLRKILLLSFLDKDK |
| | | AKNIVSGLLEQCEKHWKDLSENLFDAIRTELQKEFPVPLIRYTL |
| | | PRSKGGKLVSSKLADKQEKYESEFERRKEKLTEILSEKDFDLSQI |
| | | PRRMIDEWLNVLPTSREKKLKGYVETLKLDCRERLRVFEKREK |
| | | GEHPLPPRIGEMATDLAKDIIRMVIDQGVKQRITSAYYSEIQRCL |
| | | AQYAGDDNRRHLDSIIRELRLKDTKNGHPFLGKVLRPGLGHTE |
| | | KLYQRYFEEKKEWLEATFYPAASPKRVPRFVNPPTGKQKELPLI |
| | | IRNLMKERPEWRDWKQRKNSHPIDLPSQLFENEICRLLKDKIGK |
| | | EPSGKLKWNEMFKLYWDKEFPNGMQRFYRCKRRVEVFDKVV |
| | | EYEYSEEGGNYKKYYEALIDEVVRQKISSSKEKSKLQVEDLTLS |
| | | VRRVFKRAINEKEYQLRLLCEDDRLLFMAVRDLYDWKEAQLD |
| | | LDKIDNMLGEPVSVSQVIQLEGGQPDAVIKAECKLKDVSKLMR |
| | | YCYDGRVKGLMPYFANHEATQEQVEMELRHYEDHRRRVFNW |
| | | VFALEKSVLKNEKLRRFYEESQGGCEHRRCIDALRKASLVSEEE |
| | | YEFLVHIRNKSAHNQFPDLEIGKLPPNVTSGFCECIWSKYKAIIC |
| | | RIIPFIDPERRFFGKLLEQK |
| 143 | Cas13c | MTEKKSIIFKNKSSVEIVKKDIFSQTPDNMIRNYKITLKISEKNPR |
| | | VVEAEIEDLMNSTILKDGRRSARREKSMTERKLIEEKVAENYSL |
| | | LANCPMEEVDSIKIYKIKRFLTYRSNMLLYFASINSFLCEGIKGK |
| | | DNETEEIWHLKDNDVRKEKVKENFKNKLIQSTENYNSSLKNQIE |
| | | EKEKLLRKESKKGAFYRTIIKKLQQERIKELSEKSLTEDCEKIIK |
| | | LYSELRHPLMHYDYQYFENLFENKENSELTKNLNLDIFKSLPLV |
| | | RKMKLNNKVNYLEDNDTLFVLQKTKKAKTLYQIYDALCEQKN |
| | | GFNKFINDFFVSDGEENTVFKQIINEKFQSEMEFLEKRISESEKK |
| | | NEKLKKKFDSMKAHFHNINSEDTKEAYFWDIHSSSNYKTKYNE |
| | | RKNLVNEYTELLGSSKEKKLLREEITQINRKLLKLKQEMEEITK |
| | | KNSLFRLEYKMKIAFGFLFCEFDGNISKFKDEFDASNQEKIIQYH |
| | | KNGEKYLTYFLKEEEKEKFNLEKMQKIIQKTEEEDWLLPETKN |
| | | NLFKFYLLTYLLLPYELKGDFLGFVKKHYYDIKNVDFMDENQN |
| | | NIQVSQTVEKQEDYFYHKIRLFEKNTKKYEIVKYSIVPNEKLKQ |
| | | YFEDLGIDIKYLTGSVESGEKWLGENLGIDIKYLTVEQKSEVSEE |
| | | KIKKFL |
| 144 | Cas13c | MEKDKKGEKIDISQEMIEEDLRKILILFSRLRHSMVHYDYEFYQ |
| | | ALYSGKDFVISDKNNLENRMISQLLDLNIFKELSKVKLIKDKAIS |
| | | NYLDKNTTIHVLGQDIKAIRLLDIYRDICGSKNGFNKFINTMITIS |
| | | GEEDREYKEKVIEHFNKKMENLSTYLEKLEKQDNAKRNNKRV |
| | | YNLLKQKLIEQQKLKEWFGGPYVYDIHSSKRYKELYIERKKLV |
| | | DRHSKLFEEGLDEKNKKELTKINDELSKLNSEMKEMTKLNSKY |
| | | RLQYKLQLAFGFILEEFDLNIDTFINNFDKDKDLIISNFMKKRDI |
| | | YLNRVLDRGDNRLKNIIKEYKFRDTEDIFCNDRDNNLVKLYILM |
| | | YILLPVEIRGDFLGFVKKNYYDMKHVDFIDKKDKEDKDTFFHD |
| | | LRLFEKNIRKLEITDYSLSSGFLSKEHKVDIEKKINDFINRNGAM |
| | | KLPEDITIEEFNKSLILPIMKNYQINFKLLNDIEISALFKIAKDRSI |
| | | TFKQAIDEIKNEDIKKNSKKNDKNNHKDKNINFTQLMKRALHE |
| | | KIPYKAGMYQIRNNISHIDMEQLYIDPLNSYMNSNKNNITISEQIE |
| | | KIIDVCVTGGVTGKELNNNIINDYYMKKEKLVFNLKLRKQNDIV |
| | | SIESQEKNKREEFVFKKYGLDYKDGEINIIEVIQKVNSLQEELRN |
| | | IKETSKEKLKNKETLFRDISLINGTIRKNINFKIKEMVLDIVRMD |
| | | EIRHINIHIYYKGENYTRSNIIKFKYAIDGENKKYYLKQHEINDIN |
| | | LELKDKFVTLICNMDKHPNKNKQTINLESNYIQNVKFIIP |
| 145 | Cas13c | MENKGNNKKIDFDENYNILVAQIKEYFTKEIENYNNRIDNIIDKK |
| | | ELLKYSEKKEESEKNKKLEELNKLKSQKLKILTDEEIKADVIKII |
| | | KIFSDLRHSLMHYEYKYFENLFENKKNEELAELLNLNLFKNLTL |
| | | LRQMKIENKTNYLEGREEFNIIGKNIKAKEVLGHYNLLAEQKN |
| | | GFNNFINSFFVQDGTENLEFKKLIDEHFVNAKKRLERNIKKSKK |
| | | LEKELEKMEQHYQRLNCAYVWDIHTSTTYKKLYNKRKSLIEEY |
| | | NKQINEIKDKEVITAINVELLRIKKEMEEITKSNSLFRLKYKMQI |
| | | AYAFLEIEFGGNIAKFKDEFDCSKMEEVQKYLKKGVKYLKYYK |
| | | DKEAQKNYEFPFEEIFENKDTHNEEWLENTSENNLFKFYILTYL |
| | | LLPMEFKGDFLGVVKKHYYDIKNVDFTDESEKELSQVQLDKMI |
| | | GDSFFHKIRLFEKNTKRYEIIKYSILTSDEIKRYFRLLELDVPYFE |
| | | YEKGTDEIGIFNKNIILTIFKYYQIIFRLYNDLEIHGLFNISSDLDK |
| | | ILRDLKSYGNKNINFREFLYVIKQNNNSSTEEEYRKIWENLEAK |
| | | YLRLHLLTPEKEEIKTKTKEELEKLNEISNLRNGICHLNYKEIIE |
| | | EILKTEISEKNKEATLNEKIRKVINFIKENELDKVELGFNFINDFF |
| | | MKKEQFMFGQIKQVKEGNSDSITTERERKEKNNKKLKETYELN |
| | | CDNLSEFYETSNNLRERANSSSLLEDSAFLKKIGLYKVKNNKVN |
| | | SKVKDEEKRIENIKRKLLKDSSDIMGMYKAEVVKKLKEKLILIF |
| | | KHDEEKRIYVTVYDTSKAVPENISKEILVKRNNSKEEYFFEDNN |
| | | KKYVTEYYTLEITETNELKVIPAKKLEGKEFKTEKNKENKLML |
| | | NNHYCFNVKIIY |
| 146 | Cas13c | MEEIKHKKNKSSIIRVIVSNYDMTGIKEIKVLYQKQGGVDTFNL |
| | | KTIINLESGNLEIISCKPKEREKYRYEFNCKTEINTISITKKDKVL |
| | | KKEIRKYSLELYFKNEKKDTVVAKVTDLLKAPDKIEGERNHLR |
| | | KLSSSTERKLLSKTLCKNYSEISKTPIEEIDSIKIYKIKRFLNYRSN |
| | | FLIYFALINDFLCAGVKEDDINEVWLIQDKEHTAFLENRIEKITD |
| | | YIFDKLSKDIENKKNQFEKRIKKYKTSLEELKTETLEKNKTFYID |
| | | SIKTKITNLENKITELSLYNSKESLKEDLIKIISIFTNLRHSLMHYD |
| | | YKSFENLFENIENEELKNLLDLNLFKSIRMSDEFKTKNRTNYLD |
| | | GTESFTIVKKHQNLKKLYTYYNNLCDKKNGFNTFINSFFVTDGI |
| | | ENTDFKNLIILHFEKEMEEYKKSIEYYKIKISNEKNKSKKEKLKE |
| | | KIDLLQSELINMREHKNLLKQIYFFDIHNSIKYKELYSERKNLIE |
| | | QYNLQINGVKDVTAINHINTKLLSLKNKMDKITKQNSLYRLKY |
| | | KLKIAYSFLMIEFDGDVSKFKNNFDPTNLEKRVEYLDKKEEYLN |
| | | YTAPKNKFNFAKLEEELQKIQSTSEMGADYLNVSPENNLFKFYI |
| | | LTYIMLPVEFKGDFLGFVKNHYYNIKNVDFMDESLLDENEVDSN |
| | | KLNEKIENLKDSSFFNKIRLFEKNIKKYEIVKYSVSTQENMKEYF |
| | | KQLNLDIPYLDYKSTDEIGIFNKNMILPIFKYYQNVFKLCNDIEIH |
| | | ALLALANKKQQNLEYAIYCCSKKNSLNYNELLKTFNRKTYQNL |
| | | SFIRNKIAHLNYKELFSDLFNNELDLNTKVRCLIEFSQNNKFDQI |
| | | DLGMNFINDYYMKKTRFIFNQRRLRDLNVPSKEKIIDGKRKQQ |
| | | NDSNNELLKKYGLSRTNIKDIFNKAWY |
| 147 | Cas13c | MKVRYRKQAQLDTFIIKTEIVNNDIFIKSIIEKAREKYRYSFLFDG |
| | | EEKYHFKNKSSVEIVKNDIFSQTPDNMIRNYKITLKISEKNPRVV |
| | | EAEIEDLMNSTILKDGRRSARREKSMTERKLIEEKVAENYSLLA |
| | | NCPIEEVDSIKIYKIKRFLTYRSNMLLYFASINSFLCEGIKGKDNE |
| | | TEEIWHLKDNDVRKEKVKENFKNKLIQSTENYNSSLKNQIEEKE |
| | | KLSSKEFKKGAFYRTIIKKLQQERIKELSEKSLTEDCEKIIKLYSE |
| | | LRHPLMHYDYQYFENLFENKENSELTKNLNLDIFKSLPLVRKM |
| | | KLNNKVNYLEDNDTLFVLQKTKKAKTLYQIYDALCEQKNGFN |
| | | KFINDFFVSDGEENTVFKQIINEKFQSEMEFLEKRISESEKKNEK |
| | | LKKKLDSMKAHFRNINSEDTKEAYFWDIHSSRNYKTKYNERKN |
| | | LVNEYTKLLGSSKEKKLLREEITKINRQLLKLKQEMEEITKKNS |
| | | LFRLEYKMKIAFGFLFCEFDGNISKFKDEFDASNQEKIIQYHKN |
| | | GEKYLTSFLKEEEKEKFNLEKMQKIIQKTEEEDWLLPETKNNL |
| | | FKFYLLTYLLLPYELKGDFLGFVKKHYYDIKNVDFMDENQNNI |
| | | QVSQTVEKQEDYFYHKIRLFEKNTKKYEIVKYSIVPNEKLKQYF |
| | | EDLGIDIKYLTGSVESGEKWLGENLGIDIKYLTVEQKSEVSEEK |
| | | NKKVSLKNNGMFNKTILLFVFKYYQIAFKLFNDIELYSLFFLRE |
| | | KSEKPFEVFLEELKDKMIGKQLNFGQLLYVVYEVLVKNKDLDK |
| | | ILSKKIDYRKDKSFSPEIAYLRNFLSHLNYSKFLDNFMKINTNKS |
| | | DENKEVLIPSIKIQKMIQFIEKCNLQNQIDFDFNFVNDFYMRKEK |
| | | MFFIQLKQIFPDINSTEKQKKSEKEEILRKRYHLINKKNEQIKDE |
| | | HEAQSQLYEKILSLQKIFSCDKNNFYRRLKEEKLLFLEKQGKKK |
| | | ISMKEIKDKIASDISDLLGILKKEITRDIKDKLTEKFRYCEEKLLN |
| | | ISFYNHQDKKKEEGIRVFLIRDKNSDNFKFESILDDGSNKIFISKN |
| | | GKEITIQCCDKVLETLMIEKNTLKISSNGKIISLIPHYSYSIDVKY |

The programmable nuclease can be Cas13. Sometimes the Cas13 can be Cas13a, Cas13b, Cas13c, Cas13d, or Cas13e. In some cases, the programmable nuclease can be Mad7 or Mad2. In some cases, the programmable nuclease can be Cas12. Sometimes the Cas12 can be Cas12a, Cas12b, Cas12c, Cas12d, or Cas12e. In some cases, the programmable nuclease can be Csm1, Cas9, C2c4, C2c8, C2c5, C2c10, C2c9, or CasZ. Sometimes, the Csm1 can also be also called smCms1, miCms1, obCms1, or suCms1. Sometimes Cas13a can also be also called C2c2. Sometimes CasZ can also be called Cas14a, Cas14b, Cas14c, Cas14d, Cas14e, Cas14f, Cas14g, or Cas14h. Sometimes, the programmable nuclease can be a type V CRISPR-Cas system. In some cases, the programmable nuclease can be a type VI CRISPR-Cas system. Sometimes the programmable nuclease can be a type III CRISPR-Cas system. In some cases, the programmable nuclease can be from at least one of Leptotrichia shahii (Lsh), Listeria seeligeri (Lse), Leptotrichia buccalis (Lbu), Leptotrichia wadei (Lwa), Rhodobacter capsulatus (Rca), Herbinix hemicellulosilytica (Hhe), Paludibacter propionicigenes (Ppr), Lachnospiraceae bacterium (Lba), [Eubacterium] rectale (Ere), Listeria newyorkensis (Lny), Clostridium aminophilum (Cam), Prevotella sp. (Psm), Capnocytophaga canimorsus (Cca, Lachnospiraceae bacterium (Lba), Bergeyella zoohelcum (Bzo), Prevotella intermedia (Pin), Prevotella buccae (Pbu), Alistipes sp. (Asp), Riemerella anatipestifer (Ran), Prevotella aurantiaca (Pau), Prevotella saccharolytica (Psa), Prevotella intermedia (Pint), Capnocytophaga canimorsus (Cca), Porphyromonas gulae (Pgu), Prevotella sp. (Psp), Porphyromonas gingivalis (Pig), Prevotella intermedia (Pini), Enterococcus italicus (E1), Lactobacillus salivarius (Ls), or Thermus thermophilus (Tt). Sometimes the Cas13 is at least one of LbuCas13a, LwaCas13a, LbaCas13a, HheCas13a, PprCas13a, EreCas13a, CamCas13a, or LshCas13a. The trans cleavage activity of the CRISPR enzyme can be activated when the crRNA is complexed with the target nucleic acid. The trans cleavage activity of the CRISPR enzyme can be activated when the guide nucleic acid comprising a tracrRNA and crRNA are complexed with the target nucleic acid. In some embodiments, the target nucleic acid is RNA.

In some embodiments, the programmable nuclease comprises Cas12, wherein the Cas12 enzyme binds and cleaves double stranded DNA and single stranded DNA. In some embodiments, programmable nucleases comprise Cas13, wherein the Cas13 enzyme binds and cleaves single stranded RNA. In some embodiments, programmable nucleases comprise Cas14, wherein the Cas14 enzyme binds and cleaves both double stranded DNA and single stranded DNA.

B. Guide Nucleic Acids

A guide nucleic acid can comprise a sequence that is reverse complementary to the sequence of a target nucleic acid. A guide nucleic acid can include a crRNA. Sometimes, a guide nucleic acid comprises a crRNA and tracrRNA. The guide nucleic acid can bind specifically to the target nucleic acid. In some cases, the guide nucleic acid is not naturally occurring and is instead made by artificial combination of otherwise separate segments of sequence. Often, the artificial combination is performed by chemical synthesis, by genetic engineering techniques, or by the artificial manipulation of isolated segments of nucleic acids. The target nucleic acid can be designed and made to provide desired functions. In some cases, the targeting region of a guide nucleic acid is 20 nucleotides in length. The targeting region of the guide nucleic acid may have a length of at least 10, 11, 12, 13, 14, 15, 16, 17, 18, 19, 20, 21, 22, 23, 24, 25, 26, 27, 28, 29, or 30 nucleotides in length. In some instances, the targeting region of the guide nucleic acid is 10, 11, 12, 13, 14, 15, 16, 17, 18, 19, 20, 21, 22, 23, 24, 25, 26, 27, 28, 29, or 30 nucleotides in length. In some cases, the targeting region of a guide nucleic acid has a length from exactly or about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. It is understood that the sequence of a polynucleotide need not be 100% complementary to that of its target nucleic acid to be specifically hybridizable or hybridizable or able to bind specifically. The guide nucleic acid can have a sequence comprising at least one uracil in a region from nucleic acid residue 5 to 20 that is reverse complementary to a modification variable region in the target nucleic acid. The guide nucleic acid, in some cases, has a sequence comprising at least one uracil in a region from nucleic acid residue 5 to 9, 10 to 14, or 15 to 20 that is reverse complementary to a modification variable region in the target nucleic acid. The guide nucleic acid can have a sequence comprising at least one uracil in a region from nucleic acid residue 5 to 20 that is reverse complementary to a methylation variable region in the target nucleic acid. The guide nucleic acid, in some cases, has a sequence comprising at least one uracil in a region from nucleic acid residue 5 to 9, 10 to 14, or 15 to 20 that is reverse complementary to a methylation variable region in the target nucleic acid.

The guide nucleic acid can be selected from a group of guide nucleic acids that have been tiled against the nucleic acid sequence of a strain of an infection or genomic locus of interest. The guide nucleic acid can be selected from a group of guide nucleic acids that have been tiled against the nucleic acid sequence of a strain of interest. Often, guide nucleic acids that are tiled against the nucleic acid of a strain of an infection or genomic locus of interest can be pooled for use in a method described herein. Often, these guide nucleic acids are pooled for detecting a target nucleic acid in a single assay. The pooling of guide nucleic acids that are tiled against a single target nucleic acid can enhance the detection of the target nucleic acid using the methods described herein. The pooling of guide nucleic acids that are tiled against a single target nucleic acid can ensure broad coverage of the target nucleic acid within a single reaction using the methods described herein. The tiling, for example, is sequential along the target nucleic acid. Sometimes, the tiling is overlapping along the target nucleic acid. In some instances, the tiling comprises gaps between the tiled guide nucleic acids along the target nucleic acid. In some instances the tiling of the guide nucleic acids is non-sequential. Often, a method for detecting a target nucleic acid comprises contacting a target nucleic acid to a pool of guide nucleic acids and a programmable nuclease, wherein a guide nucleic acid of the pool of guide nucleic acids has a sequence selected from a group of tiled guide nucleic acid that correspond to nucleic acids of a target nucleic acid; and assaying for a signal produce by cleavage of at least some reporters of a population of reporters. Pooling of guide nucleic acids can ensure broad spectrum identification, or broad coverage, of a target species within a single reaction. This can be particularly helpful in diseases or indications, like sepsis, that may be caused by multiple organisms.

C. Reporters

Also disclosed herein are reporters and methods detecting a target nucleic acid using the reporters. Often, the reporter is a protein-nucleic acid. For example, a method of assaying for a target nucleic acid in a sample comprises contacting the sample to a complex comprising a) a guide nucleic acid comprising a segment that is reverse complementary to a segment of the target nucleic acid and b) a programmable nuclease that exhibits sequence independent cleavage upon forming an activated complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. Often, the protein-nucleic acid is an enzyme-nucleic acid or an enzyme substrate-nucleic acid. Sometimes, the protein-nucleic acid is attached to a solid support. The nucleic acid can be DNA, RNA, or a DNA/RNA hybrid. The methods described herein use a programmable nuclease, such as the CRISPR/Cas system, to detect a target nucleic acid. A method of assaying for a target nucleic acid in a sample, for example, comprises: a) contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; b) contacting the complex to a substrate; c) contacting the substrate to a reagent that differentially reacts with a cleaved substrate; and d) assaying for a signal indicating cleavage of the substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid.

Cleavage of the protein-nucleic acid produces a signal. The lateral flow devices disclosed herein can be used to detect these signals, which indicate whether a target nucleic acid is present in the sample.

Described herein are reagents comprising a reporter further comprising a single stranded nucleic acid and a detection moiety, wherein the single stranded nucleic acid is capable of being cleaved by the activated programmable nuclease, thereby generating a first detectable signal. In some cases, the reporter is a single-stranded nucleic acid comprising deoxyribonucleotides. In other cases, the reporter is a single-stranded nucleic acid comprising ribonucleotides. The reporter can be a single-stranded nucleic acid comprising at least one deoxyribonucleotide and at least one ribonucleotide. In some cases, the reporter is a single-stranded nucleic acid comprising at least one ribonucleotide residue at an internal position that functions as a cleavage site. In some cases, the reporter comprises at least 2, 3, 4, 5, 6, 7, 8, 9, or 10 ribonucleotide residues at an internal position. Sometimes the ribonucleotide residues are continuous. Alternatively, the ribonucleotide residues are interspersed in between non-ribonucleotide residues. In some cases, the reporter has only ribonucleotide residues. In some cases, the reporter has only deoxyribonucleotide residues. In some cases, the reporter comprises nucleotides resistant to cleavage by the programmable nuclease described herein. In some cases, the reporter comprises synthetic nucleotides. In some cases, the reporter comprises at least one ribonucleotide residue and at least one non-ribonucleotide residue. In some cases, reporter is 5-20, 5-15, 5-10, 7-20, 7-15, or 7-10 nucleotides in length. In some cases, the reporter comprises at least one uracil ribonucleotide. In some cases, the reporter comprises at least two uracil ribonucleotides. Sometimes the reporter has only uracil ribonucleotides. In some cases, the reporter comprises at least one adenine ribonucleotide. In some cases, the reporter comprises at least two adenine ribonucleotide. In some cases, the reporter has only adenine ribonucleotides. In some cases, the reporter comprises at least one cytosine ribonucleotide. In some cases, the reporter comprises at least two cytosine ribonucleotide. In some cases, the reporter comprises at least one guanine ribonucleotide. In some cases, the reporter comprises at least two guanine ribonucleotide. A reporter can comprise only unmodified ribonucleotides, only unmodified deoxyribonucleotides, or a combination thereof. In some cases, the reporter is from 5 to 12 nucleotides in length. In some cases, the reporter is at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some cases, the reporter is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. For cleavage by a programmable nuclease comprising Cas13, a reporter can be 5, 8, or 10 nucleotides in length. For cleavage by a programmable nuclease comprising Cas12, a reporter can be 10 nucleotides in length.

In some embodiments, the single stranded reporter comprises a detection moiety capable of generating a first detectable signal. Sometimes the reporter comprises a protein capable of generating a signal. A signal can be optical (e.g., fluorescent, colorometric, etc.). In some cases, a detection moiety is on one side of the cleavage site. Optionally, a quenching moiety is on the other side of the cleavage site. Sometimes the quenching moiety is a fluorescence quenching moiety. In some cases, the quenching moiety is 5′ to the cleavage site and the detection moiety is 3′ to the cleavage site. In some cases, the detection moiety is 5′ to the cleavage site and the quenching moiety is 3′ to the cleavage site. Sometimes the quenching moiety is at the 5′ terminus of the reporter. Sometimes the detection moiety is at the 3′ terminus of the reporter. In some cases, the detection moiety is at the 5′ terminus of the reporter. In some cases, the quenching moiety is at the 3′ terminus of the reporter. In some cases, the single-stranded reporter is at least one population of the single-stranded nucleic acid capable of generating a first detectable signal. In some cases, the single-stranded reporter is a population of the single stranded nucleic acid capable of generating a first detectable signal. Optionally, there is more than one population of single-stranded reporter. In some cases, there are 2, 3, 4, 5, 6, 7, 8, 9, 10, 11, 12, 13, 14, 15, 20, 30, 40, 50, or greater than 50, or any number spanned by the range of this list of different populations of single-stranded reporters capable of generating a detectable signal.

TABLE 4
Exemplary Single Stranded Reporter

| 5′ Detection Moiety* | Sequence (SEQ ID NO:) | 3′ Quencher* |
|---|---|---|
| /56-FAM/ | rUrUrUrUrU | /3IABkFQ/ |
| /5IRD700/ | rUrUrUrUrU | /3IRQC1N/ |
| /5TYE665/ | rUrUrUrUrU | /3IAbRQSp/ |
| /5Alex594N/ | rUrUrUrUrU | /3IAbRQSp/ |
| /5ATTO633N/ | rUrUrUrUrU | /3IAbRQSp/ |
| /56-FAM/ | rUrUrUrUrUrUrUrU | /3IABkFQ/ |
| /5IRD700/ | rUrUrUrUrUrUrUrU | /3IRQC1N/ |
| /5TYE665/ | rUrUrUrUrUrUrUrU | /3IAbRQSp/ |
| /5Alex594N/ | rUrUrUrUrUrUrUrU | /3IAbRQSp/ |
| /5ATTO633N/ | rUrUrUrUrUrUrUrU | /3IAbRQSp/ |
| /56-FAM/ | rUrUrUrUrUrUrUrUrUrU (SEQ ID | /3IABkFQ/ |
| | NO: 3) | |
| /5IRD700/ | rUrUrUrUr UrUrUrUrUrU (SEQ ID | /3IRQC1N/ |
| | NO: 3) | |
| /5TYE665/ | rUrUrUrUrUrUrUrUrUrU (SEQ ID | /3IAbRQSp/ |
| | NO: 3) | |
| /5Alex594N/ | rUrUrUrUrUrUrUrUrUrU (SEQ ID | /3IAbRQSp/ |
| | NO: 3) | |
| /5ATTO633N/ | rUrUrUrUrUrUrUrUrUrU (SEQ ID | /3IAbRQSp/ |
| | NO: 3) | |
| /56-FAM/ | TTTTrUrUTTTT (SEQ ID NO: 4) | /3IABkFQ/ |
| /5IRD700/ | TTTTrUrUTTTT (SEQ ID NO: 4) | /3IRQC1N/ |
| /5TYE665/ | TTTTrUrUTTTT (SEQ ID NO: 4) | /3IAbRQSp/ |
| /5Alex594N/ | TTTTrUrUTTTT (SEQ ID NO: 4) | /3IAbRQSp/ |
| /5ATTO633N/ | TTTTrUrUTTTT (SEQ ID NO: 4) | /3IAbRQSp/ |
| /56-FAM/ | TTrUrUTT | /3IABkFQ/ |
| /5IRD700/ | TTrUrUTT | /3IRQC1N/ |
| /5TYE665/ | TTrUrUTT | /3IAbRQSp/ |
| /5Alex594N/ | TTrUrUTT | /3IAbRQSp/ |
| /5ATTO633N/ | TTrUrUTT | /3IAbRQSp/ |
| /56-FAM/ | TArArUGC | /3IABkFQ/ |
| /5IRD700/ | TArArUGC | /3IRQC1N/ |
| /5TYE665/ | TArArUGC | /3IAbRQSp/ |
| /5Alex594N/ | TArArUGC | /3IAbRQSp/ |
| /5ATTO633N/ | TArArUGC | /3IAbRQSp/ |
| /56-FAM/ | TArUrGGC | /3IABkFQ/ |
| /5IRD700/ | TArUrGGC | /3IRQC1N/ |
| /5TYE665/ | TArUrGGC | /3IAbRQSp/ |
| /5Alex594N/ | TArUrGGC | /3IAbRQSp/ |
| /5ATTO633N/ | TArUrGGC | /3IAbRQSp/ |
| /56-FAM/ | rUrUrUrUrU | /3IABkFQ/ |
| /5IRD700/ | rUrUrUrUrU | /3IRQC1N/ |
| /5TYE665/ | rUrUrUrUrU | /3IAbRQSp/ |
| /5Alex594N/ | rUrUrUrUrU | /3IAbRQSp/ |
| /5ATTO633N/ | rUrUrUrUrU | /3IAbRQSp/ |
| /56-FAM/ | TTATTATT | /3IABkFQ/ |
| /56-FAM/ | TTATTATT | /3IABkFQ/ |
| /5IRD700/ | TTATTATT | /3IRQC1N/ |
| /5TYE665/ | TTATTATT | /3IAbRQSp/ |
| /5Alex594N/ | TTATTATT | /3IAbRQSp/ |
| /5ATTO633N/ | TTATTATT | /3IAbRQSp/ |
| /56-FAM/ | TTTTTT | /3IABkFQ/ |
| /56-FAM/ | TTTTTTTT | /3IABkFQ/ |
| /56-FAM/ | TTTTTTTTTT (SEQ ID NO: 12) | /3IABkFQ/ |
| /56-FAM/ | TTTTTTTTTTTT (SEQ ID NO: 13) | /3IABkFQ/ |
| /56-FAM/ | TTTTTTTTTTTTTT (SEQ ID NO: 14) | /3IABkFQ/ |
| /56-FAM/ | AAAAAA | /3IABkFQ/ |
| /56-FAM/ | CCCCCC | /3IABkFQ/ |
| /56-FAM/ | GGGGGG | /3IABkFQ/ |
| /56-FAM/ | TTATTATT | /3IABkFQ/ |

/56-FAM/: 5′ 6-Fluorescein (Integrated DNA Technologies)
/3IABkFQ/: 3′ Iowa Black FQ (Integrated DNA Technologies)
/5IRD700/: 5′ IRDye 700 (Integrated DNA Technologies)
/5TYE665/: 5′ TYE 665 (Integrated DNA Technologies)
/5Alex594N/: 5′ Alexa Fluor 594 (NHS Ester) (Integrated DNA Technologies)
/5ATTO633N/: 5′ ATTO TM 633 (NHS Ester) (Integrated DNA Technologies)
/3IRQC1N/: 3′ IRDye QC-1 Quencher (Li-Cor)
/3IAbRQSp/: 3′ Iowa Black RQ (Integrated DNA Technologies)
rU: uracil ribonucleotide
rG: guanine ribonucleotide
*This Table refers to the detection moiety and quencher moiety as their tradenames and their source is identified. However, alternatives, generics, or non-tradename moieties with similar function from other sources can also be used.

A detection moiety can be an fluorophore. The detection moiety can be a fluorophore that emits fluorescence in the visible spectrum. In some embodiments, the detection moiety can be a fluorophore that emits fluorescence in the visible spectrum. In some embodiments, the detection moiety can be a fluorophore that emits fluorescence in the near-IR spectrum. In some embodiments, the detection moiety can be a fluorophore that emits fluorescence in the IR spectrum. A detection moiety can be a fluorophore that emits fluorescence in the range from 500 nm and 720 nm. In some cases, the detection moiety emits fluorescence at a wavelength of 700 nm or higher. In other cases, the detection moiety emits fluorescence at about 660 nm or about 670 nm. In some cases, the detection moiety emits fluorescence at in the range from 500 to 520, 500 to 540, 500 to 590, 590 to 600, 600 to 610, 610 to 620, 620 to 630, 630 to 640, 640 to 650, 650 to 660, 660 to 670, 670 to 680, 680 to 690, 690 to 700, 700 to 710, 710 to 720, or 720 to 730 nm. A detection moiety can be a fluorophore that emits a fluorescence in the same range as 6-Fluorescein, IR Dye 700, Alexa Fluor 488, TYE 665, or ATTO™ 633 (NHS Ester). A detection moiety can be fluorescein amidite, 6-Fluorescein, IRDye 700, TYE 665, Alexa Fluor 594, or ATTO™ 633 (NHS Ester). A detection moiety can be a fluorophore that emits a fluorescence in the same range as 6-Fluorescein (Integrated DNA Technologies), IRDye 700 (Integrated DNA Technologies), TYE 665 (Integrated DNA Technologies), Alexa Fluor 594 (Integrated DNA Technologies), or ATTO™ 633 (NHS Ester) (Integrated DNA Technologies). A detection moiety can be fluorescein amidite, 6-Fluorescein (Integrated DNA Technologies), IRDye 700 (Integrated DNA Technologies), TYE 665 (Integrated DNA Technologies), Alexa Fluor 594 (Integrated DNA Technologies), or ATTO™ 633 (NHS Ester) (Integrated DNA Technologies). Any of the detection moieties described herein can be from any commercially available source, can be an alternative with a similar function, a generic, or a non-tradename of the detection moieties listed.

A detection moiety can be chosen for use based on the type of sample to be tested. For example, a detection moiety that is an infrared fluorophore is used with a urine sample. As another example, SEQ ID NO: 1 with a fluorophore that emits around 520 nm is used for testing in non-urine samples, and SEQ ID NO: 8 with a fluorophore that emits a fluorescence around 700 nm is used for testing in urine samples.

A quenching moiety can be chosen based on its ability to quench the detection moiety. A quenching moiety can be a non-fluorescent fluorescence quencher. A quenching moiety can quench a detection moiety that emits fluorescence in the range from 500 nm and 720 nm. In some cases, the quenching moiety quenches a detection moiety that emits fluorescence at a wavelength of 700 nm or higher. In other cases, the quenching moiety quenches a detection moiety that emits fluorescence at about 660 nm or about 670 nm. In some cases, the quenching moiety quenches a detection moiety that emits fluorescence at in the range of from 500 to 520, 500 to 540, 500 to 590, 590 to 600, 600 to 610, 610 to 620, 620 to 630, 630 to 640, 640 to 650, 650 to 660, 660 to 670, 670 to 680, 680 to 690, 690 to 700, 700 to 710, 710 to 720, or 720 to 730 nm. A quenching moiety can quench fluorescein amidite, 6-Fluorescein, IRDye 700, TYE 665, Alexa Fluor 594, or ATTO™ 633 (NHS Ester). A quenching moiety can be Iowa Black RQ, Iowa Black FQ or IRDye QC-1 Quencher. A quenching moiety can quench fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO™ 633 (NHS Ester) (Integrated DNA Technologies). A quenching moiety can be Iowa Black RQ (Integrated DNA Technologies), Iowa Black FQ (Integrated DNA Technologies) or IRDye QC-1 Quencher (LiCor). Any of the quenching moieties described herein can be from any commercially available source, can be an alternative with a similar function, a generic, or a non-tradename of the quenching moieties listed.

The generation of the detectable signal indicative of the release of the detection moiety indicates that cleavage by the programmable nuclease has occurred and that the sample contains the target nucleic acid. In some cases, the detection moiety comprises a fluorescent dye. Sometimes the detection moiety comprises a fluorescence resonance energy transfer (FRET) pair. In some cases, the detection moiety comprises an infrared (IR) dye. In some cases, the detection moiety comprises an ultraviolet (UV) dye. Alternatively or in combination, the detection moiety comprises a polypeptide. Sometimes the detection moiety comprises a biotin. Sometimes the detection moiety comprises at least one of avidin or streptavidin. In some instances, the detection moiety comprises a polysaccharide, a polymer, or a nanoparticle. In some instances, the detection moiety comprises a gold nanoparticle or a latex nanoparticle.

A detection moiety can be any moiety capable of generating a calorimetric, potentiometric, amperometric, optical (e.g., fluorescent, colorimetric, etc.), or piezo-electric signal. A reporter, sometimes, is a protein-nucleic acid that is capable of generating a calorimetric, potentiometric, amperometric, optical (e.g., fluorescent, colorimetric, etc.), or piezo-electric signal via cleavage of the nucleic acid. Often a calorimetric signal is heat produced after cleavage of the reporters. Sometimes, a calorimetric signal is heat absorbed after cleavage of the reporters. A potentiometric signal, for example, is electrical potential produced after cleavage of the reporters. An amperometric signal can be movement of electrons produced after the cleavage of reporter. Often, the signal is an optical signal, such as a colorimetric signal or a fluorescence signal. An optical signal is, for example, a light output produced after the cleavage of the reporters. Sometimes, an optical signal is a change in light absorbance, emission or both, as measured before and after the cleavage of the reporters. Sometimes, an optical signal is an increase in light absorbance, emission, or both, as measured from before and after the cleavage of the reporter. Sometimes, an optical signal is a decrease in light absorbance, emission, or both, as measured from before and after the cleavage of the reporter. Often, a piezo-electric signal is a change in mass between before and after the cleavage of the reporter by the programmable nuclease.

Often, the protein-nucleic acid is an enzyme-nucleic acid. The enzyme may be sterically hindered when present as in the enzyme-nucleic acid, but then functional upon cleavage from the nucleic acid by the programmable nuclease. Often, the enzyme is an enzyme that produces a reaction with an enzyme substrate. An enzyme can be invertase. Often, the substrate of invertase is sucrose and DNS reagent.

Sometimes the protein-nucleic acid is a substrate-nucleic acid. Often the substrate is a substrate that produces a reaction with an enzyme. Release of the substrate upon cleavage by the programmable nuclease may free the substrate to react with the enzyme.

A protein-nucleic acid may be attached to a solid support. The solid support, for example, is a surface. A surface can be an electrode. Sometimes the solid support is a bead. Often the bead is a magnetic bead. Upon cleavage, the protein is liberated from the solid and interacts with other mixtures. For example, the protein is an enzyme, and upon cleavage of the nucleic acid of the enzyme-nucleic acid, the enzyme flows through a chamber into a mixture comprising the substrate. When the enzyme meets the enzyme substrate, a reaction occurs, such as a colorimetric reaction, which is then detected. As another example, the protein is an enzyme substrate, and upon cleavage of the nucleic acid of the enzyme substrate-nucleic acid, the enzyme flows through a chamber into a mixture comprising the enzyme. When the enzyme substrate meets the enzyme, a reaction occurs, such as a calorimetric reaction, which is then detected.

In some embodiments, the reporter comprises a nucleic acid conjugated to an affinity molecule which is in turn conjugated to the fluorophore (e.g., nucleic acid-affinity molecule-fluorophore) or the nucleic acid conjugated to the fluorophore which is in turn conjugated to the affinity molecule (e.g., nucleic acid-fluorophore-affinity molecule). In some embodiments, a linker conjugates the nucleic acid to the affinity molecule. In some embodiments, a linker conjugates the affinity molecule to the fluorophore. In some embodiments, a linker conjugates the nucleic acid to the fluorophore. A linker can be any suitable linker known in the art. In some embodiments, the nucleic acid of the reporter can be directly conjugated to the affinity molecule and the affinity molecule can be directly conjugated to the fluorophore or the nucleic acid can be directly conjugated to the fluorophore and the fluorophore can be directly conjugated to the affinity molecule. In this context, “directly conjugated” indicates that no intervening molecules, polypeptides, proteins, or other moieties are present between the two moieties directly conjugated to each other. For example, if a reporter comprises a nucleic acid directly conjugated to an affinity molecule and an affinity molecule directly conjugated to a fluorophore—no intervening moiety is present between the nucleic acid and the affinity molecule and no intervening moiety is present between the affinity molecule and the fluorophore. The affinity molecule can be biotin, avidin, streptavidin, or any similar molecule.

In some cases, the reporter comprises a substrate-nucleic acid. The substrate may be sequestered from its cognate enzyme when present as in the substrate-nucleic acid, but then is released from the nucleic acid upon cleavage, wherein the released substrate can contact the cognate enzyme to produce a detectable signal. Often, the substrate is sucrose and the cognate enzyme is invertase, and a DNS reagent can be used to monitor invertase activity.

A reporter may be a hybrid nucleic acid reporter. A hybrid nucleic acid reporter comprises a nucleic acid with at least one deoxyribonucleotide and at least one ribonucleotide. In some embodiments, the nucleic acid of the hybrid nucleic acid reporter can be of any length and can have any mixture of DNAs and RNAs. For example, in some cases, longer stretches of DNA can be interrupted by a few ribonucleotides. Alternatively, longer stretches of RNA can be interrupted by a few deoxyribonucleotides. Alternatively, every other base in the nucleic acid may alternate between ribonucleotides and deoxyribonucleotides. A major advantage of the hybrid nucleic acid reporter is increased stability as compared to a pure RNA nucleic acid reporter. For example, a hybrid nucleic acid reporter can be more stable in solution, lyophilized, or vitrified as compared to a pure DNA or pure RNA reporter.

The reporter can be lyophilized or vitrified. The reporter can be suspended in solution or immobilized on a surface. For example, the reporter can be immobilized on the surface of a chamber in a device as disclosed herein. In some cases, the reporter is immobilized on beads, such as magnetic beads, in a chamber of a device as disclosed herein where they can be held in position by a magnet placed below the chamber.

Additionally, target nucleic acid can optionally be amplified before binding to the guide nucleic acid (e.g., crRNA) of the programmable nuclease (e.g., CRISPR enzyme). This amplification can be PCR amplification or isothermal amplification. This nucleic acid amplification of the sample can improve at least one of sensitivity, specificity, or accuracy of the detection the target RNA. The reagents for nucleic acid amplification can comprise a recombinase, a oligonucleotide primer, a single-stranded DNA binding (SSB) protein, and a polymerase. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). The nucleic acid amplification can be performed for no greater than 1, 2, 3, 4, 5, 6, 7, 8, 9, 10, 11, 12, 13, 14, 15, 16, 17, 18, 19, 20, 25, 30, 40, 50, or 60 minutes. Sometimes, the nucleic acid amplification reaction is performed at a temperature of around 20-45° C. Sometimes, the nucleic acid amplification reaction is performed at a temperature of around 45-65° C. The nucleic acid amplification reaction can be performed at a temperature no greater than 20° C., 25° C., 30° C., 35° C., 37° C., 40° C., 45° C., 50° C., 55° C., 60° C., or 65° C. The nucleic acid amplification reaction can be performed at a temperature of at least 20° C., 25° C., 30° C., 35° C., 37° C., 40° C., 45° C., 50° C., 55° C., 60° C., or 65° C.

Disclosed herein are methods of assaying for a target nucleic acid as described herein wherein a signal is detected. For example, a method of assaying for a target nucleic acid in a sample comprises contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. As another example, a method of assaying for a target nucleic acid in a sample, for example, comprises: a) contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; b) contacting the complex to a reaction substrate; c) contacting the reaction substrate to a reagent that differentially reacts with a cleaved substrate; and d) assaying for a signal indicating cleavage of the reaction substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid.

A programmable nuclease can comprise a programmable nuclease capable of being activated when complexed with a guide nucleic acid and target nucleic acid. The programmable nuclease can become activated after binding of a guide nucleic acid with a target nucleic acid, in which the activated programmable nuclease can cleave the target nucleic acid and can have trans cleavage activity. Trans cleavage activity can be non-specific cleavage of nearby nucleic acids by the activated programmable nuclease, such as trans cleavage of reporters with a detection moiety. Once the reporter is cleaved by the activated programmable nuclease, the detection moiety can be released from the reporter and can generate a signal. The signal can be detected from a detection spot on a support medium, wherein the detection spot comprises capture probes for cleaved reporter fragments. The signal can be visualized to assess whether a target nucleic acid comprises a modification.

Often, the signal is a colorimetric signal or a signal visible by eye. In some cases, the first detection signal is generated by binding of the detection moiety to a capture molecule in a detection region, where the first detection signal indicates that the sample contained the target nucleic acid. Sometimes the system is capable of detecting more than one type of target nucleic acid, wherein the system comprises more than one type of guide nucleic acid and more than one type of reporter capable of directly or indirectly generating at least a first detection signal and a second detection signal. In some cases, the detectable signal is generated directly by the cleavage event. Alternatively or in combination, the detectable signal is generated indirectly by the signal event. Sometimes the detectable signal is not a fluorescent signal. In some instances, the detectable signal is a colorimetric or color-based signal. In some cases, the detected target nucleic acid is identified based on the spatial location of the detectable signal on the detection region of the support medium. In some cases, the second detectable signal is generated in a spatially distinct location than the first generated signal.

In some cases, the threshold of detection, for a subject method of detecting a single stranded target nucleic acid in a sample, is less than or equal to 10 nM. The term “threshold of detection” is used herein to describe the minimal amount of target nucleic acid that must be present in a sample in order for detection to occur. For example, when a threshold of detection is 10 nM, then a signal can be detected when a target nucleic acid is present in the sample at a concentration of 10 nM or more. In some cases, the threshold of detection is less than or equal to 5 nM, 1 nM, 0.5 nM, 0.1 nM, 0.05 nM, 0.01 nM, 0.005 nM, 0.001 nM, 0.0005 nM, 0.0001 nM, 0.00005 nM, 0.00001 nM, 10 pM, 1 pM, 500 fM, 250 fM, 100 fM, 50 fM, 10 fM, 5 fM, 1 fM, 500 attomole (aM), 100 aM, 50 aM, 10 aM, or 1 aM. In some cases, the threshold of detection is in a range of from 1 aM to 1 nM, 1 aM to 500 pM, 1 aM to 200 pM, 1 aM to 100 pM, 1 aM to 10 pM, 1 aM to 1 pM, 1 aM to 500 fM, 1 aM to 100 fM, 1 aM to 1 fM, 1 aM to 500 aM, 1 aM to 100 aM, 1 aM to 50 aM, 1 aM to 10 aM, 10 aM to 1 nM, 10 aM to 500 pM, 10 aM to 200 pM, 10 aM to 100 pM, 10 aM to 10 pM, 10 aM to 1 pM, 10 aM to 500 fM, 10 aM to 100 fM, 10 aM to 1 fM, 10 aM to 500 aM, 10 aM to 100 aM, 10 aM to 50 aM, 100 aM to 1 nM, 100 aM to 500 pM, 100 aM to 200 pM, 100 aM to 100 pM, 100 aM to 10 pM, 100 aM to 1 pM, 100 aM to 500 fM, 100 aM to 100 fM, 100 aM to 1 fM, 100 aM to 500 aM, 500 aM to 1 nM, 500 aM to 500 pM, 500 aM to 200 pM, 500 aM to 100 pM, 500 aM to 10 pM, 500 aM to 1 pM, 500 aM to 500 fM, 500 aM to 100 fM, 500 aM to 1 fM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, from 1 pM to 1 nM, 1 pM to 500 pM, 1 pM to 200 pM, 1 pM to 100 pM, or 1 pM to 10 pM. In some cases, the threshold of detection in a range of from 800 fM to 100 pM, 1 pM to 10 pM, 10 fM to 500 fM, 10 fM to 50 fM, 50 fM to 100 fM, 100 fM to 250 fM, or 250 fM to 500 fM. In some cases, the minimum concentration at which a single stranded target nucleic acid is detected in a sample is in a range of from 1 aM to 1 nM, 10 aM to 1 nM, 100 aM to 1 nM, 500 aM to 1 nM, 1 fM to 1 nM, 1 fM to 500 pM, 1 fM to 200 pM, 1 fM to 100 pM, 1 fM to 10 pM, 1 fM to 1 pM, 10 fM to 1 nM, 10 fM to 500 pM, 10 fM to 200 pM, 10 fM to 100 pM, 10 fM to 10 pM, 10 fM to 1 pM, 500 fM to 1 nM, 500 fM to 500 pM, 500 fM to 200 pM, 500 fM to 100 pM, 500 fM to 10 pM, 500 fM to 1 pM, 800 fM to 1 nM, 800 fM to 500 pM, 800 fM to 200 pM, 800 fM to 100 pM, 800 fM to 10 pM, 800 fM to 1 pM, 1 pM to 1 nM, 1 pM to 500 pM, from 1 pM to 200 pM, 1 pM to 100 pM, or 1 pM to 10 pM. In some cases, the minimum concentration at which a single stranded target nucleic acid can be detected in a sample is in a range of from 1 aM to 100 pM. In some cases, the minimum concentration at which a single stranded target nucleic acid can be detected in a sample is in a range of from 1 fM to 100 pM. In some cases, the minimum concentration at which a single stranded target nucleic acid can be detected in a sample is in a range of from 10 fM to 100 pM. In some cases, the minimum concentration at which a single stranded target nucleic acid can be detected in a sample is in a range of from 800 fM to 100 pM. In some cases, the minimum concentration at which a single stranded target nucleic acid can be detected in a sample is in a range of from 1 pM to 10 pM. In some cases, the streamlined lateral flow devices and methods described herein detect a target single-stranded nucleic acid in a sample comprising a plurality of nucleic acids such as a plurality of non-target nucleic acids, where the target single-stranded nucleic acid is present at a concentration as low as 1 aM, 10 aM, 100 aM, 500 aM, 1 fM, 10 fM, 500 fM, 800 fM, 1 pM, 10 pM, 100 pM, or 1 pM.

In some cases, the streamlined lateral flow devices and methods described herein detect a target single-stranded nucleic acid in a sample where the sample is contacted with the reagents for a predetermined length of time sufficient for the trans cleavage to occur or cleavage reaction to reach completion. In some cases, the streamlined lateral flow devices and methods described herein detect a target single-stranded nucleic acid in a sample where the sample is contacted with the reagents for no greater than 60 minutes. Sometimes the sample is contacted with the reagents for no greater than 120 minutes, 110 minutes, 100 minutes, 90 minutes, 80 minutes, 70 minutes, 60 minutes, 55 minutes, 50 minutes, 45 minutes, 40 minutes, 35 minutes, 30 minutes, 25 minutes, 20 minutes, 15 minutes, 10 minutes, 5 minutes, 4 minutes, 3 minutes, 2 minutes, or 1 minute. Sometimes the sample is contacted with the reagents for at least 120 minutes, 110 minutes, 100 minutes, 90 minutes, 80 minutes, 70 minutes, 60 minutes, 55 minutes, 50 minutes, 45 minutes, 40 minutes, 35 minutes, 30 minutes, 25 minutes, 20 minutes, 15 minutes, 10 minutes, or 5 minutes. In some cases, the streamlined lateral flow devices and methods described herein can detect a target nucleic acid in a sample in less than 10 hours, less than 9 hours, less than 8 hours, less than 7 hours, less than 6 hours, less than 5 hours, less than 4 hours, less than 3 hours, less than 2 hours, less than 1 hour, less than 50 minutes, less than 45 minutes, less than 40 minutes, less than 35 minutes, less than 30 minutes, less than 25 minutes, less than 20 minutes, less than 15 minutes, less than 10 minutes, less than 9 minutes, less than 8 minutes, less than 7 minutes, less than 6 minutes, or less than 5 minutes.

When a guide nucleic acid binds to a target nucleic acid, the programmable nuclease's trans cleavage activity can be initiated, and reporters can be cleaved, resulting in the detection of a detectable signal (e.g., fluorescence). Some methods as described herein can be a method of assaying for a target nucleic acid in a sample comprises contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some reporters of a population of reporters (e.g., at least some protein-nucleic acids of a population of protein-nucleic acids), wherein the signal indicates a presence of the target nucleic acid in the sample and wherein absence of the signal indicates an absence of the target nucleic acid in the sample. The cleaving of the reporter using the programmable nuclease may cleave with an efficiency of at least about 50% as measured by a change in a signal that is optical (e.g., fluorescent, colorimetric, etc.), as non-limiting examples. Some methods as described herein can be a method of detecting a target nucleic acid in a sample comprising contacting the sample comprising the target nucleic acid with a guide nucleic acid targeting a target nucleic acid segment, a programmable nuclease capable of being activated when complexed with the guide nucleic acid and the target nucleic acid segment, a single stranded reporter comprising a detection moiety, wherein the reporter is capable of being cleaved by the activated programmable nuclease, thereby generating a first detectable signal, cleaving the single stranded reporter using the programmable nuclease that cleaves as measured by a change in color, and measuring the first detectable signal on the support medium. The cleaving of the single stranded reporter using the programmable nuclease may cleave with an efficiency of at least about 50% as measured by a change in color of a detection region or spot as described herein. In some cases, the cleavage efficiency is at least 40%, 50%, 60%, 70%, 80%, 90%, or 95% as measured by a change in color. The change in color may be a detectable colorimetric signal or a signal visible by eye. The change in color may be measured as a first detectable signal. The first detectable signal can be detectable within 5 minutes of contacting the sample comprising the target nucleic acid with a guide nucleic acid targeting a target nucleic acid segment, a programmable nuclease capable of being activated when complexed with the guide nucleic acid and the target nucleic acid segment, and a single stranded reporter comprising a detection moiety, wherein the reporter is capable of being cleaved by the activated nuclease. The first detectable signal can be detectable within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the sample comprising the target nucleic acid with a guide nucleic acid targeting a target nucleic acid segment.

In some cases, the streamlined lateral flow device and methods described herein detect a target single-stranded nucleic acid with a programmable nuclease and a single-stranded reporter in a sample where the sample is contacted with the reagents for a predetermined length of time sufficient for trans cleavage of the single stranded reporter. For example, a programmable nuclease is LbuCas13a that detects a target nucleic acid and a single stranded reporter comprising two adjacent uracil nucleotides with a green detectable moiety that is detected upon cleavage. As another example, a programmable nuclease is LbaCas13a that detects a target nucleic acid and a single-stranded reporter comprises two adjacent adenine nucleotides with a red detectable moiety that is detected upon cleavage.

In some cases, the streamlined lateral flow devices and methods described herein detect two different target single-stranded nucleic acids with two different programmable nucleases and two different single-stranded reporters in a sample where the sample is contacted with the reagents for a predetermined length of time sufficient for trans cleavage of the at least two single-stranded reporters. For example, a first programmable nuclease is LbuCas13a, which is activated by a first single-stranded target nucleic acid and upon activation, cleaves a first single-stranded reporter comprising two adjacent uracil nucleotides with a green detectable moiety that is detected upon cleavage, and a second programmable nuclease is LbaCas13a, which is activated by a second single-stranded target nucleic acid and upon activation, cleaves a second single-stranded reporter comprising two adjacent adenine nucleotides with a red detectable moiety that is detected upon cleavage. In some cases, the activation of both programmable nucleases to cleave their respective single-stranded nucleic acids, for example LbuCas13a to cleave a first single-stranded reporter comprising two adjacent uracil nucleotides with a green detectable moiety that is detected upon cleavage and LbaCas13a to cleave a second single-stranded reporter comprises two adjacent adenine nucleotides with a red detectable moiety that is detected upon cleavage, results in the subsequent detection of a yellow signal indicates that the first single-stranded target nucleic acid and the second single-stranded target nucleic are both present in the sample.

Alternatively, the streamlined lateral flow devices and methods described herein can comprise a first programmable nuclease that detects the presence of a first single-stranded target nucleic acid in a sample and a second programmable nuclease that is used as a control. For example, a first programmable nuclease is LbuCas13a, which cleaves a first single-stranded reporter comprising two adjacent uracil nucleotides with a green detectable moiety that is detected upon cleavage and which is activated by a first single-stranded target nucleic acid if it is present in the sample, and a second programmable nuclease is LbaCas13a, which cleaves a second single-stranded reporter comprising two adjacent adenine nucleotides with a red detectable moiety that is detected upon cleavage and which is activated by a second single-stranded target nucleic acid that is not found (and would not be expected to ever be found) in the sample and serves as a control. In this case, the detection of a red signal or a yellow signal indicates there is a problem with the test (e.g., the sample contains a high level of other RNAses that are cleaving the single-stranded reporters in the absence of activation of the second programmable nuclease), but the detection of a green signal alone indicates the test is working correctly and the first target single-stranded nucleic acid of the first programmable nuclease is present in the sample.

As additional examples, the streamlined lateral flow devices and methods described herein detect at least two different target single-stranded nucleic acids with at least two different programmable nucleases and at least two different single stranded reporters in a sample where the sample is contacted with the reagents for a predetermined length of time sufficient for trans cleavage of the at least two single stranded detector nucleic acids. For example, a first programmable nuclease is a Cas13a protein, which cleaves a first single-stranded detector nucleic acid that is detected upon cleavage and which is activated by a first single-stranded target nucleic acid from a sepsis RNA biomarker if it is present in the sample, and a second programmable nuclease is a Cas14 protein, which cleaves a second single-stranded reporter that is detected upon cleavage and which is activated by a second single-stranded target nucleic acid.

The reagents described herein can also include buffers, which are compatible with the streamlined lateral flow devices and methods disclosed herein. These buffers are compatible with the other reagents, samples, and support mediums as described herein for detection of an ailment, such as a disease, including those caused by viruses. The methods described herein can also include the use of buffers, which are compatible with the methods disclosed herein. Compatible buffers and buffer components may include HEPES, KCl, MgCl2, glycerol, Igepal Ca-630, BSA, and imidazole.

A number of detection devices and methods are consistent with methods disclosed herein. For example, any device that can measure or detect a calorimetric, potentiometric, amperometric, optical (e.g., fluorescent, colorometric, etc.), or piezo-electric signal. Often a calorimetric signal is heat produced after cleavage of the reporters. Sometimes, a calorimetric signal is heat absorbed after cleavage of the reporters. A potentiometric signal, for example, is electrical potential produced after cleavage of the reporters. An amperometric signal can be movement of electrons produced after the cleavage of reporter. Often, the signal is an optical signal, such as a colorometric signal or a fluorescence signal. An optical signal is, for example, a light output produced after the cleavage of the reporters. Sometimes, an optical signal is a change in light absorbance between before and after the cleavage of reporters. Often, a piezo-electric signal is a change in mass between before and after the cleavage of the reporter. Sometimes, the reporter is protein-nucleic acid. Often, the protein-nucleic acid is an enzyme-nucleic acid.

The results from the detection region from a completed assay can be detected and analyzed in various ways, for example, by a glucometer. In some cases, the positive control spot and the detection spot in the detection region is visible by eye, and the results can be read by the user. In some cases, the positive control spot and the detection spot in the detection region is visualized by an imaging device or other device depending on the type of signal. Often, the imaging device is a digital camera, such a digital camera on a mobile device. The mobile device may have a software program or a mobile application that can capture an image of the support medium, identify the assay being performed, detect the detection region and the detection spot, provide image properties of the detection spot, analyze the image properties of the detection spot, and/or provide a result. Alternatively, or in combination, the imaging device can capture fluorescence, ultraviolet (UV), infrared (IR), or visible wavelength signals. The imaging device may have an excitation source to provide the excitation energy and captures the emitted signals. In some cases, the excitation source can be a camera flash and optionally a filter. In some cases, the imaging device is used together with an imaging box that is placed over the support medium of the device to create a dark room to improve imaging. The imaging box can be a cardboard box that the imaging device can fit into before imaging. In some instances, the imaging box has optical lenses, mirrors, filters, or other optical elements to aid in generating a more focused excitation signal or to capture a more focused emission signal. Often, the imaging box and the imaging device are small, low profile, handheld, and/or portable to facilitate the transport and use of the assay in remote or low resource settings.

The assay described herein can be visualized and analyzed by a mobile application (app) or a software program. Using the graphic user interface (GUI) of the app or program, an individual can take an image of the support medium, including the detection region, barcode, reference color scale, and/or fiduciary markers on the housing, using a camera on a mobile device. The program or app reads the barcode or identifiable label for the test type, locates the fiduciary marker to orient the sample, and reads the detectable signals, compares against the reference color grid, and determines the presence or absence of the target nucleic acid, which indicates the presence of the gene, virus, or the agent responsible for the disease or condition or state. The mobile application can present the results of the test to the individual. The mobile application can store the test results in the mobile application. The mobile application can communicate with a remote device and transfer the data of the test results. The test results can be viewable remotely from the remote device by another individual, including a healthcare professional. A remote user can access the results and use the information to recommend action for treatment, intervention, clean up of an environment.

### Samples

A number of samples are consistent with the streamlined lateral flow devices and methods disclosed herein. These samples are, for example, consistent with streamlined lateral flow devices disclosed herein for detection of a target nucleic acid within the sample, wherein the streamlined lateral flow devices may comprise sample preparation regions, optional amplification regions for amplifying a target nucleic acid within the sample, regions for mixing the sample with a programmable nuclease, and detection regions for assaying a detectable signal arising due to cleavage of reporters by the programmable nuclease within the streamlined lateral flow device itself. These samples can comprise a target nucleic acid for detection of an condition or species, such as a disease, pathogen, or virus. Generally, a sample from an individual, an animal, or an environmental sample can be obtained to test for presence of a condition, disease, virus, pathogen, or any mutation of interest. A biological sample from the individual may be blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, or tissue. A tissue sample may be dissociated or liquefied prior to application to detection system of the present disclosure. A sample from an environment may be from soil, air, or water. In some instances, the environmental sample is taken as a swab from a surface of interest or taken directly from the surface or environment of interest. In some instances, the raw sample is applied to the detection system. In some instances, the sample is a) diluted with a buffer or a fluid or concentrated prior to application to the detection system or b) applied without alteration (i.e., “neat”) to the detection system. Sometimes, the sample is contained in no more 20 uL. The sample, in some cases, is contained in no more than 1, 5, 10, 15, 20, 25, 30, 35, 40, 45, 50, 55, 60, 65, 70, 75, 80, 90, 100, 200, 300, 400, 500 uL, or any of value from 1 uL to 500 uL or range bounded by any value from 1 uL to 500 uL. Sometimes, the sample is contained in more than 500 uL.

In some instances, the sample is taken from single-cell eukaryotic organisms; a plant or a plant cell; an algal cell; a fungal cell; an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. In some instances, the sample is taken from nematodes, protozoans, helminths, or malarial parasites. In some cases, the sample comprises nucleic acids from a cell lysate from a eukaryotic cell, a mammalian cell, a human cell, a prokaryotic cell, or a plant cell. In some cases, the sample comprises nucleic acids expressed from a cell.

The sample may comprise at least one target sequence that can bind to a guide nucleic acid of the reagents described herein. In some cases, the target sequence is a portion of a nucleic acid. A portion of a nucleic acid can be from a genomic locus, a transcribed mRNA, or a reverse transcribed cDNA. A portion of a nucleic acid can be from 5 to 100, 5 to 90, 5 to 80, 5 to 70, 5 to 60, 5 to 50, 5 to 40, 5 to 30, 5 to 25, 5 to 20, 5 to 15, or 5 to 10 nucleotides in length. A portion of a nucleic acid can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 nucleotides in length. The target sequence can be reverse complementary to a guide nucleic acid.

In some cases, the target sequence is a portion of a nucleic acid from a virus or a bacterium or other agents responsible for a disease or condition in the sample. The target sequence, in some cases, is a portion of a nucleic acid from a sexually transmitted infection or a contagious disease, in the sample. The target sequence, in some cases, is a portion of a nucleic acid from an upper respiratory tract infection, a lower respiratory tract infection, or a contagious disease, in the sample. The target sequence, in some cases, is a portion of a nucleic acid from a hospital acquired infection or a contagious disease, in the sample. The target sequence, in some cases, is an ssRNA. These target sequences may be from a disease, and the disease may include but is not limited to, influenza virus including influenza A virus (IAV) or influenza B virus (IBV), rhinovirus, cold viruses, a respiratory virus, an upper respiratory virus, a lower respiratory virus, SARS-CoV-2, or respiratory syncytial virus. Pathogens include viruses, fungi, helminths, protozoa, and parasites. Pathogenic viruses include but are not limited to influenza virus and the like. Pathogens include, e.g., Mycobacterium tuberculosis, Streptococcus agalactiae, methicillin-resistant Staphylococcus aureus, Legionella pneumophila, Streptococcus pyogenes, Escherichia coli, Neisseria meningitidis, Pneumococcus, Hemophilus influenzae B, influenza virus, respiratory syncytial virus (RSV), M. pneumoniae, Streptococcus intermdius, Streptococcus pneumoniae, and Streptococcus pyogenes. Often the target nucleic acid comprises a sequence from a virus or a bacterium or other agents responsible for a disease that can be found in the sample. Pathogenic viruses include but are not limited to influenza virus; RSV; coronavirus, an ssRNA virus, a respiratory virus, an upper respiratory virus, a lower respiratory virus, or a rhinovirus. Pathogens include, e.g., Mycobacterium tuberculosis, Streptococcus agalactiae, Legionella pneumophila, Streptococcus pyogenes, Hemophilus influenzae B influenza virus, respiratory syncytial virus (RSV), or Mycobacterium tuberculosis

In some cases, the target nucleic acid can be indicative of the presence of cancer (e.g., colorectal cancer, breast cancer, brain cancer, or other cancers) or any other disease. In some aspects, the target nucleic acid can be indicative of a certain phenotype of interest (e.g., eye or hair color). In some aspects, the target nucleic acid can be indicative of any genetic disorder or disease, such as muscular dystrophy. In some aspects, the lateral flow devices disclosed herein can include reagents that allow a user to distinguish between heterozygous and homozygous alleles for targets of interest. In some aspects, the lateral flow devices disclosed herein can detect target nucleic acids of interest from complex samples (e.g., a cell lysate, a tumor biopsy). In some aspects, the lateral flow devices disclosed herein can detect target nucleic acids of interest from a purified sample.

The sample can be used for identifying a disease status or condition. For example, a sample is any sample described herein, and is obtained from a subject for use in identifying a disease status of a subject. Sometimes, a method comprises obtaining a serum sample from a subject and identifying a disease status or condition of the subject. Sometimes, a method comprises obtaining a nasal swab from a subject and identifying a disease status or condition of the subject.

In some instances, the target nucleic acid is a single stranded nucleic acid. Alternatively, or in combination, the target nucleic acid is a double stranded nucleic acid and is prepared into single stranded nucleic acids before or upon contacting the reagents. The target nucleic acid may be a RNA, DNA, synthetic nucleic acids, or nucleic acids found in biological or environmental samples. The target nucleic acids include but are not limited to mRNA, rRNA, tRNA, non-coding RNA, long non-coding RNA, and microRNA (miRNA). In some cases, the target nucleic acid is mRNA. In some cases, the target nucleic acid is from a virus, a parasite, or a bacterium described herein. In some cases, the target nucleic acid is transcribed from a gene as described herein.

A number of target nucleic acids are consistent with the methods and compositions disclosed herein. Some methods described herein can detect a target nucleic acid present in the sample in various concentrations or amounts as a target nucleic acid population. In some cases, the sample has at least two copies of the target nucleic acids. In some cases, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 target nucleic acid copies. In some cases, the method detects target nucleic acid present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids.

A number of target nucleic acid populations are consistent with the methods and compositions disclosed herein. Some methods described herein can detect two or more target nucleic acid populations present in the sample in various concentrations or amounts. In some cases, the sample has at least two different target nucleic acid populations. In some cases, the sample has at least 3, 4, 5, 6, 7, 8, 9, 10, 20, 30, 40, or 50 target nucleic acid populations. In some cases, the method detects target nucleic acid populations that are present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids. The target nucleic acid populations can be present at different concentrations or amounts in the sample.

Any of the above disclosed samples are consistent with the systems, assays, and programmable nucleases disclosed herein and can be used as a companion diagnostic with any of the diseases disclosed herein, or can be used in reagent kits, point-of-care diagnostics, or over-the-counter diagnostics.

### Applications

A. Detection of a Mutation in a Target Nucleic Acid

Disclosed herein are methods of assaying for a target nucleic acid as described herein that can be used for detection of a mutation in a target nucleic acid. For example, a method of assaying for a target nucleic acid in a sample comprises contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. The detection of the signal can indicate the presence of the target nucleic acid. Sometimes, the target nucleic acid comprises a mutation. Often, the mutation is a single nucleotide mutation. As another example, a method of assaying for a target nucleic acid in a sample, for example, comprises: a) contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; b) contacting the complex to a substrate; c) contacting the substrate to a reagent that differentially reacts with a cleaved substrate; and d) assaying for a signal indicating cleavage of the substrate, wherein the signal indicates a presence of the target nucleic acid in the sample and wherein absence of the signal indicates an absence of the target nucleic acid in the sample. Often, the substrate is an enzyme-nucleic acid and the reagent is the enzyme substrate. Sometimes, the substrate is an enzyme substrate-nucleic acid and the reagent is the enzyme.

Methods described herein can be used to identify a mutation in a target nucleic acid. The methods can be used to identify a single nucleotide mutation of a target nucleic acid that affects the expression of a gene. A mutation that affects the expression of gene can be a single nucleotide mutation of a target nucleic acid within the gene, a single nucleotide mutation of a target nucleic acid comprising RNA associated with the expression of a gene, or a target nucleic acid comprising a single nucleotide mutation of a nucleic acid associated with regulation of expression of a gene, such as an RNA or a promoter, enhancer, or repressor of the gene. Often, a status of a mutation is used to diagnose or identify diseases associated with the mutation of target nucleic acid. Detection of target nucleic acids having a mutation are applicable to a number of fields, such as clinically, as a diagnostic, in laboratories as a research tool, and in agricultural applications. Often, the mutation is a single nucleotide mutation. The mutation may result in a mutated strain of a virus.

B. Disease Detection

Disclosed herein are methods of assaying for a target nucleic acid as described herein that can be used for disease detection. For example, a method of assaying for a target nucleic acid in a sample comprises contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. The detection of the signal can indicate the presence of the target nucleic acid. Sometimes, the target nucleic acid comprises a mutation. Often, the mutation is a single nucleotide mutation. As another example, a method of assaying for a target nucleic acid in a sample, for example, comprises: a) contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; b) contacting the complex to a substrate; c) contacting the substrate to a reagent that differentially reacts with a cleaved substrate; and d) assaying for a signal indicating cleavage of the substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid.

Methods described herein can be used to identify a mutation in a target nucleic acid from a bacteria, virus, or microbe. The methods can be used to identify a mutation of a target nucleic acid that affects the expression of a gene. A mutation that affects the expression of gene can be a mutation of a target nucleic acid within the gene, a mutation of a target nucleic acid comprising RNA associated with the expression of a gene, or a target nucleic acid comprising a mutation of a nucleic acid associated with regulation of expression of a gene, such as an RNA or a promoter, enhancer, or repressor of the gene. Sometimes, a status of a target nucleic acid mutation is used to determine a pathogenicity of a bacteria, virus, or microbe or treatment resistance, such as resistance to antibiotic treatment. Often, a status of a mutation is used to diagnose or identify diseases associated with the mutation of target nucleic acids in the bacteria, virus, or microbe. Often, the mutation is a single nucleotide mutation.

C. Detection as a Research Tool, Point-of-Care, or Over-the-Counter

Disclosed herein are methods of assaying for a target nucleic acid as described herein that can be used as a research tool, and can be provided as reagent kits. For example, a method of assaying for a target nucleic acid in a sample comprises contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. The detection of the signal can indicate the presence of the target nucleic acid. Sometimes, the target nucleic acid comprises a mutation. Often, the mutation is a single nucleotide mutation. As another example, a method of assaying for a target nucleic acid in a sample, for example, comprises: a) contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; b) contacting the complex to a substrate; c) contacting the substrate to a reagent that differentially reacts with a cleaved substrate; and d) assaying for a signal indicating cleavage of the substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid.

The methods as described herein can be used to identify a single nucleotide mutation in a target nucleic acid. The methods can be used to identify mutation of a target nucleic acid that affects the expression of a gene. A mutation that affects the expression of gene can be a single nucleotide mutation of a target nucleic acid within the gene, a mutation of a target nucleic acid comprising RNA associated with the expression of a gene, or a target nucleic acid comprising a mutation of a nucleic acid associated with regulation of expression of a gene, such as an RNA or a promoter, enhancer, or repressor of the gene. Often, the mutation is a single nucleotide mutation.

The reagent kits or research tools can be used to detect any number of target nucleic acids, mutations, or other indications disclosed herein in a laboratory setting. Reagent kits can be provided as reagent packs for open box instrumentation.

In other embodiments, any of the systems, assay formats, Cas reporters, programmable nucleases, or other reagents can be used in a point-of-care (POC) test, which can be carried out at a decentralized location such as a hospital, physician's office laboratory (POL), or clinic. These point-of-care tests can be used to diagnose any of the indications disclosed herein, or can be used to measure the presence or absence of a particular mutation in a target nucleic acid (e.g., EGFR). POC tests can be provided as small instruments with a consumable test card, wherein the test card is any of the assay formats (e.g., a lateral flow assay) disclosed herein.

In still other embodiments, any of the systems, assay formats, Cas reporters, programmable nucleases, or other reagents can be used in an over-the-counter (OTC), readerless format, which can be used at remote sites or at home to diagnose a range of indications. OTC products can include a consumable test card, wherein the test card is any of the assay formats (e.g., a lateral flow assay) disclosed herein. In an OTC product, the test card can be interpreted visually or using a mobile phone.

D. Target Amplification and Detection

The lateral flow devices disclosed herein can optionally include regions of the device and reagents for amplification. However, variations of said lateral flow devices not including a region or reagents for amplification are also consistent with the present disclosure. A number of target amplification and detection methods are consistent with the methods, compositions, reagents, enzymes, and kits disclosed herein. As described herein, a target nucleic acid may be detected using a DNA-activated programmable RNA nuclease (e.g., a Cas13), a DNA-activated programmable DNA nuclease (e.g., a Cas12), or an RNA-activated programmable RNA nuclease (e.g., a Cas13) and other reagents disclosed herein (e.g., RNA components). The target nucleic acid may be detected using DETECTR™, as described herein. The target nucleic acid may be an RNA, reverse transcribed RNA, DNA, DNA amplicon, amplified DNA, synthetic nucleic acids, or nucleic acids found in biological or environmental samples. In some cases, the target nucleic acid is amplified prior to or concurrent with detection. In some cases, the target nucleic acid is reverse transcribed prior to amplification. The target nucleic acid may be amplified via loop mediated isothermal amplification (LAMP) of a target nucleic acid sequence. In some cases, the target nucleic acid is amplified using LAMP coupled with reverse transcription (RT-LAMP). The LAMP amplification may be performed independently (e.g., off device or in a separate chamber or region of the device), or the LAMP amplification may be coupled to DETECTR™ for detection of the target nucleic acid. The RT-LAMP amplification may be performed independently, or the RT-LAMP amplification may be coupled to DETECTR™ for detection of the target nucleic acid. The DETECTR™ reaction may be performed using any method consistent with the methods disclosed herein.

(a) Amplification and Detection Reaction Mixtures

In some embodiments, a LAMP amplification reaction comprises a plurality of primers, dNTPs, and a DNA polymerase. LAMP may be used to amplify DNA with high specificity under isothermal conditions. The DNA may be single stranded DNA or double stranded DNA. In some cases, a target nucleic acid comprising RNA may be reverse transcribed into DNA using a reverse transcriptase prior to LAMP amplification. A reverse transcription reaction may comprise primers, dNTPs, and a reverse transcriptase. In some cases, the reverse transcription reaction and the LAMP amplification reaction may be performed in the same reaction. A combined RT-LAMP reaction may comprise LAMP primers, reverse transcription primers, dNTPs, a reverse transcriptase, and a DNA polymerase. In some case, the LAMP primers may comprise the reverse transcription primers.

A DETECTR™ reaction to detect the target nucleic acid sequence may comprise a guide nucleic acid comprising a segment that is reverse complementary to a segment of the target nucleic acid and a programmable nuclease. The programmable nuclease when activated, as described elsewhere herein, exhibits sequence-independent cleavage of a reporter (e.g., a nucleic acid comprising a moiety that becomes detectable upon cleavage of the nucleic acid by the programmable nuclease). The programmable nuclease is activated upon the guide nucleic acid hybridizing to the target nucleic acid. A combined LAMP DETECTR™ reaction may comprise a plurality of primers, dNTPs, a DNA polymerase, a guide nucleic acid, a programmable nuclease, and a substrate nucleic acid. A combined RT-LAMP DETECTR™ reaction may comprise LAMP primers, reverse transcription primers, dNTPs, a reverse transcriptase, a DNA polymerase, a guide nucleic acid, a programmable nuclease, and a substrate nucleic acid. In some case, the LAMP primers may comprise the reverse transcription primers. LAMP and DETECTR™ can be carried out in the same sample or reaction volume. LAMP and DETECTR™ can be carried out concurrently in separate sample or reaction volumes or in the same sample volume. RT-LAMP and DETECTR™ can be carried out in the same sample or reaction volume. RT-LAMP and DETECTR™ can be carried out concurrently in separate sample or reaction volumes or in the same sample volume.

(b) Amplification and Detection of a Single Nucleotide Polymorphism Allele

A DETECTR™ reaction may be used to detect the presence of a specific single nucleotide polymorphism (SNP) allele in a sample. The DETECTR™ reaction may produce a detectable signal, as described elsewhere herein, indicative of the presence of a target nucleic acid comprising a specific SNP allele. The DETECTR™ reaction may not produce a signal in the absence of the target nucleic acid or in the presence of a nucleic acid sequence that does not comprise the specific SNP allele or comprises a different SNP allele. In some cases, a DETECTR™ reaction may comprise a guide RNA reverse complementary to a portion of a target nucleic acid sequence comprising a specific SNP allele. The guide RNA and the target nucleic acid comprising the specific SNP allele may bind to and activate a programmable nuclease, thereby producing a detectable signal as described elsewhere herein. The guide RNA and a nucleic acid sequence that does not comprise the specific SNP allele may not bind to or activate the programmable nuclease and may not produce a detectable signal. In some cases, a target nucleic acid sequence that may or may not comprise a specific SNP allele may be amplified using, for example, a LAMP amplification reaction. In some cases, the LAMP amplification reaction may be combined with a reverse transcription reaction, a DETECTR™ reaction, or both. For example, the LAMP reaction may be an RT-LAMP reaction, a LAMP DETECTR™ reaction, or an RT-LAMP DETECTR™ reaction.

A DETECTR™ reaction, as described elsewhere herein, may produce a detectable signal specifically in the presence of a target nucleic acid sequence comprising a specific SNP allele. For example, the DETECTR™ reaction may produce a detectable signal in the presence of a target nucleic acid comprising a G nucleic acid at a location of a SNP but not in the presence of a nucleic acid comprising a C, a T, or an A nucleic acid at the location of the SNP. The DETECTR™ reaction may produce a detectable signal in the presence of a target nucleic acid comprising a T nucleic acid at a location of a SNP but not in the presence of a nucleic acid comprising a G, a C, or an A nucleic acid at the location of the SNP. The DETECTR™ reaction may produce a detectable signal in the presence of a target nucleic acid comprising a C nucleic acid at a location of a SNP but not in the presence of a nucleic acid comprising a G, a T, or an A nucleic acid at the location of the SNP. The DETECTR™ reaction may produce a detectable signal in the presence of a target nucleic acid comprising an A nucleic acid at a location of a SNP but not in the presence of a nucleic acid comprising a G, a T, or a C nucleic acid at the location of the SNP. In addition to the DETECTR™ reaction, the target nucleic acid having the SNP may be concurrently, sequentially, concurrently together in a sample, or sequentially together in a sample be carried out alongside LAMP or RT-LAMP. For example, the reactions can comprise LAMP and DETECTR™ reactions, or RT-LAMP and DETECTR™ reactions. Performing a DETECTR™ reaction in combination with a LAMP reaction may result in an increased detectable signal as compared to the DETECTR™ reaction in the absence of the LAMP reaction.

In some cases, the detectable signal produced in the DETECTR™ reaction may be higher in the presence of a target nucleic acid comprising a specific SNP allele than in the presence of a nucleic acid that does not comprise the specific SNP allele. In some cases, the DETECTR™ reaction may produce a detectable signal that is at least 1-fold, at least 2-fold, at least 3-fold, at least 4-fold, at least 5-fold, at least 10-fold, at least 20-fold, at least 30-fold, at least 40-fold, at least 50-fold, at least 100-fold, at least 200-fold, at least 300-fold, at last 400-fold, at least 500-fold, at least 1000-fold, at least 2000-fold, at least 3000-fold, at least 4000-fold, at least 5000-fold, at least 6000-fold, at least 7000-fold, at least 8000-fold, at least 9000-fold, at least 10000-fold, at least 50000-fold, at least 100000-fold, at least 500000-fold, or at least 1000000-fold greater in the presence of a target nucleic acid comprising a specific SNP allele than in the presence of a nucleic acid that does not comprise the specific SNP allele. In some cases, the DETECTR™ reaction may produce a detectable signal that is from 1-fold to 2-fold, from 2-fold to 3-fold, from 3-fold to 4-fold, from 4-fold to 5-fold, from 5-fold to 10-fold, from 10-fold to 20-fold, from 20-fold to 30-fold, from 30-fold to 40-fold, from 40-fold to 50-fold, from 50-fold to 100-fold, from 100-fold to 500-fold, from 500-fold to 1000-fold, from 1000-fold to 10,000-fold, from 10,000-fold to 100,000-fold, or from 100,000-fold to 1,000,000-fold greater in the presence of a target nucleic acid comprising a specific SNP allele than in the presence of a nucleic acid that does not comprise the specific SNP allele.

A DETECTR™ reaction may be used to detect the presence of a SNP allele associated with a disease or a condition in a nucleic acid sample. The DETECTR™ reaction may be used to detect the presence of a SNP allele associated with an increased likelihood of developing a disease or a condition in a nucleic acid sample. The DETECTR™ reaction may be used to detect the presence of a SNP allele associated with a phenotype in a nucleic acid sample. For example, a DETECTR™ reaction may be used to detect a SNP allele associated with a disease such as phenylketonuria (PKU), cystic fibrosis, sickle-cell anemia, albinism, Huntington's disease, myotonic dystrophy type 1, hypercholesterolemia, neurofibromatosis, polycystic kidney disease, hemophilia, muscular dystrophy, hypophosphatemic rickets, Rett's syndrome, or spermatogenic failure. A DETECTR™ reaction may be used to detect a SNP allele associated with an increased risk of cancer, for example bladder cancer, brain cancer, breast cancer, cervical cancer, colon cancer, colorectal cancer, gallbladder cancer, stomach cancer, leukemia, liver cancer, lung cancer, oral cancer, esophageal cancer, ovarian cancer, pancreatic cancer, prostate cancer, skin cancer, testicular cancer, thyroid cancer, neuroblastoma, or lymphoma. A DETECTR™ reaction may be used to detect a SNP allele associated with an increased risk of a disease, for example Alzheimer's disease, Parkinson's disease, amyloidosis, heterochromatosis, celiac disease, macular degeneration, or hypercholesterolemia. A DETECTR™ reaction may be used to detect a SNP allele associated with a phenotype, for example, eye color, hair color, height, skin color, race, alcohol flush reaction, caffeine consumption, deep sleep, genetic weight, lactose intolerance, muscle composition, saturated fat and weight, or sleep movement.

A. Support Medium

A number of support mediums are consistent with the streamlined lateral flow devices and methods disclosed herein. These support mediums are, for example, consistent with streamlined lateral flow devices disclosed herein for detection of a target nucleic acid within the sample, wherein the streamlined lateral flow device may comprise one or more regions for sample preparation, optionally amplifying a target nucleic acid within the sample, mixing with a programmable nuclease, and detection of a detectable signal arising from cleavage of reporters by the programmable nuclease within the streamlined lateral flow device itself. These support mediums are compatible with the samples, reagents, and streamlined lateral flow devices described herein for detection of an ailment or condition, such as a viral infection. A support medium described herein can provide a way to present the results from the interaction between the reagents and the sample. The support medium provides a medium to present the detectable signal in a detectable format. Optionally, the support medium concentrates the detectable signal to a detection spot in a detection region to increase the sensitivity, specificity, or accuracy of the assay. The support mediums can present the results of the assay and indicate the presence or absence of the disease of interest targeted by the target nucleic acid. The result on the support medium can be read by eye or using a machine. The support medium helps to stabilize the detectable signal generated by the cleaved detector molecule on the surface of the support medium. In some instances, the support medium is a lateral flow assay strip. In some instances, the support medium is a PCR plate. The PCR plate can have 96 wells or 384 wells. The PCR plate can have a subset number of wells of a 96 well plate or a 384 well plate. A subset number of wells of a 96 well PCR plate is, for example, 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, or 95 wells. For example, a PCR subset plate can have 4 wells wherein a well is the size of a well from a 96 well PCR plate (e.g., a 4 well PCR subset plate wherein the wells are the size of a well from a 96 well PCR plate). A subset number of wells of a 384 well PCR plate is, for example, 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 110, 120, 130, 140, 150, 160, 170, 180, 190, 200, 220, 240, 260, 280, 300, 320, 340, 360, or 380 wells. For example, a PCR subset plate can have 20 wells wherein a well is the size of a well from a 384 well PCR plate (e.g., a 20 well PCR subset plate wherein the wells are the size of a well from a 384 well PCR plate). The PCR plate or PCR subset plate can be paired with a fluorescent light reader, a visible light reader, or other imaging device. Often, the imaging device is a digital camera, such a digital camera on a mobile device. The mobile device may have a software program or a mobile application that can capture an image of the PCR plate or PCR subset plate, identify the assay being performed, detect the individual wells and the sample therein, provide image properties of the individuals wells comprising the assayed sample, analyze the image properties of the contents of the individual wells, and provide a result.

The support medium has at least one specialized zone or region to present the detectable signal. The regions comprise at least one of a sample pad region, a nucleic acid amplification region, a conjugate pad region, a detection region, and a collection pad region. In some instances, the regions are overlapping completely, overlapping partially, or in series and in contact only at the edges of the regions, where the regions are in fluid communication with its adjacent regions. In some instances, the support medium has a sample pad located upstream of the other regions; a conjugate pad region having a means for specifically labeling the detector moiety; a detection region located downstream from sample pad; and at least one matrix which defines a flow path in fluid connection with the sample pad. In some instances, the support medium has an extended base layer on top of which the various zones or regions are placed. The extended base layer may provide a mechanical support for the zones or regions.

Described herein are sample pad that provide an area to apply the sample to the support medium. The sample may be applied to the support medium by a dropper or a pipette on top of the sample pad, by pouring or dispensing the sample on top of the sample pad region, or by dipping the sample pad into a reagent chamber holding the sample. The sample can be applied to the sample pad prior to reaction with the reagents which occurs the reagents are placed on the support medium or be reacted with the reagents prior to application on the sample pad. The sample pad region can transfer the reacted reagents and sample into the other zones of the support medium. Transfer of the reacted reagents and sample may be by capillary action, diffusion, convection or active transport aided by a pump. In some cases, the support medium is integrated with or overlayed by microfluidic channels to facilitate the fluid transport.

The dropper or the pipette may dispense a predetermined volume. In some cases, the predetermined volume may range from about 1 μl to about 1000 μl about 1 μl to about 500 μl about 1 μl to about 100 μl, or about 1 μl to about 50 μl. In some cases, the predetermined volume may be at least 1 μl, 2 μl, 3 μl, 4 μl, 5 μl, 6 μl, 7 μl, 8 μl, 9 μl, 10 μl, 25 μl, 50 μl, 75 μl, 100 μl, 250 μl, 500 μl, 750 μl, or 1000 μl. The predetermined volume may be no more than 5 μl, 10 μl, 25 μl, 50 μl, 75 μl, 100 μl, 250 μl, 500 μl, 750 μl, or 1000 μl. The dropper or the pipette may be disposable or be single-use.

Optionally, a buffer or a fluid may also be applied to the sample pad to help drive the movement of the sample along the support medium. In some cases, the volume of the buffer or the fluid may range from about 1 μl to about 1000 μl, about 1 μl to about 500 μl, about 1 μl to about 100 μl, or about 1 μl to about 50 μl. In some cases, the volume of the buffer or the fluid may be at least 1 μl, 2 μl, 3 μl, 4 μl, 5 μl, 6 μl, 7 μl, 8 μl, 9 μl, 10 μl, 25 μl, 50 μl, 75 μl, 100 μl, 250 μl, 500 μl, 750 μl, or 1000 μl. The volume of the buffer or the fluid may be no more than than 5 μl, 10 μl, 25 μl, 50 μl, 75 μl, 100 μl, 250 μl, 500 μl, 750 μl, or 1000 μl. In some cases, the buffer or fluid may have a ratio of the sample to the buffer or fluid of at least 1:1, 1:2, 1:3, 1:4, 1:5, 1:6, 1:7, 1:8, 1:9, or 1:10.

The sample pad can be made from various materials that facilitate transfer of most of the applied reacted reagents and samples to the subsequent regions. The sample pad may comprise cellulose fiber filters, woven meshes, porous plastic membranes, glass fiber filters, aluminum oxide coated membranes, nitrocellulose, paper, polyester filter, or polymer-based matrices. The material for the sample pad region may be hydrophilic and have low non-specific binding. The material for the sample pad may range from about 50 μm to about 1000 μm thick, about 50 μm to about 750 μm, about 50 μm to about 500 μm, or about 100 μm to about 500 μm.

The sample pad can be treated with chemicals to improve the presentation of the reaction results on the support medium. The sample pad can be treated to enhance extraction of nucleic acid in the sample, to control the transport of the reacted reagents and sample or the conjugate to other regions of the support medium, or to enhance the binding of the cleaved detection moiety to the conjugate binding molecule on the surface of the conjugate or to the capture molecule in the detection region. The chemicals may comprise detergents, surfactants, buffers, salts, viscosity enhancers, or polypeptides. In some instances, the chemical comprises bovine serum albumin.

Described herein are conjugate pads that provide a region on the support medium comprising conjugates coated on its surface by conjugate binding molecules that can bind to the detector moiety from the cleaved detector molecule or to the control molecule. The conjugate pad can be made from various materials that facilitate binding of the conjugate binding molecule to the detection moiety on a cleaved detector molecule and subsequent transfer of most of the conjugate-bound detection moiety to the subsequent regions. The conjugate pad may comprise the same material as the sample pad or other zones or a different material than the sample pad. The conjugate pad may comprise glass fiber filters, porous plastic membranes, aluminum oxide coated membranes, paper, cellulose fiber filters, woven meshes, polyester filter, or polymer-based matrices. The material for the conjugate pad region may be hydrophilic, have low non-specific binding, or have consistent fluid flow properties across the conjugate pad. In some cases, the material for the conjugate pad may range from about 50 μm to about 1000 μm thick, about 50 μm to about 750 μm, about 50 μm to about 500 μm, or about 100 μm to about 500 μm.

Further described herein are conjugates that are placed on the conjugate pad and immobilized to the conjugate pad until the sample is applied to the support medium. The conjugates may comprise a nanoparticle, a gold nanoparticle, a latex nanoparticle, a quantum dot, a chemiluminescent nanoparticle, a carbon nanoparticle, a selenium nanoparticle, a fluorescent nanoparticle, a liposome, or a dendrimer. The surface of the conjugate may be coated by a conjugate binding molecule that binds to the detection moiety of the cleaved detector molecule.

The conjugate binding molecules described herein coat the surface of the conjugates and can bind to detection moiety. The conjugate binding molecule binds selectively to the detection moiety cleaved from the reporter. Some suitable conjugate binding molecules comprise an antibody, a polypeptide, or a single stranded nucleic acid. In some cases, the conjugate binding molecule binds a dye and/or a fluorophore. Some such conjugate binding molecules that bind to a dye or a fluorophore can quench their signal. In some cases, the conjugate binding molecule is a monoclonal antibody. In some cases, an antibody, also referred to as an immunoglobulin, includes any isotype, variable regions, constant regions, Fc region, Fab fragments, F(ab′)2 fragments, and Fab′ fragments. Alternatively, the conjugate binding molecule is a non-antibody compound that specifically binds the detection moiety. Sometimes, the conjugate binding molecule is a polypeptide that can bind to the detection moiety. Sometimes, the conjugate binding molecule is avidin or a polypeptide that binds biotin. Sometimes, the conjugate binding molecule is a detector moiety binding nucleic acid.

The diameter of the conjugate may be selected to provide a desired surface to volume ratio. In some instances, a high surface area to volume ratio may allow for more conjugate binding molecules that are available to bind to the detection moiety per total volume of the conjugates. In some cases, the diameter of the conjugate may range from about 1 nm to about 1000 nm, about 1 nm to about 500 nm, about 1 nm to about 100 nm, or about 1 nm to about 50 nm. In some cases, the diameter of the conjugate may be at least 1 nm, 2 nm, 3 nm, 4 nm, 5 nm, 6 nm, 7 nm, 8 nm, 9 nm, 10 nm, 15 nm, 20 nm, 25 nm, 30 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 200 nm, 300 nm, 400 nm, 500 nm, 600 nm, 700 nm, 800 nm, 900 nm, or 1000 nm. In some cases, the diameter of the conjugate may be no more than 1 nm, 2 nm, 3 nm, 4 nm, 5 nm, 6 nm, 7 nm, 8 nm, 9 nm, 10 nm, 15 nm, 20 nm, 25 nm, 30 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 200 nm, 300 nm, 400 nm, 500 nm, 600 nm, 700 nm, 800 nm, 900 nm, or 1000 nm.

The ratio of conjugate binding molecules to the conjugates can be tailored to achieve desired binding properties between the conjugate binding molecules and the detection moiety. In some instances, the molar ratio of conjugate binding molecules to the conjugates is at least 1:1, 1:5, 1:10, 1:20, 1:30, 1:40, 1:50, 1:60, 1:70, 1:80, 1:90, 1:100, 1:110, 1:120, 1:130, 1:140, 1:150, 1:160, 1:170, 1:180, 1:190, 1:200, 1:250, 1:300, 1:350, 1:400, 1:450, or 1:500. In some instances, the mass ratio of conjugate binding molecules to the conjugates is at least 1:1, 1:5, 1:10, 1:20, 1:30, 1:40, 1:50, 1:60, 1:70, 1:80, 1:90, 1:100, 1:110, 1:120, 1:130, 1:140, 1:150, 1:160, 1:170, 1:180, 1:190, 1:200, 1:250, 1:300, 1:350, 1:400, 1:450, or 1:500. In some instances, the number of conjugate binding molecules per conjugate is at least 1, 10, 50, 100, 500, 1000, 5000, or 10000.

The conjugate binding molecules can be bound to the conjugates by various approaches. Sometimes, the conjugate binding molecule can be bound to the conjugate by passive binding. Some such passive binding comprises adsorption, absorption, hydrophobic interaction, electrostatic interaction, ionic binding, or surface interactions. In some cases, the conjugate binding molecule can be bound to the conjugate covalently. Sometimes, the covalent bonding of the conjugate binding molecule to the conjugate is facilitated by EDC/NHS chemistry or thiol chemistry.

Described herein is a detection region on the support medium that provides a region for presenting the assay results to a user or detection device. The detection region can be made from various materials that facilitate binding of the conjugate-bound detection moiety from cleaved detector molecule to the capture molecule specific for the detection moiety. The detection pad may comprise the same material as other zones or a different material than the other zones. The detection region may comprise nitrocellulose, paper, cellulose, cellulose fiber filters, glass fiber filters, porous plastic membranes, aluminum oxide coated membranes, woven meshes, polyester filter, or polymer-based matrices. Often the detection region may comprise nitrocellulose. The material for the region pad region may be hydrophilic, have low non-specific binding, or have consistent fluid flow properties across the region pad. The material for the conjugate pad may range from about 10 μm to about 1000 μm, about 10 μm to about 750 μm, about 10 μm to about 500 μm, or about 10 μm to about 300 μm.

The detection region comprises at least one capture area with a high density of a capture molecule that can bind to the detection moiety on the cleaved detection molecule and at least one area with a high density of a positive control capture molecule. The capture area with a high density of capture molecule or a positive control capture molecule may be a line, a circle, an oval, a rectangle, a triangle, a plus sign, or any other shapes. In some instances, the detection region comprise more than one capture area with high densities of more than one capture molecules, where each capture area comprises one type of capture molecule that specifically binds to one type of detection moiety or conjugate on the cleaved detection molecule and are different from the capture molecules in the other capture areas. The capture areas with different capture molecules may be overlapping completely, overlapping partially, or spatially separate from each other. In some instances, the capture areas may overlap and produce a combined detectable signal distinct from the detectable signals generated by the individual capture areas. Usually, the positive control spot is spatially distinct from any of the detection spot.

The capture molecules described herein bind to a detection moiety and immobilized in the detection spot in the detection region. Some suitable capture molecules comprise an antibody, a polypeptide, or a single stranded nucleic acid. In some cases, the capture molecule binds a dye and/or a fluorophore. Some such capture molecules that bind to a dye or a fluorophore can quench their signal. Sometimes, the capture molecule is an antibody that that binds to a dye or a fluorophore can quench their signal. In some cases, the capture molecule is a monoclonal antibody. In some cases, an antibody, also referred to as an immunoglobulin, includes any isotype, variable regions, constant regions, Fc region, Fab fragments, F(ab′)2 fragments, and Fab′ fragments. Alternatively, the capture molecule is a non-antibody compound that specifically binds the detection moiety. Sometimes, the capture molecule is a polypeptide that can bind to the detection moiety. In some instances, the detection moiety from cleaved detection molecule has a conjugate bound to the detection moiety, and the conjugate-detection moiety complex may bind to the capture molecule specific to the detection moiety or conjugate on the detection region. Sometimes, the capture molecule is a polypeptide that can bind to the detection moiety. Sometimes, the capture molecule is avidin or a polypeptide that binds biotin. Sometimes, the capture molecule is a detector moiety binding nucleic acid.

The detection region described herein comprises at least one area with a high density of a positive control capture molecule. The positive control spot in the detection region provides a validation of the assay and a confirmation of completion of the assay. If the positive control spot is not detectable by the visualization methods described herein, the assay is not valid and should be performed again with a new system or kit. The positive control capture molecule binds at least one of the conjugate, the conjugate binding molecule, or detection moiety and is immobilized in the positive control spot in the detect region. Some suitable positive control capture molecules comprise an antibody, a polypeptide, or a single stranded nucleic acid. In some cases, the positive control capture molecule binds to the conjugate binding molecule. Some such positive control capture molecules that bind to a dye or a fluorophore can quench their signal. Sometimes, the positive control capture molecule is an antibody that that binds to a dye or a fluorophore can quench their signal. In some cases, the positive control capture molecule is a monoclonal antibody. In some cases, an antibody includes any isotype, variable regions, constant regions, Fc region, Fab fragments, F(ab′)2 fragments, and Fab′ fragments. Alternatively, the positive control capture molecule is a non-antibody compound that specifically binds the detection moiety. Sometimes, the positive control capture molecule is a polypeptide that can bind to at least one of the conjugate, the conjugate binding molecule, or detection moiety. In some instances, the conjugate unbound to the detection moiety binds to the positive control capture molecule specific to at least one of the conjugate, the conjugate binding molecule.

The kit or system described herein may also comprise a positive control sample to determine the activity of at least one of programmable nuclease, a guide nucleic acid, or a single stranded reporter. Often, the positive control sample comprises a target nucleic acid that binds to the guide nucleic acid. The positive control sample is contacted with the reagents in the same manner as the test sample and visualized using the support medium. The visualization of the positive control spot and the detection spot for the positive control sample provides a validation of the reagents and the assay.

The kit or system for detection of a target nucleic acid described herein further can comprises reagents protease treatment of the sample. The sample can be treated with protease, such as Proteinase K, before amplification or before assaying for a detectable signal. Often, a protease treatment is for no more than 15 minutes. Sometimes, the protease treatment is for no more than 1, 5, 10, 15, 20, 25, 30, or more minutes, or any value from 1 to 30 minutes.

The kit or system for detection of a target nucleic acid described herein further comprises reagents for nucleic acid amplification of target nucleic acids in the sample. Isothermal nucleic acid amplification allows the use of the kit or system in remote regions or low resource settings without specialized equipment for amplification. Often, the reagents for nucleic acid amplification comprise a recombinase, a oligonucleotide primer, a single-stranded DNA binding (SSB) protein, and a polymerase. Sometimes, nucleic acid amplification of the sample improves at least one of sensitivity, specificity, or accuracy of the assay in detecting the target nucleic acid. In some cases, the nucleic acid amplification is performed in a nucleic acid amplification region on the support medium. Alternatively, or in combination, the nucleic acid amplification is performed in a reagent chamber, and the resulting sample is applied to the support medium. Sometimes, the nucleic acid amplification is isothermal nucleic acid amplification. In some cases, the nucleic acid amplification is transcription mediated amplification (TMA). Nucleic acid amplification is helicase dependent amplification (HDA) or circular helicase dependent amplification (cHDA) in other cases. In additional cases, nucleic acid amplification is strand displacement amplification (SDA). In some cases, nucleic acid amplification is by recombinase polymerase amplification (RPA). In some cases, nucleic acid amplification is by at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). Often, the nucleic acid amplification is performed for no greater than 1, 2, 3, 4, 5, 6, 7, 8, 9, 10, 11, 12, 13, 14, 15, 16, 17, 18, 19, 20, 25, 30, 40, 50, or 60 minutes, or any value from 1 to 60 minutes. Sometimes, the nucleic acid amplification reaction is performed at a temperature of around 20-45° C. In some cases, the nucleic acid amplification reaction is performed at a temperature no greater than 20° C., 25° C., 30° C., 35° C., 37° C., 40° C., 45° C., or any value from 20° C. to 45° C. In some cases, the nucleic acid amplification reaction is performed at a temperature of at least 20° C., 25° C., 30° C., 35° C., 37° C., 40° C., or 45° C., or any value from 20° C. to 45° C.

Sometimes, the total time for the performing the method described herein is no greater than 3 hours, 2 hours, 1 hour, 50 minutes, 40 minutes, 30 minutes, 20 minutes, or any value from 3 hours to 20 minutes. Often, a method of nucleic acid detection from a raw or complex sample comprises protease treating the sample for no more than 15 minutes, amplifying (also referred to as pre-amplifying) the sample for no more than 15 minutes, subjecting the sample to a programmable nuclease-mediated detection, and assaying nuclease mediated detection. The total time for performing this method, sometimes, is no greater than 3 hours, 2 hours, 1 hour, 50 minutes, 40 minutes, 30 minutes, 20 minutes, or any value from 3 hours to 20 minutes. Often, the protease treatment is Proteinase K. Often the amplifying is thermal cycling amplification. Sometimes, the amplifying is isothermal amplification.

Described herein is a collection pad region that provides a region to collect a sample that flows along or from the support medium. Often the collection pads are placed downstream of the detection region and comprise an absorbent material. The collection pad can increase the total volume of sample that enters the support medium by collecting and removing the sample from other smaller volume regions of the support medium. This increased volume can be used to wash unbound conjugates away from the detection region to lower the background noise and enhance assay sensitivity. When the design of the support medium does not include a collection pad, the volume of sample analyzed in the support medium may be determined by the bed volume of the support medium. The collection pad may provide a reservoir for excess or depleted sample volume and may help to provide capillary force for the flow of the sample along the support medium.

The collection pad may be prepared from various materials that are highly absorbent and able to retain fluids. Often the collection pads comprise cellulose filters. In some instances, the collection pads comprise cellulose, cotton, woven meshes, polymer-based matrices. The dimension of the collection pad, usually the length of the collection pad, may be adjusted to change the overall volume absorbed by the support medium.

The support medium described herein may have a barrier around the edge of the support medium. Often the barrier is a hydrophobic barrier that facilitates the maintenance of the sample within the support medium or flow of the sample within the support medium. Usually, the transport rate of the sample in the hydrophobic barrier is much lower than through the regions of the support medium. In some cases, the hydrophobic barrier is prepared by contacting a hydrophobic material around the edge of the support medium. Sometimes, the hydrophobic barrier comprises at least one of wax, polydimethylsiloxane, rubber, or silicone.

Any of the regions on the support medium can be treated with chemicals to improve the visualization of the detection spot and positive control spot on the support medium. The regions can be treated to enhance extraction of nucleic acid in the sample, to control the transport of the reacted reagents and sample or the conjugate to other regions of the support medium, or to enhance the binding of the cleaved detection moiety to the conjugate binding molecule on the surface of the conjugate or to the capture molecule in the detection region. The chemicals may comprise detergents, surfactants, buffers, salts, viscosity enhancers, or polypeptides. In some instances, the chemical comprises bovine serum albumin. In some cases, the chemicals or physical agents enhance flow of the sample with a more even flow across the width of the region. In some cases, the chemicals or physical agents provide a more even mixing of the sample across the width of the region. In some cases, the chemicals or physical agents control flow rate to be faster or slower in order to improve performance of the assay. Sometimes, the performance of the assay is measured by at least one of shorter assay time, longer times during cleavage activity, longer or shorter binding time with the conjugate, sensitivity, specificity, or accuracy.

B. Housing or Enclosure

A support medium as described herein can be house or enclosed in a number of ways that are consistent with the streamlined lateral flow devices and methods disclosed herein. The enclosure for the support medium are, for example, consistent with streamlined lateral flow devices disclosed herein for detection of a target nucleic acid within the sample. For example, the streamlined lateral flow device may comprise support mediums to channel the flow of fluid from one chamber to another and wherein the entire streamlined lateral flow device is encased within the enclosure or housing described herein. Typically, the support medium described herein is encased in an enclosure to protect the support medium from contamination and from intentional or unintentional disassembly. The enclosure can be made of more than one part and be assembled to encase the support medium. In some instances, a single enclosure can encase more than one support medium. The enclosure can be made from cardboard, plastics, polymers, or materials that provide mechanical protection for the support medium. Often, the material for the enclosure is inert or does not react with the support medium or the reagents placed on the support medium. The enclosure may have an upper part which, when in place, exposes the sample pad to receive the sample and has an opening or window above the detection region to allow the results of the lateral flow assay to be read. The enclosure may have guide pins on its inner surface that are placed around and on the support medium to help secure the compartments and the support medium in place within the enclosure. In some cases, the enclosure encases the entire support medium. Alternatively, the sample pad of the support medium is not encased and is left exposed to facilitate the receiving of the sample while the rest of the support medium is encased in the enclosure.

The enclosure and the support medium encased within the enclosure may be sized to be small, low profile, portable, and/or hand held. The small size of the enclosure and the support medium would facilitate the transport and use of the assay in remote regions and/or low resource settings. In some cases, the enclosure has a length of no more than 30 cm, 25 cm, 20 cm, 15 cm, 10 cm, or 5 cm. In some cases, the enclosure has a length of at least 1 cm, 5 cm, 10 cm, 15 cm, 20 cm, 25 cm, or 30 cm. In some cases, the enclosure has a width of no more than 30 cm, 25 cm, 20 cm, 15 cm, 10 cm, 5 cm, 4 cm, 3 cm, 2 cm, or 1 cm. In some cases, the enclosure has a width of at least 1 cm, 2 cm, 3 cm, 4 cm, 5 cm, 10 cm, 15 cm, 20 cm, 25 cm, or 30 cm. In some cases, the enclosure has a height of no more than 10 cm, 9 cm, 8 cm, 7 cm, 6 cm, 5 cm, 4 cm, 3 cm, 2 cm, or 1 cm. In some cases, the enclosure has a height of at least 1 cm, 2 cm, 3 cm, 4 cm, 5 cm, 6 cm, 7 cm, 8 cm, 9 cm, or 10 cm. Typically, the enclosure is rectangular in shape.

In some instances, the enclosure provides additional information on the outer surface of the upper cover to facilitate the identification of the test type, visualization of the detection region, and analysis of the results. The upper outer enclosure may have identification label including but not limited to barcodes, QR codes, identification label, or other visually identifiable labels. In some instances, the identification label is imaged by a camera on a mobile device, and the image is analyzed to identify the disease that is being tested for. The correct identification of the test is important to accurately visualize and analyze the results. In some instances, the upper outer enclosure has fiduciary markers to orient the detection region to distinguish the positive control spot from the detection spots. In some instances, the upper outer enclosure has a color reference guide. When the detection region is imaged with the color reference guide, the detection spots, located using the fiduciary marker, can be compared with the positive control spot and the color reference guide to determine various image properties of the detection spot such as color, color intensity, and size of the spot. In some instances, the color reference guide has red, green, blue, black, and white colors. In some cases, the image of the detection spot can be normalized to at least one of the reference colors of the color reference guide, compared to at least two of the reference colors of the color reference guide, and generate a value for the detection spot. Sometimes, the comparison to at least two of the reference colors is comparison to a standard reference scale. In some instances, the image of the detection spot in some instance undergoes transformation or filtering prior to analysis. Analysis of the image properties of the detection spot can provide information regarding presence or absence of the target nucleic acid targeted by the assay and the disease associated with the target nucleic acid. In some instances, the analysis provides a qualitative result of presence or absence of the target nucleic acid in the sample. In some instances, the analysis provides a semi-quantitative or quantitative result of the level of the target nucleic acid present in the sample. Quantification may be performed by having a set of standards in spots/wells and comparing the test sample to the range of standards. A more semi-quantitative approach may be performed by calculating the color intensity of 2 spots/well compared to each other and measuring if one spot/well is more intense than the other. Sometimes, quantification is of quantification of circulating nucleic acid. The circulating nucleic acid can comprise a target nucleic acid. For example, a method of circulating nucleic acid quantification comprises assaying for a target nucleic acid of circulating nucleic acid in a first aliquot of a sample, assaying for a control nucleic acid in a second aliquot of the sample, and quantifying the target nucleic acid target in the first aliquot by measuring a signal produced by cleavage of a reporter. Sometimes, a method of circulating RNA quantification comprises assaying for a target nucleic acid of the circulating RNA in a first aliquot of a sample, assaying for a control nucleic acid in a second aliquot of the sample, and quantifying the target nucleic acid target in the first aliquot by measuring a signal produced by cleavage of a reporter. Often, the output comprises fluorescence/second. The reaction rate, sometimes, is log linear for output signal and target nucleic acid concentration. In some instances, the signal output is correlated with the target nucleic acid concentration. Sometimes, the circulating nucleic acid is DNA.

C. Detection/Visualization Devices

A number of detection or visualization devices and methods are consistent with the streamlined lateral flow devices and methods disclosed herein. Methods of detection/visualization are, for example, consistent with streamlined lateral flow devices disclosed herein for detection of a target nucleic acid within the sample. For example, the streamlined lateral flow device may comprise an incubation and detection chamber or a stand-alone detection chamber, in which a colorimetric, fluorescence, electrochemical, or electrochemiluminescence signal is generated for detection/visualization. Sometimes, the signal generated for detection is a calorimetric, potentiometric, amperometric, optical (e.g., fluorescent, colorimetric, etc.), or piezo-electric signal. Often a calorimetric signal is heat produced after cleavage of the reporters. Sometimes, a calorimetric signal is heat absorbed after cleavage of the reporters. A potentiometric signal, for example, is electrical potential produced after cleavage of the reporters. An amperometric signal can be movement of electrons produced after the cleavage of reporter. Often, the signal is an optical signal, such as a colorimetric signal or a fluorescence signal. An optical signal is, for example, a light output produced after the cleavage of the reporters. Sometimes, an optical signal is a change in light absorbance between before and after the cleavage of reporters. Often, a piezo-electric signal is a change in mass between before and after the cleavage of the reporter. Sometimes, the reporter is protein-nucleic acid. Often, the protein-nucleic acid is an enzyme-nucleic acid. The detection/visualization can be analyzed using various methods, as further described below. The results from the detection region from a completed assay can be visualized and analyzed in various ways. In some cases, the positive control spot and the detection spot in the detection region is visible by eye, and the results can be read by the user. In some cases, the positive control spot and the detection spot in the detection region is visualized by an imaging device. Often, the imaging device is a digital camera, such a digital camera on a mobile device. The mobile device may have a software program or a mobile application that can capture an image of the support medium, identify the assay being performed, detect the detection region and the detection spot, provide image properties of the detection spot, analyze the image properties of the detection spot, and provide a result. Alternatively or in combination, the imaging device can capture fluorescence, ultraviolet (UV), infrared (IR), or visible wavelength signals. The imaging device may have an excitation source to provide the excitation energy and captures the emitted signals. In some cases, the excitation source can be a camera flash and optionally a filter. In some cases, the imaging device is used together with an imaging box that is placed over the support medium to create a dark room to improve imaging. The imaging box can be a cardboard box that the imaging device can fit into before imaging. In some instances, the imaging box has optical lenses, mirrors, filters, or other optical elements to aid in generating a more focused excitation signal or to capture a more focused emission signal. Often, the imaging box and the imaging device are small, handheld, and portable to facilitate the transport and use of the assay in remote or low resource settings.

The assay described herein can be visualized and analyzed by a mobile application (app) or a software program. Using the graphic user interface (GUI) of the app or program, an individual can take an image of the support medium, including the detection region, barcode, reference color scale, and fiduciary markers on the enclosure, using a camera on a mobile device. The program or app reads the barcode or identifiable label for the test type, locate the fiduciary marker to orient the sample, and read the detectable signals, compare against the reference color grid, and determine the presence or absence of the target nucleic acid, which indicates the presence of the gene, virus, or the agent responsible for the disease. The mobile application can present the results of the test to the individual. The mobile application can store the test results in the mobile application. The mobile application can communicate with a remote device and transfer the data of the test results. The test results can be viewable remotely from the remote device by another individual, including a healthcare professional. A remote user can access the results and use the information to recommend action for treatment, intervention, clean up of an environment.

### Spin-Through Filter Device

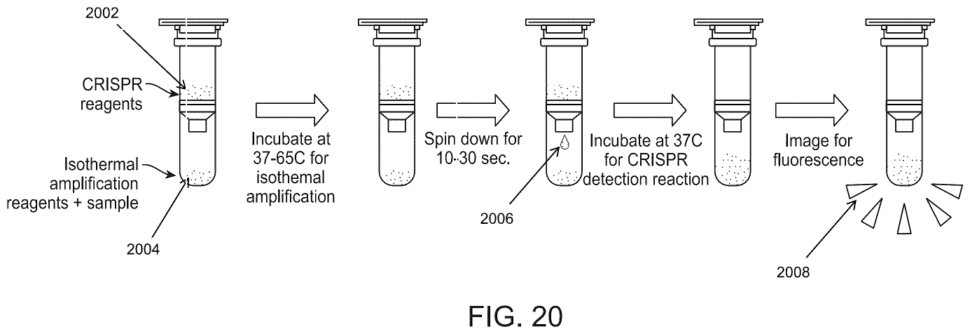

Described herein are various devices for programmable nuclease based-detection preventing contamination and utilizing a spin-through assay as shown in FIG. 20. In some embodiments, spin-through filter devices comprise: an enclosure comprising programmable nuclease-based detection reagents, amplification reagents, and a sample, wherein the detection reagents are physically separated from a mixture comprising the amplification reagents and the sample by a filter, wherein the filter is configured to maintain separation between the detection reagents and the mixture until the enclosure is centrifuged, thereby allowing for amplification of the sample by the amplification reagents; wherein the filter is configured to facilitate transfer of the detection reagents in to the mixture upon centrifugation of the enclosure; and wherein the enclosure is configured to allow for visualization of one or more signals produced the mixture. In some embodiments, the enclosure comprises a transparent or translucent material configured to allow fluorescent light to pass therethrough. In some embodiments, the enclosure comprises a transparent or translucent material configured to allow visible light to pass therethrough.

### Multiplexing

The streamlined lateral flow devices and methods described herein can be multiplexed in a number of ways. These methods of multiplexing are, for example, consistent with streamlined lateral flow devices disclosed herein for detection of a target nucleic acid within the sample.

Methods consistent with the present disclosure include a multiplexing method of assaying for two or more target nucleic acids in a sample. A multiplexing method comprises contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. As another example, multiplexing method of assaying for a target nucleic acid in a sample, for example, comprises: a) contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; b) contacting the complex to a substrate; c) contacting the substrate to a reagent that differentially reacts with a cleaved substrate; and d) assaying for a signal indicating cleavage of the substrate, wherein the signal indicates a presence of the target nucleic acid in the sample and wherein absence of the signal indicates an absence of the target nucleic acid in the sample. Often, the substrate is an enzyme-nucleic acid. Sometimes, the substrate is an enzyme substrate-nucleic acid.

Multiplexing can be either spatial multiplexing wherein multiple different target nucleic acids are detected from the same sample at the same time, but the reactions are spatially separated. Often, the multiple target nucleic acids are detected using the same programmable nuclease, but different guide nucleic acids. The multiple target nucleic acids sometimes are detected using the different programmable nucleases. Sometimes, multiplexing can be single reaction multiplexing wherein multiple different target acids are detected in a single reaction volume. Often, at least two different programmable nucleases are used in single reaction multiplexing. For example, multiplexing can be enabled by immobilization of multiple categories of reporters within a streamlined lateral flow device, to enable detection of multiple target nucleic acids within a single streamlined lateral flow device. Multiplexing allows for detection of multiple target nucleic acids in one kit or system. In some cases, the multiple target nucleic acids comprise different target nucleic acids to a virus. In some cases, the multiple target nucleic acids comprise different target nucleic acids associated with at least a first disease and a second disease. Multiplexing for one disease increases at least one of sensitivity, specificity, or accuracy of the assay to detect the presence of the disease in the sample. In some cases, the multiple target nucleic acids comprise target nucleic acids directed to different viruses, bacteria, or pathogens responsible for more than one disease. In some cases, multiplexing allows for discrimination between multiple target nucleic acids, such as target nucleic acids that comprise different genotypes of the same bacteria or pathogen responsible for a disease, for example, for a wild-type genotype of a bacteria or pathogen and for genotype of a bacteria or pathogen comprising a mutation, such as a single nucleotide polymorphism (SNP) that can confer resistance to a treatment, such as antibiotic treatment. For example, multiplexing comprises method of assaying comprising a single assay for a microorganism species using a first programmable nuclease and an antibiotic resistance pattern in a microorganism using a second programmable nuclease. Sometimes, multiplexing allows for discrimination between multiple target nucleic acids of different influenza strains, for example, influenza A and influenza B. Often, multiplexing allows for discrimination between multiple target nucleic acids, such as target nucleic acids that comprise different genotypes, for example, for a wild-type genotype and for a mutant (e.g., SNP) genotype. Multiplexing for multiple viral infections can provide the capability to test a panel of diseases from a single sample. For example, multiplexing for multiple diseases can be valuable in a broad panel testing of a new patient or in epidemiological surveys. Often multiplexing is used for identifying bacterial pathogens in sepsis or other diseases associated with multiple pathogens.

Furthermore, signals from multiplexing can be quantified. For example, a method of quantification for a disease panel comprises assaying for a plurality of unique target nucleic acids in a plurality of aliquots from a sample, assaying for a control nucleic acid control in another aliquot of the sample, and quantifying a plurality of signals of the plurality of unique target nucleic acids by measuring signals produced by cleavage of reporters compared to the signal produced in the second aliquot. Often the plurality of unique target nucleic acids are from a plurality of viruses in the sample. Sometimes the quantification of a signal of the plurality correlates with a concentration of a unique target nucleic acid of the plurality for the unique target nucleic acid of the plurality that produced the signal of the plurality. The disease panel can be for any disease.

The streamlined lateral flow devices and methods described herein can be multiplexed by various configurations of the reagents and the support medium. In some cases, the kit or system is designed to have multiple support mediums encased in a single enclosure. Sometimes, the multiple support mediums housed in a single enclosure share a single sample pad. The single sample pad may be connected to the support mediums in various designs such as a branching or a radial formation. Alternatively, each of the multiple support mediums has its own sample pad. In some cases, the kit or system is designed to have a single support medium encased in a enclosure, where the support medium comprises multiple detection spots for detecting multiple target nucleic acids. Sometimes, the reagents for multiplexed assays comprise multiple guide nucleic acids, multiple programmable nucleases, and multiple single stranded reporters, where a combination of one of the guide nucleic acids, one of the programmable nucleases, and one of the single stranded reporters detects one target nucleic acid and can provide a detection spot on the detection region. In some cases, the combination of a guide nucleic acid, a programmable nuclease, and a single stranded reporter configured to detect one target nucleic acid is mixed with at least one other combination in a single reagent chamber. In some cases, the combination of a guide nucleic acid, a programmable nuclease, and a single stranded reporter configured to detect one target nucleic acid is mixed with at least one other combination of reagents on a single support medium. When these combinations of reagents are contacted with the sample, the reaction for the multiple target nucleic acids occurs simultaneously in the same medium or reagent chamber. Sometimes, this reacted sample is applied to the multiplexed support medium described herein.

In some cases, the combination of a guide nucleic acid, a programmable nuclease, and a single stranded reporter configured to detect one target nucleic acid is provided in its own reagent chamber or its own support medium. In this case, multiple reagent chambers or support mediums are provided in the device, kit, or system, where one reagent chamber is designed to detect one target nucleic acid. In this case, multiple support mediums are used to detect the panel of viral infections, or other diseases of interest.

In some instances, the multiplexed streamlined lateral flow devices and methods detect at least 2 different target nucleic acids in a single reaction. In some instances, the multiplexed streamlined lateral flow devices and methods detect at least 3 different target nucleic acids in a single reaction. In some instances, the multiplexed streamlined lateral flow devices and methods detect at least 4 different target nucleic acids in a single reaction. In some instances, the multiplexed streamlined lateral flow devices and methods detect at least 5 different target nucleic acids in a single reaction. In some cases, the multiplexed streamlined lateral flow devices and methods detect at least 6, 7, 8, 9, or 10 different target nucleic acids in a single reaction. In some instances, the multiplexed kits detect at least 2 different target nucleic acids in a single kit. In some instances, the multiplexed kits detect at least 3 different target nucleic acids in a single kit. In some instances, the multiplexed kits detect at least 4 different target nucleic acids in a single kit. In some instances, the multiplexed kits detect at least 5 different target nucleic acids in a single kit. In some instances, the multiplexed kits detect at least 6, 7, 8, 9, or 10 different target nucleic acids in a single kit.

### Manufacturing

The support medium may be assembled with a variety of materials and reagents. Reagents may be dispensed or coated on to the surface of the material for the support medium. The material for the support medium may be laminated to a backing card, and the backing card may be singulated or cut into individual test strips. The device may be manufactured by completely manual, batch-style processing; or a completely automated, in-line continuous process; or a hybrid of the two processing approaches. The batch process may start with sheets or rolls of each material for the support medium. Individual zones of the support medium may be processed independently for dispensing and drying, and the final support medium may be assembled with the independently prepared zones and cut. The batch processing scheme may have a lower cost of equipment, and a higher labor cost than more automated in-line processing, which may have higher equipment costs. In some instances, batch processing may be preferred for low volume production due to the reduced capital investment. In some instances, automated in-line processing may be preferred for high volume production due to reduced production time. Both approaches may be scalable to production level.

In some instances, the support mediums are prepared using various instruments, including an XYZ-direction motion system with dispensers, impregnation tanks, drying ovens, a manual or semi-automated laminator, and cutting methods for reducing roll or sheet stock to appropriate lengths and widths for lamination. For dispensing the conjugate binding molecules for the conjugate zone and capture molecules for the detection zones, an XYZ-direction motion system with dispensers may be used. In some embodiments, the dispenser may dispense by a contact method or a non-contact method.

In automated or semi-automated preparation of the support medium, the support medium may be prepared from rolls of membranes for each region that are ordered into the final assembled order and unfurled from the rolls. For example, the membranes can be ordered from sample pad region to collection pad region from left to right with one membrane corresponding to a region on the support medium, all onto an adhesive cardstock. The dispenser places the reagents, conjugates, detection molecules, and other treatments for the membrane onto the membrane. The dispensed fluids are dried onto the membranes by heat, in a low humidity chamber, or by freeze drying to stabilize the dispensed molecules. The membranes are cut into strips and placed into the enclosure or housing and packaged.

### Kit

Disclosed herein are kits of streamlined lateral flow devices for use to detect a target nucleic acid. In some embodiments, the kit comprises at least one of the reagents described herein and the support medium. The reagent may be provided in a reagent chamber or on the support medium. Alternatively, the reagent may be placed into the reagent chamber or the support medium by the individual using the kit. Optionally, the kit further comprises a buffer and a dropper. The reagent chamber be a test well or container. The opening of the reagent chamber may be large enough to accommodate the support medium. The buffer may be provided in a dropper bottle for ease of dispensing. The dropper can be disposable and transfer a fixed volume. The dropper can be used to place a sample into the reagent chamber or on the support medium.

In some embodiments, a kit for detecting a target nucleic acid comprising a support medium; a guide nucleic acid targeting a target nucleic acid segment; a programmable nuclease capable of being activated when complexed with the guide nucleic acid and the target nucleic acid segment; and a single stranded reporter comprising a detection moiety, wherein the reporter is capable of being cleaved by the activated nuclease, thereby generating a first detectable signal.

In some embodiments, a kit for detecting a target nucleic acid comprising a PCR plate; a guide nucleic acid targeting a target nucleic acid segment; a programmable nuclease capable of being activated when complexed with the guide nucleic acid and the target nucleic acid segment; and a single stranded reporter comprising a detection moiety, wherein the reporter is capable of being cleaved by the activated nuclease, thereby generating a first detectable signal. The wells of the PCR plate can be pre-aliquoted with the guide nucleic acid targeting a target nucleic acid segment, a programmable nuclease capable of being activated when complexed with the guide nucleic acid and the target sequence, and at least one population of a single stranded reporter comprising a detection moiety. A user can thus add the biological sample of interest to a well of the pre-aliquoted PCR plate and measure for the detectable signal with a fluorescent light reader or a visible light reader.

In some instances, such kits may include a package, carrier, or container that is compartmentalized to receive one or more containers such as vials, tubes, and the like, each of the container(s) comprising one of the separate elements to be used in a method described herein. Suitable containers include, for example, test wells, bottles, vials, and test tubes. In one embodiment, the containers are formed from a variety of materials such as glass, plastic, or polymers.

The kit or systems described herein contain packaging materials. Examples of packaging materials include, but are not limited to, pouches, blister packs, bottles, tubes, bags, containers, bottles, and any packaging material suitable for intended mode of use.

A kit typically includes labels listing contents and/or instructions for use, and package inserts with instructions for use. A set of instructions will also typically be included. In one embodiment, a label is on or associated with the container. In some instances, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

After packaging the formed product and wrapping or boxing to maintain a sterile barrier, the product may be terminally sterilized by heat sterilization, gas sterilization, gamma irradiation, or by electron beam sterilization. Alternatively, the product may be prepared and packaged by aseptic processing.

### Stability

Disclosed herein are stable compositions of the reagents and the programmable nuclease system for use in the methods as discussed above. The reagents and programmable nuclease system described herein may be stable in various storage conditions including refrigerated, ambient, and accelerated conditions. Disclosed herein are stable reagents. The stability may be measured for the reagents and programmable nuclease system themselves or the reagents and programmable nuclease system present on the support medium.

In some instances, stable as used herein refers to a reagents having about 5% w/w or less total impurities at the end of a given storage period. Stability may be assessed by HPLC or any other known testing method. The stable reagents may have about 10% w/w, about 5% w/w, about 4% w/w, about 3% w/w, about 2% w/w, about 1% w/w, or about 0.5% w/w total impurities at the end of a given storage period.

In some embodiments, stable as used herein refers to a reagents and programmable nuclease system having about 10% or less loss of detection activity at the end of a given storage period and at a given storage condition. Detection activity can be assessed by known positive sample using a known method. Alternatively, or combination, detection activity can be assessed by the sensitivity, accuracy, or specificity. In some embodiments, the stable reagents has about 10%, about 9%, about 8%, about 7%, about 6%, about 5%, about 4%, about 3%, about 2%, about 1%, or about 0.5% loss of detection activity at the end of a given storage period.

In some embodiments, the stable composition has zero loss of detection activity at the end of a given storage period and at a given storage condition. The given storage condition may comprise humidity of equal to or less than 10%, 20%, 30%, 40%, 50%, 60%, 70%, 80%, 90%, or 100% relative humidity. The controlled storage environment may comprise humidity between 0% and 50% relative humidity, 0% and 40% relative humidity, 0% and 30% relative humidity, 0% and 20% relative humidity, or 0% and 10% relative humidity. The controlled storage environment may comprise temperatures of −100° C., −80° C., −20° C., 4° C., about 25° C. (room temperature), or 40° C. The controlled storage environment may comprise temperatures between −80° C. and 25° C., or −100° C. and 40° C. The controlled storage environment may protect the system or kit from light or from mechanical damage. The controlled storage environment may be sterile or aseptic or maintain the sterility of the light conduit. The controlled storage environment may be aseptic or sterile.

The kit or system can be packaged to be stored for extended periods of time prior to use. The kit or system may be packaged to avoid degradation of the kit or system. The packaging may include desiccants or other agents to control the humidity within the packaging. The packaging may protect the kit or system from mechanical damage or thermal damage. The packaging may protect the kit or system from contamination of the reagents and programmable nuclease system. The kit or system may be transported under conditions similar to the storage conditions that result in high stability of the reagent or little loss of reagent activity. The packaging may be configured to provide and maintain sterility of the kit or system. The kit or system can be compatible with standard manufacturing and shipping operations.

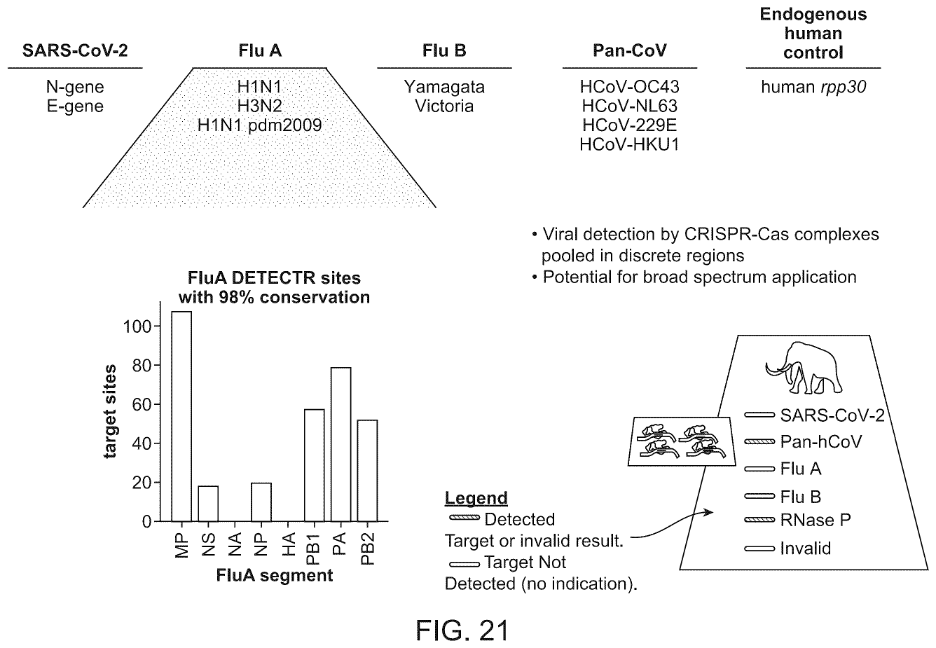

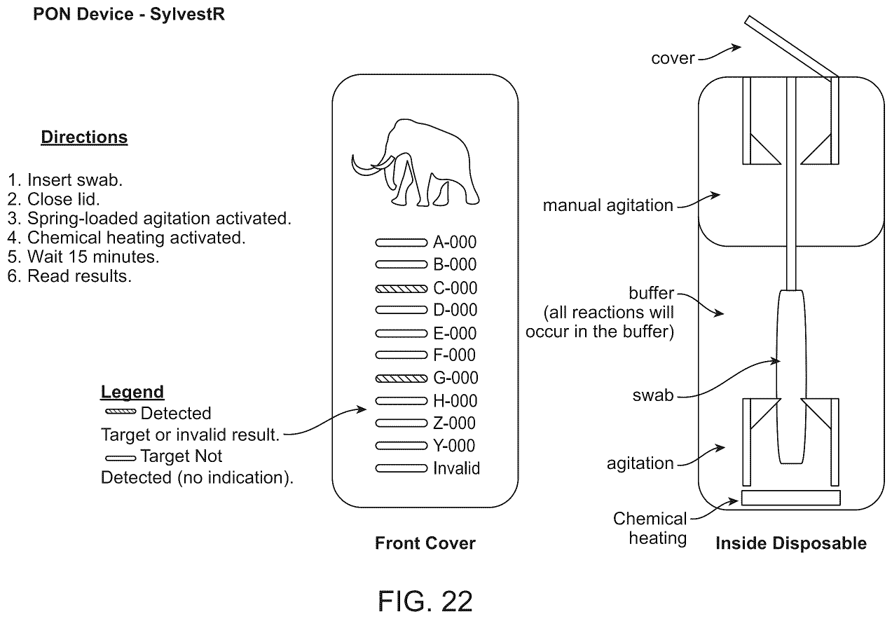

Described herein are various embodiments, for point of need (PON) programmable nuclease-based devices. In some embodiments, the PON device is configured for a 5-plex respiratory panel as shown in in FIG. 21. FIG. 21 shows an exemplary assay design for a PON 5-plex panel comprising pooled CRISPR-Cas complexes in discrete regions for viral detection. The discrete regions are for detection of: (1) SARS-CoV-2, (2) Flu A, (3) Flu B, (4) Pan-CoV, and (5) Endogenous human control. The (1) SARS-CoV-2 region comprises gRNA for detecting N-gene targets and E-gene targets, the (2) Flu A region comprises gRNA for detecting H1N1 targets, H3N2 targets, and H1N1 pdm2009 targets, the (3) Flu B region comprises gRNA for detecting Yamagata targets and Victoria targets, the (4) Pan-CoV region comprises gRNA for detecting HCoV-OC43 targets, HCoV-NL63 targets, HCoV-229E targets, and HCoV-HKU1 targets, and the (5) Endogenous human control region comprises gRNA for human rpp30 targets. Each region can comprise pooled gRNA. For example, the gRNAs for the Flu A region bind to target sites that are 98% conserved among H1N1, H3N2, and H1N1 pdm2009, such as Matrix Protein 1 (MP), Nonstructural Protein 1 (NS), Neuraminidase (NA), Nucleoprotein (NP), Hemagglutinin (HA), PB1, Polymerase Acidic Protein (PA), and Polymerase Basic Protein 2 (PB2). Detected signal from each region can indicate the detection of a target within that region. In some embodiments, PON programmable nuclease-based devices are disposable, as shown in FIG. 22.

### Multiplexed DETECTR™ Assay-Based Lateral Flow Assay

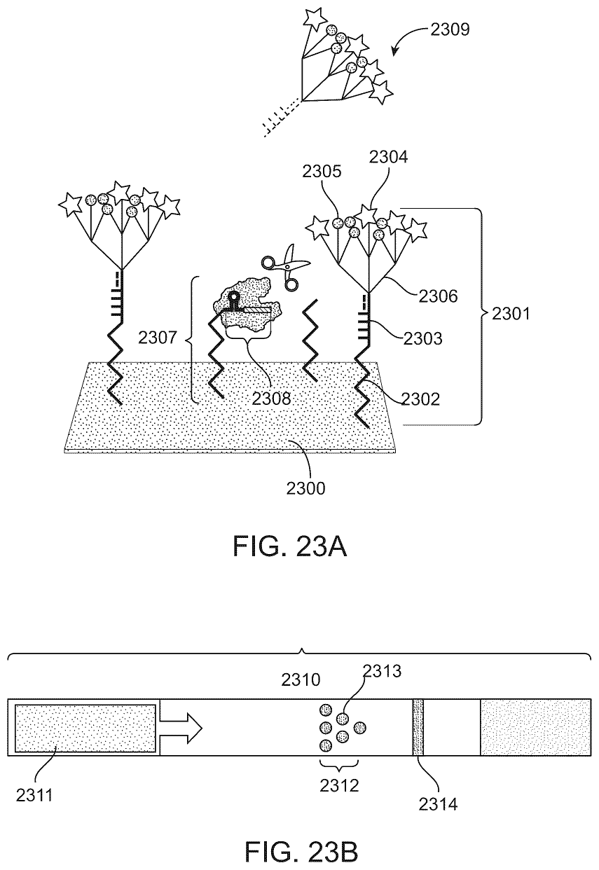

Described here are various devices and methods for a DETECTR™ assay based multiplex lateral flow strip as illustrated in FIG. 23. In some embodiments, reporters (2301) are immobilized to a surface (2300) of a solid support. In some embodiments, programmable nuclease (e.g., Cas-complex) probes (2307) are immobilized to a surface (2300). In some embodiments, programmable nuclease probes (2307) comprise guide nucleic acid such as a single guide RNA (sgRNA) (2308). In some embodiments, a programmable nuclease probe (2307) comprises a sgRNA (2308) that is designed to be a compliment for a target nucleic acid of a sample. In some embodiments, programmable nuclease probes (2307) and reporters (2301) are both immobilized to a surface (2300). In some embodiments, programmable nuclease probes (2307) and reporters (2301) are both immobilized to a surface (2300) in close enough proximity that the reporter (2301) can be cleaved by the programmable nuclease of the programmable nuclease probe (2307). In some embodiments, programmable nuclease probes (2307) and reporters (2301) are both immobilized to a surface (2300) in close enough proximity that the reporter (2301) can be cleaved (2309) by the programmable nuclease of the programmable nuclease probe (2307) upon binding of a target nucleic acid to an sgRNA (2308) of the programmable nuclease probe (2307) when target nucleic acid and sgRNA (2308) are compliments. In such an embodiment, this indicates the presence of and is a “hit” for the target. In some embodiments, binding of a target nucleic acid that is complimentary to a sgRNA (2308) of the programmable nuclease probe (2307) results in the programmable nuclease of the programmable nuclease probe (2307) initiating cleavage of nucleic acids within a close enough proximity of the programmable nuclease. In some embodiments, the surface (2300) is in the bottom of a well. In some embodiments, a collection of a first programmable nuclease probe (2307) and a first reporter (2301) are immobilized to a surface at one location of the surface (2300).

In some embodiments, as illustrated in FIG. 23A the reporter (2301) comprises a surface linker (2302), a nucleic acid (2303), a second linker (2306), an detection moiety (e.g., a label) (2304), and an affinity molecule (e.g., a binding moiety) (2305). In some embodiments, the binding moiety (2305) is biotin. In some embodiments, there is more than one copy of the same reporter (2301) immobilized to the surface.

In some embodiments, lateral flow assay strips (2310) are used to detect cleaved reporters (2309). In some embodiments, cleaved reporters (2309) are contacted to the sample pad (2311) of the lateral flow strip (2310). In some embodiments, the cleaved reporters (2309) bind to conjugate particles present in the sample pad. In some embodiments, the conjugate particles are gold nanoparticles. In some embodiments, the gold nanoparticles are functionalized with anti-biotin. In some embodiments, the anti-biotin functionalized gold nanoparticles bind to the cleaved reporter which contains one or more biotins in the binding moiety (2305).

In some embodiments, the reporter contains a second linker. In some embodiments, the second linker links one or more binding moieties to the nucleic acid. In some embodiments, the second linker links one or more labels to the nucleic acid. In some embodiments, the second linker links both one or more binding moieties and one or more labels to the nucleic acid of the reporter. In some embodiments, the reporter is a dendrimer or trebler molecule.

In some embodiments, the reporter contains a label. In some embodiments, label is FITC, DIG, TAMRA, Cy5, AF594, Cy3, or any appropriate label for a lateral flow assay.

In some embodiments, the reporter comprises chemical functional group for binding. In some embodiments, the chemical functional group is biotin. In some embodiments, the chemical functional group is complimentary to a capture probe on the flowing capture probe (e.g. conjugate particle or capture molecule). In some embodiments, the flowing capture probe is a gold nanoparticle functionalized with anti-biotin. In some embodiments, the flow capture probe is located in the sample pad. In some embodiments, the flowing capture probe is located in a conjugate pad in contact with the sample pad, wherein both lateral flow assay strip comprises both the sample pad and conjugate pad, further wherein both the sample pad and the conjugate pad are in fluid communication with the detection region.

In some embodiments, the lateral flow assay strip (2310) contains a detection region (2312). In some embodiments, the detection region (2312) comprises one or more detection spots. In some embodiments, the detection spots contain a stationary capture probe (e.g., capture molecule). In some embodiments, the stationary capture probe comprises one or more capture antibodies. In some embodiments, the capture antibodies are anti-FITC, anti-DIG, anti-TAMRA, anti-Cy5, anti-AF594, or any other appropriate capture antibody capable of binding the detection moiety or conjugate.

In some embodiments, the flowing capture probe comprising FITC is captured by a stationary capture probe comprising anti-FITC antibody. In some embodiments, the flowing capture probe comprising TAMRA is captured by a stationary capture probe comprising anti-TAMRA antibody. In some embodiments, the flowing capture probe comprising DIG is captured by the stationary capture probe comprising anti-DIG antibody. In some embodiments, the flowing capture probe comprising Cy5 is captured by the stationary capture probe comprising anti-Cy5 antibody. In some embodiments, the flowing capture probe comprising AF574 is captured by the stationary capture probe comprising anti-AF594 antibody.

In some embodiments, the lateral flow assay strip (2310) comprises a control line (2314). In some embodiments, the control line (2314) comprises anti-IgG that is complimentary to all flowing capture probes. In some embodiments, when a flowing capture probe does not bind to a reporter the flowing capture probe will be captured by the anti-IgG on the control line, ensuring the user that the device is working properly even no signal is read from the test line.

In some embodiments, the lateral flow assay strip (2310) comprises a sample pad. In some embodiments, the flowing capture probe comprises anti-biotin. In some embodiments, the flowing capture probe comprises HRP. In some embodiments, the flowing capture probe comprises HRP-anti-biotin. In some embodiments, the flowing capture probe is HRP-anti-biotin DAB/TMB.

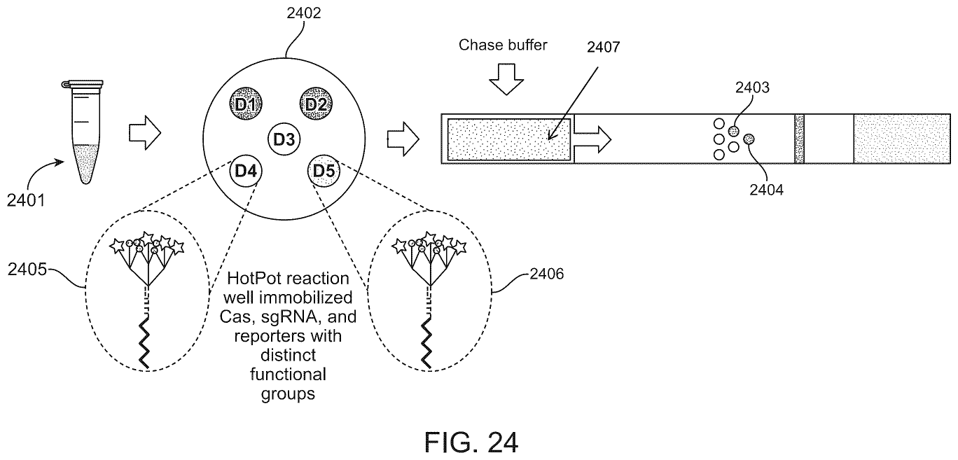

Described here are various devices and methods for a DETECTR™ assay based multiplex lateral flow strip as illustrated in FIG. 24. FIG. 24 depicts a non-limiting exemplary workflow for a DETECTR™ assay read out on a lateral flow assay strip. In some embodiments, a sample (2401) contains one or more target nucleic acid sequences. In some embodiments, a sample (2401) (e.g., a sample solution) contains at least first and second target nucleic acid sequences. In some embodiments, the sample (2401) is introduced into a well (2402) (e.g., D1-D5) where at one or more locations there are different guide nucleic acids such as sgRNAs immobilized to the surface of the well. In some embodiments, the sgRNAs are part of a programmable nuclease probe immobilized to a surface. In some embodiments, a sgRNA is designed to specifically bind to a target nucleic acid in the sample. In some embodiments, there are different sgRNAs corresponding to different locations (e.g., locations D1-D5) on the surface of the well, where each different sgRNA is complimentary for a different target nucleic acid sequence that may or may not be present in the sample. In some embodiments, in addition to the programmable nuclease probes containing sgRNAs, each location is functionalized with one or more reporter probes having distinct functional groups. In some embodiments, the reporter probes are in close enough proximity to be cleaved by the programmable nuclease probes. In some embodiments, as described in example 7, binding between a particular sgRNA and the target nucleic acid to which the sgRNA is designed to specifically bind allows for a section of one or more reporters are cleaved from a corresponding nucleic acid and released into the sample solution. In some embodiments, the reporter is functionalized with a label. In some embodiments, the lateral flow assay strip contains a detection region comprising detection spots (e.g., 2403, 2404), where each detection spot contains a different type of capture antibody. In some embodiments, each capture antibody type specifically binds to a particular label type of a reporter. In some embodiments, a first detection spot (2403) contains the capture antibody anti-FITC. In some embodiments, location D5 on the surface of the well (2402) contains a first immobilized programmable nuclease probe including the sgRNA specific to the first target nucleic acid sequence. In some embodiments, D5 additionally contains the immobilized first reporter (2406), which is labeled with FITC. In some embodiments, upon binding of the first target nucleic acid sequence to the programmable nuclease probe causes a the cleavable nucleic acid of the first reporter (2406) to be cleaved and released into solution. Alternatively, or in combination, in some embodiments, a second detection spot (2404) contains the capture antibody anti-DIG. In some embodiments, a second location D4 contains the immobilized programmable nuclease probe including the sgRNA specific to the second target nucleic acid sequence. In some embodiments, D4 additionally contains the immobilized second reporter (2405), which is labeled with DIG. Therefore, in some embodiments, upon binding of the second target nucleic acid sequence, the immobilized second reporter (2405) is cleaved and released into solution. In some embodiments, the solution containing cleaved first and second reporters (2405) and (2406) is contacted to the sample pad of the lateral flow assay strip along with chase buffer. In some embodiments, the sample pad has one or more flowing capture probes (e.g., anti-biotin-AuNP) disposed thereon. In some embodiments, the sample solution containing the cleaved first and second reporter molecules, along with the chase buffer, flow across the sample pad, where the reporters are bound to conjugates (e.g., anti-biotin-gold nanoparticles). In some embodiments, the solution containing the cleaved reporters is contacted to the sample pad by manually pipetting. In some embodiments, the solution containing the cleaved reporters is contacted to the sample pad being drawn from a chamber in fluid connection with the sample pad. In some embodiments, the solution containing the cleaved reporters is contacted to the sample pad by being drawn from a chamber in which the assay resulting in the cleaved reporter solution occurs. In some embodiments, the reporters are cleaved in the sample pad. In some embodiments, the reporters are cleaved in the sample pad by a DETECTR™ assay. In some embodiments, the solution is drawn into and out of the sample pad by capillary action, or wicking. In some embodiments, the capillary action or wicking is caused by the liquid being drawn into an absorption pad. In some embodiments, the capillary action or wicking is caused by the liquid being drawn into an absorption pad, not requiring electrical power. In some cases, the solution is drawn into or out of the sample pad by a pressure gradient. In some embodiments, the gold nanoparticle-reporter conjugates having reporter (2406) labeled with FITC will selectively bind to the first detection spot (2403) containing the capture antibody anti-FITC, thus indicating the presence of the first target nucleic acid sequence in the sample. In some embodiments, the AuNP-reporter conjugates having mostly reporter (2405) labeled with DIG will selectively bind to the second detection spot (2404) containing the capture antibody anti-DIG, thus indicating the presence of the second target nucleic acid sequence in the sample. In this manner, for some embodiments, parallel detection of two or more target nucleic acid sequences present in a multiplexed sample is enabled.

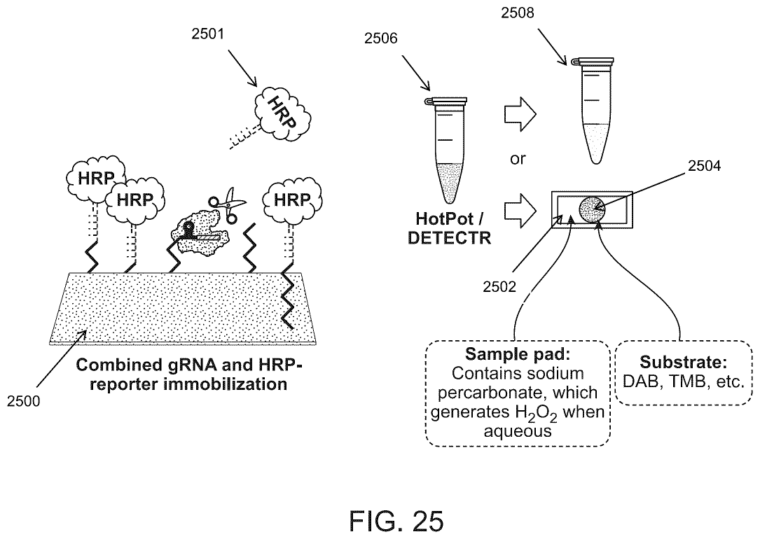

Described herein are various embodiments of lateral flow-based detection as illustrated in FIG. 25. In some embodiments, horse radish peroxidase (HRP) (2501) is used to enhance detection in lateral flow based DETECTR™ assays. In some embodiments, a sample containing a target(s) nucleic acid sequence is exposed to a surface (2500) upon which programmable nuclease probes and reporter probes are immobilized on the surface. In some embodiments, the reporter probes contain HRP molecules. In some embodiments, upon cleavage of the reporter by the programmable nuclease following a specific binding event between the target and the guide RNA, the cleaved portion of the reporter is released into the sample solution (2506). In some embodiments, the sample solution is then exposed to a lateral flow assay strip (2502) comprising or adjacent to a sample pad containing sodium percarbonate (2504), which generates H₂O₂ when exposed to an aqueous solution. In some embodiments, the rehydration of the sodium percarbonate to form H₂O₂ occurs when the sample is wicked through the region. In some embodiments, the substrate contains DAB, TMB, or any other sufficient substrate. In some embodiments, the “spot” changes from blue to red, indicating the presence of HRP, and in turn a “hit” for the target nucleic acid sequence. In some embodiments, the readout is accomplished in solution, upon a color change of the sample solution (2508).

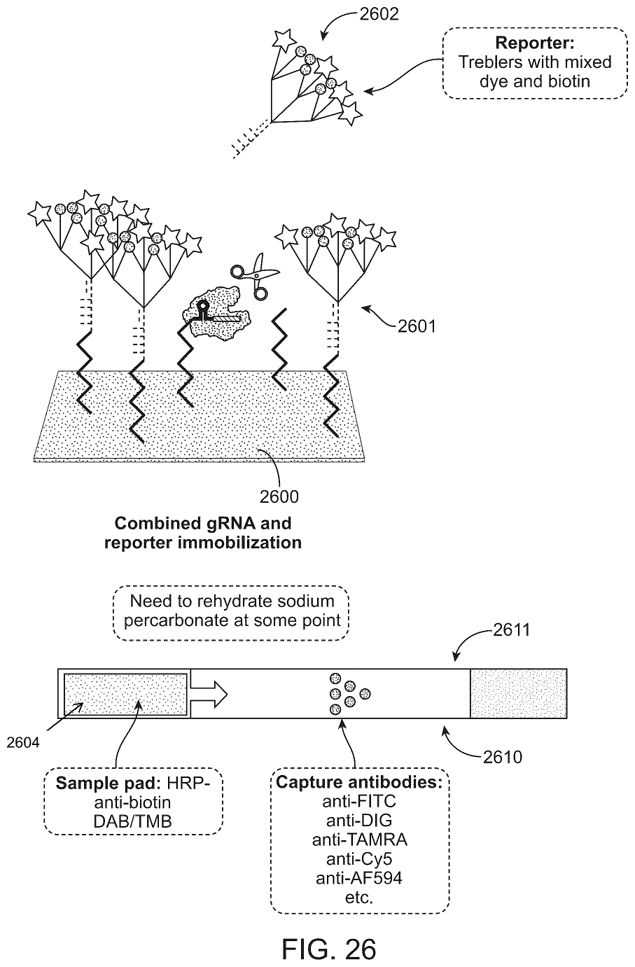

Described here are various methods and devices utilizing HRP-enhanced multiplexed DETECTR™ assays utilizing lateral flow assay strips for readout. In some embodiments, an HRP-signal enhanced multiplexed lateral flow assay as illustrated in FIG. 26. In some embodiments, the immobilized surface (2600) of a support medium and detection on the lateral flow assay strip (2610) are carried out as described in FIGS. 23-26 with the exception that signal transduction is not carried out by gold nanoparticles scattering light. Instead, in some embodiments, the anti-biotin labeled AuNP are supplanted by HRP-anti-biotin DAB/TMB. In some embodiments, the HRP is activated by sodium percarbonate present in the lateral flow assay strip which is rehydrated by the reaction and or chase buffer. In some embodiments, HRP allows for strong enough signal so as not to require sample amplification such as PCR.

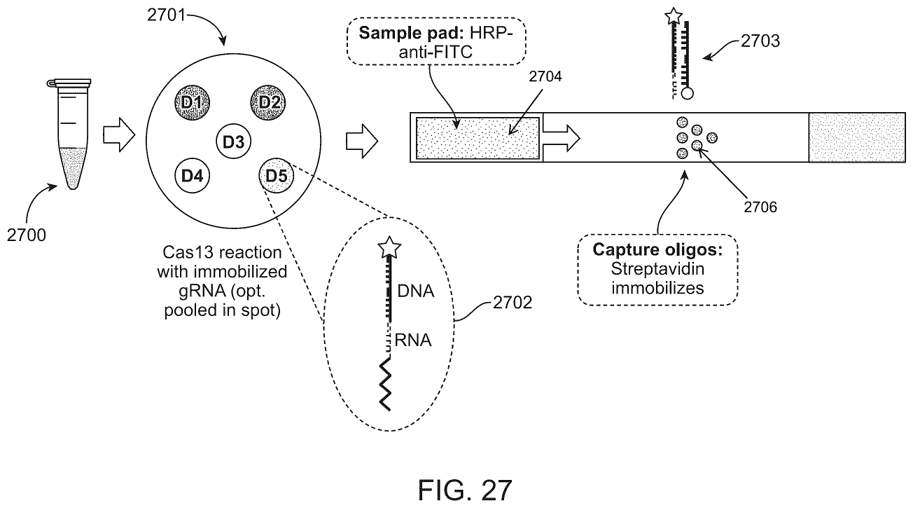

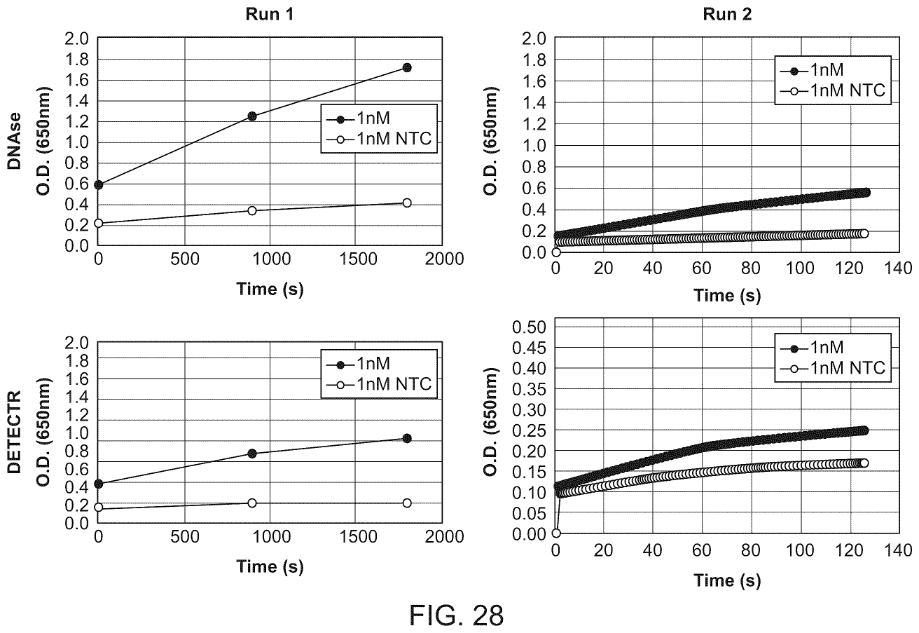

Described herein are various embodiments for multiplexed target nucleic acid detection utilizing Cas13 RNA cleaving specificity over DNA, HRP-signal enhancement, and capture oligo probe specificity. In some embodiments, as shown in FIG. 27, the sample (2700) contains different target nucleic acids. In some embodiments, the sample (2700) is then contacted to the surface of the well (2701) that is functionalized at one or more locations (e.g., five locations, D1-D5). In some embodiments, there are one or more locations. In some embodiments, Cas13 enzyme is present in the programmable nuclease probe. In some embodiments, Cas13 cleaves RNA but not DNA, enabling the use of a reporter (2702) that contains nucleic acid sequences with both DNA and RNA strands. In some embodiments, upon binding of the target nucleic acid to the sgRNA, the RNA of the reporter is cleaved by the Cas13 enzyme and a fragment containing a portion of the RNA, the complete DNA sequence, and a (FITC label is released into solution. In some embodiments, this action is repeated in parallel at each location, or spot with different reporters. In some embodiments, this action is repeated in parallel at locations D1 through D5 for five different target nucleic acids, producing five distinct reporter fragments. In some embodiments, the solution is then contacted to the sample pad of the lateral flow assay strip, where the sample pad contains HRP-anti-FITC. In some embodiments, the FITC-labeled reporter fragment then binds to the HRP-anti-FITC, forming a complex (2703) and is carried downstream across the detection region, binding specifically to the detection spot containing a capture oligo that has been designed to be the compliment for the oligo in the complex (2703). FIG. 28 shows results for both DNAse and DETECTR™ based assays for two replicate runs performed a week apart.

### Guide RNA Pooling for Signal Enhancement

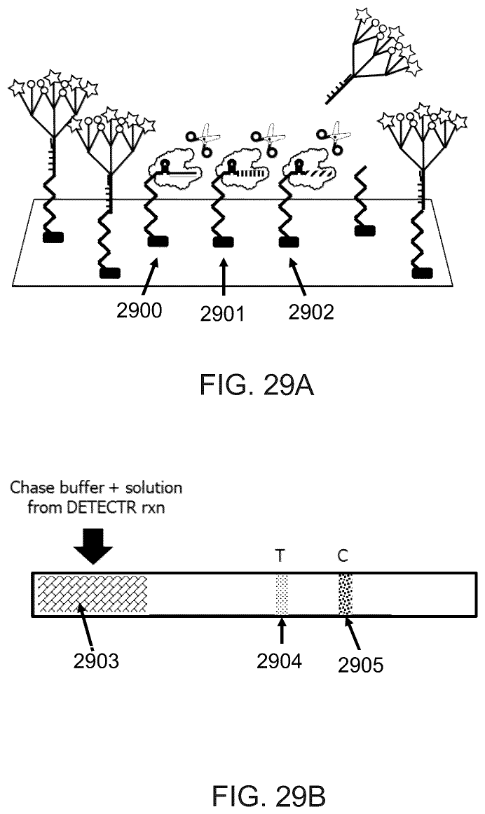

In some embodiments, one or more Cas-complex probes (2900-2902) are used for guide pooling to achieve enhanced signal detection in lateral flow assays as shown in FIG. 29A. In some embodiments, a first Cas-complex probe (2900) comprises a first sgRNA that is complimentary for a first segment of a target nucleic acid. In some embodiments, a second Cas-complex probe (2901) comprises a second sgRNA that is complimentary for a second segment of a same target nucleic acid. In some embodiments, a third Cas-complex probes (2902) comprises a third sgRNA that is complimentary for a third segment of the same target nucleic acid. In some embodiments, the first Cas-complex probe, the second Cas-complex probe, and the third Cas-complex probe are all located close enough to allow for sufficient cleaving of a reporter that is labeled to indicate the presence of the target nucleic acid. FIG. 29B shows a typical lateral flow assay strip comprising a sample pad (2903), a test line (2904), and a control line (2905).

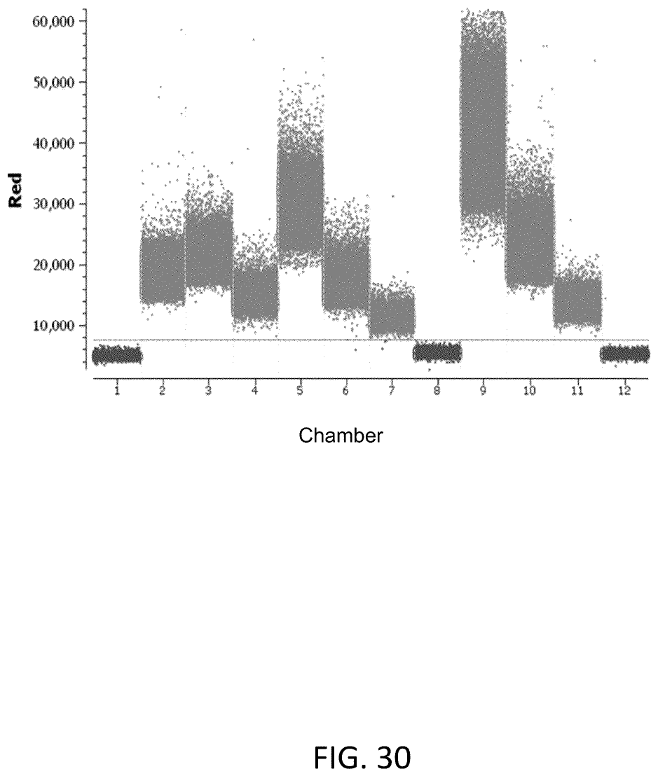

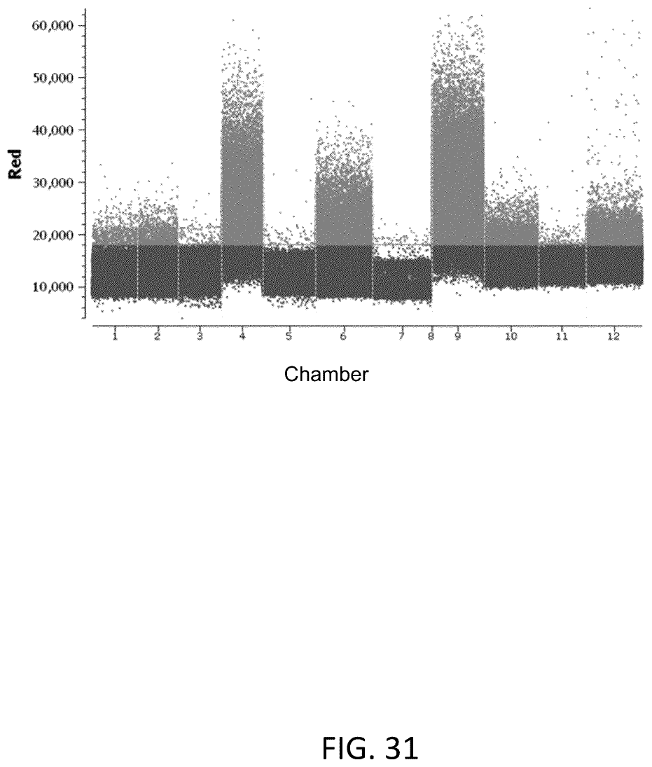

Described herein are various embodiments of guide pooling to achieve enhanced signal detection in lateral flow assays. For some embodiments as described herein, guide pooling shows enhanced Cas12a activity. FIG. 30 (described in Example 14) depicts results of a DETECTR™ assay showing enhanced Cas12a-based detection of the GF184 target using a pooled-guide (pooled-gRNA) format compared to DETECTR™ Cas12a-based assay using an individual gRNA format. In FIG. 30 the y-axis, labeled “Red” displays units of intensity and the x-axis shows the chamber number wherein a different DETECTR™ reaction occurred. FIG. 31 (described in Example 14) depicts results of a DETECTR™ assay showing enhanced sensitivity of the Cas13a-based detection of the SC2 target using a pooled-guide format compared to the Cas13a-based assays using an individual guide format.

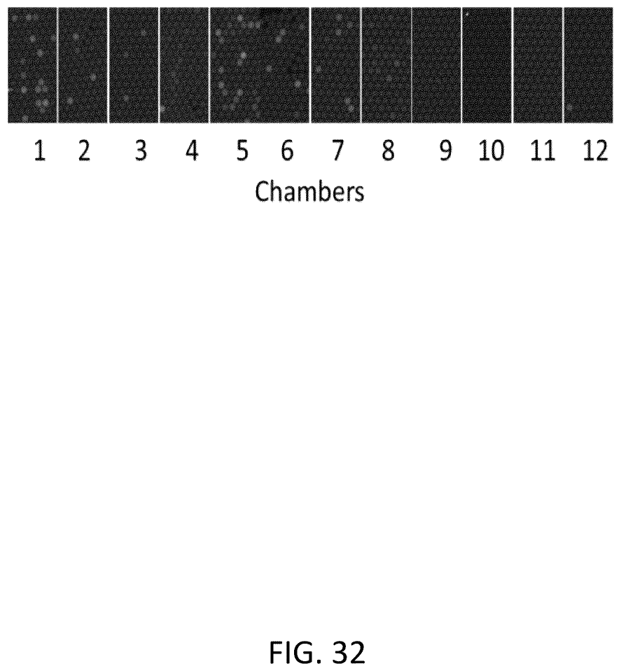

FIG. 32 (described in Example 14) shows images corresponding to each chamber, used to count the number of positive droplets, showing that the Cas13a-DETECTR™ assay samples containing the pooled guide RNAs generated more crystals containing the amplified products per starting copy of the target RNA than the Cas13a-DETECTR™ assay samples containing the guide RNAs in individual format.

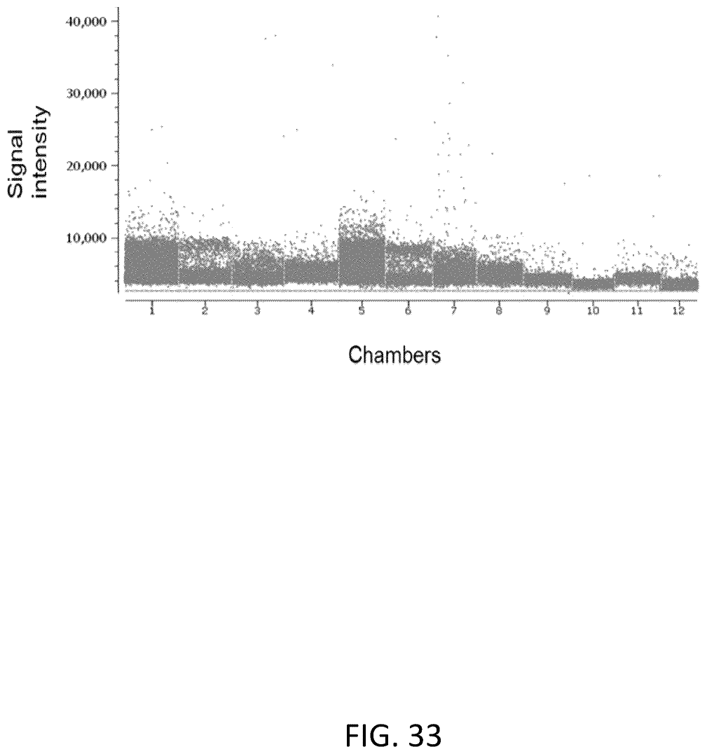

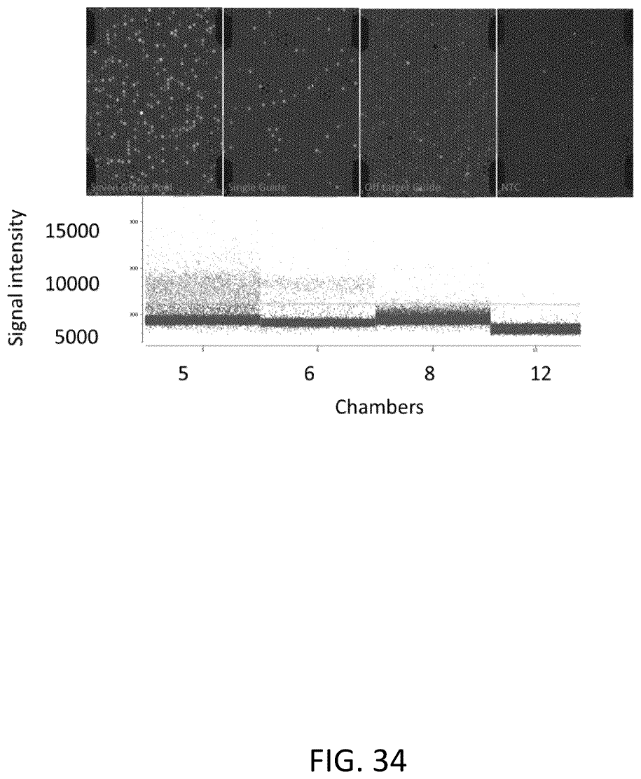

FIG. 33 (described in Example 14) shows that measurement of signal intensity following amplification showed that the Cas13a-DETECTR™ assay samples containing the pooled guide RNAs generated more signal intensity per starting copy of the target template RNA than the Cas13a-DETECTR™ assay samples containing the guide RNAs in individual format. FIG. 34 (described in Example 14) shows that measurement of signal intensity following amplification showed that the Cas13a-DETECTR™ assay samples containing the pooled guide RNAs generated more signal intensity per starting copy of the target template RNA than the Cas13a-DETECTR™ assay samples containing the guide RNAs in individual format. FIG. 34 also shows that relative quantification performed by counting the number of positive droplets showed that the Cas13a-DETECTR™ assay samples containing the pooled guide RNAs generated more crystals containing the amplified products per starting copy of the target template RNA than the Cas13a-DETECTR™ assay samples containing the guide RNAs in individual format.

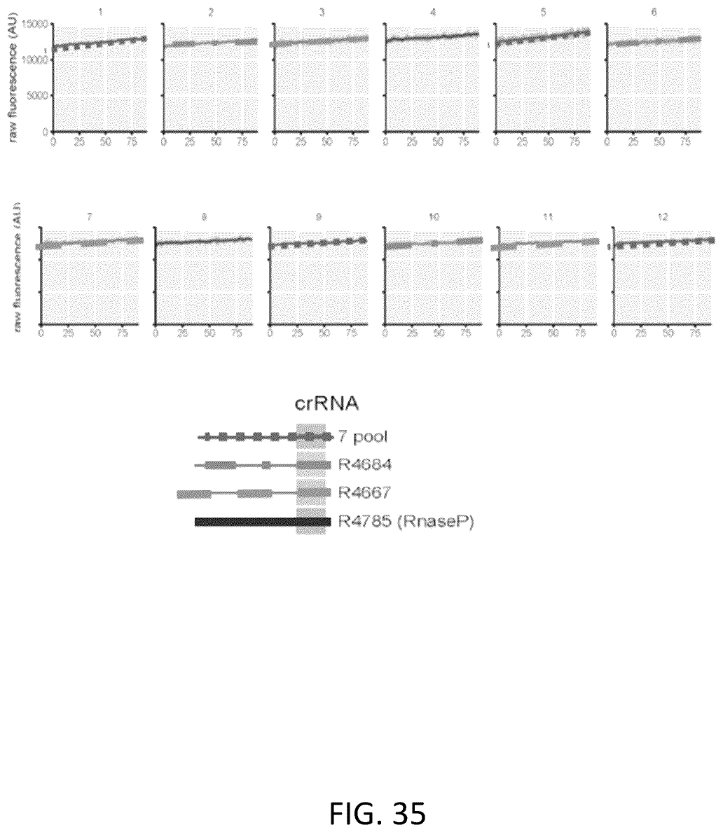

FIG. 35 (described in Example 14) shows that Cas13a DETECTR™ assay samples containing the pooled guides (R4637, R4638, R4667, R4676, R4684, R4689, R4691) did not exhibit higher target detection sensitivity per starting copy of the target than the Cas13a DETECTR™ samples containing the single guides R4684, R4667, or R4785 (RNAseP guide) in individual format.

Disclosed herein are non-naturally occurring compositions and systems comprising at least one of an engineered programmable nuclease (e.g., engineered Cas protein) and an engineered guide nucleic acid, which may simply be referred to herein as a Cas protein and a guide nucleic acid, respectively. In general, an engineered Cas protein and an engineered guide nucleic acid refer to a Cas protein and a guide nucleic acid, respectively, that are not found in nature. In some instances, systems and compositions comprise at least one non-naturally occurring component. For example, compositions and systems may comprise a guide nucleic acid, wherein the sequence of the guide nucleic acid is different or modified from that of a naturally-occurring guide nucleic acid. In some instances, compositions and systems comprise at least two components that do not naturally occur together. For example, compositions and systems may comprise a guide nucleic acid comprising a repeat region and a spacer region which do not naturally occur together. Also, by way of example, composition and systems may comprise a guide nucleic acid and a Cas protein that do not naturally occur together. Conversely, and for clarity, a Cas protein or guide nucleic acid that is “natural,” “naturally-occurring,” or “found in nature” includes Cas proteins and guide nucleic acids from cells or organisms that have not been genetically modified by a human or machine.

In some instances, the guide nucleic acid comprises a non-natural nucleobase sequence. In some instances, the non-natural sequence is a nucleobase sequence that is not found in nature. The non-natural sequence may comprise a portion of a naturally occurring sequence, wherein the portion of the naturally occurring sequence is not present in nature absent the remainder of the naturally-occurring sequence. In some instances, the guide nucleic acid comprises two naturally occurring sequences arranged in an order or proximity that is not observed in nature. In some instances, compositions and systems comprise a ribonucleotide complex comprising a CRISPR/Cas effector protein and a guide nucleic acid that do not occur together in nature. Engineered guide nucleic acids may comprise a first sequence and a second sequence that do not occur naturally together. For example, an engineered guide nucleic acid may comprise a sequence of a naturally occurring repeat region and a spacer region that is complementary to a naturally occurring eukaryotic sequence. The engineered guide nucleic acid may comprise a sequence of a repeat region that occurs naturally in an organism and a spacer region that does not occur naturally in that organism. An engineered guide nucleic acid may comprise a first sequence that occurs in a first organism and a second sequence that occurs in a second organism, wherein the first organism and the second organism are different. The guide nucleic acid may comprise a third sequence disposed at a 3′ or 5′ end of the guide nucleic acid, or between the first and second sequences of the guide nucleic acid. For example, an engineered guide nucleic acid may comprise a naturally occurring crRNA and tracrRNA coupled by a linker sequence.

In some instances, compositions and systems described herein comprise an engineered Cas protein that is similar to a naturally occurring Cas protein. The engineered Cas protein may lack a portion of the naturally occurring Cas protein. The Cas protein may comprise a mutation relative to the naturally-occurring Cas protein, wherein the mutation is not found in nature. The Cas protein may also comprise at least one additional amino acid relative to the naturally-occurring Cas protein. For example, the Cas protein may comprise an addition of a nuclear localization signal relative to the natural occurring Cas protein. In certain embodiments, the nucleotide sequence encoding the Cas protein is codon optimized (e.g., for expression in a eukaryotic cell) relative to the naturally occurring sequence.

In some instances, compositions and systems provided herein comprise a multi-vector system encoding a Cas protein and a guide nucleic acid described herein, wherein the guide nucleic acid and the Cas protein are encoded by the same or different vectors. In some embodiments, the engineered guide and the engineered Cas protein are encoded by different vectors of the system.

### Definitions

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

As used herein, the term “comprising” and its grammatical equivalents specifies the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

As used herein the terms “individual,” “subject,” and “patient” are used interchangeably and include any member of the animal kingdom, including humans.

As used herein the term “antibody” refers to, but not limited to, a monoclonal antibody, a synthetic antibody, a polyclonal antibody, a multispecific antibody (including a bi-specific antibody), a human antibody, a humanized antibody, a chimeric antibody, a single-chain Fvs (scFv) (including bi-specific scFvs), a single chain antibody, a Fab fragment, a F(ab′) fragment, a disulfide-linked Fvs (sdFv), or an epitope-binding fragment thereof. In some cases, the antibody is an immunoglobulin molecule or an immunologically active portion of an immunoglobulin molecule. In some instances, an antibody is animal in origin including birds and mammals. Alternately, an antibody is human or a humanized monoclonal antibody.

### EXAMPLES

The following examples are illustrative and non-limiting to the scope of the streamlined lateral flow devices and methods described herein.

### Example 1: Lateral Flow Device for Detection of Nucleic Acids

This example describes a streamlined lateral flow device for detection of nucleic acids.

A biological sample from an individual can be tested to determine whether the individual has a target nucleic acid of interest. The biological sample can be tested to detect the presence or absence of the target nucleic acid sequence.

A device is configured to accommodate multiple single-use cartridges containing reagents for sample preparation, optional amplification, and detection of the target nucleic acid. The device performs the following steps: (1) sample preparation (e.g., elution from a swab at room temperature), (2) isothermal nucleic acid amplification at 60° C., (3) CRISPR-based reaction (e.g., a DETECTR™ reaction) at 37° C., and (4) visual readout of results using lateral flow strips (FIG. 1). A diagram of said streamlined lateral flow device is shown in FIG. 2.

Sample preparation includes chemical lysis with a solution stored on-board the prototype. The entire volume of sample lysate (˜200 μL) is distributed into at least two lateral flow strips. Ramping temperatures include 62° C.: 2-5 minutes with preheating, so the reaction starts immediately with sample addition and/or 37° C.: 2-5 minutes with preheating, so the reaction starta immediately with sample addition. The lateral flow device is battery operated and is single use. The operator (e.g., a clinician or laboratory technician) is unable to remove the swab once inserted into device, thereby reducing contamination, sample leakage, sample loss, etc. A user inserts a sample into the lateral flow device, allows for the device to process the sample, amplify target nucleic acids, and yield a result indicating presence or absence of the target nucleic acid.

A summary of target metrics, uses, microorganism targets, and additional features is shown below in TABLE 5.

TABLE 5

| | | | | |
|---|---|---|---|---|
| Targeted | Detection: 5,000 copies | Detection: 1,000 copies | Detection: 100 copies | Detection: 10 copies |
| Metrics | ≥80% Sens/Spec. | ≥85% Sens/Spec. | ≥90% Sens/Spec. | ≥98% Sens/Spec. |
| | Handheld: | Handheld: | Handheld: | Handheld: |
| | 5-plex, 100 units | 10-plex, 500 units | 10-plex, 5,000 units | 10-plex, 50,000 units |
| Use Case | Trained personnel in | Trained personnel in | Ruggedized device | Ruggedized device |
| | clinical lab with | clinical lab with all | for use in Tier 1 or | for use in Tier 1 or |
| | some off-device steps | steps automated on- | Tier 2 conditions by | Tier 2 conditions |
| | | device | trained users | |
| Targets | Initially, COVID-19 | | | |
| | and RNase P gene | | | |
| | endogenous control | | | |
| | Expansion to 5-plex | | | |
| | with Pan-CoV, IAV | | | |
| | and IBV | | | |
| Additional | Single sample input | Additional sub- | Power-free solutions | Complete removal |
| Features | Integration of a | modules for other | explored for reaction | of power by end of |
| | CRISPR-based gene | sample types | heating and metering | program |
| | sensor to a single | | Removal of | |
| | device from one | | amplification step | |
| | sample type | | | |

### Example 2: Improved Lateral Flow Device for Detection of Nucleic Acids

This example describes improved lateral flow devices for detection of nucleic acids. A lateral flow device of EXAMPLE 1 is modified and used for CRISPR based detection of nucleic acids of interest. The lateral flow device of EXAMPLE 1 is modified for (1) power-free function with the use of magnesium oxide crystals, or other agents, (2) removal of the amplification step and, thereby, removal of the region corresponding to amplification, and (3) use of other sample types apart from an NP swab (e.g., saliva and blood). It may be adapted for war-fighter needs. A user inserts a sample into the lateral flow device, allows for the device to process the sample and yield a result indicating presence or absence of the target nucleic acid.

### Example 3: Guide Pooling with Lateral Flow Devices for Detection of Nucleic Acids

This example describes guide pooling with lateral flow devices for detection of nucleic acids. A lateral flow device of EXAMPLE 1 or EXAMPLE 2 is used for detection of nucleic acids. A multiplexed assay is run on these lateral flow devices wherein a 5-plex or 10-plex guide pool is used to target, bind, and detect 5 or 10 separate targets, respectively. Guide pools are designed to target different regions of the same nucleic acid in a target or are designed to target different microorganisms altogether. A user inserts a sample into the lateral flow device, allows for the device to process the sample, optionally amplify target nucleic acids, and yield a result indicating presence or absence of each target nucleic acid.

### Example 4: Concept Designs for a Point-of-Need CRISPR Device

At-home testing provides considerable benefits for both patients and the community, including rapid test results, improved disease management, and reduced risk of disease transmission to healthcare providers. Two of the most widely used applications of at-home testing are glucose monitoring (Tonyushkina & Nichols, 2009) and pregnancy tests (Gnoth & Johnson, 2014). Technologies such as lateral flow assays (Koczula & Gallotta, 2016) enable rapid testing for proteins in body fluids such as blood, saliva, and urine. A commercial example of protein detection through lateral flow, using a swab for sample collection is the SavvyCheck™ Vaginal Yeast Test (“SavvyCheck™ Vaginal Yeast Test—Savyon Diagnostics,” n.d.). Despite the promise of at-home testing, existing testing methods are confined to detection of small molecules and proteins and no technologies currently exist for point-of-need molecular testing. This example describes ideations for a point-of need device for at-home testing that uses the DETECTR™ assay and a swab input.

In this example, the following steps are performed: (1) sample preparation (elution from a swab at room temperature), (2) isothermal nucleic acid amplification at ˜60° C., (3) CRISPR-based reaction at 37° C., and (4) visual readout of results using lateral flow strips. FIG. 1 shows a workflow of the processes taking place on the device.

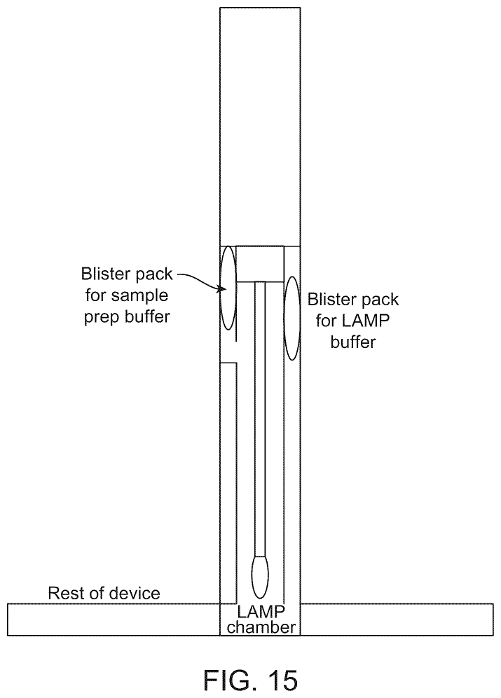

To use the device, the user would swab their nostril (or cheek if buccal cell target), then screw the swab into the device. When the lid is screwed into the device, the device locks (using a child safety lock-like mechanism), which prevents the fluid from leaking out of the device. After passing the lock, continuing to screw on the lid will puncture blister packs that will release the lysis buffer, which is stored in a blister pack near the top of the sample port, and the LAMP buffer. A cross-sectional schematic of the sample prep portion of the device is shown below in FIG. 15.

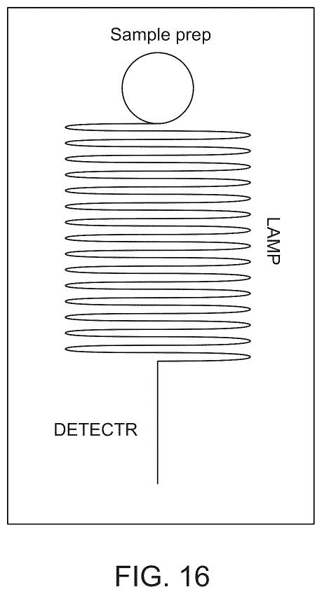

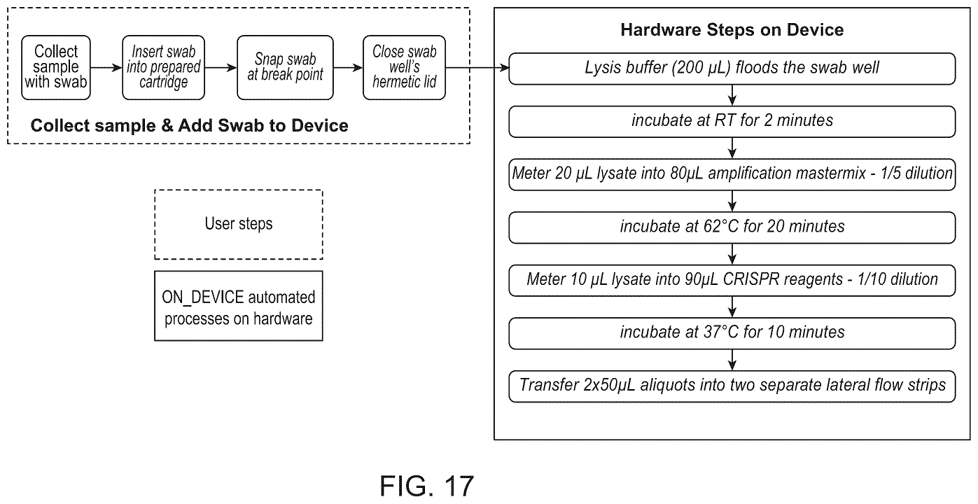

After the sample preparation portion of the device, there are different strategies that could be used for the remainder of the assay. One option is to use a continuous microfluidic channel, with a serpentine channel for the LAMP portion as illustrated. The flow in this channel can be driven by capillary flow, when the surface of the channel is sufficiently hydrophilic. FIG. 16 is a plan view schematic illustrating looking at the microfluidic device from the top. The sample preparation region (such as the scheme described above in FIG. 1) is labeled but is drawn as a circle for simplicity. The rest of the microfluidic architecture is illustrated by the black line. When multiplexing is required for DETECTR™, the design can be altered to include a branching DETECTR™ channel with one target in each branch. For the reaction, the fluid can flow through the LAMP channel slowly and travel over a heater, resulting in amplification of the target. The sample can then encounter lyophilized DETECTR™ reagents and incubate over another heater, where there can be optical systems for detection. If LAMP multiplexing is required, this can be accomplished through having multiple LAMP channels. FIG. 17 provides process step details for the device described in this example.

### Example 5: Closed Lateral Flow Assay Format for Running CRISPR Diagnostic Reactions while Preventing Amplicon Contamination

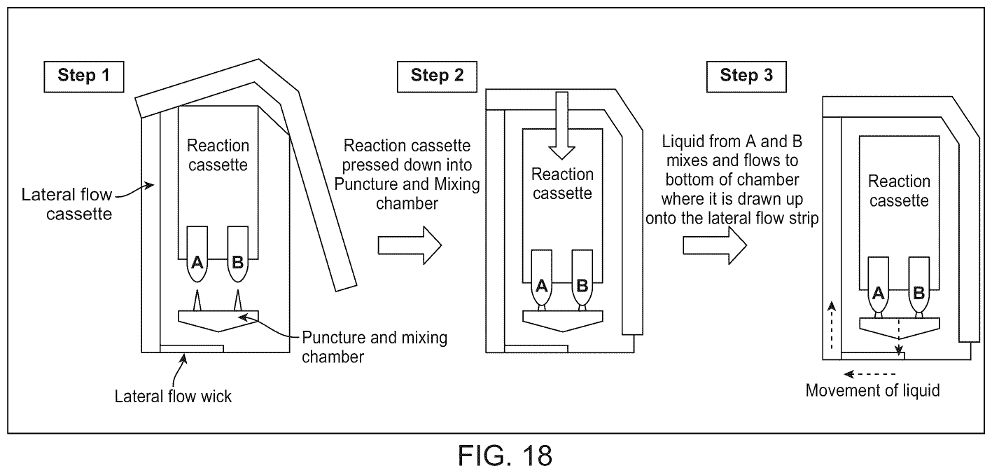

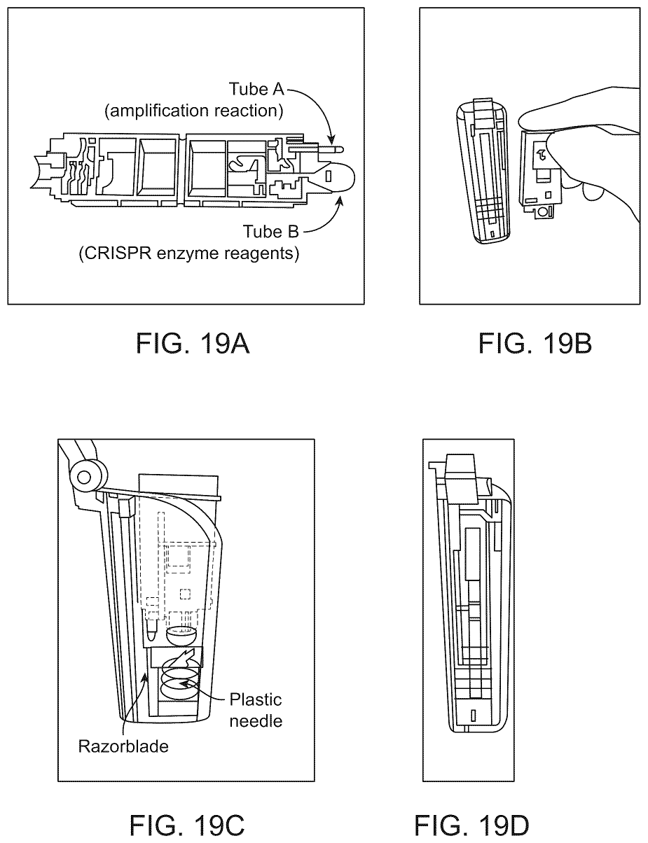

CRISPR diagnostic reactions as described herein can involve two steps. The first is an optional nucleic acid amplification step that can enable single-molecule sensitivity for the assay. This nucleic acid amplification step can involve methods such as PCR, which uses thermocycling and thermostable polymerases, or isothermal methods such as RPA, LAMP, SDA, or NEAR, which amplify nucleic acid targets at a single temperature. The second step in CRISPR diagnostics is the CRISPR enzyme reaction which uses programmable nuclease proteins such as Cas12, Cas13, or Cas14 to recognize the amplicon generated by the nucleic acid amplification step using a programmable guide RNA (gRNA). One of the main risks of running CRISPR diagnostic reactions in clinical laboratories or other settings is the potential for nucleic acid amplicons to contaminate the laboratory and lead to false-positives in the assay. For PCR and other nucleic acid amplification methods, this can generally be controlled by preventing the opening of assay plates after amplification. However, for some CRISPR diagnostic workflows, especially those that use lateral flow as a readout, the nucleic acid amplicon reaction volume is typically opened to combine it with the CRISPR enzymes. Devices have been developed to contain nucleic acid amplification reactions that are loaded onto lateral flow strips. In this example, a device that is designed to mix the two components of a CRISPR diagnostic reaction (the nucleic acid amplification reaction and the CRISPR enzyme components) while simultaneously preventing the contamination of a laboratory or other environment with nucleic acid amplicons. Briefly, this device uses a reaction cassette that holds one tube that contains the nucleic acid amplification reaction, and another tube that contains the CRISPR enzyme components. The user of the device inserts the nucleic acid amplification reaction tube after the completion of amplification into the reaction cassette. The reaction cassette also contains a second tube that contains the CRISPR enzyme reagents. The reaction cassette is inserted into the device and external arm of the device forces the reaction cassette down and seals the device. When the reaction cassette is forced down, it is pushed onto two needle-like or razorblade-like projections that pierce the two tubes as seen in FIG. 18. The contents of the two tubes pool together in a mixing chamber and drip down onto a paper wick. This wick draws the reagents up and into a cassette inside of the device that contains a lateral flow strip as can be seen in FIG. 19. The lateral flow strip is used to assess the outcome of the CRISPR diagnostic reaction. Other mechanisms (e.g., actuators, microfluidics, pumps, etc.) may be used to bring the contents of the reaction chamber into contact with the lateral flow assay strip(s).

### Example 6: Closed Spin Column Format for Running CRISPR Diagnostic Reactions while Preventing Amplicon Contamination

In this example a second format of the assay that uses a spin-column to separate the two parts of the reaction, as seen in FIG. 20, is described. The nucleic acid amplification reaction can occur in the bottom of the main tube, whereas the CRISPR enzyme reagents are held separate from the main reaction in the spin-column. When using a CRISPR enzyme that is capable of withstanding higher temperatures (e.g., 55-60° C.) or using isothermal methods that do not require temperatures that denature the CRISPR proteins, the isothermal amplification can be physically separated from the CRISPR components without inhibiting the performance of the CRISPR diagnostic reaction. The reactions can then be combined by spinning the tube in a microcentrifuge tube, or by using a syringe to push the CRISPR reagents into the bottom tube. The result of the assay can be seen using a blue light and filter that enables visual detection of the fluorescence output of the assay.

### Example 7: DETECTR™-Based Lateral Flow Assay Strip

The purpose of this example is to demonstrate a DETECTR™-based multiplexed assay using a lateral flow assay (LFA) strip for parallel readout as illustrated in FIG. 23A-B. To perform the DETECTR™ assay, a surface (2300) is immobilized with programmable nuclease probes (2307) and reporter probes (2301). In this example, the surface is the bottom of a well, separate from the LFA strip. The reporter probes (2301) contain a surface linker (2302), a cleavable nucleic acid sequence (2303), a label (2304) and binding moiety for the flowing capture probe (2305) of the strip. In this example the binding moiety is biotin. The label and binding moiety are attached to the nucleic acid (2303) by a second linker (2306), where in this example the linker is a dendrimer or trebler molecule. The programmable nuclease probe (2307) contains a surface linker and a programmable nuclease (e.g., Cas enzyme) that in turn contains an sgRNA (2308). The sgRNA contains a repeat unit (or hair pin) and a recognition sequence. The recognition sequence is the compliment for a target nucleic acid of the sample.

Anti-biotin labeled gold nanoparticles are located in the sample pad (2311) of the LFA strip (2310) as shown in FIG. 23B.

In this example, the first step is to contact the surface (2300) with a sample containing target nucleic acids. Upon binding of a target nucleic acid that is complimentary to the sgRNA (2308) of the immobilized programmable nuclease probe (2307), the reporters (2301) immobilized in near proximity, are cleaved by the programmable nuclease, releasing a cleaved section of the reporter (2309) into solution.

The sample solution now containing cleaved reporters corresponding to target nucleic acids that were present in the sample, is then contacted to the sample pad (2311) of the lateral flow assay strip (2310). In this example, the sample pad has flowing capture probes (e.g., anti-biotin labeled gold nanoparticles) disposed thereon. Once the sample solution containing released sections of reporter molecules is contacted with the sample pad, said sample solution then flows across the sample pad (for e.g., by being wicked). The cleaved reporters in the sample solution contact and bind to the anti-biotin gold nanoparticle flowing capture probes upon contact in solution. The complex of reporter and nanoparticle is then carried downstream with the rest of the liquid sample by capillary action through the detection region (2312) of the lateral flow assay strip. In this example, the detection region (2312) comprises six detection spots (2313), where each detection spot (2313) contains a different capture antibody type that is specific for a reporter's dye (2304). In this example, the dye (2304) is FITC and the stationary capture probe is an anti-FITC antibody functionalized to the detection spot (2313) allowing for the specific detection of the FITC labeled reporter among other reporters that are specific for other target nucleic acids. A control line (2314) is present, functionalized with anti-IgG so that all flowing capture probes, not bound to a FITC labeled reporter fragment, (e.g. detection moiety) are captured and detected. It should be noted that in this example, multiple labels and binding moieties were present via the dendritic linker of the detection moiety (2306) to amplify the signal. In this example, multiple detections spots (2313) are present, allowing for the possibility parallel detection of multiplexed samples, as explained in Example 8.

### Example 8: Multiplexed DETECTR™-Based Lateral Flow Assay Strip

The purpose of this example is to demonstrate a lateral flow assay strip workflow utilizing a multiplex “Hotpot” assay as illustrated in FIG. 24. In this example, a sample (2401) contains target nucleic acid sequences 1 and 2. The sample (2401) is contacted to the surface (2402) of a well where at each of five locations, D1 through D5, there are different sgRNA's immobilized to the surface. For example, each of the 5 different sgRNA's are part of 5 different programmable nuclease probe (e.g., see FIG. 23) immobilized in the five different locations D1 through D5, as depicted in FIG. 24. Additionally, each of the 5 different sgRNA's are designed to specifically bind to different target nucleic acids in the sample, thus allowing for sample multiplexing. In addition to the immobilized programmable nuclease probes containing sgRNAs, each location, D1 through D5, is functionalized with reporter probes having distinct functional groups. The reporter probes are in close enough proximity to be cleaved by the programmable nuclease probes. Therefore, as described in example 7, reporters are cleaved and released into the solution upon binding between a sgRNA and the target nucleic acid that the sgRNA is designed to bind specifically to. In this example, D4 and D5 each contain reporters labeled with two different labels or capture antibody recognition elements. Once the sample has contacted with the wells (D1 to D5), the respective cleaved sections of reporter molecules are released into the sample solution (as described in Example 7). The sample solution with the released reporter molecules are then contacted with a sample pad, wherein in this example, is situated on lateral flow assay. In this example each detection spot contains a different type of capture antibody, where each capture antibody type specifically binds to a particular label of a reporter. For this example, detection spot (2403) contains the capture antibody anti-FITC, whereas well D5 contains 1) the immobilized Cas-complex including the sgRNA specific to a first target nucleic acid sequence, and 2) the immobilized reporter molecule (2406), which is labeled with FITC. Therefore, upon binding of the first target nucleic acid sequence with the corresponding sgRNA, the linkage between the corresponding immobilized reporter molecule (2406) and corresponding nucleic acid (for example see ref. char. 2301 in FIG. 23) is cleaved, thereby releasing the reporter molecule into solution. By contrast, for this example, detection spot (2404) contains the capture antibody anti-DIG, whereas well D4 contains 1) the immobilized Cas complex including the sgRNA specific to a second target nucleic acid sequence, and 2) the immobilized reporter molecule (2405), which is labeled with DIG. Therefore, upon binding of the second target nucleic acid sequence with the corresponding sgRNA, the linkage between the corresponding immobilized reporter molecule (2405) and corresponding nucleic acid (for example see ref. char. 2301 in FIG. 23) is cleaved, thereby releasing the reporter molecule the solution. The solution now containing cleaved reporter molecules (2405 and 2406) is then contacted to the sample pad of the LFA strip along with chase buffer, where the reporter molecules bind with and pick up flowing capture probes (e.g., anti-biotin-AuNPs) that are disposed on the sample pad. The AuNP-reporter conjugates having reporter (2406) labeled with FITC will selectively bind to detection spot (2403) containing the capture antibody anti-FITC, thus indicating the presence of the first target nucleic acid sequence in the sample. The AuNP-reporter conjugates having reporter (2405) labeled with DIG will selectively bind to detection spot (2404) containing the capture antibody anti-DIG, thus indicating the presence of the second target nucleic acid sequence in the sample. In this manner, parallel detection of 2 or more target nucleic acid sequences present in a multiplexed sample is enabled. In some examples, the detection spots are spaced apart from each other in prescribed locations, such that detection of a reporter molecule at a given detection spot will correlate with a specific target nucleic acid.

### Example 9: HRP-Enhanced DETECTR™-Based Lateral Flow Assay Strip

The purpose of this example is to demonstrate horse radish peroxidase (HRP) paper-based detection as illustrated in FIG. 25. Here, “paper-based” refers to a lateral flow assay strip. As in examples 7 and 8, a liquid, blue color sample, containing a target(s) nucleic acid sequence is exposed to a surface upon which CRISPR-Cas/gRNA probes and reporter probes are immobilized. In this example, the reporter probes contain HRP molecules. Upon cleavage of the reporter by the Cas enzyme following a specific binding event between the target and the guide RNA, the cleaved portion of the reporter is released into the sample solution. In this example, the sample solution having the released sections of reported molecules is then contacted with a lateral flow assay strip comprising a sample pad containing sodium percarbonate, which generates H₂O₂ when hydrated. The rehydration of the sodium percarbonate to form H₂O₂ occurs when the sample is wicked through the sample pad and the lateral flow assay to a detection spot, as described in the previous examples 7 and 8. In this example, the lateral flow assay strip contains the chemical substrates DAB and TMB which activate a color change from blue to red, indicating the presence of HRP, and in turn a “hit” for the target nucleic acid sequence. The chemical catalytic nature of HRP enables signal amplification. Alternatively, the readout can also be accomplished in solution, upon a color change of the sample solution from blue to red.

### Example 10: HRP-Enhanced Multiplexed DETECTR™-Based Lateral Flow Assay Strip

The purpose of this example is to demonstrate an HRP-signal enhanced multiplexed lateral flow assay as illustrated in FIG. 26. The immobilized surface (2600) and detection on the lateral flow assay strip (2610) are carried out as described in the previous examples with the exception that signal transduction is not carried out by gold nanoparticles scattering light. Instead, the anti-biotin labeled AuNP are supplanted by HRP-anti-biotin DAB/TMB. The HRP is activated by sodium percarbonate present in the LFA strip which is rehydrated by the reaction and or chase buffer. In this manner, HRP allows for strong enough signal so as not to require sample amplification such as PCR. Multiplexed detection is accomplished in the same manner as described in Example 8.

### Example 11: Cas13 Based, HRP-Enhanced DETECTR™ LFA with Capture Oligo Probe Specificity

The purpose of this example is to demonstrate multiplexed target nucleic acid detection utilizing Cas13 RNA cleaving specificity over DNA, HRP-signal enhancement, and capture oligo probe specificity as shown in FIG. 27. In this example, the sample (2700) contains different target nucleic acids. The sample (2700) is then contacted to the surface (2701) of the well that is functionalized at five locations, D1-D5, as described in Example 8. The difference here is that the Cas13 enzyme is present in the Cas-complex probe. Cas13 cleaves RNA but not DNA. This enables the use of a reporter (2702) that contains nucleic acid sequences, one composed of DNA and the other composed of RNA. Upon binding of the target nucleic acid to the sgRNA, the RNA of the reporter is cleaved by the Cas13 enzyme and a fragment containing: a portion of the RNA; the complete DNA sequence; and the FITC label is released into solution. This action is repeated in parallel at each spot D1 through D5 for five different target nucleic acids, producing 5 distinct reporter fragments. The solution is then contacted to the sample pad of the LFA strip, where the sample pad contains HRP-anti-FITC. The FITC labeled reporter fragment then binds to the HRP-anti-FITC, forming a complex (2703) and is carried downstream across the detection region, binding specifically to the detection spot containing a capture oligo that has been designed to be the compliment for the oligo in the complex (2703). In this manner, parallel detection of multiplexed samples as described in Example 8 is possible.

### Example 12: HRP-Enhanced DETECTR™-Based Lateral Flow Assay Strip Utilizing Cas14

The purpose of this example is to demonstrate horse radish peroxidase (HRP) paper-based detection as illustrated in FIG. 25. Here, “paper-based” refers to a lateral flow assay strip. As in examples 7 and 8, a liquid, blue color sample, containing a target(s) nucleic acid sequence is exposed to a surface upon which programmable nuclease probes and reporter probes are immobilized. In this example, the reporter probes contain HRP molecules. Upon cleavage of the reporter by the Cas14 enzyme following a specific binding event between the target and the guide nucleic acid, the cleaved portion of the reporter is released into the sample solution. In this example, the sample solution is then exposed to a lateral flow assay strip comprising a sample pad containing sodium percarbonate, which generates H₂O₂ when hydrated. The rehydration of the sodium percarbonate to form H₂O₂ occurs when the sample is wicked through the region, as described in the previous examples 7 and 8. Since the substrate contains DAB, TMB, etc. . . . the “spot” changes from blue to red, indicating the presence of HRP, and in turn a “hit” for the target nucleic acid sequence. In this manner HRP enables signal amplification. Alternatively, as shown in FIG. 25, the readout can also be accomplished in solution, upon a color change of the sample solution from blue to red.

### Example 13: HRP-T20-Biotin DETECTR™-Based Assay

The purpose of this example was to demonstrate an HRP and DETECTR™-based assay. In this example, reporters were cleaved by a Cas complex, or a DNAse enzyme in solution. The cleaved reporter was reacted to HRP-T20-biotin. The supernatant solution was then added to a reaction volume that contained TMB and H₂O₂ to generate a color signal. The cleaved reporter-HRP conjugate was then detected by optical density measurement of the solution. Optical density measurements were acquired from the beginning of the reaction. The experiment was performed in two sets comprising 2 runs each, where each set was run 1 week apart. DNase and DETECTR™ were used separately in each run of each set. In the DNase runs, 1 nM of HRP target oligo was used in the blue series. Results are shown in FIG. 28. The significance of this example is that multiple turnovers of both HRP and DETECTR™ enable alternative signal amplification to sample amplification such as PCR.

### Example 14: Guide Pooling for Enhanced Target Detection Signal in DETECTR™ Assays

Guide RNAs that were designed to bind to a different region within a single target molecule were pooled as a strategy for enhancing the target detection signal from DETECTR™ assays. For example, in this strategy, each DETECTR™ ™ reaction contained a pool of CRISPR-Cas RNP complexes each of which targeted a different region within a single target nucleic acid molecule. As discussed herein, this strategy resulted in increased sensitivity to target detection by using increased number of complexes/single target such that the signal is strong enough to detect within a Poisson distribution (sub-one copy/droplet) and provide a quantitative evaluation of target numbers within a sample.

To test the effect of guide pooling on target detection using the Cas12a nuclease, first, a Cas12a complexing mix was prepared wherein the R1965 (off-target guide), R1767, R3164, R3178 guides were present in either a pooled-gRNA format (a pool of two or more of the three guides selected from R1767, R3164, or R3178) or in a single-gRNA format (wherein R1767, R3164, R3178 were present individually) and the mix was incubated for 20 minutes at 37° C. A 2-fold dilution series for the template RNA (GF184) was created from a starting dilution concentration (wherein 5.4 μl of GF184 at 0.1 ng/μL was added to 44.6 μl of nuclease-free water). DETECTR™ master mixes which included the Cas12 complex, Reporter substrate, Fluorescein, Buffer, and diluted template (GF184 or off-target template GF577) were then assembled as shown in Table 6. The DETECTR™ mixes were then loaded into a Stilla Sapphire chip and placed into the Naica Geode. Crystals were created from thousands of droplets from each sample. No amplification step was performed. The signal from the Sapphire chips was measured in the Red channel. The results of the DETECTR™ assay showed enhanced Cas12a-based detection of the GF184 target using a pooled-guide format compared to DETECTR™ Cas12a-based assay using an individual guide format. For example, the DETECTR™ assays showed an enhanced signal from chamber 5 containing a pool of two guides R1767 and R3178, compared to the signal from chamber 2 or chamber 4 which contained the R1767 and R3178 in individual guide format respectively (FIG. 30). Similarly, the DETECTR™ assays showed an enhanced signal from chamber 9 containing a pool of three guides (R1767, R3164, and R3178), compared to the signal from chamber 5 which contained a pool of two guides (R1767 and R3178) and compared to the signal from chamber 2, chamber 3, or chamber 4 which contained the R1767, R3164, and R3178 in individual guide format respectively (FIG. 30).

TABLE 6

| | | Copies/ | # | copies/ |
|---|---|---|---|---|
| Chamber | Condition | Chamber | Droplets | droplet |
| 1 | Off Target | 2.5 × 10⁷ | 29336 | 852 |
| | Guide (1965) | | | |
| 2 | Single R1767 | 2.5 × 10⁷ | 26838 | 931 |
| 3 | Single R3164 | 2.5 × 10⁷ | 29590 | 845 |
| 4 | Single R3178 | 2.5 × 10⁷ | 27769 | 900 |
| 5 | 2x pool | 2.5 × 10⁷ | 27929 | 895 |
| | (R1767, | | | |
| | R3178) | | | |
| 6 | 2x pool | 1.25 × 10⁷ | 28787 | 434 |
| | R1767, | | | |
| | R3178) | | | |
| 7 | 2x pool | 6.125 × 10⁶ | 27503 | 223 |
| | (R1767, | | | |
| | R3178) | | | |
| 8 | 2x pool | 0 | 28814 | 0 |
| | (R1767, | | | |
| | R3178) | | | |
| 9 | 3x Pool | 2.5 × 10⁷ | 27881 | 897 |
| | (R1767, | | | |
| | R3164, R3178 | | | |
| 10 | 3x Pool | 1.25 × 10⁷ | 29523 | 423 |
| | (R1767, | | | |
| | R3164, R3178) | | | |
| 11 | 3x Pool | 6.125 × 10⁶ | 28957 | 211 |
| | (R1767, | | | |
| | R3164, R3178) | | | |
| 12 | 3x Pool | 0 | 29087 | 0 |
| | (R1767, | | | |
| | R3164, R3178) | | | |

Enhanced sensitivity to target detection with guide-pooling was observed in the case of Cas13a nuclease also. In these assays, a Cas13a complexing mix was prepared wherein the R002(off-target guide), R4517, R4519, R4530 guides were present in either a pooled-gRNA format (a pool of two or more of the three guides R4517, R4519, and R4530) or single-gRNA format (wherein R4517, R4519, and R4530 were present individually) and the mix was incubated for 20 minutes at 37° C. DETECTR™ master mixes which included the Cas13a complex, FAM-U5 Reporter substrate, Buffer, and diluted template SC2 RNA (or off-target template 5S-87) was then assembled as shown in Table 7. The DETECTR™ mixes were then loaded into a Stilla Sapphire chip and placed into the Naica® Geode system. Crystals were generated from the droplets from each of the samples and incubated at 37° C. (no amplification step was performed). The signal from the Sapphire chips was measured in the Cy5 channel. The results of the DETECTR™ assay showed enhanced Cas13a-based detection of the SC2 target RNA using a pooled-guide format compared to a Cas13a-based detection of the SC2 target RNA using a single-guide format. For example, the DETECTR™ assays showed an enhanced signal from chamber 8 (saturated—not displayed), containing the template at a concentration of 1×10⁶ copies, and a pool of the three guides R4517, R4519, and R4530, compared to the signal from chamber 2, chamber 4, or chamber 6 which contained the template at a concentration of 1×10⁶ copies, and the guides R4517, R4519, and R4530 in individual guide format respectively (FIG. 31). Similarly, the DETECTR™ assays showed an enhanced signal from chamber 9 which contained the template at a concentration of 1×10⁵ copies and a pool of three guides (R1767, R3164, and R3178), compared to the signal from chamber 2, chamber 6, or chamber 4, which contained the template at a concentration of 1×10⁶ copies, and which contained the R1767, R3164, and R3178 in individual guide format respectively (FIG. 31).

TABLE 7

| | | Copies/ | # | copies/ |
|---|---|---|---|---|
| Chamber | Condition | Chamber | Droplets | droplet |
| 1 | Off Target | 1 × 10⁶ | 19960 | 50 |
| | Guide (R002) | | | |
| 2 | Single R4517 | 1 × 10⁶ | 18102 | 55 |
| 3 | Single R4517 | 0 | 19146 | 0 |
| 4 | Single R4519 | 1 × 10⁶ | 18289 | 55 |
| 5 | Single R4519 | 0 | 23324 | 0 |
| 6 | Single R4530 | 1 × 10⁶ | 25402 | 39 |
| 7 | Single R4530 | 0 | 26285 | 0 |
| 8 | 3 pool | 1 × 10⁶ | saturated | ~40 |
| 9 | 3 pool | 1 × 10⁵ | 23209 | 4.3 |
| 10 | 3 pool | 1 × 10⁴ | 24064 | 0.41 |
| 11 | 3 pool | 0 | 21137 | 0 |
| 12 | 3 pool | 1 × 10⁶ | 24885 | 40 |

Next, the sensitivity of a target detection in Cas13a digital droplet DETECTR™ assays containing guide RNA in either a pooled-guide format versus a single guide format was assayed. DETECTR™ reaction master-mixes was prepared for each gRNA (R4637, R4638, R4667, R4676, R4684, R4689, R4691, or R4785 (RNaseP)) and included, in addition to the gRNA, the Cas13a nuclease, and the reporter substrate. After complexing, 2 μL of each RNP was combined in either a pooled-gRNA format (a pool of the seven gRNAs, i.e., R4637, R4638, R4676, R4689, R4691, R4667, and R4684) or remained in the single-gRNA format (wherein R4667, R4684, and R4785 (RNAse P were present individually). The template RNAs (Twist SC2, ATCC SC2, and 5s-87 off-target) were diluted to obtain a series of template concentrations. DETECTR™ reactions directed to the detection of the template RNAs (Twist SC2, ATCC SC2, and 5s-87 off-target template RNAs) were assembled by combining the Cas13a-gRNA RNPs with the diluted template RNA from the previous step as shown in Table 8. The assembled DETECTR™ reactions were loaded into chambers on a Stilla Sapphire Chip. The Chips were placed into the Naica® Geode system and crystals were generated using the droplet generation program and imaged to reveal droplets that contain detected targets.

The sensitivity of target detection by the DETECTR™ assays containing the pooled guides (R4637, R4638, R4667, R4676, R4684, R4689, R4691) was compared with the sensitivity of target detection by the DETECTR™ assays containing the single guides R4684, R4667, R4785 (RNAseP guide) in individual format. Relative quantification performed by counting the number of these positive droplets showed that the samples containing the pooled guide RNAs generated more crystals containing the amplified products per copy of starting target RNA than the samples containing the guide RNAs in individual format (FIG. 32). For example, the number of positive droplets from chamber 1 is higher than the number of droplets in chamber 2 and 3; and the number of droplets from chamber 5 is higher than the number of droplets in chambers 6 and 7 (FIG. 32). Measurement of the target detection signal intensity from the chips also confirmed that the sensitivity of target detection per copy of starting target RNA by the DETECTR™ assays containing the pooled guides (R4637, R4638, R4667, R4676, R4684, R4689, R4691) was higher than the sensitivity of target detection by the DETECTR™ assays containing the single guides R4684, R4667, R4785 (RNAseP guide) in individual format (FIG. 33). For example, signal intensity from chamber 1 (containing the seven-guide pool and the Twist SC2 template RNA is higher than the signal intensity in chamber 2 and 3 (containing the R4684, and the R4667 gRNAs in individual format respectively in the presence of the Twist SC2 RNA); and the signal intensity from chamber 5 (containing the seven-guide pool and the ATCC SC2 template RNA) is higher than the signal intensity in chambers 6 and 7 (containing the R4684, and the R4667 gRNAs in individual format respectively, in the presence of the ATCC SC2 RNA) (FIG. 33). Similarly, the signal intensity from chamber 5 (containing the seven-guide pool and the ATCC SC2 template RNA) is higher than the signal intensity in chamber 6 (containing the gRNA R4684 in individual format and the ATCC SC2 RNA), the signal intensity from chamber 8 (containing the control RNaseP gRNA in individual format with the ATCC SC2 template RNA) and the signal intensity from chamber 12 (containing the seven pooled gRNAs with no template RNA) (FIG. 33).

Relative quantification of the number of droplets containing amplified target (per copy of starting target RNA) observed in chamber 5 (containing the seven-guide pool and the ATCC SC2 template RNA) is higher than the number of droplets observed in chamber 6 (containing the gRNA R4684 in individual format and the ATCC SC2 RNA), the number of droplets observed in chamber 8 (containing the control RNaseP gRNA in individual format with the ATCC SC2 template RNA) and the number of droplets observed in chamber 12 (containing the seven pooled gRNAs with no template RNA) FIG. 34. The sensitivity of target detection by the assays containing the pooled guides (R4637, R4638, R4667, R4676, R4684, R4689, R4691) was compared with the sensitivity of target detection by the assays containing the single guides R4684, R4667, R4785 (RNAseP guide) in individual format, when the assays were conducted in a benchtop assay format FIG. 35. Results from the bench top assay showed that the samples containing the pooled guides (R4637, R4638, R4667, R4676, R4684, R4689, R4691) was not higher than the sensitivity of target detection by the in the samples containing the single guides R4684, R4667, or R4785 (RNAseP guide) in individual format FIG. 35.

TABLE 8

| Chamber | Guide | Template |
|---|---|---|
| 1 | 7 pool | 5000 copies Twist SC2 |
| 2 | R4684 | 5000 copies Twist SC2 |
| 3 | R4667 | 5000 copies Twist SC2 |
| 4 | R4785(RNaseP) | 5000 copies Twist SC2 |
| 5 | 7 pool | 5000 copies ATCC SC2 |
| 6 | R4684 | 5000 copies ATCC SC2 |
| 7 | R4667 | 5000 copies ATCC SC2 |
| 8 | R4785(RNaseP) | 5000 copies ATCC SC2 |
| 9 | 7 pool | 5000 copies 5s-87 |
| 10 | R4684 | 5000 copies 5s-87 |
| 11 | R4667 | 5000 copies 5s-87 |
| 12 | 7 pool | NTC |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A system for detection of a target nucleic acid, comprising:
a. an enclosure;
b. a reagent chamber disposed within the enclosure;
c. a programmable nuclease and a guide nucleic acid disposed within the reagent chamber, wherein the guide nucleic acid is complementary to the target nucleic acid or a portion thereof, wherein the programmable nuclease is configured to be activated through binding of the guide nucleic acid to the target nucleic acid;
d. a reporter disposed within the reagent chamber, wherein the reporter comprises a cleavable nucleic acid and a detection moiety, wherein cleavage of the cleavable nucleic acid by the activated programmable nuclease releases the detection moiety from the cleavable nucleic acid; and
e. a lateral flow assay strip disposed within the enclosure; the lateral flow assay strip comprising a sample pad and a detection region, wherein the detection region comprises a stationary capture probe disposed thereon and configured to capture the released detection moiety, wherein release of the detection moiety directly or indirectly produces a detectable signal at a location corresponding to the stationary capture probe.

2. (canceled)

3. (canceled)

4. The system of claim 1, further comprising amplification reagents.

5. The system of claim 4, wherein (i) the amplification reagents are located within the reagent chamber, or (ii) the amplification reagents are located within are amplification chamber located between the reagent chamber and the detection region.

6.-8. (canceled)

9. The system of claim 1, wherein the lateral flow assay strip comprises a flowing capture probe.

10. The system of claim 9, wherein the flowing capture probe comprises an anti-biotin functionalized gold nanoparticle or an antibody.

11. The system of claim 1, wherein the lateral flow assay strip further comprises a conjugation pad, wherein the sample pad is in fluid communication with the conjugation pad.

12.-17. (canceled)

18. The system of claim 1, wherein (i) the system comprises an absorption pad configured to draw a sample comprising the target nucleic acid through the lateral flow assay strip by capillary action, or (ii) the system is configured to drive a sample comprising the target nucleic acid through the lateral flow assay strip by an external pressure.

19.-21. (canceled)

22. The system of claim 1, wherein the stationary capture probe comprises an antibody.

23. (canceled)

24. The system of claim 1, wherein the detection moiety comprises a chemical functional group or a label.

25.-28. (canceled)

29. The system of claim 1, wherein the programmable nuclease is selected from the group consisting of Cas 12, Cas 13, Cas 14, and CasPhi.

30. The system of claim 1, further comprising a plurality of programmable nucleases and a plurality of different guide nucleic acids.

31. The system of claim 30, wherein each guide nucleic acid is complimentary toward (i) a different corresponding target nucleic acid or (ii) a different corresponding segment of the target nucleic acid.

32.-35. (canceled)

36. The system of claim 1, further comprising an amplification chamber located between the reagent chamber and the detection region.

37. (canceled)

38. The system of claim 1, wherein the detection region comprises one or more detection spots.

39. The system of claim 38, wherein each detection spot of the one or more detection spots comprises a corresponding stationary capture probe of a plurality of stationary capture probes.

40.-42. (canceled)

43. The system of claim 1, wherein (i) the reagent chamber comprises a surface, and (ii) the programmable nuclease, the guide nucleic acid, and/or the reporter is immobilized to the surface by covalent, non-covalent, or electrostatic force.

44. The system of claim 43, wherein the programmable nuclease, the guide nucleic acid, and/or the reporter is immobilized to the surface by a linker.

45. (canceled)

46. (canceled)

47. The system of claim 38, further comprising a control spot, wherein (i) the one or more detection spots are located between the reagent chamber and the control spot, or (ii) the control spot is located between the reagent chamber and the one or more detection spots.

48.-60. (canceled)

61. The system of claim 1, further comprising a valve located between the reagent chamber and the detection region, wherein the valve in an open position allows for fluid to pass therethrough and from the reagent chamber to the detection region.

62.-65. (canceled)

66. A method for detecting a target nucleic acid, the method comprising the steps of:
(a) providing a device, comprising:
i. a reagent chamber, comprising:
1. a programmable nuclease and a guide nucleic acid disposed within the reagent chamber, wherein the guide nucleic acid is complementary to the target nucleic acid or a portion thereof, wherein the programmable nuclease is configured to be activated through binding of the guide nucleic acid to the target nucleic acid, and
2. a reporter molecule comprising a cleavable nucleic acid and a detection moiety, wherein cleavage of the cleavable nucleic acid by the activated programmable nuclease releases the detection moiety from the cleavable nucleic acid;
ii. a detection region in fluid communication with the reagent chamber, wherein the detection region comprises a stationary capture probe, to capture the released detection moiety, wherein release of the detection moiety directly or indirectly produces a detectable signal at a location corresponding to the stationary capture probe; and
iii. a housing containing the reagent chamber and detection region,
(b) introducing a sample into the reagent chamber, thereby enabling the detection moiety to be released via the cleavage of the cleavable nucleic acid to produce a detection moiety solution;
(c) contacting the detection moiety solution with the detection region; and
(d) identifying a presence of the target nucleic acid in the sample via the detectable signal produced at the location corresponding to the stationary capture probe.


SEQUENCE LISTING (147 sequences)

SEQ ID NO: 1-2 - skipped (2 entries)

SEQ ID NO: 3 - RNA, 10 nt, synthetic construct
  source 1..10: mol_type = other RNA; note = Description of Artificial Sequence: Synthetic oligonucleotide
tttttttttt

SEQ ID NO: 4 - DNA, 10 bp, synthetic construct
  source 1..10: mol_type = other DNA; note = Description of Artificial Sequence: Synthetic oligonucleotide; note = Description of Combined DNA/RNA Molecule: Synthetic oligonucleotide
  misc_feature 1..4: note = DNA
  misc_feature 5..6: note = RNA
  misc_feature 7..10: note = DNA
tttttttttt

SEQ ID NO: 5-11 - skipped (7 entries)

SEQ ID NO: 12 - DNA, 10 bp, synthetic construct
  source 1..10: mol_type = other DNA; note = Description of Artificial Sequence: Synthetic oligonucleotide
tttttttttt

SEQ ID NO: 13 - DNA, 12 bp, synthetic construct
  source 1..12: mol_type = other DNA; note = Description of Artificial Sequence: Synthetic oligonucleotide
tttttttttttt

SEQ ID NO: 14 - DNA, 14 bp, synthetic construct
  source 1..14: mol_type = other DNA; note = Description of Artificial Sequence: Synthetic oligonucleotide
tttttttttttttt

SEQ ID NO: 15-26 - skipped (12 entries)

SEQ ID NO: 27 - protein, 1228 aa, unidentified
  source 1..1228: note = Description of Unknown: Lachnospiraceae bacterium sequence
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 28 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 29 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 30 - protein, 1246 aa, Porphyromonas macacae
MKTQHFFEDFTSLYSLSKTIRFELKPIGKTLENIKKNGLIRRDEQRLDDYEKLKKVIDEYHEDFIANILSSFSFSEEILQSYIQNLSESEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQIIGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 31 - protein, 1373 aa, Moraxella bovoculi
MLFQDFTHLYPLSKTVRFELKPIDRTLEHIHAKNFLSQDETMADMHQKVKVILDDYHRDFIADMMGEVKLTKLAEFYDVYLKFRKNPKDDELQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFDKINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWTICSHGDKRYVYDKTANQNKGAAKGINVNDELKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 32 - protein, 1259 aa, Moraxella bovoculi
MGIHGVPAALFQDFTHLYPLSKTVRFELKPIGRTLEHIHAKNFLSQDETMADMYQKVKVILDDYHRDFIADMMGEVKLTKLAEFYDVYLKFRKNPKDDGLQKQLKDLQAVLRKESVKPIGSGGKYKTGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNRIIGEVNGYTNKHNQICHKSERIAKLRPLHKQILSDGMGVSFLPSKFADDSEMCQAVNEFYRHYTDVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHHTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKNVYQKMVYKLLPGPNKMLPKVFFAKSNLDYYNPSAELLDKYAKGTHKKGDNFNLKDCHALIDFFKAGINKHPEWQHFGFKFSPTSSYRDLSDFYREVEPQGYQVKFVDINADYIDELVEQGKLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQVTTPYHKILDKREIERLNARVGWGEIETIKELKSGYLSHVVHQINQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNTDKGYFEFHIDYAKFTDKAKNSRQKWAICSHGDKRYVYDKTANQNKGAAKGINVNDELKSLFARYHINDKQPNLVMDICQNNDKEFHKSLMCLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 33 - protein, 1269 aa, Moraxella bovoculi
MGIHGVPAALFQDFTHLYPLSKTVRFELKPIGKTLEHIHAKNFLNQDETMADMYQKVKAILDDYHRDFIADMMGEVKLTKLAEFYDVYLKFRKNPKDDGLQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILATIKQKHSALYDQIINELTASGLDVSLASHLDGYHKLLTQEGITAYNTLLGGISGEAGSRKIQGINELINSHHNQHCHKSERIAKLRPLHKQILSDGMGVSFLPSKFADDSEVCQAVNEFYRHYADVFAKVQSLFDGFDDYQKDGIYVEYKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSDKSPEIRQLKELLDNALNVAHFAKLLTTKTTLHNQDGNFYGEFGALYDELAKIATLYNKVRDYLSQKPFSTEKYKLNFGNPTLLNGWDLNKEKDNFGVILQKDGCYYLALLDKAHKKVFDNAPNTGKSVYQKMIYKLLPGPNKMLPKVFFAKSNLDYYNPSAELLDKYAQGTHKKGDNFNLKDCHALIDFFKAGINKHPEWQHFGFKFSPTSSYQDLSDFYREVEPQGYQVKFVDINADYINELVEQGQLYLFQIYNKDFSPKAHGKPNLHTLYFKALFSEDNLVNPIYKLNGEAEIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQRSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADRGYFEFHIDYAKFNDKAKNSRQIWKICSHGDKRYVYDKTANQNKGATIGVNVNDELKSLFTRYHINDKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 34 - protein, 1306 aa, Thiomicrospira sp.
MGIHGVPAATKTFDSEFFNLYSLQKTVRFELKPVGETASFVEDFKNEGLKRVVSEDERRAVDYQKVKEIIDDYHRDFIEESLNYFPEQVSKDALEQAFHLYQKLKAAKVEEREKALKEWEALQKKLREKVVKCFSDSNKARFSRIDKKELIKEDLINWLVAQNREDDIPTVETFNNFTTYFTGFHENRKNIYSKDDHATAISFRLIHENLPKFFDNVISFNKLKEGFPELKFDKVKEDLEVDYDLKHAFEIEYFVNFVTQAGIDQYNYLLGGKTLEDGTKKQGMNEQINLFKQQQTRDKARQIPKLIPLFKQILSERTESQSFIPKQFESDQELFDSLQKLHNNCQDKFTVLQQAILGLAEADLKKVFIKTSDLNALSNTIFGNYSVFSDALNLYKESLKTKKAQEAFEKLPAHSIHDLIQYLEQFNSSLDAEKQQSTDTVLNYFIKTDELYSRFIKSTSEAFTQVQPLFELEALSSKRRPPESEDEGAKGQEGFEQIKRIKAYLDTLMEAVHFAKPLYLVKGRKMIEGLDKDQSFYEAFEMAYQELESLIIPIYNKARSYLSRKPFKADKFKINFDNNTLLSGWDANKETANASILFKKDGLYYLGIMPKGKTFLFDYFVSSEDSEKLKQRRQKTAEEALAQDGESYFEKIRYKLLPGASKMLPKVFFSNKNIGFYNPSDDILRIRNTASHTKNGTPQKGHSKVEFNLNDCHKMIDFFKSSIQKHPEWGSFGFTFSDTSDFEDMSAFYREVENQGYVISFDKIKETYIQSQVEQGNLYLFQIYNKDFSPYSKGKPNLHTLYWKALFEEANLNNVVAKLNGEAEIFFRRHSIKASDKVVHPANQAIDNKNPHTEKTQSTFEYDLVKDKRYTQDKFFFHVPISLNFKAQGVSKFNDKVNGFLKGNPDVNIIGIDRGERHLLYFTVVNQKGEILVQESLNTLMSDKGHVNDYQQKLDKKEQERDAARKSWTTVENIKELKEGYLSHVVHKLAHLIIKYNAIVCLEDLNFGFKRGRFKVEKQVYQKFEKALIDKLNYLVFKEKELGEVGHYLTAYQLTAPFESFKKLGKQSGILFYVPADYTSKIDPTTGFVNFLDLRYQSVEKAKQLLSDFNAIRFNSVQNYFEFEIDYKKLTPKRKVGTQSKWVICTYGDVRYQNRRNQKGHWETEEVNVTEKLKALFASDSKTTTVIDYANDDNLIDVILEQDKASFFKELLWLLKLTMTLRHSKIKSEDDFILSPVKNEQGEFYDSRKAGEVWPKDADANGAYHIALKGLWNLQQINQWEKGKTLNLAIKNQDWFSFIQEKPYQE

SEQ ID NO: 35 - protein, 1214 aa, Butyrivibrio sp.
MGIHGVPAAYYQNLTKKYPVSKTIRNELIPIGKTLENIRKNNILESDVKRKQDYEHVKGIMDEYHKQLINEALDNYMLPSLNQAAEIYLKKHVDVEDREEFKKTQDLLRREVTGRLKEHENYTKIGKKDILDLLEKLPSISEEDYNALESFRNFYTYFTSYNKVRENLYSDEEKSSTVAYRLINENLPKFLDNIKSYAFVKAAGVLADCIEEEEQDALFMVETFNMTLTQEGIDMYNYQIGKVNSAINLYNQKNHKVEEFKKIPKMKVLYKQILSDREEVFIGEFKDDETLLSSIGAYGNVLMTYLKSEKINIFFDALRESEGKNVYVKNDLSKTTMSNIVFGSWSAFDELLNQEYDLANENKKKDDKYFEKRQKELKKNKSYTLEQMSNLSKEDISPIENYIERISEDIEKICIYNGEFEKIVVNEHDSSRKLSKNIKAVKVIKDYLDSIKELEHDIKLINGSGQELEKNLVVYVGQEEALEQLRPVDSLYNLTRNYLTKKPFSTEKVKLNFNKSTLLNGWDKNKETDNLGILFFKDGKYYLGIMNTTANKAFVNPPAAKTENVFKKVDYKLLPGSNKMLPKVFFAKSNIGYYNPSTELYSNYKKGTHKKGPSFSIDDCHNLIDFFKESIKKHEDWSKFGFEFSDTADYRDISEFYREVEKQGYKLTFTDIDESYINDLIEKNELYLFQIYNKDFSEYSKGKLNLHTLYFMMLFDQRNLDNVVYKLNGEAEVFYRPASIAENELVIHKAGEGIKNKNPNRAKVKETSTFSYDIVKDKRYSKYKFTLHIPITMNFGVDEVRRFNDVINNALRTDDNVNVIGIDRGERNLLYVVVINSEGKILEQISLNSIINKEYDIETNYHALLDEREDDRNKARKDWNTIENIKELKTGYLSQVVNVVAKLVLKYNAIICLEDLNFGFKRGRQKVEKQVYQKFEKMLIEKLNYLVIDKSREQVSPEKMGGALNALQLTSKFKSFAELGKQSGIIYYVPAYLTSKIDPTTGFVNLFYIKYENIEKAKQFFDGFDFIRFNKKDDMFEFSFDYKSFTQKACGIRSKWIVYTNGERIIKYPNPEKNNLFDEKVINVTDEIKGLFKQYRIPYENGEDIKEIIISKAEADFYKRLFRLLHQTLQMRNSTSDGTRDYIISPVKNDRGEFFCSEFSEGTMPKDADANGAYNIARKGLWVLEQIRQKDEGEKVNLSMTNAEWLKYAQLHLL

SEQ ID NO: 36 - protein, 1129 aa, Alicyclobacillus acidoterrestris
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 37 - protein, 1224 aa, synthetic construct
  source 1..1224: note = Description of Artificial Sequence: Synthetic polypeptide
MKKIDNFVGCYPVSKTLRFKAIPIGKTQENIEKKRLVEEDEVRAKDYKAVKKLIDRYHREFIEGVLDNVKLDGLEEYYMLFNKSDREESDNKKIEIMEERFRRVISKSFKNNEEYKKIFSKKIIEEILPNYIKDEEEKELVKGFKGFYTAFVGYAQNRENMYSDEKKSTAISYRIVNENMPRFITNIKVFEKAKSILDVDKINEINEYILNNDYYVDDFFNIDFFNYVLNQKGIDIYNAIIGGIVTGDGRKIQGLNECINLYNQENKKIRLPQFKPLYKQILSESESMSFYIDEIESDDMLIDMLKESLQIDSTINNAIDDLKVLFNNIFDYDLSGIFINNGLPITTISNDVYGQWSTISDGWNERYDVLSNAKDKESEKYFEKRRKEYKKVKSFSISDLQELGGKDLSICKKINEIISEMIDDYKSKIEEIQYLFDIKELEKPLVTDLNKIELIKNSLDGLKRIERYVIPFLGTGKEQNRDEVFYGYFIKCIDAIKEIDGVYNKTRNYLTKKPYSKDKFKLYFENPQLMGGWDRNKESDYRSTLLRKNGKYYVAIIDKSSSNCMMNIEEDENDNYEKINYKLLPGPNKMLPKVFFSKKNREYFAPSKEIERIYSTGTFKKDTNFVKKDCENLITFYKDSLDRHEDWSKSFDFSFKESSAYRDISEFYRDVEKQGYRVSFDLLSSNAVNTLVEEGKLYLFQLYNKDFSEKSHGIPNLHTMYFRSLFDDNNKGNIRLNGGAEMFMRRASLNKQDVTVHKANQPIKNKNLLNPKKTTTLPYDVYKDKRFTEDQYEVHIPITMNKVPNNPYKINHMVREQLVKDDNPYVIGIDRGERNLIYVVVVDGQGHIVEQLSLNEIINENNGISIRTDYHTLLDAKERERDESRKQWKQIENIKELKEGYISQVVHKICELVEKYDAVIALEDLNSGFKNSRVKVEKQVYQKFEKMLITKLNYMVDKKKDYNKPGGVLNGYQLTTQFESFSKMGTQNGIMFYIPAWLTSKMDPTTGFVDLLKPKYKNKADAQKFFSQFDSIRYDNQEDAFVFKVNYTKFPRTDADYNKEWEIYTNGERIRVFRNPKKNNEYDYETVNVSERMKELFDSYDLLYDKGELKETICEMEESKFFEELIKLFRLTLQMRNSISGRTDVDYLISPVKNSNGYFYNSNDYKKEGAKYPKDADANGAYNIARKVLWAIEQFKMADEDKLDKTKISIKNQEWLEYAQTHCE

SEQ ID NO: 38 - protein, 500 aa, synthetic construct
  source 1..500: note = Description of Artificial Sequence: Synthetic polypeptide
MEVQKTVMKTLSLRILRPLYSQEIEKEIKEEKERRKQAGGTGELDGGFYKKLEKKHSEMFSFDRLNLLLNQLQREIAKVYNHAISELYIATIAQGNKSNKHYISSIVYNRAYGYFYNAYIALGICSKVEANFRSNELLTQQSALPTAKSDNFPIVLHKQKGAEGEDGGFRISTEGSDLIFEIPIPFYEYNGENRKEPYKWVKKGGQKPVLKLILSTFRRQRNKGWAKDEGTDAEIRKVTEGKYQVSQIEINRGKKLGEHQKWFANFSIEQPIYERKPNRSIVGGLDVGIRSPLVCAINNSFSRYSVDSNDVFKFSKQVFAFRRRLLSKNSLKRKGHGAAHKLEPITEMTEKNDKFRKKIIERWAKEVTNFFVKNQVGIVQIEDLSTMKDREDHFFNQYLRGFWPYYQMQTLIENKLKEYGIEVKRVQAKYTSQLCSNPNCRYWNNYFNFEYRKVNKFPKFKCEKCNLEISADYNAARNLSTPDIEKFVAKATKGINLPEK

SEQ ID NO: 39 - protein, 507 aa, synthetic construct
  source 1..507: note = Description of Artificial Sequence: Synthetic polypeptide
MEEAKTVSKTLSLRILRPLYSAEIEKEIKEEKERRKQGGKSGELDSGFYKKLEKKHTQMFGWDKLNLMLSQLQRQIARVFNQSISELYIETVIQGKKSNKHYTSKIVYNRAYSVFYNAYLALGITSKVEANFRSTELLMQKSSLPTAKSDNFPILLHKQKGVEGEEGGFKISADGNDLIFEIPIPFYEYDSANKKEPFKWIKKGGQKPTIKLILSTFRRQRNKGWAKDEGTDAEIRKVIEGKYQVSHIEINRGKKLGDHQKWFVNFTIEQPIYERKLDKNIIGGIDVGIKSPLVCAVNNSFARYSVDSNDVLKFSKQAFAFRRRLLSKNSLKRSGHGSKNKLDPITRMTEKNDRFRKKIIERWAKEVTNFFIKNQVGTVQIEDLSTMKDRQDNFFNQYLRGFWPYYQMQNLIENKLKEYGIETKRIKARYTSQLCSNPSCRHWNSYFSFDHRKTNNFPKFKCEKCALEISADYNAARNISTPDIEKFVAKATKGINLPDKNENVILE

SEQ ID NO: 40 - protein, 529 aa, synthetic construct
  source 1..529: note = Description of Artificial Sequence: Synthetic polypeptide
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 41 - protein, 726 aa, synthetic construct
  source 1..726: note = Description of Artificial Sequence: Synthetic polypeptide
MERQKVPQIRKIVRVVPLRILRPKYSDVIENALKKFKEKGDDTNTNDFWRAIRDRDTEFFRKELNFSEDEINQLERDTLFRVGLDNRVLFSYFDFLQEKLMKDYNKIISKLFINRQSKSSFENDLTDEEVEELIEKDVTPFYGAYIGKGIKSVIKSNLGGKFIKSVKIDRETKKVTKLTAINIGLMGLPVAKSDTFPIKIIKTNPDYITFQKSTKENLQKIEDYETGIEYGDLLVQITIPWFKNENKDFSLIKTKEAIEYYKLNGVGKKDLLNINLVLTTYHIRKKKSWQIDGSSQSLVREMANGELEEKWKSFFDTFIKKYGDEGKSALVKRRVNKKSRAKGEKGRELNLDERIKRLYDSIKAKSFPSEINLIPENYKWKLHFSIEIPPMVNDIDSNLYGGIDFGEQNIATLCVKNIEKDDYDFLTIYGNDLLKHAQASYARRRIMRVQDEYKARGHGKSRKTKAQEDYSERMQKLRQKITERLVKQISDFFLWRNKFHMAVCSLRYEDLNTLYKGESVKAKRMRQFINKQQLFNGIERKLKDYNSEIYVNSRYPHYTSRLCSKCGKLNLYFDFLKFRTKNIIIRKNPDGSEIKYMPFFICEFCGWKQAGDKNASANIADKDYQDKLNKEKEFCNIRKPKSKKEDIGEENEEERDYSRRFNRNSFIYNSLKKDNKLNQEKLFDEWKNQLKRKIDGRNKFEPKEYKDRFSYLFAYYQEIIKNESES

SEQ ID NO: 42 - protein, 517 aa, synthetic construct
  source 1..517: note = Description of Artificial Sequence: Synthetic polypeptide
MVPTELITKTLQLRVIRPLYFEEIEKELAELKEQKEKEFEETNSLLLESKKIDAKSLKKLKRKARSSAAVEFWKIAKEKYPDILTKPEMEFIFSEMQKMMARFYNKSMTNIFIEMNNDEKVNPLSLISKASTEANQVIKCSSISSGLNRKIAGSINKTKFKQVRDGLISLPTARTETFPISFYKSTANKDEIPISKINLPSEEEADLTITLPFPFFEIKKEKKGQKAYSYFNIIEKSGRSNNKIDLLLSTHRRQRRKGWKEEGGTSAEIRRLMEGEFDKEWEIYLGEAEKSEKAKNDLIKNMTRGKLSKDIKEQLEDIQVKYFSDNNVESWNDLSKEQKQELSKLRKKKVEELKDWKHVKEILKTRAKIGWVELKRGKRQRDRNKWFVNITITRPPFINKELDDTKFGGIDLGVKVPFVCAVHGSPARLIIKENEILQFNKMVSARNRQITKDSEQRKGRGKKNKFIKKEIFNERNELFRKKIIERWANQIVKFFEDQKCATVQIENLESFDRTSYK

SEQ ID NO: 43 - protein, 481 aa, synthetic construct
  source 1..481: note = Description of Artificial Sequence: Synthetic polypeptide
MKSDTKDKKIIIHQTKTLSLRIVKPQSIPMEEFTDLVRYHQMIIFPVYNNGAIDLYKKLFKAKIQKGNEARAIKYFMNKIVYAPIANTVKNSYIALGYSTKMQSSFSGKRLWDLRFGEATPPTIKADFPLPFYNQSGFKVSSENGEFIIGIPFGQYTKKTVSDIEKKTSFAWDKFTLEDTTKKTLIELLLSTKTRKMNEGWKNNEGTEAEIKRVMDGTYQVTSLEILQRDDSWFVNFNIAYDSLKKQPDRDKIAGIHMGITRPLTAVIYNNKYRALSIYPNTVMHLTQKQLARIKEQRTNSKYATGGHGRNAKVTGTDTLSEAYRQRRKKIIEDWIASIVKFAINNEIGTIYLEDISNTNSFFAAREQKLIYLEDISNTNSFLSTYKYPISAISDTLQHKLEEKAIQVIRKKAYYVNQICSLCGHYNKGFTYQFRRKNKFPKMKCQGCLEATSTEFNAAANVANPDYEKLLIKHGLLQLKK

SEQ ID NO: 44 - protein, 358 aa, synthetic construct
  source 1..358: note = Description of Artificial Sequence: Synthetic polypeptide
MSTITRQVRLSPTPEQSRLLMAHCQQYISTVNVLVAAFDSEVLTGKVSTKDFRAALPSAVKNQALRDAQSVFKRSVELGCLPVLKKPHCQWNNQNWRVEGDQLILPICKDGKTQQERFRCAAVALEGKAGILRIKKKRGKWIADLTVTQEDAPESSGSAIMGVDLGIKVPAVAHIGGKGTRFFGNGRSQRSMRRRFYARRKTLQKAKKLRAVRKSKGKEARWMKTINHQLSRQIVNHAHALGVGTIKIEALQGIRKGTTRKSRGAAARKNNRMTNTWSFSQLTLFITYKAQRQGITVEQVDPAYTSQDCPACRARNGAQDRTYVCSECGWRGHRDTVGAINISRRAGLSGHRRGATGA

SEQ ID NO: 45 - protein, 507 aa, synthetic construct
  source 1..507: note = Description of Artificial Sequence: Synthetic polypeptide
MIAQKTIKIKLNPTKEQIIKLNSIIEEYIKVSNFTAKKIAEIQESFTDSGLTQGTCSECGKEKTYRKYHLLKKDNKLFCITCYKRKYSQFTLQKVEFQNKTGLRNVAKLPKTYYTNAIRFASDTFSGFDEIIKKKQNRLNSIQNRLNFWKELLYNPSNRNEIKIKVVKYAPKTDTREHPHYYSEAEIKGRIKRLEKQLKKFKMPKYPEFTSETISLQRELYSWKNPDELKISSITDKNESMNYYGKEYLKRYIDLINSQTPQILLEKENNSFYLCFPITKNIEMPKIDDTFEPVGIDWGITRNIAVVSILDSKTKKPKFVKFYSAGYILGKRKHYKSLRKHFGQKKRQDKINKLGTKEDRFIDSNIHKLAFLIVKEIRNHSNKPIILMENITDNREEAEKSMRQNILLHSVKSRLQNYIAYKALWNNIPTNLVKPEHTSQICNRCGHQDRENRPKGSKLFKCVKCNYMSNADFNASINIARKFYIGEYEPFYKDNEKMKSGVNSISM

SEQ ID NO: 46 - protein, 534 aa, synthetic construct
  source 1..534: note = Description of Artificial Sequence: Synthetic polypeptide
LKLSEQENITTGVKFKLKLDKETSEGLNDYFDEYGKAINFAIKVIQKELAEDRFAGKVRLDENKKPLLNEDGKKIWDFPNEFCSCGKQVNRYVNGKSLCQECYKNKFTEYGIRKRMYSAKGRKAEQDINIKNSTNKISKTHFNYAIREAFILDKSIKKQRKERFRRLREMKKKLQEFIEIRDGNKILCPKIEKQRVERYIHPSWINKEKKLEDFRGYSMSNVLGKIKILDRNIKREEKSLKEKGQINFKARRLMLDKSVKFLNDNKISFTISKNLPKEYELDLPEKEKRLNWLKEKIKIIKNQKPKYAYLLRKDDNFYLQYTLETEFNLKEDYSGIVGIDRGVSHIAVYTFVHNNGKNERPLFLNSSEILRLKNLQKERDRFLRRKHNKKRKKSNMRNIEKKIQLILHNYSKQIVDFAKNKNAFIVFEKLEKPKKNRSKMSKKSQYKLSQFTFKKLSDLVDYKAKREGIKVLYISPEYTSKECSHCGEKVNTQRPFNGNSSLFKCNKCGVELNADYNASINIAKKGLNILNSTN

SEQ ID NO: 47 - protein, 537 aa, synthetic construct
  source 1..537: note = Description of Artificial Sequence: Synthetic polypeptide
MEESIITGVKFKLRIDKETTKKLNEYFDEYGKAINFAVKIIQKELADDRFAGKAKLDQNKNPILDENGKKIYEFPDEFCSCGKQVNKYVNNKPFCQECYKIRFTENGIRKRMYSAKGRKAEHKINILNSTNKISKTHFNYAIREAFILDKSIKKQRKKRNERLRESKKRLQQFIDMRDGKREICPTIKGQKVDRFIHPSWITKDKKLEDFRGYTLSIINSKIKILDRNIKREEKSLKEKGQIIFKAKRLMLDKSIRFVGDRKVLFTISKTLPKEYELDLPSKEKRLNWLKEKIEIIKNQKPKYAYLLRKNIESEKKPNYEYYLQYTLEIKPELKDFYDGAIGIDRGINHIAVCTFISNDGKVTPPKFFSSGEILRLKNLQKERDRFLLRKHNKNRKKGNMRVIENKINLILHRYSKQIVDMAKKLNASIVFEELGRIGKSRTKMKKSQRYKLSLFIFKKLSDLVDYKSRREGIRVTYVPPEYTSKECSHCGEKVNTQRPFNGNYSLFKCNKCGIQLNSDYNASINIAKKGLKIPNST

SEQ ID NO: 48 - protein, 540 aa, synthetic construct
  source 1..540: note = Description of Artificial Sequence: Synthetic polypeptide
LWTIVIGDFIEMPKQDLVTTGIKFKLDVDKETRKKLDDYFDEYGKAINFAVKIIQKNLKEDRFAGKIALGEDKKPLLDKDGKKIYNYPNESCSCGNQVRRYVNAKPFCVDCYKLKFTENGIRKRMYSARGRKADSDINIKNSTNKISKTHFNYAIREGFILDKSLKKQRSKRIKKLLELKRKLQEFIDIRQGQMVLCPKIKNQRVDKFIHPSWLKRDKKLEEFRGYSLSVVEGKIKIFNRNILREEDSLRQRGHVNFKANRIMLDKSVRFLDGGKVNFNLNKGLPKEYLLDLPKKENKLSWLNEKISLIKLQKPKYAYLLRREGSFFIQYTIENVPKTFSDYLGAIGIDRGISHIAVCTFVSKNGVNKAPVFFSSGEILKLKSLQKQRDLFLRGKHNKIRKKSNMRNIDNKINLILHKYSRNIVNLAKSEKAFIVFEKLEKIKKSRFKMSKSLQYKLSQFTFKKLSDLVEYKAKIEGIKVDYVPPEYTSKECSHCGEKVDTQRPFNGNSSLFKCNKCRVQLNADYNASINIAKKSLNISN

SEQ ID NO: 49 - protein, 542 aa, synthetic construct
  source 1..542: note = Description of Artificial Sequence: Synthetic polypeptide
MSKTTISVKLKIIDLSSEKKEFLDNYFNEYAKATTFCQLRIRRLLRNTHWLGKKEKSSKKWIFESGICDLCGENKELVNEDRNSGEPAKICKRCYNGRYGNQMIRKLFVSTKKREVQENMDIRRVAKLNNTHYHRIPEEAFDMIKAADTAEKRRKKNVEYDKKRQMEFIEMFNDEKKRAARPKKPNERETRYVHISKLESPSKGYTLNGIKRKIDGMGKKIERAEKGLSRKKIFGYQGNRIKLDSNWVRFDLAESEITIPSLFKEMKLRITGPTNVHSKSGQIYFAEWFERINKQPNNYCYLIRKTSSNGKYEYYLQYTYEAEVEANKEYAGCLGVDIGCSKLAAAVYYDSKNKKAQKPIEIFTNPIKKIKMRREKLIKLLSRVKVRHRRRKLMQLSKTEPIIDYTCHKTARKIVEMANTAKAFISMENLETGIKQKQQARETKKQKFYRNMFLFRKLSKLIEYKALLKGIKIVYVKPDYTSQTCSSCGADKEKTERPSQAIFRCLNPTCRYYQRDINADFNAAVNIAKKALNNTEVVTTLL

SEQ ID NO: 50 - protein, 564 aa, synthetic construct
  source 1..564: note = Description of Artificial Sequence: Synthetic polypeptide
MARAKNQPYQKLTTTTGIKFKLDLSEEEGKRFDEYFSEYAKAVNFCAKVIYQLRKNLKFAGKKELAAKEWKFEISNCDFCNKQKEIYYKNIANGQKVCKGCHRTNFSDNAIRKKMIPVKGRKVESKFNIHNTTKKISGTHRHWAFEDAADIIESMDKQRKEKQKRLRREKRKLSYFFELFGDPAKRYELPKVGKQRVPRYLHKIIDKDSLTKKRGYSLSYIKNKIKISERNIERDEKSLRKASPIAFGARKIKMSKLDPKRAFDLENNVFKIPGKVIKGQYKFFGTNVANEHGKKFYKDRISKILAGKPKYFYLLRKKVAESDGNPIFEYYVQWSIDTETPAITSYDNILGIDAGITNLATTVLIPKNLSAEHCSHCGNNHVKPIFTKFFSGKELKAIKIKSRKQKYFLRGKHNKLVKIKRIRPIEQKVDGYCHVVSKQIVEMAKERNSCIALEKLEKPKKSKFRQRRREKYAVSMFVFKKLATFIKYKAAREGIEIIPVEPEGTSYTCSHCKNAQNNQRPYFKPNSKKSWTSMFKCGKCGIELNSDYNAAFNIAQKALNMTSA

SEQ ID NO: 51 - protein, 610 aa, synthetic construct
  source 1..610: note = Description of Artificial Sequence: Synthetic polypeptide
MDEKHFFCSYCNKELKISKNLINKISKGSIREDEAVSKAISIHNKKEHSLILGIKFKLFIENKLDKKKLNEYFDNYSKAVTFAARIFDKIRSPYKFIGLKDKNTKKWTFPKAKCVFCLEEKEVAYANEKDNSKICTECYLKEFGENGIRKKIYSTRGRKVEPKYNIFNSTKELSSTHYNYAIRDAFQLLDALKKQRQKKLKSIFNQKLRLKEFEDIFSDPQKRIELSLKPHQREKRYIHLSKSGQESINRGYTLRFVRGKIKSLTRNIEREEKSLRKKTPIHFKGNRLMIFPAGIKFDFASNKVKISISKNLPNEFNFSGTNVKNEHGKSFFKSRIELIKTQKPKYAYVLRKIKREYSKLRNYEIEKIRLENPNADLCDFYLQYTIETESRNNEEINGIIGIDRGITNLACLVLLKKGDKKPSGVKFYKGNKILGMKIAYRKHLYLLKGKRNKLRKQRQIRAIEPKINLILHQISKDIVKIAKEKNFAIALEQLEKPKKARFAQRKKEKYKLALFTFKNLSTLIEYKSKREGIPVIYVPPEKTSQMCSHCAINGDEHVDTQRPYKKPNAQKPSYSLFKCNKCGIELNADYNAAFNIAQKGLKTLMLNHSH

SEQ ID NO: 52 - protein, 369 aa, synthetic construct
  source 1..369: note = Description of Artificial Sequence: Synthetic polypeptide
MLQTLLVKLDPSKEQYKMLYETMERFNEACNQIAETVFAIHSANKIEVQKTVYYPIREKFGLSAQLTILAIRKVCEAYKRDKSIKPEFRLDGALVYDQRVLSWKGLDKVSLVTLQGRQIIPIKFGDYQKARMDRIRGQADLILVKGVFYLCVVVEVSEESPYDPKGVLGVDLGIKNLAVDSDGEVHSGEQTTNTRERLDSLKARLQSKGTKSAKRHLKKLSGRMAKFSKDVNHCISKKLVAKAKGTLMSIALEDLQGIRDRVTVRKAQRRNLHTWNFGLLRMFVDYKAKIAGVPLVFVDPRNTSRTCPSCGHVAKANRPTRDEFRCVSCGFAGAADHIAAMNIAFRAEVSQPIVTRFFVQSQAPSFRVG

SEQ ID NO: 53 - protein, 552 aa, synthetic construct
  source 1..552: note = Description of Artificial Sequence: Synthetic polypeptide
MDEEPDSAEPNLAPISVKLKLVKLDGEKLAALNDYFNEYAKAVNFCELKMQKIRKNLVNIRGTYLKEKKAWINQTGECCICKKIDELRCEDKNPDINGKICKKCYNGRYGNQMIRKLFVSTNKRAVPKSLDIRKVARLHNTHYHRIPPEAADIIKAIETAERKRRNRILFDERRYNELKDALENEEKRVARPKKPKEREVRYVPISKKDTPSKGYTMNALVRKVSGMAKKIERAKRNLNKRKKIEYLGRRILLDKNWVRFDFDKSEISIPTMKEFFGEMRFEITGPSNVMSPNGREYFTKWFDRIKAQPDNYCYLLRKESEDETDFYLQYTWRPDAHPKKDYTGCLGIDIGGSKLASAVYFDADKNRAKQPIQIFSNPIGKWKTKRQKVIKVLSKAAVRHKTKKLESLRNIEPRIDVHCHRIARKIVGMALAANAFISMENLEGGIREKQKAKETKKQKFSRNMFVFRKLSKLIEYKALMEGVKVVYIVPDYTSQLCSSCGTNNTKRPKQAIFMCQNTECRYFGKNINADFNAAINIAKKALNRKDIVRELS

SEQ ID NO: 54 - protein, 534 aa, synthetic construct
  source 1..534: note = Description of Artificial Sequence: Synthetic polypeptide
MEKNNSEQTSITTGIKFKLKLDKETKEKLNNYFDEYGKAINFAVRIIQMQLNDDRLAGKYKRDEKGKPILGEDGKKILEIPNDFCSCGNQVNHYVNGVSFCQECYKKRFSENGIRKRMYSAKGRKAEQDINIKNSTNKISKTHFNYAIREAFNLDKSIKKQREKRFKKLKDMKRKLQEFLEIRDGKRVICPKIEKQKVERYIHPSWINKEKKLEEFRGYSLSIVNSKIKSFDRNIQREEKSLKEKGQINFKAQRLMLDKSVKFLKDNKVSFTISKELPKTFELDLPKKEKKLNWLNEKLEIIKNQKPKYAYLLRKENNIFLQYTLDSIPEIHSEYSGAVGIDRGVSHIAVYTFLDKDGKNERPFFLSSSGILRLKNLQKERDKFLRKKHNKIRKKGNMRNIEQKINLILHEYSKQIVNFAKDKNAFIVFELLEKPKKSRERMSKKIQYKLSQFTFKKLSDLVDYKAKREGIKVIYVEPAYTSKDCSHCGERVNTQRPFNGNFSLFKCNKCGIVLNSDYNASLNIARKGLNISAN

SEQ ID NO: 55 - protein, 577 aa, synthetic construct
  source 1..577: note = Description of Artificial Sequence: Synthetic polypeptide
MAEEKFFFCEKCNKDIKIPKNYINKQGAEEKARAKHEHRVHALILGIKFKIYPKKEDISKLNDYFDEYAKAVTFTAKIVDKLKAPFLFAGKRDKDTSKKKWVFPVDKCSFCKEKTEINYRTKQGKNICNSCYLTEFGEQGLLEKIYATKGRKVSSSFNLFNSTKKLTGTHNNYVVKESLQLLDALKKQRSKRLKKLSNTRRKLKQFEEMFEKEDKRFQLPLKEKQRELRFIHVSQKDRATEFKGYTMNKIKSKIKVLRRNIEREQRSLNRKSPVFFRGTRIRLSPSVQFDDKDNKIKLTLSKELPKEYSFSGLNVANEHGRKFFAEKLKLIKENKSKYAYLLRRQVNKNNKKPIYDYYLQYTVEFLPNIITNYNGILGIDRGINTLACIVLLENKKEKPSFVKFFSGKGILNLKNKRRKQLYFLKGVHNKYRKQQKIRPIEPRIDQILHDISKQIIDLAKEKRVAISLEQLEKPQKPKFRQSRKAKYKLSQFNFKTLSNYIDYKAKKEGIRVIYIAPEMTSQNCSRCAMKNDLHVNTQRPYKNTSSLFKCNKCGVELNADYNAAFNIAQKGLKILNS

SEQ ID NO: 56 - protein, 613 aa, synthetic construct
  source 1..613: note = Description of Artificial Sequence: Synthetic polypeptide
MISLKLKLLPDEEQKKLLDEMFWKWASICTRVGFGRADKEDLKPPKDAEGVWFSLTQLNQANTDINDLREAMKHQKHRLEYEKNRLEAQRDDTQDALKNPDRREISTKRKDLFRPKASVEKGFLKLKYHQERYWVRRLKEINKLIERKTKTLIKIEKGRIKFKATRITLHQGSFKIRFGDKPAFLIKALSGKNQIDAPFVVVPEQPICGSVVNSKKYLDEITTNFLAYSVNAMLFGLSRSEEMLLKAKRPEKIKKKEEKLAKKQSAFENKKKELQKLLGRELTQQEEAIIEETRNQFFQDFEVKITKQYSELLSKIANELKQKNDFLKVNKYPILLRKPLKKAKSKKINNLSPSEWKYYLQFGVKPLLKQKSRRKSRNVLGIDRGLKHLLAVTVLEPDKKTFVWNKLYPNPITGWKWRRRKLLRSLKRLKRRIKSQKHETIHENQTRKKLKSLQGRIDDLLHNISRKIVETAKEYDAVIVVEDLQSMRQHGRSKGNRLKTLNYALSLFDYANVMQLIKYKAGIEGIQIYDVKPAGTSQNCAYCLLAQRDSHEYKRSQENSKIGVCLNPNCQNHKKQIDADLNAARVIASCYALKINDSQPFGTRKRFKKRTTN

SEQ ID NO: 57 - protein, 615 aa, synthetic construct
  source 1..615: note = Description of Artificial Sequence: Synthetic polypeptide
METLSLKLKLNPSKEQLLVLDKMFWKWASICTRLGLKKAEMSDLEPPKDAEGVWFSKTQLNQANTDVNDLRKAMQHQGKRIEYELDKVENRRNEIQEMLEKPDRRDISPNRKDLFRPKAAVEKGYLKLKYHKLGYWSKELKTANKLIERKRKTLAKIDAGKMKFKPTRISLHTNSFRIKFGEEPKIALSTTSKHEKIELPLITSLQRPLKTSCAKKSKTYLDAAILNFLAYSTNAALFGLSRSEEMLLKAKKPEKIEKRDRKLATKRESFDKKLKTLEKLLERKLSEKEKSVFKRKQTEFFDKFCITLDETYVEALHRIAEELVSKNKYLEIKKYPVLLRKPESRLRSKKLKNLKPEDWTYYIQFGFQPLLDTPKPIKTKTVLGIDRGVRHLLAVSIFDPRTKTFTFNRLYSNPIVDWKWRRRKLLRSIKRLKRRLKSEKHVHLHENQFKAKLRSLEGRIEDHFHNLSKEIVDLAKENNSVIVVENLGGMRQHGRGRGKWLKALNYALSHFDYAKVMQLIKYKAELAGVFVYDVAPAGTSINCAYCLLNDKDASNYTRGKVINGKKNTKIGECKTCKKEFDADLNAARVIALCYEKRLNDPQPFGTRKQFKPKKP

SEQ ID NO: 58 - protein, 775 aa, synthetic construct
  source 1..775: note = Description of Artificial Sequence: Synthetic polypeptide
MKALKLQLIPTRKQYKILDEMFWKWASLANRVSQKGESKETLAPKKDIQKIQFNATQLNQIEKDIKDLRGAMKEQQKQKERLLLQIQERRSTISEMLNDDNNKERDPHRPLNFRPKGWRKFHTSKHWVGELSKILRQEDRVKKTIERIVAGKISFKPKRIGIWSSNYKINFFKRKISINPLNSKGFELTLMTEPTQDLIGKNGGKSVLNNKRYLDDSIKSLLMFALHSRFFGLNNTDTYLLGGKINPSLVKYYKKNQDMGEFGREIVEKFERKLKQEINEQQKKIIMSQIKEQYSNRDSAFNKDYLGLINEFSEVFNQRKSERAEYLLDSFEDKIKQIKQEIGESLNISDWDFLIDEAKKAYGYEEGFTEYVYSKRYLEILNKIVKAVLITDIYFDLRKYPILLRKPLDKIKKISNLKPDEWSYYIQFGYDSINPVQLMSTDKFLGIDRGLTHLLAYSVFDKEKKEFIINQLEPNPIMGWKWKLRKVKRSLQHLERRIRAQKMVKLPENQMKKKLKSIEPKIEVHYHNISRKIVNLAKDYNASIVVESLEGGGLKQHGRKKNARNRSLNYALSLFDYGKIASLIKYKADLEGVPMYEVLPAYTSQQCAKCVLEKGSFVDPEIIGYVEDIGIKGSLLDSLFEGTELSSIQVLKKIKNKIELSARDNHNKEINLILKYNFKGLVIVRGQDKEEIAEHPIKEINGKFAILDFVYKRGKEKVGKKGNQKVRYTGNKKVGYCSKHGQVDADLNASRVIALCKYLDINDPILFGEQRKSFK

SEQ ID NO: 59 - protein, 777 aa, synthetic construct
  source 1..777: note = Description of Artificial Sequence: Synthetic polypeptide
MVTRAIKLKLDPTKNQYKLLNEMFWKWASLANRFSQKGASKETLAPKDGTQKIQFNATQLNQIKKDVDDLRGAMEKQGKQKERLLIQIQERLLTISEILRDDSKKEKDPHRPQNFRPFGWRRFHTSAYWSSEASKLTRQVDRVRRTIERIKAGKINFKPKRIGLWSSTYKINFLKKKINISPLKSKSFELDLITEPQQKIIGKEGGKSVANSKKYLDDSIKSLLIFAIKSRLFGLNNKDKPLFENIITPNLVRYHKKGQEQENFKKEVIKKFENKLKKEISQKQKEIIFSQIERQYENRDATFSEDYLRAISEFSEIFNQRKKERAKELLNSFNEKIRQLKKEVNGNISEEDLKILEVEAEKAYNYENGFIEWEYSEQFLGVLEKIARAVLISDNYFDLKKYPILIRKPTNKSKKITNLKPEEWDYYIQFGYGLINSPMKIETKNFMGIDRGLTHLLAYSIFDRDSEKFTINQLELNPIKGWKWKLRKVKRSLQHLERRMRAQKGVKLPENQMKKRLKSIEPKIESYYHNLSRKIVNLAKANNASIVVESLEGGGLKQHGRKKNSRHRALNYALSLFDYGKIASLIKYKSDLEGVPMYEVLPAYTSQQCAKCVLKKGSFVEPEIIGYIEEIGFKENLLTLLFEDTGLSSVQVLKKSKNKMTLSARDKEGKMVDLVLKYNFKGLVISQEKKKEEIVEFPIKEIDGKFAVLDSAYKRGKERISKKGNQKLVYTGNKKVGYCSVHGQVDADLNASRVIALCKYLGINEPIVFGEQRKSFK

SEQ ID NO: 60 - protein, 610 aa, synthetic construct
  source 1..610: note = Description of Artificial Sequence: Synthetic polypeptide
LDLITEPIQPHKSSSLRSKEFLEYQISDFLNFSLHSLFFGLASNEGPLVDFKIYDKIVIPKPEERFPKKESEEGKKLDSFDKRVEEYYSDKLEKKIERKLNTEEKNVIDREKTRIWGEVNKLEEIRSIIDEINEIKKQKHISEKSKLLGEKWKKVNNIQETLLSQEYVSLISNLSDELTNKKKELLAKKYSKFDDKIKKIKEDYGLEFDENTIKKEGEKAFLNPDKFSKYQFSSSYLKLIGEIARSLITYKGFLDLNKYPIIFRKPINKVKKIHNLEPDEWKYYIQFGYEQINNPKLETENILGIDRGLTHILAYSVFEPRSSKFILNKLEPNPIEGWKWKLRKLRRSIQNLERRWRAQDNVKLPENQMKKNLRSIEDKVENLYHNLSRKIVDLAKEKNACIVFEKLEGQGMKQHGRKKSDRLRGLNYKLSLFDYGKIAKLIKYKAEIEGIPIYRIDSAYTSQNCAKCVLESRRFAQPEEISCLDDFKEGDNLDKRILEGTGLVEAKIYKKLLKEKKEDFEIEEDIAMFDTKKVIKENKEKTVILDYVYTRRKEIIGTNHKKNIKGIAKYTGNTKIGYCMKHGQVDADLNASRTIALCKNFDINNPEIWK

SEQ ID NO: 61 - protein, 632 aa, synthetic construct
  source 1..632: note = Description of Artificial Sequence: Synthetic polypeptide
MSDESLVSSEDKLAIKIKIVPNAEQAKMLDEMFKKWSSICNRISRGKEDIETLRPDEGKELQFNSTQLNSATMDVSDLKKAMARQGERLEAEVSKLRGRYETIDASLRDPSRRHTNPQKPSSFYPSDWDISGRLTPRFHTARHYSTELRKLKAKEDKMLKTINKIKNGKIVFKPKRITLWPSSVNMAFKGSRLLLKPFANGFEMELPIVISPQKTADGKSQKASAEYMRNALLGLAGYSINQLLFGMNRSQKMLANAKKPEKVEKFLEQMKNKDANFDKKIKALEGKWLLDRKLKESEKSSIAVVRTKFFKSGKVELNEDYLKLLKHMANEILERDGFVNLNKYPILSRKPMKRYKQKNIDNLKPNMWKYYIQFGYEPIFERKASGKPKNIMGIDRGLTHLLAVAVFSPDQQKFLFNHLESNPIMHWKWKLRKIRRSIQHMERRIRAEKNKHIHEAQLKKRLGSIEEKTEQHYHIVSSKIINWAIEYEAAIVLESLSHMKQRGGKKSVRTRALNYALSLFDYEKVARLITYKARIRGIPVYDVLPGMTSKTCATCLLNGSQGAYVRGLETTKAAGKATKRKNMKIGKCMVCNSSENSMIDADLNAARVIAICKYKNLNDPQPAGSRKVFKRF

SEQ ID NO: 62 - protein, 625 aa, synthetic construct
  source 1..625: note = Description of Artificial Sequence: Synthetic polypeptide
MLALKLKIMPTEKQAEILDAMFWKWASICSRIAKMKKKVSVKENKKELSKKIPSNSDIWFSKTQLCQAEVDVGDHKKALKNFEKRQESLLDELKYKVKAINEVINDESKREIDPNNPSKFRIKDSTKKGNLNSPKFFTLKKWQKILQENEKRIKKKESTIEKLKRGNIFFNPTKISLHEEEYSINFGSSKLLLNCFYKYNKKSGINSDQLENKFNEFQNGLNIICSPLQPIRGSSKRSFEFIRNSIINFLMYSLYAKLFGIPRSVKALMKSNKDENKLKLEEKLKKKKSSFNKTVKEFEKMIGRKLSDNESKILNDESKKFFEIIKSNNKYIPSEEYLKLLKDISEEIYNSNIDFKPYKYSILIRKPLSKFKSKKLYNLKPTDYKYYLQLSYEPFSKQLIATKTILGIDRGLKHLLAVSVFDPSQNKFVYNKLIKNPVFKWKKRYHDLKRSIRNRERRIRALTGVHIHENQLIKKLKSMKNKINVLYHNVSKNIVDLAKKYESTIVLERLENLKQHGRSKGKRYKKLNYVLSNFDYKKIESLISYKAKKEGVPVSNINPKYTSKTCAKCLLEVNQLSELKNEYNRDSKNSKIGICNIHGQIDADLNAARVIALCYSKNLNEPHFK

SEQ ID NO: 63 - protein, 517 aa, synthetic construct
  source 1..517: note = Description of Artificial Sequence: Synthetic polypeptide
VINLFGYKFALYPNKTQEELLNKHLGECGWLYNKAIEQNEYYKADSNIEEAQKKFELLPDKNSDEAKVLRGNISKDNYVYRTLVKKKKSEINVQIRKAVVLRPAETIRNLAKVKKKGLSVGRLKFIPIREWDVLPFKQSDQIRLEENYLILEPYGRLKFKMHRPLLGKPKTFCIKRTATDRWTISFSTEYDDSNMRKNDGGQVGIDVGLKTHLRLSNENPDEDPRYPNPKIWKRYDRRLTILQRRISKSKKLGKNRTRLRLRLSRLWEKIRNSRADLIQNETYEILSENKLIAIEDLNVKGMQEKKDKKGRKGRTRAQEKGLHRSISDAAFSEFRRVLEYKAKRFGSEVKPVSAIDSSKECHNCGNKKGMPLESRIYECPKCGLKIDRDLNSAKVILARATGVRPGSNARADTKISATAGASVQTEGTVSEDFRQQMETSDQKPMQGEGSKEPPMNPEHKSSGRGSKHVNIGCKNKVGLYNEDENSRSTEKQIMDENRSTTEDMVEIGALHSPVLTT

SEQ ID NO: 64 - protein, 410 aa, synthetic construct
  source 1..410: note = Description of Artificial Sequence: Synthetic polypeptide
MIASIDYEAVSQALIVFEFKAKGKDSQYQAIDEAIRSYRFIRNSCLRYWMDNKKVGKYDLNKYCKVLAKQYPFANKLNSQARQSAAECSWSAISRFYDNCKRKVSGKKGFPKFKKHARSVEYKTSGWKLSENRKAITFTDKNGIGKLKLKGTYDLHFSQLEDMKRVRLVRRADGYYVQFCISVDVKVETEPTGKAIGLDVGIKYFLADSSGNTIENPQFYRKAEKKLNRANRRKSKKYIRGVKPQSKNYHKARCRYARKHLRVSRQRKEYCKRVAYCVIHSNDVVAYEDLNVKGMVKNRHLAKSISDVAWSTFRHWLEYFAIKYGKLTIPVAPHNTSQNCSNCDKKVPKSLSTRTHICHHCGYSEDRDVNAAKNILKKALSTVGQTGSLKLGEIEPLLVLEQSCTRKFDL

SEQ ID NO: 65 - protein, 486 aa, synthetic construct
  source 1..486: note = Description of Artificial Sequence: Synthetic polypeptide
LAEENTLHLTLAMSLPLNDLPENRTRSELWRRQWLPQKKLSLLLGVNQSVRKAAADCLRWFEPYQELLWWEPTDPDGKKLLDKEGRPIKRTAGHMRVLRKLEEIAPFRGYQLGSAVKNGLRHKVADLLLSYAKRKLDPQFTDKTSYPSIGDQFPIVWTGAFVCYEQSITGQLYLYLPLFPRGSHQEDITNNYDPDRGPALQVFGEKEIARLSRSTSGLLLPLQFDKWGEATFIRGENNPPTWKATHRRSDKKWLSEVLLREKDFQPKRVELLVRNGRIFVNVACEIPTKPLLEVENFMGVSFGLEHLVTVVVINRDGNVVHQRQEPARRYEKTYFARLERLRRRGGPFSQELETFHYRQVAQIVEEALRFKSVPAVEQVGNIPKGRYNPRLNLRLSYWPFGKLADLTSYKAVKEGLPKPYSVYSATAKMLCSTCGAANKEGDQPISLKGPTVYCGNCGTRHNTGFNTALNLARRAQELFVKGVVAR

SEQ ID NO: 66 - protein, 602 aa, synthetic construct
  source 1..602: note = Description of Artificial Sequence: Synthetic polypeptide
MSQSLLKWHDMAGRDKDASRSLQKSAVEGVLLHLTASHRVALEMLEKSVSQTVAVTMEAAQQRLVIVLEDDPTKATSRKRVISADLQFTREEFGSLPNWAQKLASTCPEIATKYADKHINSIRIAWGVAKESTNGDAVEQKLQWQIRLLDVTMFLQQLVLQLADKALLEQIPSSIRGGIGQEVAQQVTSHIQLLDSGTVLKAELPTISDRNSELARKQWEDAIQTVCTYALPFSRERARILDPGKYAAEDPRGDRLINIDPMWARVLKGPTVKSLPLLFVSGSSIRIVKLTLPRKHAAGHKHTFTATYLVLPVSREWINSLPGTVQEKVQWWKKPDVLATQELLVGKGALKKSANTLVIPISAGKKRFFNHILPALQRGFPLQWQRIVGRSYRRPATHRKWFAQLTIGYTNPSSLPEMALGIHFGMKDILWWALADKQGNILKDGSIPGNSILDFSLQEKGKIERQQKAGKNVAGKKYGKSLLNATYRVVNGVLEFSKGISAEHASQPIGLGLETIRFVDKASGSSPVNARHSNWNYGQLSGIFANKAGPAGFSVTEITLKKAQRDLSDAEQARVLAIEATKRFASRIKRLATKRKDDTLFV

SEQ ID NO: 67 - protein, 494 aa, synthetic construct
  source 1..494: note = Description of Artificial Sequence: Synthetic polypeptide
VEPVEKERFYYRTYTFRLDGQPRTQNLTTQSGWGLLTKAVLDNTKHYWEIVHHARIANQPIVFENPVIDEQGNPKLNKLGQPRFWKRPISDIVNQLRALFENQNPYQLGSSLIQGTYWDVAENLASWYALNKEYLAGTATWGEPSFPEPHPLTEINQWMPLTFSSGKVVRLLKNASGRYFIGLPILGENNPCYRMRTIEKLIPCDGKGRVTSGSLILFPLVGIYAQQHRRMTDICESIRTEKGKLAWAQVSIDYVREVDKRRRMRRTRKSQGWIQGPWQEVFILRLVLAHKAPKLYKPRCFAGISLGPKTLASCVILDQDERVVEKQQWSGSELLSLIHQGEERLRSLREQSKPTWNAAYRKQLKSLINTQVFTIVTFLRERGAAVRLESIARVRKSTPAPPVNFLLSHWAYRQITERLKDLAIRNGMPLTHSNGSYGVRFTCSQCGATNQGIKDPTKYKVDIESETFLCSICSHREIAAVNTATNLAKQLLDE

SEQ ID NO: 68 - protein, 526 aa, synthetic construct
  source 1..526: note = Description of Artificial Sequence: Synthetic polypeptide
MNDTETSETLTSHRTVCAHLHVVGETGSLPRLVEAALAELITLNGRATQALLSLAKNGLVLRRDKEENLIAAELTLPCRKNKYADVAAKAGEPILATRINNKGKLVTKKWYGEGNSYHIVRFTPETGMFTVRVFDRYAFDEELLHLHSEVVFGSDLPKGIKAKTDSLPANFLQAVFTSFLELPFQGFPDIVVKPAMKQAAEQLLSYVQLEAGENQQAEYPDTNERDPELRLVEWQKSLHELSVRTEPFEFVRARDIDYYAETDRRGNRFVNITPEWTKFAESPFARRLPLKIPPEFCILLRRKTEGHAKIPNRIYLGLQIFDGVTPDSTLGVLATAEDGKLFWWHDHLDEFSNLEGKPEPKLKNKPQLLMVSLEYDREQRFEESVGGDRKICLVTLKETRNFRRGWNGRILGIHFQHNPVITWALMDHDAEVLEKGFIEGNAFLGKALDKQALNEYLQKGGKWVGDRSFGNKLKGITHTLASLIVRLAREKDAWIALEEISWVQKQSADSVANHEIVEQPHHSLTR

SEQ ID NO: 69 - protein, 649 aa, synthetic construct
  source 1..649: note = Description of Artificial Sequence: Synthetic polypeptide
MNDTETSETLTSHRTVCAHLHVVGETGSLPRLVEAALAELITLNGRATQALLSLAKNGLVLRRDKEENLIAAELTLPCRKNKYADVAAKAGEPILATRINNKGKLVTKKWYGEGNSYHIVRFTPETGMFTVRVFDRYAFDEELLHLHSEVVFGSDLPKGIKAKTDSLPANFLQAVFTSFLELPFQGFPDIVVKPAMKQAAEQLLSYVQLEAGENQQAEYPDTNERDPELRLVEWQKSLHELSVRTEPFEFVRARDIDYYAETDRRGNRFVNITPEWTKFAESPFARRLPLKIPPEFCILLRRKTEGHAKIPNRIYLGLQIFDGVTPDSTLGVLATAEDGKLFWWHDHLDEFSNLEGKPEPKLKNKPQLLMVSLEYDREQRFEESVGGDRKICLVTLKETRNFRRGRHGHTRTDRLPAGNTLWRADFATSAEVAAPKWNGRILGIHFQHNPVITWALMDHDAEVLEKGFIEGNAFLGKALDKQALNEYLQKGGKWVGDRSFGNKLKGITHTLASLIVRLAREKDAWIALEEISWVQKQSADSVANRRFSMWNYSRLATLIEWLGTDIATRDCGTAAPLAHKVSDYLTHFTCPECGACRKAGQKKEIADTVRAGDILTCRKCGFSGPIPDNFIAEFVAKKALERMLKKKPV

SEQ ID NO: 70 - protein, 414 aa, synthetic construct
  source 1..414: note = Description of Artificial Sequence: Synthetic polypeptide
MAKRNFGEKSEALYRAVRFEVRPSKEELSILLAVSEVLRMLFNSALAERQQVFTEFIASLYAELKSASVPEEISEIRKKLREAYKEHSISLFDQINALTARRVEDEAFASVTRNWQEETLDALDGAYKSFLSLRRKGDYDAHSPRSRDSGFFQKIPGRSGFKIGEGRIALSCGAGRKLSFPIPDYQQGRLAETTKLKKFELYRDQPNLAKSGRFWISVVYELPKPEATTCQSEQVAFVALGASSIGVVSQRGEEVIALWRSDKHWVPKIEAVEERMKRRVKGSRGWLRLLNSGKRRMHMISSRQHVQDEREIVDYLVRNHGSHFVVTELVVRSKEGKLADSSKPERGGSLGLNWAAQNTGSLSRLVRQLEEKVKEHGGSVRKHKLTLTEAPPARGAENKLWMARKLRESFLKEV

SEQ ID NO: 71 - protein, 413 aa, synthetic construct
  source 1..413: note = Description of Artificial Sequence: Synthetic polypeptide
LAKNDEKELLYQSVKFEIYPDESKIRVLTRVSNILVLVWNSALGERRARFELYIAPLYEELKKFPRKSAESNALRQKIREGYKEHIPTFFDQLKKLLTPMRKEDPALLGSVPRAYQEETLNTLNGSFVSFMTLRRNNDMDAKPPKGRAEDRFHEISGRSGFKIDGSEFVLSTKEQKLRFPIPNYQLEKLKEAKQIKKFTLYQSRDRRFWISIAYEIELPDQRPFNPEEVIYIAFGASSIGVISPEGEKVIDFWRPDKHWKPKIKEVENRMRSCKKGSRAWKKRAAARRKMYAMTQRQQKLNHREIVASLLRLGFHFVVTEYTVRSKPGKLADGSNPKRGGAPQGFNWSAQNTGSFGEFILWLKQKVKEQGGTVQTFRLVLGQSERPEKRGRDNKIEMVRLLREKYLESQTIVV

SEQ ID NO: 72 - protein, 449 aa, synthetic construct
  source 1..449: note = Description of Artificial Sequence: Synthetic polypeptide
MAKGKKKEGKPLYRAVRFEIFPTSDQITLFLRVSKNLQQVWNEAWQERQSCYEQFFGSIYERIGQAKKRAQEAGFSEVWENEAKKGLNKKLRQQEISMQLVSEKESLLQELSIAFQEHGVTLYDQINGLTARRIIGEFALIPRNWQEETLDSLDGSFKSFLALRKNGDPDAKPPRQRVSENSFYKIPGRSGFKVSNGQIYLSFGKIGQTLTSVIPEFQLKRLETAIKLKKFELCRDERDMAKPGRFWISVAYEIPKPEKVPVVSKQITYLAIGASRLGVVSPKGEFCLNLPRSDYHWKPQINALQERLEGVVKGSRKWKKRMAACTRMFAKLGHQQKQHGQYEVVKKLLRHGVHFVVTELKVRSKPGALADASKSDRKGSPTGPNWSAQNTGNIARLIQKLTDKASEHGGTVIKRNPPLLSLEERQLPDAQRKIFIAKKLREEFLADQK

SEQ ID NO: 73 - protein, 711 aa, synthetic construct
  source 1..711: note = Description of Artificial Sequence: Synthetic polypeptide
MAKREKKDDVVLRGTKMRIYPTDRQVTLMDMWRRRCISLWNLLLNLETAAYGAKNTRSKLGWRSIWARVVEENHAKALIVYQHGKCKKDGSFVLKRDGTVKHPPRERFPGDRKILLGLFDALRHTLDKGAKCKCNVNQPYALTRAWLDETGHGARTADIIAWLKDFKGECDCTAISTAAKYCPAPPTAELLTKIKRAAPADDLPVDQAILLDLFGALRGGLKQKECDHTHARTVAYFEKHELAGRAEDILAWLIAHGGTCDCKIVEEAANHCPGPRLFIWEHELAMIMARLKAEPRTEWIGDLPSHAAQTVVKDLVKALQTMLKERAKAAAGDESARKTGFPKFKKQAYAAGSVYFPNTTMFFDVAAGRVQLPNGCGSMRCEIPRQLVAELLERNLKPGLVIGAQLGLLGGRIWRQGDRWYLSCQWERPQPTLLPKTGRTAGVKIAASIVFTTYDNRGQTKEYPMPPADKKLTAVHLVAGKQNSRALEAQKEKEKKLKARKERLRLGKLEKGHDPNALKPLKRPRVRRSKLFYKSAARLAACEAIERDRRDGFLHRVTNEIVHKFDAVSVQKMSVAPMMRRQKQKEKQIESKKNEAKKEDNGAAKKPRNLKPVRKLLRHVAMARGRQFLEYKYNDLRGPGSVLIADRLEPEVQECSRCGTKNPQMKDGRRLLRCIGVLPDGTDCDAVLPRNRNAARNAEKRLRKHREAHNA

SEQ ID NO: 74 - protein, 574 aa, synthetic construct
  source 1..574: note = Description of Artificial Sequence: Synthetic polypeptide
MNEVLPIPAVGEDAADTIMRGSKMRIYPSVRQAATMDLWRRRCIQLWNLLLELEQAAYSGENRRTQIGWRSIWATVVEDSHAEAVRVAREGKKRKDGTFRKAPSGKEIPPLDPAMLAKIQRQMNGAVDVDPKTGEVTPAQPRLFMWEHELQKIMARLKQAPRTHWIDDLPSHAAQSVVKDLIKALQAMLRERKKRASGIGGRDTGFPKFKKNRYAAGSVYFANTQLRFEAKRGKAGDPDAVRGEFARVKLPNGVGWMECRMPRHINAAHAYAQATLMGGRIWRQGENWYLSCQWKMPKPAPLPRAGRTAAIKIAAAIPITTVDNRGQTREYAMPPIDRERIAAHAAAGRAQSRALEARKRRAKKREAYAKKRHAKKLERGIAAKPPGRARIKLSPGFYAAAAKLAKLEAEDANAREAWLHEITTQIVRNFDVIAVPRMEVAKLMKKPEPPEEKEEQVKAPWQGKRRSLKAARVMMRRTAMALIQTTLKYKAVDLRGPQAYEEIAPLDVTAAACSGCGVLKPEWKMARAKGREIMRCQEPLPGGKTCNTVLTYTRNSARVIGRELAVRLAERQKA

SEQ ID NO: 75 - protein, 400 aa, synthetic construct
  source 1..400: note = Description of Artificial Sequence: Synthetic polypeptide
MTTQKTYNFCFYDQRFFELSKEAGEVYSRSLEEFWKIYDETGVWLSKFDLQKHMRNKLERKLLHSDSFLGAMQQVHANLASWKQAKKVVPDACPPRKPKFLQAILFKKSQIKYKNGFLRLTLGTEKEFLYLKWDINIPLPIYGSVTYSKTRGWKINLCLETEVEQKNLSENKYLSIDLGVKRVATIFDGENTITLSGKKFMGLMHYRNKLNGKTQSRLSHKKKGSNNYKKIQRAKRKTTDRLLNIQKEMLHKYSSFIVNYAIRNDIGNIIIGDNSSTHDSPNMRGKTNQKISQNPEQKLKNYIKYKFESISGRVDIVPEPYTSRKCPHCKNIKKSSPKGRTYKCKKCGFIFDRDGVGAINIYNENVSFGQIISPGRIRSLTEPIGMKFHNEIYFKSYVAA

SEQ ID NO: 76 - protein, 743 aa, synthetic construct
  source 1..743: note = Description of Artificial Sequence: Synthetic polypeptide
MSVRSFQARVECDKQTMEHLWRTHKVFNERLPEIIKILFKMKRGECGQNDKQKSLYKSISQSILEANAQNADYLLNSVSIKGWKPGTAKKYRNASFTWADDAAKLSSQGIHVYDKKQVLGDLPGMMSQMVCRQSVEAISGHIELTKKWEKEHNEWLKEKEKWESEDEHKKYLDLREKFEQFEQSIGGKITKRRGRWHLYLKWLSDNPDFAAWRGNKAVINPLSEKAQIRINKAKPNKKNSVERDEFFKANPEMKALDNLHGYYERNFVRRRKTKKNPDGFDHKPTFTLPHPTIHPRWFVFNKPKTNPEGYRKLILPKKAGDLGSLEMRLLTGEKNKGNYPDDWISVKFKADPRLSLIRPVKGRRVVRKGKEQGQTKETDSYEFFDKHLKKWRPAKLSGVKLIFPDKTPKAAYLYFTCDIPDEPLTETAKKIQWLETGDVTKKGKKRKKKVLPHGLVSCAVDLSMRRGTTGFATLCRYENGKIHILRSRNLWVGYKEGKGCHPYRWTEGPDLGHIAKHKREIRILRSKRGKPVKGEESHIDLQKHIDYMGEDRFKKAARTIVNFALNTENAASKNGFYPRADVLLLENLEGLIPDAEKERGINRALAGWNRRHLVERVIEMAKDAGFKRRVFEIPPYGTSQVCSKCGALGRRYSIIRENNRREIRFGYVEKLFACPNCGYCANADHNASVNLNRRFLIEDSFKSYYDWKRLSEKKQKEEIETIESKLMDKLCAMHKISRGSISK

SEQ ID NO: 77 - protein, 769 aa, synthetic construct
  source 1..769: note = Description of Artificial Sequence: Synthetic polypeptide
MHLWRTHCVFNQRLPALLKRLFAMRRGEVGGNEAQRQVYQRVAQFVLARDAKDSVDLLNAVSLRKRSANSAFKKKATISCNGQAREVTGEEVFAEAVALASKGVFAYDKDDMRAGLPDSLFQPLTRDAVACMRSHEELVATWKKEYREWRDRKSEWEAEPEHALYLNLRPKFEEGEAARGGRFRKRAERDHAYLDWLEANPQLAAWRRKAPPAVVPIDEAGKRRIARAKAWKQASVRAEEFWKRNPELHALHKIHVQYLREFVRPRRTRRNKRREGFKQRPTFTMPDPVRHPRWCLFNAPQTSPQGYRLLRLPQSRRTVGSVELRLLTGPSDGAGFPDAWVNVRFKADPRLAQLRPVKVPRTVTRGKNKGAKVEADGFRYYDDQLLIERDAQVSGVKLLFRDIRMAPFADKPIEDRLLSATPYLVFAVEIKDEARTERAKAIRFDETSELTKSGKKRKTLPAGLVSVAVDLDTRGVGFLTRAVIGVPEIQQTHHGVRLLQSRYVAVGQVEARASGEAEWSPGPDLAHIARHKREIRRLRQLRGKPVKGERSHVRLQAHIDRMGEDRFKKAARKIVNEALRGSNPAAGDPYTRADVLLYESLETLLPDAERERGINRALLRWNRAKLIEHLKRMCDDAGIRHFPVSPFGTSQVCSKCGALGRRYSLARENGRAVIRFGWVERLFACPNPECPGRRPDRPDRPFTCNSDHNASVNLHRVFALGDQAVAAFRALAPRDSPARTLAVKRVEDTLRPQLMRVHKLADAGVDSPF

SEQ ID NO: 78 - protein, 666 aa, synthetic construct
  source 1..666: note = Description of Artificial Sequence: Synthetic polypeptide
MATLVYRYGVRAHGSARQQDAVVSDPAMLEQLRLGHELRNALVGVQHRYEDGKRAVWSGFASVAAADHRVTTGETAVAELEKQARAEHSADRTAATRQGTAESLKAARAAVKQARADRKAAMAAVAEQAKPKIQALGDDRDAEIKDLYRRFCQDGVLLPRCGRCAGDLRSDGDCTDCGAAHEPRKLYWATYNAIREDHQTAVKLVEAKRKAGQPARLRFRRWTGDGTLTVQLQRMHGPACRCVTCAEKLTRRARKTDPQAPAVAADPAYPPTDPPRDPALLASGQGKWRNVLQLGTWIPPGEWSAMSRAERRRVGRSHIGWQLGGGRQLTLPVQLHRQMPADADVAMAQLTRVRVGGRHRMSVALTAKLPDPPQVQGLPPVALHLGWRQRPDGSLRVATWACPQPLDLPPAVADVVVSHGGRWGEVIMPARWLADAEVPPRLLGRRDKAMEPVLEALADWLEAHTEACTARMTPALVRRWRSQGRLAGLTNRWRGQPPTGSAEILTYLEAWRIQDKLLWERESHLRRRLAARRDDAWRRVASWLARHAGVLVVDDADIAELRRRDDPADTDPTMPASAAQAARARAALAAPGRLRHLATITATRDGLGVHTVASAGLTRLHRKCGHQAQPDPRYAASAVVTCPGCGNGYDQDYNAAMLMLDRQQQP

SEQ ID NO: 79 - protein, 564 aa, synthetic construct
  source 1..564: note = Description of Artificial Sequence: Synthetic polypeptide
MSRVELHRAYKFRLYPTPAQVAELAEWERQLRRLYNLAHSQRLAAMQRHVRPKSPGVLKSECLSCGAVAVAEIGTDGKAKKTVKHAVGCSVLECRSCGGSPDAEGRTAHTAACSFVDYYRQGREMTQLLEEDDQLARVVCSARQETLRDLEKAWQRWHKMPGFGKPHFKKRIDSCRIYFSTPKSWAVDLGYLSFTGVASSVGRIKIRQDRVWPGDAKFSSCHVVRDVDEWYAVFPLTFTKEIEKPKGGAVGINRGAVHAIADSTGRVVDSPKFYARSLGVIRHRARLLDRKVPFGRAVKPSPTKYHGLPKADIDAAAARVNASPGRLVYEARARGSIAAAEAHLAALVLPAPRQTSQLPSEGRNRERARRFLALAHQRVRRQREWFLHNESAHYAQSYTKIAIEDWSTKEMTSSEPRDAEEMKRVTRARNRSILDVGWYELGRQIAYKSEATGAEFAKVDPGLRETETHVPEAIVRERDVDVSGMLRGEAGISGTCSRCGGLLRASASGHADAECEVCLHVEVGDVNAAVNVLKRAMFPGAAPPSKEKAKVTIGIKGRKKKRAA

SEQ ID NO: 80 - protein, 565 aa, synthetic construct
  source 1..565: note = Description of Artificial Sequence: Synthetic polypeptide
MSRVELHRAYKFRLYPTPVQVAELSEWERQLRRLYNLGHEQRLLTLTRHLRPKSPGVLKGECLSCDSTQVQEVGADGRPKTTVRHAEQCPTLACRSCGALRDAEGRTAHTVACAFVDYYRQGREMTELLAADDQLARVVCSARQEVLRDLDKAWQRWRKMPGFGKPRFKRRTDSCRIYFSTPKAWKLEGGHLSFTGAATTVGAIKMRQDRNWPASVQFSSCHVVRDVDEWYAVFPLTFVAEVARPKGGAVGINRGAVHAIADSTGRVVDSPRYYARALGVIRHRARLFDRKVPSGHAVKPSPTKYRGLSAIEVDRVARATGFTPGRVVTEALNRGGVAYAECALAAIAVLGHGPERPLTSDGRNREKARKFLALAHQRVRRQREWFLHNESAHYARTYSKIAIEDWSTKEMTASEPQGEETRRVTRSRNRSILDVGWYELGRQLAYKTEATGAEFAQVDPGLKETETNVPKAIADARDVDVSGMLRGEAGISGTCSKCGGLLRAPASGHADAECEICLNVEVGDVNAAVNVLKRAMFPGDAPPASGEKPKVSIGIKGRQKKKKAA

SEQ ID NO: 81 - protein, 499 aa, synthetic construct
  source 1..499: note = Description of Artificial Sequence: Synthetic polypeptide
MEAIATGMSPERRVELGILPGSVELKRAYKFRLYPMKVQQAELSEWERQLRRLYNLAHEQRLAALLRYRDWDFQKGACPSCRVAVPGVHTAACDHVDYFRQAREMTQLLEVDAQLSRVICCARQEVLRDLDKAWQRWRKKLGGRPRFKRRTDSCRIYLSTPKHWEIAGRYLRLSGLASSVGEIRIEQDRAFPEGALLSSCSIVRDVDEWYACLPLTFTQPIERAPHRSVGLNRGVVHALADSDGRVVDSPKFFERALATVQKRSRDLARKVSGSRNAHKARIKLAKAHQRVRRQRAAFLHQESAYYSKGFDLVALEDMSVRKMTATAGEAPEMGRGAQRDLNRGILDVGWYELARQIDYKRLAHGGELLRVDPGQTTPLACVTEEQPARGISSACAVCGIPLARPASGNARMRCTACGSSQVGDVNAAENVLTRALSSAPSGPKSPKASIKIKGRQKRLGTPANRAGEASGGDPPVRGPVEGGTLAYVVEPVSESQSDT

SEQ ID NO: 82 - protein, 560 aa, synthetic construct
  source 1..560: note = Description of Artificial Sequence: Synthetic polypeptide
MTVRTYKYRAYPTPEQAEALTSWLRFASQLYNAALEHRKNAWGRHDAHGRGFRFWDGDAAPRKKSDPPGRWVYRGGGGAHISKNDQGKLLTEFRREHAELLPPGMPALVQHEVLARLERSMAAFFQRATKGQKAGYPRWRSEHRYDSLTFGLTSPSKERFDPETGESLGRGKTVGAGTYHNGDLRLTGLGELRILEHRRIPMGAIPKSVIVRRSGKRWFVSIAMEMPSVEPAASGRPAVGLDMGVVTWGTAFTADTSAAAALVADLRRMATDPSDCRRLEELEREAAQLSEVLAHCRARGLDPARPRRCPKELTKLYRRSLHRLGELDRACARIRRRLQAAHDIAEPVPDEAGSAVLIEGSNAGMRHARRVARTQRRVARRTRAGHAHSNRRKKAVQAYARAKERERSARGDHRHKVSRALVRQFEEISVEALDIKQLTVAPEHNPDPQPDLPAHVQRRRNRGELDAAWGAFFAALDYKAADAGGRVARKPAPHTTQECARCGTLVPKPISLRVHRCPACGYTAPRTVNSARNVLQRPLEEPGRAGPSGANGRGVPHAVA

SEQ ID NO: 83 - protein, 404 aa, synthetic construct
  source 1..404: note = Description of Artificial Sequence: Synthetic polypeptide
MNCRYRYRIYPTPGQRQSLARLFGCVRVVWNDALFLCRQSEKLPKNSELQKLCITQAKKTEARGWLGQVSAIPLQQSVADLGVAFKNFFQSRSGKRKGKKVNPPRVKRRNNRQGARFTRGGFKVKTSKVYLARIGDIKIKWSRPLPSEPSSVTVIKDCAGQYFLSFVVEVKPEIKPPKNPSIGIDLGLKTFASCSNGEKIDSPDYSRLYRKLKRCQRRLAKRQRGSKRRERMRVKVAKLNAQIRDKRKDFLHKLSTKVVNENQVIALEDLNVGGMLKNRKLSRAISQAGWYEFRSLCEGKAEKHNRDFRVISRWEPTSQVCSECGYRWGKIDLSVRSIVCINCGVEHDRDDNASVNIEQAGLKVGVGHTHDSKRTGSACKTSNGAVCVEPSTHREYVQLTLFDW

SEQ ID NO: 84 - protein, 392 aa, synthetic construct
  source 1..392: note = Description of Artificial Sequence: Synthetic polypeptide
MKSRWTFRCYPTPEQEQHLARTFGCVRFVWNWALRARTDAFRAGERIGYPATDKALTLLKQQPETVWLNEVSSVCLQQALRDLQVAFSNFFDKRAAHPSFKRKEARQSANYTERGFSFDHERRILKLAKIGAIKVKWSRKAIPHPSSIRLIRTASGKYFVSLVVETQPAPMPETGESVGVDFGVARLATLSNGERISNPKHGAKWQRRLAFYQKRLARATKGSKRRMRIKRHVARIHEKIGNSRSDTLHKLSTDLVTRFDLICVEDLNLRGMVKNHSLARSLHDASIGSAIRMIEEKAERYGKNVVKIDRWFPSSKTCSDCGHIVEQLPLNVREWTCPECGTTHDRDANAAANILAVGQTVSAHGGTVRRSRAKASERKSQRSANRQGVNRA

SEQ ID NO: 85 - protein, 500 aa, synthetic construct
  source 1..500: note = Description of Artificial Sequence: Synthetic polypeptide
KEPLNIGKTAKAVFKEIDPTSLNRAANYDASIELNCKECKFKPFKNVKRYEFNFYNNWYRCNPNSCLQSTYKAQVRKVEIGYEKLKNEILTQMQYYPWFGRLYQNFFHDERDKMTSLDEIQVIGVQNKVFFNTVEKAWREIIKKRFKDNKETMETIPELKHAAGHGKRKLSNKSLLRRRFAFVQKSFKFVDNSDVSYRSFSNNIACVLPSRIGVDLGGVISRNPKREYIPQEISFNAFWKQHEGLKKGRNIEIQSVQYKGETVKRIEADTGEDKAWGKNRQRRFTSLILKLVPKQGGKKVWKYPEKRNEGNYEYFPIPIEFILDSGETSIRFGGDEGEAGKQKHLVIPFNDSKATPLASQQTLLENSRFNAEVKSCIGLAIYANYFYGYARNYVISSIYHKNSKNGQAITAIYLESIAHNYVKAIERQLQNLLLNLRDFSFMESHKKELKKYFGGDLEGTGGAQKRREKEEKIEKEIEQSYLPRLIRLSLTKMVTKQVEM

SEQ ID NO: 86 - protein, 507 aa, synthetic construct
  source 1..507: note = Description of Artificial Sequence: Synthetic polypeptide
ELIVNENKDPLNIGKTAKAVFKEIDPTSINRAANYDASIELACKECKFKPFNNTKRHDFSFYSNWHRCSPNSCLQSTYRAKIRKTEIGYEKLKNEILNQMQYYPWFGRLYQNFFNDQRDKMTSLDEIQVTGVQNKIFFNTVEKAWREIIKKRFRDNKETMRTIPDLKNKSGHGSRKLSNKSLLRRRFAFAQKSFKLVDNSDVSYRAFSNNVACVLPSKIGVDIGGIINKDLKREYIPQEITFNVFWKQHDGLKKGRNIEIHSVQYKGEIVKRIEADTGEDKAWGKNRQRRFTSLILKITPKQGGKKIWKFPEKKNASDYEYFPIPIEFILDNGDASIKFGGEEGEVGKQKHLLIPFNDSKATPLSSKQMLLETSRFNAEVKSTIGLALYANYFVSYARNYVIKSTYHKNSKKGQIVTEIYLESISQNFVRAIQRQLQSLMLNLKDWGFMQTHKKELKKYFGSDLEGSKGGQKRREKEEKIEKEIEASYLPRLIRLSLTKSVTKAEEM

SEQ ID NO: 87 - protein, 529 aa, synthetic construct
  source 1..529: note = Description of Artificial Sequence: Synthetic polypeptide
PEEKTSKLKPNSINLAANYDANEKFNCKECKFHPFKNKKRYEFNFYNNLHGCKSCTKSTNNPAVKRIEIGYQKLKFEIKNQMEAYPWFGRLRINFYSDEKRKMSELNEMQVTGVKNKIFFDAIECAWREILKKRFRESKETLITIPKLKNKAGHGARKHRNKKLLIRRRAFMKKNFHFLDNDSISYRSFANNIACVLPSKVGVDIGGIISPDVGKDIKPVDISLNLMWASKEGIKSGRKVEIYSTQYDGNMVKKIEAETGEDKSWGKNRKRRQTSLLLSIPKPSKQVQEFDFKEWPRYKDIEKKVQWRGFPIKIIFDSNHNSIEFGTYQGGKQKVLPIPFNDSKTTPLGSKMNKLEKLRFNSKIKSRLGSAIAANKFLEAARTYCVDSLYHEVSSANAIGKGKIFIEYYLEILSQNYIEAAQKQLQRFIESIEQWFVADPFQGRLKQYFKDDLKRAKCFLCANREVQTTCYAAVKLHKSCAEKVKDKNKELAIKERNNKEDAVIKEVEASNYPRVIRLKLTKTITNKAM

SEQ ID NO: 88 - protein, 726 aa, synthetic construct
  source 1..726: note = Description of Artificial Sequence: Synthetic polypeptide
SESENKIIEQYYAFLYSFRDKYEKPEFKNRGDIKRKLQNKWEDFLKEQNLKNDKKLSNYIFSNRNFRRSYDREEENEEGIDEKKSKPKRINCFEKEKNLKDQYDKDAINASANKDGAQKWGCFECIFFPMYKIESGDPNKRIIINKTRFKLFDFYLNLKGCKSCLRSTYHPYRSNVYIESNYDKLKREIGNFLQQKNIFQRMRKAKVSEGKYLTNLDEYRLSCVAMHFKNRWLFFDSIQKVLRETIKQRLKQMRESYDEQAKTKRSKGHGRAKYEDQVRMIRRRAYSAQAHKLLDNGYITLFDYDDKEINKVCLTAINQEGFDIGGYLNSDIDNVMPPIEISFHLKWKYNEPILNIESPFSKAKISDYLRKIREDLNLERGKEGKARSKKNVRRKVLASKGEDGYKKIFTDFFSKWKEELEGNAMERVLSQSSGDIQWSKKKRIHYTTLVLNINLLDKKGVGNLKYYEIAEKTKILSFDKNENKFWPITIQVLLDGYEIGTEYDEIKQLNEKTSKQFTIYDPNTKIIKIPFTDSKAVPLGMLGINIATLKTVKKTERDIKVSKIFKGGLNSKIVSKIGKGIYAGYFPTVDKEILEEVEEDTLDNEFSSKSQRNIFLKSIIKNYDKMLKEQLFDFYSFLVRNDLGVRFLTDRELQNIEDESFNLEKRFFETDRDRIARWFDNTNTDDGKEKFKKLANEIVDSYKPRLIRLPVVRVIKRIQPVKQREM

SEQ ID NO: 89 - protein, 517 aa, synthetic construct
  source 1..517: note = Description of Artificial Sequence: Synthetic polypeptide
KYSTRDFSELNEIQVTACKQDEFFKVIQNAWREIIKKRFLENRENFIEKKIFKNKKGRGKRQESDKTIQRNRASVMKNFQLIENEKIILRAPSGHVACVFPVKVGLDIGGFKTDDLEKNIFPPRTITINVFWKNRDRQRKGRKLEVWGIKARTKLIEKVHKWDKLEEVKKKRLKSLEQKQEKSLDNWSEVNNDSFYKVQIDELQEKIDKSLKGRTMNKILDNKAKESKEAEGLYIEWEKDFEGEMLRRIEASTGGEEKWGKRRQRRHTSLLLDIKNNSRGSKEIINFYSYAKQGKKEKKIEFFPFPLTITLDAEEESPLNIKSIPIEDKNATSKYFSIPFTETRATPLSILGDRVQKFKTKNISGAIKRNLGSSISSCKIVQNAETSAKSILSLPNVKEDNNMEIFINTMSKNYFRAMMKQMESFIFEMEPKTLIDPYKEKAIKWFEVAASSRAKRKLKKLSKADIKKSELLLSNTEEFEKEKQEKLEALEKEIEEFYLPRIVRLQLTKTILETPVM

SEQ ID NO: 90 - protein, 481 aa, synthetic construct
  source 1..481: note = Description of Artificial Sequence: Synthetic polypeptide
KKLQLLGHKILLKEYDPNAVNAAANFETSTAELCGQCKMKPFKNKRRFQYTFGKNYHGCLSCIQNVYYAKKRIVQIAKEELKHQLTDSIASIPYKYTSLFSNTNSIDELYILKQERAAFFSNTNSIDELYITGIENNIAFKVISAIWDEIIKKRRQRYAESLTDTGTVKANRGHGGTAYKSNTRQEKIRALQKQTLHMVTNPYISLARYKNNYIVATLPRTIGMHIGAIKDRDPQKKLSDYAINFNVFWSDDRQLIELSTVQYTGDMVRKIEAETGENNKWGENMKRTKTSLLLEILTKKTTDELTFKDWAFSTKKEIDSVTKKTYQGFPIGIIFEGNESSVKFGSQNYFPLPFDAKITPPTAEGFRLDWLRKGSFSSQMKTSYGLAIYSNKVTNAIPAYVIKNMFYKIARAENGKQIKAKFLKKYLDIAGNNYVPFIIMQHYRVLDTFEEMPISQPKVIRLSLTKTQHIIIKKDKTDSKM

SEQ ID NO: 91 - protein, 534 aa, synthetic construct
  source 1..534: note = Description of Artificial Sequence: Synthetic polypeptide
NTSNLINLGKKAINISANYDANLEVGCKNCKFLSSNGNFPRQTNVKEGCHSCEKSTYEPSIYLVKIGERKAKYDVLDSLKKFTFQSLKYQSKKSMKSRNKKPKELKEFVIFANKNKAFDVIQKSYNHLILQIKKEINRMNSKKRKKNHKRRLFRDREKQLNKLRLIESSNLFLPRENKGNNHVFTYVAIHSVGRDIGVIGSYDEKLNFETELTYQLYFNDDKRLLYAYKPKQNKIIKIKEKLWNLRKEKEPLDLEYEKPLNKSITFSIKNDNLFKVSKDLMLRRAKFNIQGKEKLSKEERKINRDLIKIKGLVNSMSYGRFDELKKEKNIWSPHIYREVRQKEIKPCLIKNGDRIEIFEQLKKKMERLRRFREKRQKKISKDLIFAERIAYNFHTKSIKNTSNKINIDQEAKRGKASYMRKRIGYETFKNKYCEQCLSKGNVYRNVQKGCSCFENPFDWIKKGDENLLPKKNEDLRVKGAFRDEALEKQIVKIAFNIAKGYEDFYDNLGESTEKDLKLKFKVGTTINEQESLKL

SEQ ID NO: 92 - protein, 537 aa, synthetic construct
  source 1..537: note = Description of Artificial Sequence: Synthetic polypeptide
TSNPIKLGKKAINISANYDSNLQIGCKNCKFLSYNGNFPRQTNVKEGCHSCEKSTYEPPVYTVRIGERRSKYDVLDSLKKFIFLSLKYRQSKKMKTRSKGIRGLEEFVISANLKKAMDVIQKSYRHLILNIKNEIVRMNGKKRNKNHKRLLFRDREKQLNKLRLIEGSSFFKPPTVKGDNSIFTCVAIHNIGRDIGIAGDYFDKLEPKIELTYQLYYEYNPKKESEINKRLLYAYKPKQNKIIEIKEKLWNLRKEKSPLDLEYEKPLTKSITFLVKRDGVFRISKDLMLRKAKFIIQGKEKLSKEERKINRDLIKIKSNIISLTYGRFDELKKDKTIWSPHIFRDVKQGKITPCIERKGDRMDIFQQLRKKSERLRENRKKRQKKISKDLIFAERIAYNFHTKSIKNTSNLINIKHEAKRGKASYMRKRIGNETFRIKYCEQCFPKNNVYKNVQKGCSCFEDPFEYIKKGNEDLIPNKNQDLKAKGAFRDDALEKQIIKVAFNIAKGYEDFYENLKKTTEKDIRLKFKVGTIISEEM

SEQ ID NO: 93 - protein, 541 aa, synthetic construct
  source 1..541: note = Description of Artificial Sequence: Synthetic polypeptide
NNSINLSKKAINISANYDANLQVRCKNCKFLSSNGNFPRQTDVKEGCHSCEKSTYEPPVYDVKIGEIKAKYEVLDSLKKFTFQSLKYQLSKSMKFRSKKIKELKEFVIFAKESKALNVINRSYKHLILNIKNDINRMNSKKRIKNHKGRLFLDRQKQLSKLKLIEGSSFFVPAKNVGNKSVFTCVAIHSIGRDIGIAGLYDSFTKPVNEITYQIFFSGERRLLYAYKPKQLKILSIKENLWSLKNEKKPLDLLYEKPLGKNLNFNVKGGDLFRVSKDLMIRNAKFNVHGRQRLSDEERLINRNFIKIKGEVVSLSYGRFEELKKDRKLWSPHIFKDVRQNKIKPCLVMQGQRIDIFEQLKRKLELLKKIRKSRQKKLSKDLIFGERIAYNFHTKSIKNTSNKINIDSDAKRGRASYMRKRIGNETFKLKYCDVCFPKANVYRRVQNGCSCSENPYNYIKKGDKDLLPKKDEGLAIKGAFRDEKLNKQIIKVAFNIAKGYEDFYDDLKKRTEKDVDLKFKIGTTVLDQKPMEIFDGIVITWL

SEQ ID NO: 94 - protein, 542 aa, synthetic construct
  source 1..542: note = Description of Artificial Sequence: Synthetic polypeptide
LLTTVVETNNLAKKAINVAANFDANIDRQYYRCTPNLCRFIAQSPRETKEKDAGCSSCTQSTYDPKVYVIKIGKLLAKYEILKSLKRFLFMNRYFKQKKTERAQQKQKIGTELNEMSIFAKATNAMEVIKRATKHCTYDIIPETKSLQMLKRRRHRVKVRSLLKILKERRMKIKKIPNTFIEIPKQAKKNKSDYYVAAALKSCGIDVGLCGAYEKNAEVEAEYTYQLYYEYKGNSSTKRILYCYNNPQKNIREFWEAFYIQGSKSHVNTPGTIRLKMEKFLSPITIESEALDFRVWNSDLKIRNGQYGFIKKRSLGKEAREIKKGMGDIKRKIGNLTYGKSPSELKSIHVYRTERENPKKPRAARKKEDNFMEIFEMQRKKDYEVNKKRRKEATDAAKIMDFAEEPIRHYHTNNLKAVRRIDMNEQVERKKTSVFLKRIMQNGYRGNYCRKCIKAPEGSNRDENVLEKNEGCLDCIGSEFIWKKSSKEKKGLWHTNRLLRRIRLQCFTTAKAYENFYNDLFEKKESSLDIIKLKVSITTKSM

SEQ ID NO: 95 - protein, 564 aa, synthetic construct
  source 1..564: note = Description of Artificial Sequence: Synthetic polypeptide
ASTMNLAKQAINFAANYDSNLEIGCKGCKFMSTWSKKSNPKFYPRQNNQANKCHSCTYSTGEPEVPIIEIGERAAKYKIFTALKKFVFMSVAYKERRRQRFKSKKPKELKELAICSNREKAMEVIQKSVVHCYGDVKQEIPRIRKIKVLKNHKGRLFYKQKRSKIKIAKLEKGSFFKTFIPKVHNNGCHSCHEASLNKPILVTTALNTIGADIGLINDYSTIAPTETDISWQVYYEFIPNGDSEAVKKRLLYFYKPKGALIKSIRDKYFKKGHENAVNTGFFKYQGKIVKGPIKFVNNELDFARKPDLKSMKIKRAGFAIPSAKRLSKEDREINRESIKIKNKIYSLSYGRKKTLSDKDIIKHLYRPVRQKGVKPLEYRKAPDGFLEFFYSLKRKERRLRKQKEKRQKDMSEIIDAADEFAWHRHTGSIKKTTNHINFKSEVKRGKVPIMKKRIANDSFNTRHCGKCVKQGNAINKYYIEKQKNCFDCNSIEFKWEKAALEKKGAFKLNKRLQYIVKACFNVAKAYESFYEDFRKGEEESLDLKFKIGTTTTLKQYPQNKARAM

SEQ ID NO: 96 - protein, 610 aa, synthetic construct
  source 1..610: note = Description of Artificial Sequence: Synthetic polypeptide
HSHNLMLTKLGKQAINFAANYDANLEIGCKNCKFLSYSPKQANPKKYPRQTDVHEDGNIACHSCMQSTKEPPVYIVPIGERKSKYEILTSLNKFTFLALKYKEKKRQAFRAKKPKELQELAIAFNKEKAIKVIDKSIQHLILNIKPEIARIQRQKRLKNRKGKLLYLHKRYAIKMGLIKNGKYFKVGSPKKDGKKLLVLCALNTIGRDIGIIGNIEENNRSETEITYQLYFDCLDANPNELRIKEIEYNRLKSYERKIKRLVYAYKPKQTKILEIRSKFFSKGHENKVNTGSFNFENPLNKSISIKVKNSAFDFKIGAPFIMLRNGKFHIPTKKRLSKEEREINRTLSKIKGRVFRLTYGRNISEQGSKSLHIYRKERQHPKLSLEIRKQPDSFIDEFEKLRLKQNFISKLKKQRQKKLADLLQFADRIAYNYHTSSLEKTSNFINYKPEVKRGRTSYIKKRIGNEGFEKLYCETCIKSNDKENAYAVEKEELCFVCKAKPFTWKKTNKDKLGIFKYPSRIKDFIRAAFTVAKSYNDFYENLKKKDLKNEIFLKFKIGLILSHEKKNHISIAKSVAEDERISGKSIKNILNKSIKLEKNCYSCFFHKEDM

SEQ ID NO: 97 - protein, 552 aa, synthetic construct
  source 1..552: note = Description of Artificial Sequence: Synthetic polypeptide
SLERVIDKRNLAKKAINIAANFDANINKGFYRCETNQCMFIAQKPRKTNNTGCSSCLQSTYDPVIYVVKVGEMLAKYEILKSLKRFVFMNRSFKQKKTEKAKQKERIGGELNEMSIFANAALAMGVIKRAIRHCHVDIRPEINRLSELKKTKHRVAAKSLVKIVKQRKTKWKGIPNSFIQIPQKARNKDADFYVASALKSGGIDIGLCGTYDKKPHADPRWTYQLYFDTEDESEKRLLYCYNDPQAKIRDFWKTFYERGNPSMVNSPGTIEFRMEGFFEKMTPISIESKDFDFRVWNKDLLIRRGLYEIKKRKNLNRKAREIKKAMGSVKRVLANMTYGKSPTDKKSIPVYRVEREKPKKPRAVRKEENELADKLENYRREDFLIRNRRKREATEIAKIIDAAEPPIRHYHTNHLRAVKRIDLSKPVARKNTSVFLKRIMQNGYRGNYCKKCIKGNIDPNKDECRLEDIKKCICCEGTQNIWAKKEKLYTGRINVLNKRIKQMKLECFNVAKAYENFYDNLAALKEGDLKVLKLKVSIPALNPEASDPEEDM

SEQ ID NO: 98 - protein, 534 aa, synthetic construct
  source 1..534: note = Description of Artificial Sequence: Synthetic polypeptide
NASINLGKRAINLSANYDSNLVIGCKNCKFLSFNGNFPRQTNVREGCHSCDKSTYAPEVYIVKIGERKAKYDVLDSLKKFTFQSLKYQIKKSMRERSKKPKELLEFVIFANKDKAFNVIQKSYEHLILNIKQEINRMNGKKRIKNHKKRLFKDREKQLNKLRLIGSSSLFFPRENKGDKDLFTYVAIHSVGRDIGVAGSYESHIEPISDLTYQLFINNEKRLLYAYKPKQNKIIELKENLWNLKKEKKPLDLEFTKPLEKSITFSVKNDKLFKVSKDLMLRQAKFNIQGKEKLSKEERQINRDFSKIKSNVISLSYGRFEELKKEKNIWSPHIYREVKQKEIKPCIVRKGDRIELFEQLKRKMDKLKKFRKERQKKISKDLNFAERIAYNFHTKSIKNTSNKINIDQEAKRGKASYMRKRIGNESFRKKYCEQCFSVGNVYHNVQNGCSCFDNPIELIKKGDEGLIPKGKEDRKYKGALRDDNLQMQIIRVAFNIAKGYEDFYNNLKEKTEKDLKLKFKIGTTISTQESNNKEM

SEQ ID NO: 99 - protein, 577 aa, synthetic construct
  source 1..577: note = Description of Artificial Sequence: Synthetic polypeptide
SNLIKLGKQAINFAANYDANLEVGCKNCKFLSSTNKYPRQTNVHLDNKMACRSCNQSTMEPAIYIVRIGEKKAKYDIYNSLTKFNFQSLKYKAKRSQRFKPKQPKELQELSIAVRKEKALDIIQKSIDHLIQDIRPEIPRIKQQKRYKNHVGKLFYLQKRRKNKLNLIGKGSFFKVFSPKEKKNELLVICALTNIGRDIGLIGNYNTIINPLFEVTYQLYYDYIPKKNNKNVQRRLLYAYKSKNEKILKLKEAFFKRGHENAVNLGSFSYEKPLEKSLTLKIKNDKDDFQVSPSLRIRTGRFFVPSKRNLSRQEREINRRLVKIKSKIKNMTYGKFETARDKQSVHIFRLERQKEKLPLQFRKDEKEFMEEFQKLKRRTNSLKKLRKSRQKKLADLLQLSEKVVYNNHTGTLKKTSNFLNFSSSVKRGKTAYIKELLGQEGFETLYCSNCINKGQKTRYNIETKEKCFSCKDVPFVWKKKSTDKDRKGAFLFPAKLKDVIKATFTVAKAYEDFYDNLKSIDEKKPYIKFKIGLILAHVRHEHKARAKEEAGQKNIYNKPIKIDKNCKECFFFKEEAM

SEQ ID NO: 100 - protein, 613 aa, synthetic construct
  source 1..613: note = Description of Artificial Sequence: Synthetic polypeptide
NTTRKKFRKRTGFPQSDNIKLAYCSAIVRAANLDADIQKKHNQCNPNLCVGIKSNEQSRKYEHSDRQALLCYACNQSTGAPKVDYIQIGEIGAKYKILQMVNAYDFLSLAYNLTKLRNGKSRGHQRMSQLDEVVIVADYEKATEVIKRSINHLLDDIRGQLSKLKKRTQNEHITEHKQSKIRRKLRKLSRLLKRRRWKWGTIPNPYLKNWVFTKKDPELVTVALLHKLGRDIGLVNRSKRRSKQKLLPKVGFQLYYKWESPSLNNIKKSKAKKLPKRLLIPYKNVKLFDNKQKLENAIKSLLESYQKTIKVEFDQFFQNRTEEIIAEEQQTLERGLLKQLEKKKNEFASQKKALKEEKKKIKEPRKAKLLMEESRSLGFLMANVSYALFNTTIEDLYKKSNVVSGCIPQEPVVVFPADIQNKGSLAKILFAPKDGFRIKFSGQHLTIRTAKFKIRGKEIKILTKTKREILKNIEKLRRVWYREQHYKLKLFGKEVSAKPRFLDKRKTSIERRDPNKLADQTDDRQAELRNKEYELRHKQHKMAERLDNIDTNAQNLQTLSFWVGEADKPPKLDEKDARGFGVRTCISAWKWFMEDLLKKQEEDPLLKLKLSIM

SEQ ID NO: 101 - protein, 615 aa, synthetic construct
  source 1..615: note = Description of Artificial Sequence: Synthetic polypeptide
PKKPKFQKRTGFPQPDNLRKEYCLAIVRAANLDADFEKKCTKCEGIKTNKKGNIVKGRTYNSADKDNLLCYACNISTGAPAVDYVFVGALEAKYKILQMVKAYDFHSLAYNLAKLWKGRGRGHQRMGGLNEVVIVSNNEKALDVIEKSLNHFHDEIRGELSRLKAKFQNEHLHVHKESKLRRKLRKISRLLKRRRWKWDVIPNSYLRNFTFTKTRPDFISVALLHRVGRDIGLVTKTKIPKPTDLLPQFGFQIYYTWDEPKLNKLKKSRLRSEPKRLLVPYKKIELYKNKSVLEEAIRHLAEVYTEDLTICFKDFFETQKRKFVSKEKESLKRELLKELTKLKKDFSERKTALKRDRKEIKEPKKAKLLMEESRSLGFLAANTSYALFNLIAADLYTKSKKACSTKLPRQLSTILPLEIKEHKSTTSLAIKPEEGFKIRFSNTHLSIRTPKFKMKGADIKALTKRKREILKNATKLEKSWYGLKHYKLKLYGKEVAAKPRFLDKRNPSIDRRDPKELMEQIENRRNEVKDLEYEIRKGQHQMAKRLDNVDTNAQNLQTKSFWVGEADKPPELDSMEAKKLGLRTCISAWKWFMKDLVLLQEKSPNLKLKLSLTEM

SEQ ID NO: 102 - protein, 775 aa, synthetic construct
  source 1..775: note = Description of Artificial Sequence: Synthetic polypeptide
KFSKRQEGFLIPDNIDLYKCLAIVRSANLDADVQGHKSCYGVKKNGTYRVKQNGKKGVKEKGRKYVFDLIAFKGNIEKIPHEAIEEKDQGRVIVLGKFNYKLILNIEKNHNDRASLEIKNKIKKLVQISSLETGEFLSDLLSGKIGIDEVYGIIEPDVFSGKELVCKACQQSTYAPLVEYMPVGELDAKYKILSAIKGYDFLSLAYNLSRNRANKKRGHQKLGGGELSEVVISANYDKALNVIKRSINHYHVEIKPEISKLKKKMQNEPLKVMKQARIRRELHQLSRKVKRLKWKWGMIPNPELQNIIFEKKEKDFVSYALLHTLGRDIGLFKDTSMLQVPNISDYGFQIYYSWEDPKLNSIKKIKDLPKRLLIPYKRLDFYIDTILVAKVIKNLIELYRKSYVYETFGEEYGYAKKAEDILFDWDSINLSEGIEQKIQKIKDEFSDLLYEARESKRQNFVESFENILGLYDKNFASDRNSYQEKIQSMIIKKQQENIEQKLKREFKEVIERGFEGMDQNKKYYKVLSPNIKGGLLYTDTNNLGFFRSHLAFMLLSKISDDLYRKNNLVSKGGNKGILDQTPETMLTLEFGKSNLPNISIKRKFFNIKYNSSWIGIRKPKFSIKGAVIREITKKVRDEQRLIKSLEGVWHKSTHFKRWGKPRFNLPRHPDREKNNDDNLMESITSRREQIQLLLREKQKQQEKMAGRLDKIDKEIQNLQTANFQIKQIDKKPALTEKSEGKQSVRNALSAWKWFMEDLIKYQKRTPILQLKLAKM

SEQ ID NO: 103 - protein, 777 aa, synthetic construct
  source 1..777: note = Description of Artificial Sequence: Synthetic polypeptide
KFSKRQEGFVIPENIGLYKCLAIVRSANLDADVQGHVSCYGVKKNGTYVLKQNGKKSIREKGRKYASDLVAFKGDIEKIPFEVIEEKKKEQSIVLGKFNYKLVLDVMKGEKDRASLTMKNKSKKLVQVSSLGTDEFLLTLLNEKFGIEEIYGIIEPEVFSGKKLVCKACQQSTYAPLVEYMPVGELDSKYKILSAIKGYDFLSLAYNLARHRSNKKRGHQKLGGGELSEVVISANNAKALNVIKRSLNHYYSEIKPEISKLRKKMQNEPLKVGKQARMRRELHQLSRKVKRLKWKWGKIPNLELQNITFKESDRDFISYALLHTLGRDIGMFNKTEIKMPSNILGYGFQIYYDWEEPKLNTIKKSKNTPKRILIPYKKLDFYNDSILVARAIKELVGLFQESYEWEIFGNEYNYAKEAEVELIKLDEESINGNVEKKLQRIKENFSNLLEKAREKKRQNFIESFESIARLYDESFTADRNEYQREIQSFIIEKQKQSIEKKLKNEFKKIVEKKFNEQEQGKKHYRVLNPTIINEFLPKDKNNLGFLRSKIAFILLSKISDDLYKKSNAVSKGGEKGIIKQQPETILDLEFSKSKLPSINIKKKLFNIKYTSSWLGIRKPKFNIKGAKIREITRRVRDVQRTLKSAESSWYASTHFRRWGFPRFNQPRHPDKEKKSDDRLIESITLLREQIQILLREKQKGQKEMAGRLDDVDKKIQNLQTANFQIKQTGDKPALTEKSAGKQSFRNALSAWKWFMENLLKYQNKTPDLKLKIARTVM

SEQ ID NO: 104 - protein, 610 aa, synthetic construct
  source 1..610: note = Description of Artificial Sequence: Synthetic polypeptide
KWIEPNNIDFNKCLAITRSANLDADVQGHKMCYGIKTNGTYKAIGKINKKHNTGIIEKRRTYVYDLIVTKEKNEKIVKKTDFMAIDEEIEFDEKKEKLLKKYIKAEVLGTGELIRKDLNDGEKFDDLCSIEEPQAFRRSELVCKACNQSTYASDIRYIPIGEIEAKYKILKAIKGYDFLSLKYNLGRLRDSKKRGHQKMGQGELKEFVICANKEKALDVIKRSLNHYLNEVKDEISRLNKKMQNEPLKVNDQARWRRELNQISRRLKRLKWKWGEIPNPELKNLIFKSSRPEFVSYALIHTLGRDIGLINETELKPNNIQEYGFQIYYKWEDPELNHIKKVKNIPKRFIIPYKNLDLFGKYTILSRAIEGILKLYSSSFQYKSFKDPNLFAKEGEKKITNEDFELGYDEKIKKIKDDFKSYKKALLEKKKNTLEDSLNSILSVYEQSLLTEQINNVKKWKEGLLKSKESIHKQKKIENIEDIISRIEELKNVEGWIRTKERDIVNKEETNLKREIKKELKDSYYEEVRKDFSDLKKGEESEKKPFREEPKPIVIKDYIKFDVLPGENSALGFFLSHLSFNLFDSIQYELFEKSRLSSSKHPQIPETILDL

SEQ ID NO: 105 - protein, 632 aa, synthetic construct
  source 1..632: note = Description of Artificial Sequence: Synthetic polypeptide
FRKFVKRSGAPQPDNLNKYKCIAIVRAANLDADIMSNESSNCVMCKGIKMNKRKTAKGAAKTTELGRVYAGQSGNLLCTACTKSTMGPLVDYVPIGRIRAKYTILRAVKEYDFLSLAYNLARTRVSKKGGRQKMHSLSELVIAAEYEIAWNIIKSSVIHYHQETKEEISGLRKKLQAEHIHKNKEARIRREMHQISRRIKRLKWKWHMIPNSELHNFLFKQQDPSFVAVALLHTLGRDIGMINKPKGSAKREFIPEYGFQIYYKWMNPKLNDINKQKYRKMPKRSLIPYKNLNVFGDRELIENAMHKLLKLYDENLEVKGSKFFKTRVVAISSKESEKLKRDLLWKGELAKIKKDFNADKNKMQELFKEVKEPKKANALMKQSRNMGFLLQNISYGALGLLANRMYEASAKQSKGDATKQPSIVIPLEMEFGNAFPKLLLRSGKFAMNVSSPWLTIRKPKFVIKGNKIKNITKLMKDEKAKLKRLETSYHRATHFRPTLRGSIDWDSPYFSSPKQPNTHRRSPDRLSADITEYRGRLKSVEAELREGQRAMAKKLDSVDMTASNLQTSNFQLEKGEDPRLTEIDEKGRSIRNCISSWKKFMEDLMKAQEANPVIKIKIALKDESSVLSEDSM

SEQ ID NO: 106 - protein, 625 aa, synthetic construct
  source 1..625: note = Description of Artificial Sequence: Synthetic polypeptide
KFHPENLNKSYCLAIVRAANLDADIQGHINCIGIKSNKSDRNYENKLESLQNVELLCKACTKSTYKPNINSVPVGEKKAKYSILSEIKKYDFNSLVYNLKKYRKGKSRGHQKLNELRELVITSEYKKALDVINKSVNHYLVNIKNKMSKLKKILQNEHIHVGTLARIRRERNRISRKLDHYRKKWKFVPNKILKNYVFKNQSPDFVSVALLHKLGRDIGLITKTAILQKSFPEYSLQLYYKYDTPKLNYLKKSKFKSLPKRILISYKYPKFDINSNYIEESIDKLLKLYEESPIYKNNSKIIEFFKKSEDNLIKSENDSLKRGIMKEFEKVTKNFSSKKKKLKEELKLKNEDKNSKMLAKVSRPIGFLKAYLSYMLFNIISNRIFEFSRKSSGRIPQLPSCIINLGNQFENFKNELQDSNIGSKKNYKYFCNLLLKSSGFNISYEEEHLSIKTPNFFINGRKLKEITSEKKKIRKENEQLIKQWKKLTFFKPSNLNGKKTSDKIRFKSPNNPDIERKSEDNIVENIAKVKYKLEDLLSEQRKEFNKLAKKHDGVDVEAQCLQTKSFWIDSNSPIKKSLEKKNEKVSVKKKMKAIRSCISAWKWFMADLIEAQKETPMIKLKLALM

SEQ ID NO: 107 - protein, 517 aa, synthetic construct
  source 1..517: note = Description of Artificial Sequence: Synthetic polypeptide
TTLVPSHLAGIEVMDETTSRNEDMIQKETSRSNEDENYLGVKNKCGINVHKSGRGSSKHEPNMPPEKSGEGQMPKQDSTEMQQRFDESVTGETQVSAGATASIKTDARANSGPRVGTARALIVKASNLDRDIKLGCKPCEYIRSELPMGKKNGCNHCEKSSDIASVPKVESGFRKAKYELVRRFESFAADSISRHLGKEQARTRGKRGKKDKKEQMGKVNLDEIAILKNESLIEYTENQILDARSNRIKEWLRSLRLRLRTRNKGLKKSKSIRRQLITLRRDYRKWIKPNPYRPDEDPNENSLRLHTKLGVDIGVQGGDNKRMNSDDYETSFSITWRDTATRKICFTKPKGLLPRHMKFKLRGYPELILYNEELRIQDSQKFPLVDWERIPIFKLRGVSLGKKKVKALNRITEAPRLVVAKRIQVNIESKKKKVLTRYVYNDKSINGRLVKAEDSNKDPLLEFKKQAEEINSDAKYYENQEIAKNYLWGCEGLHKNLLEEQTKNPYLAFKYGFLNIV

SEQ ID NO: 108 - protein, 410 aa, synthetic construct
  source 1..410: note = Description of Artificial Sequence: Synthetic polypeptide
LDFKRTCSQELVLLPEIEGLKLSGTQGVTSLAKKLINKAANVDRDESYGCHHCIHTRTSLSKPVKKDCNSCNQSTNHPAVPITLKGYKIAFYELWHRFTSWAVDSISKALHRNKVMGKVNLDEYAVVDNSHIVCYAVRKCYEKRQRSVRLHKRAYRCRAKHYNKSQPKVGRIYKKSKRRNARNLKKEAKRYFQPNEITNGSSDALFYKIGVDLGIAKGTPETEVKVDVSICFQVYYGDARRVLRVRKMDELQSFHLDYTGKLKLKGIGNKDTFTIAKRNESLKWGSTKYEVSRAHKKFKPFGKKGSVKRKCNDYFRSIASWSCEAASQRAQSNLKNAFPYQKALVKCYKNLDYKGVKKNDMWYRLCSNRIFRYSRIAEDIAQYQSDKGKAKFEFVILAQSVAEYDISAIM

SEQ ID NO: 109 - protein, 602 aa, synthetic construct
  source 1..602: note = Description of Artificial Sequence: Synthetic polypeptide
VFLTDDKRKTALRKIRSAFRKTAEIALVRAQEADSLDRQAKKLTIETVSFGAPGAKNAFIGSLQGYNWNSHRANVPSSGSAKDVFRITELGLGIPQSAHEASIGKSFELVGNVVRYTANLLSKGYKKGAVNKGAKQQREIKGKEQLSFDLISNGPISGDKLINGQKDALAWWLIDKMGFHIGLAMEPLSSPNTYGITLQAFWKRHTAPRRYSRGVIRQWQLPFGRQLAPLIHNFFRKKGASIPIVLTNASKKLAGKGVLLEQTALVDPKKWWQVKEQVTGPLSNIWERSVPLVLYTATFTHKHGAAHKRPLTLKVIRISSGSVFLLPLSKVTPGKLVRAWMPDINILRDGRPDEAAYKGPDLIRARERSFPLAYTCVTQIADEWQKRALESNRDSITPLEAKLVTGSDLLQIHSTVQQAVEQGIGGRISSPIQELLAKDALQLVLQQLFMTVDLLRIQWQLKQEVADGNTSEKAVGWAIRISNIHKDAYKTAIEPCTSALKQAWNPLSGFEERTFQLDASIVRKRSTAKTPDDELVIVLRQQAAEMTVAVTQSVSKELMELAVRHSATLHLLVGEVASKQLSRSADKDRGAMDHWKLLSQSM

SEQ ID NO: 110 - protein, 494 aa, synthetic construct
  source 1..494: note = Description of Artificial Sequence: Synthetic polypeptide
EDLLQKALNTATNVAAIERHSCISCLFTESEIDVKYKTPDKIGQNTAGCQSCTFRVGYSGNSHTLPMGNRIALDKLRETIQRYAWHSLLFNVPPAPTSKRVRAISELRVAAGRERLFTVITFVQTNILSKLQKRYAANWTPKSQERLSRLREEGQHILSLLESGSWQQKEVVREDQDLIVCSALTKPGLSIGAFCRPKYLKPAKHALVLRLIFVEQWPGQIWGQSKRTRRMRRRKDVERVYDISVQAWALKGKETRISECIDTMRRHQQAYIGVLPFLILSGSTVRGKGDCPILKEITRMRYCPNNEGLIPLGIFYRGSANKLLRVVKGSSFTLPMWQNIETLPHPEPFSPEGWTATGALYEKNLAYWSALNEAVDWYTGQILSSGLQYPNQNEFLARLQNVIDSIPRKWFRPQGLKNLKPNGQEDIVPNEFVIPQNAIRAHHVIEWYHKTNDLVAKTLLGWGSQTTLNQTRPQGDLRFTYTRYYFREKEVPEV

SEQ ID NO: 111 - protein, 649 aa, synthetic construct
  source 1..649: note = Description of Artificial Sequence: Synthetic polypeptide
VPKKKLMRELAKKAVFEAIFNDPIPGSFGCKRCTLIDGARVTDAIEKKQGAKRCAGCEPCTFHTLYDSVKHALPAATGCDRTAIDTGLWEILTALRSYNWMSFRRNAVSDASQKQVWSIEELAIWADKERALRVILSALTHTIGKLKNGFSRDGVWKGGKQLYENLAQKDLAKGLFANGEIFGKELVEADHDMLAWTIVPNHQFHIGLIRGNWKPAAVEASTAFDARWLTNGAPLRDTRTHGHRGRRFNRTEKLTVLCIKRDGGVSEEFRQERDYELSVMLLQPKNKLKPEPKGELNSFEDLHDHWWFLKGDEATALVGLTSDPTVGDFIQLGLYIRNPIKAHGETKRRLLICFEPPIKLPLRRAFPSEAFKTWEPTINVFRNGRRDTEAYYDIDRARVFEFPETRVSLEHLSKQWEVLRLEPDRENTDPYEAQQNEGAELQVYSLLQEAAQKMAPKVVIDPFGQFPLELFSTFVAQLFNAPLSDTKAKIGKPLDSGFVVESHLHLLEEDFAYRDFVRVTFMGTEPTFRVIHYSNGEGYWKKTVLKGKNNIRTALIPEGAKAAVDAYKNKRCPLTLEAAILNEEKDRRLVLGNKALSLLAQTARGNLTILEALAAEVLRPLSGTEGVVHLHACVTRHSTLTESTETDNM

SEQ ID NO: 112 - protein, 414 aa, synthetic construct
  source 1..414: note = Description of Artificial Sequence: Synthetic polypeptide
VEKLFSERLKRAMWLKNEAGRAPPAETLTLKHKRVSGGHEKVKEELQRVLRSLSGTNQAAWNLGLSGGREPKSSDALKGEKSRVVLETVVFHSGHNRVLYDVIEREDQVHQRSSIMHMRRKGSNLLRLWGRSGKVRRKMREEVAEIKPVWHKDSRWLAIVEEGRQSVVGISSAGLAVFAVQESQCTTAEPKPLEYVVSIWFRGSKALNPQDRYLEFKKLKTTEALRGQQYDPIPFSLKRGAGCSLAIRGEGIKFGSRGPIKQFFGSDRSRPSHADYDGKRRLSLFSKYAGDLADLTEEQWNRTVSAFAEDEVRRATLANIQDFLSISHEKYAERLKKRIESIEEPVSASKLEAYLSAIFETFVQQREALASNFLMRLVESVALLISLEEKSPRVEFRVARYLAESKEGFNRKAM

SEQ ID NO: 113 - protein, 413 aa, synthetic construct
  source 1..413: note = Description of Artificial Sequence: Synthetic polypeptide
VVITQSELYKERLLRVMEIKNDRGRKEPRESQGLVLRFTQVTGGQEKVKQKLWLIFEGFSGTNQASWNFGQPAGGRKPNSGDALKGPKSRVTYETVVFHFGLRLLSAVIERHNLKQQRQTMAYMKRRAAARKKWARSGKKCSRMRNEVEKIKPKWHKDPRWFDIVKEGEPSIVGISSAGFAIYIVEEPNFPRQDPLEIEYAISIWFRRDRSQYLTFKKIQKAEKLKELQYNPIPFRLKQEKTSLVFESGDIKFGSRGSIEHFRDEARGKPPKADMDNNRRLTMFSVFSGNLTNLTEEQYARPVSGLLAPDEKRMPTLLKKLQDFFTPIHEKYGERIKQRLANSEASKRPFKKLEEYLPAIYLEFRARREGLASNWVLVLINSVRTLVRIKSEDPYIEFKVSQYLLEKEDNKAL

SEQ ID NO: 114 - protein, 449 aa, synthetic construct
  source 1..449: note = Description of Artificial Sequence: Synthetic polypeptide
KQDALFEERLKKAIFIKRQADPLQREELSLLPPNRKIVTGGHESAKDTLKQILRAINGTNQASWNPGTPSGKRDSKSADALAGPKSRVKLETVVFHVGHRLLKKVVEYQGHQKQQHGLKAFMRTCAAMRKKWKRSGKVVGELREQLANIQPKWHYDSRPLNLCFEGKPSVVGLRSAGIALYTIQKSVVPVKEPKPIEYAVSIWFRGPKAMDREDRCLEFKKLKIATELRKLQFEPIVSTLTQGIKGFSLYIQGNSVKFGSRGPIKYFSNESVRQRPPKADPDGNKRLALFSKFSGDLSDLTEEQWNRPILAFEGIIRRATLGNIQDYLTVGHEQFAISLEQLLSEKESVLQMSIEQQRLKKNLGKKAENEWVESFGAEQARKKAQGIREYISGFFQEYCSQREQWAENWVQQLNKSVRLFLTIQDSTPFIEFRVARYLPKGEKKKGKAM

SEQ ID NO: 115 - protein, 711 aa, synthetic construct
  source 1..711: note = Description of Artificial Sequence: Synthetic polypeptide
ANHAERHKRLRKEANRAANRNRPLVADCDTGDPLVGICRLLRRGDKMQPNKTGCRSCEQVEPELRDAILVSGPGRLDNYKYELFQRGRAMAVHRLLKRVPKLNRPKKAAGNDEKKAENKKSEIQKEKQKQRRMMPAVSMKQVSVADFKHVIENTVRHLFGDRRDREIAECAALRAASKYFLKSRRVRPRKLPKLANPDHGKELKGLRLREKRAKLKKEKEKQAELARSNQKGAVLHVATLKKDAPPMPYEKTQGRNDYTTFVISAAIKVGATRGTKPLLTPQPREWQCSLYWRDGQRWIRGGLLGLQAGIVLGPKLNRELLEAVLQRPIECRMSGCGNPLQVRGAAVDFFMTTNPFYVSGAAYAQKKFKPFGTKRASEDGAAAKAREKLMTQLAKVLDKVVTQAAHSPLDGIWETRPEAKLRAMIMALEHEWIFLRPGPCHNAAEEVIKCDCTGGHAILWALIDEARGALEHKEFYAVTRAHTHDCEKQKLGGRLAGFLDLLIAQDVPLDDAPAARKIKTLLEATPPAPCYKAATSIATCDCEGKFDKLWAIIDATRAGHGTEDLWARTLAYPQNVNCKCKAGKDLTHRLADFLGLLIKRDGPFRERPPHKVTGDRKLVFSGDKKCKGHQYVILAKAHNEEVVRAWISRWGLKSRTNKAGYAATELNLLLNWLSICRRRWMDMLTVQRDTPYIRMKTGRLVVDDKKERKAM

SEQ ID NO: 116 - protein, 574 aa, synthetic construct
  source 1..574: note = Description of Artificial Sequence: Synthetic polypeptide
AKQREALRVALERGIVRASNRTYTLVTNCTKGGPLPEQCRMIERGKARAMKWEPKLVGCGSCAAATVDLPAIEEYAQPGRLDVAKYKLTTQILAMATRRMMVRAAKLSRRKGQWPAKVQEEKEEPPEPKKMLKAVEMRPVAIVDFNRVIQTTIEHLWAERANADEAELKALKAAAAYFGPSLKIRARGPPKAAIGRELKKAHRKKAYAERKKARRKRAELARSQARGAAAHAAIRERDIPPMAYERTQGRNDVTTIPIAAAIKIAATRGARPLPAPKPMKWQCSLYWNEGQRWIRGGMLTAQAYAHAANIHRPMRCEMWGVGNPLKVRAFEGRVADPDGAKGRKAEFRLQTNAFYVSGAAYRNKKFKPFGTDRGGIGSARKKRERLMAQLAKILDKVVSQAAHSPLDDIWHTRPAQKLRAMIKQLEHEWMFLRPQAPTVEGTKPDVDVAGNMQRQIKALMAPDLPPIEKGSPAKRFTGDKRKKGERAVRVAEAHSDEVVTAWISRWGIQTRRNEGSYAAQELELLLNWLQICRRRWLDMTAAQRVSPYIRMKSGRMITDAADEGVAPIPLVENM

SEQ ID NO: 117 - protein, 743 aa, synthetic construct
  source 1..743: note = Description of Artificial Sequence: Synthetic polypeptide
KSISGRSIKHMACLKDMLKSEITEIEEKQKKESLRKWDYYSKFSDEILFRRNLNVSANHDANACYGCNPCAFLKEVYGFRIERRNNERIISYRRGLAGCKSCVQSTGYPPIEFVRRKFGADKAMEIVREVLHRRNWGALARNIGREKEADPILGELNELLLVDARPYFGNKSAANETNLAFNVITRAAKKFRDEGMYDIHKQLDIHSEEGKVPKGRKSRLIRIERKHKAIHGLDPGETWRYPHCGKGEKYGVWLNRSRLIHIKGNEYRCLTAFGTTGRRMSLDVACSVLGHPLVKKKRKKGKKTVDGTELWQIKKATETLPEDPIDCTFYLYAAKPTKDPFILKVGSLKAPRWKKLHKDFFEYSDTEKTQGQEKGKRVVRRGKVPRILSLRPDAKFKVSIWDDPYNGKNKEGTLLRMELSGLDGAKKPLILKRYGEPNTKPKNFVFWRPHITPHPLTFTPKHDFGDPNKKTKRRRVFNREYYGHLNDLAKMEPNAKFFEDREVSNKKNPKAKNIRIQAKESLPNIVAKNGRWAAFDPNDSLWKLYLHWRGRRKTIKGGISQEFQEFKERLDLYKKHEDESEWKEKEKLWENHEKEWKKTLEIHGSIAEVSQRCVMQSMMGPLDGLVQKKDYVHIGQSSLKAADDAWTFSANRYKKATGPKWGKISVSNLLYDANQANAELISQSISKYLSKQKDNQGCEGRKMKFLIKIIEPLRENFVKHTRWLHEMTQKDCEVRAQFSRVSM

SEQ ID NO: 118 - protein, 769 aa, synthetic construct
  source 1..769: note = Description of Artificial Sequence: Synthetic polypeptide
FPSDVGADALKHVRMLQPRLTDEVRKVALTRAPSDRPALARFAAVAQDGLAFVRHLNVSANHDSNCTFPRDPRDPRRGPCEPNPCAFLREVWGFRIVARGNERALSYRRGLAGCKSCVQSTGFPSVPFHRIGADDCMRKLHEILKARNWRLLARNIGREREADPLLTELSEYLLVDARTYPDGAAPNSGRLAENVIKRAAKKFRDEGMRDIHAQLRVHSREGKVPKGRLQRLRRIERKHRAIHALDPGPSWEAEGSARAEVQGVAVYRSQLLRVGHHTQQIEPVGIVARTLFGVGRTDLDVAVSVLGAPLTKRKKGSKTLESTEDFRIAKARETRAEDKIEVAFVLYPTASLLRDEIPKDAFPAMRIDRFLLKVGSVQADREILLQDDYYRFGDAEVKAGKNKGRTVTRPVKVPRLQALRPDAKFRVNVWADPFGAGDSPGTLLRLEVSGVTRRSQPLRLLRYGQPSTQPANFLCWRPHRVPDPMTFTPRQKFGERRKNRRTRRPRVFERLYQVHIKHLAHLEPNRKWFEEARVSAQKWAKARAIRRKGAEDIPVVAPPAKRRWAALQPNAELWDLYAHDREARKRFRGGRAAEGEEFKPRLNLYLAHEPEAEWESKRDRWERYEKKWTAVLEEHSRMCAVADRTLPQFLSDPLGARMDDKDYAFVGKSALAVAEAFVEEGTVERAQGNCSITAKKKFASNASRKRLSVANLLDVSDKADRALVFQAVRQYVQRQAENGGVEGRRMAFLRKLLAPLRQNFVCHTRWLHM

SEQ ID NO: 119 - protein, 564 aa, synthetic construct
  source 1..564: note = Description of Artificial Sequence: Synthetic polypeptide
AARKKKRGKIGITVKAKEKSPPAAGPFMARKLVNVAANVDGVEVHLCVECEADAHGSASARLLGGCRSCTGSIGAEGRLMGSVDVDRERVIAEPVHTETERLGPDVKAFEAGTAESKYAIQRGLEYWGVDLISRNRARTVRKMEEADRPESSTMEKTSWDEIAIKTYSQAYHASENHLFWERQRRVRQHALALFRRARERNRGESPLQSTQRPAPLVLAALHAEAAAISGRARAEYVLRGPSANVRAAAADIDAKPLGHYKTPSPKVARGFPVKRDLLRARHRIVGLSRAYFKPSDVVRGTSDAIAHVAGRNIGVAGGKPKEIEKTFTLPFVAYWEDVDRVVHCSSFKADGPWVRDQRIKIRGVSSAVGTFSLYGLDVAWSKPTSFYIRCSDIRKKFHPKGFGPMKHWRQWAKELDRLTEQRASCVVRALQDDEELLQTMERGQRYYDVFSCAATHATRGEADPSGGCSRCELVSCGVAHKVTKKAKGDTGIEAVAVAGCSLCESKLVGPSKPRVHRQMAALRQSHALNYLRRLQREWEALEAVQAPTPYLRFKYARHLEVRSM

SEQ ID NO: 120 - protein, 565 aa, synthetic construct
  source 1..565: note = Description of Artificial Sequence: Synthetic polypeptide
AAKKKKQRGKIGISVKPKEGSAPPADGPFMARKLVNVAANVDGVEVNLCIECEADAHGSAPARLLGGCKSCTGSIGAEGRLMGSVDVDRADAIAKPVNTETEKLGPDVQAFEAGTAETKYALQRGLEYWGVDLISRNRSRTVRRTEEGQPESATMEKTSWDEIAIKSYTRAYHASENHLFWERQRRVRQHALALFKRAKERNRGDSTLPREPGHGLVAIAALACEAYAVGGRNLAETVVRGPTFGTARAVRDVEIASLGRYKTPSPKVAHGSPVKRDFLRARHRIVGLARAYYRPSDVVRGTSDAIAHVAGRNIGVAGGKPRAVEAVFTLPFVAYWEDVDRVVHCSSFQVSAPWNRDQRMKIAGVTTAAGTFSLHGGELKWAKPTSFYIRCSDTRRKFRPKGFGPMKRWRQWAKDLDRLVEQRASCVVRALQDDAALLETMERGQRYYDVFACAVTHATRGEADRLAGCSRCALTPCQEAHRVTTKPRGDAGVEQVQTSDCSLCEGKLVGPSKPRLHRTLTLLRQEHGLNYLRRLQREWESLEAVQVPTPYLRFKYARHLEVRSM

SEQ ID NO: 121 - protein, 499 aa, synthetic construct
  source 1..499: note = Description of Artificial Sequence: Synthetic polypeptide
TDSQSESVPEVVYALTGGEVPGRVPPDGGSAEGARNAPTGLRKQRGKIKISAKPSKPGSPASSLARTLVNEAANVDGVQSSGCATCRMRANGSAPRALPIGCVACASSIGRAPQEETVCALPTTQGPDVRLLEGGHALRKYDIQRALEYWGVDLIGRNLDRQAGRGMEPAEGATATMKRVSMDELAVLDFGKSYYASEQHLFAARQRRVRQHAKALKIRAKHANRSGSVKRALDRSRKQVTALAREFFKPSDVVRGDSDALAHVVGRNLGVSRHPAREIPQTFTLPLCAYWEDVDRVISCSSLLAGEPFARDQEIRIEGVSSALGSLRLYRGAIEWHKPTSLYIRCSDTRRKFRPRGGLKKRWRQWAKDLDRLVEQRACCIVRSLQADVELLQTMERAQRFYDVHDCAATHVGPVAVRCSPCAGKQFDWDRYRLLAALRQEHALNYLRRLQREWESLEAQQVKMPYLRFKYARKLEVSGPLIGLEVRREPSMGTAIAEM

SEQ ID NO: 122 - protein, 358 aa, synthetic construct
  source 1..358: note = Description of Artificial Sequence: Synthetic polypeptide
AGTAGRRHGSLGARRSINIAGVTDRHGRWGCESCVYTRDQAGNRARCAPCDQSTYAPDVQEVTIGQRQAKYTIFLTLQSFSWTNTMRNNKRAAAGRSKRTTGKRIGQLAEIKITGVGLAHAHNVIQRSLQHNITKMWRAEKGKSKRVARLKKAKQLTKRRAYFRRRMSRQSRGNGFFRTGKGGIHAVAPVKIGLDVGMIASGSSEPADEQTVTLDAIWKGRKKKIRLIGAKGELAVAACRFREQQTKGDKCIPLILQDGEVRWNQNNWQCHPKKLVPLCGLEVSRKFVSQADRLAQNKVASPLAARFDKTSVKGTLVESDFAAVLVNVTSIYQQCHAMLLRSQEPTPSLRVQRTITSM

SEQ ID NO: 123 - protein, 369 aa, synthetic construct
  source 1..369: note = Description of Artificial Sequence: Synthetic polypeptide
GVRFSPAQSQVFFRTVIPQSVEARFAINMAAIHDAAGAFGCSVCRFEDRTPRNAKAVHGCSPCTRSTNRPDVFVLPVGAIKAKYDVFMRLLGFNWTHLNRRQAKRVTVRDRIGQLDELAISMLTGKAKAVLKKSICHNVDKSFKAMRGSLKKLHRKASKTGKSQLRAKLSDLRERTNTTQEGSHVEGDSDVALNKIGLDVGLVGKPDYPSEESVEVVVCLYFVGKVLILDAQGRIRDMRAKQYDGFKIPIIQRGQLTVLSVKDLGKWSLVRQDYVLAGDLRFEPKISKDRKYAECVKRIALITLQASLGFKERIPYYVTKQVEIKNASHIAFVTEAIQNCAENFREMTEYLMKYQEKSPDLKVLLTQLM

SEQ ID NO: 124 - protein, 486 aa, synthetic construct
  source 1..486: note = Description of Artificial Sequence: Synthetic polypeptide
RAVVGKVFLEQARRALNLATNFGTNHRTGCNGCYVTPGKLSIPQDGEKNAAGCTSCLMKATASYVSYPKPLGEKVAKYSTLDALKGFPWYSLRLNLRPNYRGKPINGVQEVAPVSKFRLAEEVIQAVQRYHFTELEQSFPGGRRRLRELRAFYTKEYRRAPEQRQHVVNGDRNIVVVTVLHELGFSVGMFNEVELLPKTPIECAVNVFIRGNRVLLEVRKPQFDKERLLVESLWKKDSRRHTAKWTPPNNEGRIFTAEGWKDFQLPLLLGSTSRSLRAIEKEGFVQLAPGRDPDYNNTIDEQHSGRPFLPLYLYLQGTISQEYCVFAGTWVIPFQDGISPYSTKDTFQPDLKRKAYSLLLDAVKHRLGNKVASGLQYGRFPAIEELKRLVRMHGATRKIPRGEKDLLKKGDPDTPEWWLLEQYPEFWRLCDAAAKRVSQNVGLLLSLKKQPLWQRRWLESRTRNEPLDNLPLSMALTLHLTNEEAL

SEQ ID NO: 125 - protein, 400 aa, synthetic construct
  source 1..400: note = Description of Artificial Sequence: Synthetic polypeptide
AAVYSKFYIENHFKMGIPETLSRIRGPSIIQGFSVNENYINIAGVGDRDFIFGCKKCKYTRGKPSSKKINKCHPCKRSTYPEPVIDVRGSISEFKYKIYNKLKQEPNQSIKQNTKGRMNPSDHTSSNDGIIINGIDNRIAYNVIFSSYKHLMEKQINLLRDTTKRKARQIKKYNNSGKKKHSLRSQTKGNLKNRYHMLGMFKKGSLTITNEGDFITAVRKVGLDISLYKNESLNKQEVETELCLNIKWGRTKSYTVSGYIPLPINIDWKLYLFEKETGLTLRLFGNKYKIQSKKFLIAQLFKPKRPPCADPVVKKAQKWSALNAHVQQMAGLFSDSHLLKRELKNRMHKQLDFKSLWVGTEDYIKWFEELSRSYVEGAEKSLEFFRQDYFCFNYTKQTTM

SEQ ID NO: 126 - protein, 666 aa, synthetic construct
  source 1..666: note = Description of Artificial Sequence: Synthetic polypeptide
PQQQRDLMLMAANYDQDYGNGCGPCTVVASAAYRPDPQAQHGCKRHLRTLGASAVTHVGLGDRTATITALHRLRGPAALAARARAAQAASAPMTPDTDAPDDRRRLEAIDADDVVLVGAHRALWSAVRRWADDRRAALRRRLHSEREWLLKDQIRWAELYTLIEASGTPPQGRWRNTLGALRGQSRWRRVLAPTMRATCAETHAELWDALAELVPEMAKDRRGLLRPPVEADALWRAPMIVEGWRGGHSVVVDAVAPPLDLPQPCAWTAVRLSGDPRQRWGLHLAVPPLGQVQPPDPLKATLAVSMRHRGGVRVRTLQAMAVDADAPMQRHLQVPLTLQRGGGLQWGIHSRGVRRREARSMASWEGPPIWTGLQLVNRWKGQGSALLAPDRPPDTPPYAPDAAVAPAQPDTKRARRTLKEACTVCRCAPGHMRQLQVTLTGDGTWRRFRLRAPQGAKRKAEVLKVATQHDERIANYTAWYLKRPEHAAGCDTCDGDSRLDGACRGCRPLLVGDQCFRRYLDKIEADRDDGLAQIKPKAQEAVAAMAAKRDARAQKVAARAAKLSEATGQRTAATRDASHEARAQKELEAVATEGTTVRHDAAAVSAFGSWVARKGDEYRHQVGVLANRLEHGLRLQELMAPDSVVADQQRASGHARVGYRYVLTAM

SEQ ID NO: 127 - protein, 560 aa, synthetic construct
  source 1..560: note = Description of Artificial Sequence: Synthetic polypeptide
AVAHPVGRGNAGSPGARGPEELPRQLVNRASNVTRPATYGCAPCRHVRLSIPKPVLTGCRACEQTTHPAPKRAVRGGADAAKYDLAAFFAGWAADLEGRNRRRQVHAPLDPQPDPNHEPAVTLQKIDLAEVSIEEFQRVLARSVKHRHDGRASREREKARAYAQVAKKRRNSHAHGARTRRAVRRQTRAVRRAHRMGANSGEILVASGAEDPVPEAIDHAAQLRRRIRACARDLEGLRHLSRRYLKTLEKPCRRPRAPDLGRARCHALVESLQAAERELEELRRCDSPDTAMRRLDAVLAAAASTDATFATGWTVVGMDLGVAPRGSAAPEVSPMEMAISVFWRKGSRRVIVSKPIAGMPIRRHELIRLEGLGTLRLDGNHYTGAGVTKGRGLSEGTEPDFREKSPSTLGFTLSDYRHESRWRPYGAKQGKTARQFFAAMSRELRALVEHQVLAPMGPPLLEAHERRFETLLKGQDNKSIHAGGGGRYVWRGPPDSKKRPAADGDWFRFGRGHADHRGWANKRHELAANYLQSAFRLWSTLAEAQEPTPYARYKYTRVTM

SEQ ID NO: 128 - protein, 404 aa, synthetic construct
  source 1..404: note = Description of Artificial Sequence: Synthetic polypeptide
WDFLTLQVYERHTSPEVCVAGNSTKCASGTRKSDHTHGVGVKLGAQEINVSANDDRDHEVGCNICVISRVSLDIKGWRYGCESCVQSTPEWRSIVRFDRNHKEAKGECLSRFEYWGAQSIARSLKRNKLMGGVNLDELAIVQNENVVKTSLKHLFDKRKDRIQANLKAVKVRMRERRKSGRQRKALRRQCRKLKRYLRSYDPSDIKEGNSCSAFTKLGLDIGISPNKPPKIEPKVEVVFSLFYQGACDKIVTVSSPESPLPRSWKIKIDGIRALYVKSTKVKFGGRTFRAGQRNNRRKVRPPNVKKGKRKGSRSQFFNKFAVGLDAVSQQLPIASVQGLWGRAETKKAQTICLKQLESNKPLKESQRCLFLADNWVVRVCGFLRALSQRQGPTPYIRYRYRCNM

SEQ ID NO: 129 - protein, 392 aa, synthetic construct
  source 1..392: note = Description of Artificial Sequence: Synthetic polypeptide
ARNVGQRNASRQSKRESAKARSRRVTGGHASVTQGVALINAAANADRDHTTGCEPCTWERVNLPLQEVIHGCDSCTKSSPFWRDIKVVNKGYREAKEEIMRIASGISADHLSRALSHNKVMGRLNLDEVCILDFRTVLDTSLKHLTDSRSNGIKEHIRAVHRKIRMRRKSGKTARALRKQYFALRRQWKAGHKPNSIREGNSLTALRAVGFDVGVSEGTEPMPAPQTEVVLSVFYKGSATRILRISSPHPIAKRSWKVKIAGIKALKLIRREHDFSFGRETYNASQRAEKRKFSPHAARKDFFNSFAVQLDRLAQQLCVSSVENLWVTEPQQKLLTLAKDTAPYGIREGARFADTRARLAWNWVFRVCGFTRALHQEQEPTPYCRFTWRSKM

SEQ ID NO: 130 - protein, 1120 aa, Listeria seeligeri
MWISIKTLIHHLGVLFFCDYMYNRREKKIIEVKTMRITKVEVDRKKVLISRDKNGGKLVYENEMQDNTEQIMHHKKSSFYKSVVNKTICRPEQKQMKKLVHGLLQENSQEKIKVSDVTKLNISNFLNHRFKKSLYYFPENSPDKSEEYRIEINLSQLLEDSLKKQQGTFICWESFSKDMELYINWAENYISSKTKLIKKSIRNNRIQSTESRSGQLMDRYMKDILNKNKPFDIQSVSEKYQLEKLTSALKATFKEAKKNDKEINYKLKSTLQNHERQIIEELKENSELNQFNIEIRKHLETYFPIKKTNRKVGDIRNLEIGEIQKIVNHRLKNKIVQRILQEGKLASYEIESTVNSNSLQKIKIEEAFALKFINACLFASNNLRNMVYPVCKKDILMIGEFKNSFKEIKHKKFIRQWSQFFSQEITVDDIELASWGLRGAIAPIRNEIIHLKKHSWKKFFNNPTFKVKKSKIINGKTKDVTSEFLYKETLFKDYFYSELDSVPELIINKMESSKILDYYSSDQLNQVFTIPNFELSLLTSAVPFAPSFKRVYLKGFDYQNQDEAQPDYNLKLNIYNEKAFNSEAFQAQYSLFKMVYYQVFLPQFTTNNDLFKSSVDFILTLNKERKGYAKAFQDIRKMNKDEKPSEYMSYIQSQLMLYQKKQEEKEKINHFEKFINQVFIKGFNSFIEKNRLTYICHPTKNTVPENDNIEIPFHTDMDDSNIAFWLMCKLLDAKQLSELRNEMIKFSCSLQSTEEISTFTKAREVIGLALLNGEKGCNDWKELFDDKEAWKKNMSLYVSEELLQSLPYTQEDGQTPVINRSIDLVKKYGTETILEKLFSSSDDYKVSAKDIAKLHEYDVTEKIAQQESLHKQWIEKPGLARDSAWTKKYQNVINDISNYQWAKTKVELTQVRHLHQLTIDLLSRLAGYMSIADRDFQFSSNYILERENSEYRVTSWILLSENKNKNKYNDYELYNLKNASIKVSSKNDPQLKVDLKQLRLTLEYLELFDNRLKEKRNNISHFNYLNGQLGNSILELFDDARDVLSYDRKLKNAVSKSLKEILSSHGMEVTFKPLYQTNHHLKIDKLQPKKIHHLGEKSTVSSNQVSNEYCQLVRTLLTMK

SEQ ID NO: 131 - protein, 1159 aa, Leptotrichia buccalis
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 132 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 133 - protein, 1285 aa, Rhodobacter capsulatus
MQIGKVQGRTISEFGDPAGGLKRKISTDGKNRKELPAHLSSDPKALIGQWISGIDKIYRKPDSRKSDGKAIHSPTPSKMQFDARDDLGEAFWKLVSEAGLAQDSDYDQFKRRLHPYGDKFQPADSGAKLKFEADPPEPQAFHGRWYGAMSKRGNDAKELAAALYEHLHVDEKRIDGQPKRNPKTDKFAPGLVVARALGIESSVLPRGMARLARNWGEEEIQTYFVVDVAASVKEVAKAAVSAAQAFDPPRQVSGRSLSPKVGFALAEHLERVTGSKRCSFDPAAGPSVLALHDEVKKTYKRLCARGKNAARAFPADKTELLALMRHTHENRVRNQMVRMGRVSEYRGQQAGDLAQSHYWTSAGQTEIKESEIFVRLWVGAFALAGRSMKAWIDPMGKIVNTEKNDRDLTAAVNIRQVISNKEMVAEAMARRGIYFGETPELDRLGAEGNEGFVFALLRYLRGCRNQTFHLGARAGFLKEIRKELEKTRWGKAKEAEHVVLTDKTVAAIRAIIDNDAKALGARLLADLSGAFVAHYASKEHFSTLYSEIVKAVKDAPEVSSGLPRLKLLLKRADGVRGYVHGLRDTRKHAFATKLPPPPAPRELDDPATKARYIALLRLYDGPFRAYASGITGTALAGPAARAKEAATALAQSVNVTKAYSDVMEGRSSRLRPPNDGETLREYLSALTGETATEFRVQIGYESDSENARKQAEFIENYRRDMLAFMFEDYIRAKGFDWILKIEPGATAMTRAPVLPEPIDTRGQYEHWQAALYLVMHFVPASDVSNLLHQLRKWEALQGKYELVQDGDATDQADARREALDLVKRFRDVLVLFLKTGEARFEGRAAPFDLKPFRALFANPATFDRLFMATPTTARPAEDDPEGDGASEPELRVARTLRGLRQIARYNHMAVLSDLFAKHKVRDEEVARLAEIEDETQEKSQIVAAQELRTDLHDKVMKCHPKTISPEERQSYAAAIKTIEEHRFLVGRVYLGDHLRLHRLMMDVIGRLIDYAGAYERDTGTFLINASKQLGAGADWAVTIAGAANTDARTQTRKDLAHFNVLDRADGTPDLTALVNRAREMMAYDRKRKNAVPRSILDMLARLGLTLKWQMKDHLLQDATITQAAIKHLDKVRLTVGGPAAVTEARFSQDYLQMVAAVFNGSVQNPKPRRRDDGDAWHKPPKPATAQSQPDQKPPNKAPSAGSRLPPPQVGEVYEGVVVKVIDTGSLGFLAVEGVAGNIGLHISRLRRIREDAIIVGRRYRFRVEIYVPPKSNTSKLNAADLVRID

SEQ ID NO: 134 - protein, 1175 aa, Carnobacterium gallinarum
MRITKVKIKLDNKLYQVTMQKEEKYGTLKLNEESRKSTAEILRLKKASFNKSFHSKTINSQKENKNATIKKNGDYISQIFEKLVGVDTNKNIRKPKMSLTDLKDLPKKDLALFIKRKFKNDDIVEIKNLDLISLFYNALQKVPGEHFTDESWADFCQEMMPYREYKNKFIERKIILLANSIEQNKGFSINPETFSKRKRVLHQWAIEVQERGDFSILDEKLSKLAEIYNFKKMCKRVQDELNDLEKSMKKGKNPEKEKEAYKKQKNFKIKTIWKDYPYKTHIGLIEKIKENEELNQFNIEIGKYFEHYFPIKKERCTEDEPYYLNSETIATTVNYQLKNALISYLMQIGKYKQFGLENQVLDSKKLQEIGIYEGFQTKFMDACVFATSSLKNIIEPMRSGDILGKREFKEAIATSSFVNYHHFFPYFPFELKGMKDRESELIPFGEQTEAKQMQNIWALRGSVQQIRNEIFHSFDKNQKFNLPQLDKSNFEFDASENSTGKSQSYIETDYKFLFEAEKNQLEQFFIERIKSSGALEYYPLKSLEKLFAKKEMKFSLGSQVVAFAPSYKKLVKKGHSYQTATEGTANYLGLSYYNRYELKEESFQAQYYLLKLIYQYVFLPNFSQGNSPAFRETVKAILRINKDEARKKMKKNKKFLRKYAFEQVREMEFKETPDQYMSYLQSEMREEKVRKAEKNDKGFEKNITMNFEKLLMQIFVKGFDVFLTTFAGKELLLSSEEKVIKETEISLSKKINEREKTLKASIQVEHQLVATNSAISYWLFCKLLDSRHLNELRNEMIKFKQSRIKFNHTQHAELIQNLLPIVELTILSNDYDEKNDSQNVDVSAYFEDKSLYETAPYVQTDDRTRVSFRPILKLEKYHTKSLIEALLKDNPQFRVAATDIQEWMHKREEIGELVEKRKNLHTEWAEGQQTLGAEKREEYRDYCKKIDRFNWKANKVTLTYLSQLHYLITDLLGRMVGFSALFERDLVYFSRSFSELGGETYHISDYKNLSGVLRLNAEVKPIKIKNIKVIDNEENPYKGNEPEVKPFLDRLHAYLENVIGIKAVHGKIRNQTAHLSVLQLELSMIESMNNLRDLMAYDRKLKNAVTKSMIKILDKHGMILKLKIDENHKNFEIESLIPKEIIHLKDKAIKTNQVSEEYCQLVLALLTTNPGNQLN

SEQ ID NO: 135 - protein, 1285 aa, unidentified
  source 1..1285: note = Description of Unknown: Herbinix hemicellulosilytica sequence
MKLTRRRISGNSVDQKITAAFYRDMSQGLLYYDSEDNDCTDKVIESMDFERSWRGRILKNGEDDKNPFYMFVKGLVGSNDKIVCEPIDVDSDPDNLDILINKNLTGFGRNLKAPDSNDTLENLIRKIQAGIPEEEVLPELKKIKEMIQKDIVNRKEQLLKSIKNNRIPFSLEGSKLVPSTKKMKWLFKLIDVPNKTFNEKMLEKYWEIYDYDKLKANITNRLDKTDKKARSISRAVSEELREYHKNLRTNYNRFVSGDRPAAGLDNGGSAKYNPDKEEFLLFLKEVEQYFKKYFPVKSKHSNKSKDKSLVDKYKNYCSYKVVKKEVNRSIINQLVAGLIQQGKLLYYFYYNDTWQEDFLNSYGLSYIQVEEAFKKSVMTSLSWGINRLTSFFIDDSNTVKFDDITTKKAKEAIESNYFNKLRTCSRMQDHFKEKLAFFYPVYVKDKKDRPDDDIENLIVLVKNAIESVSYLRNRTFHFKESSLLELLKELDDKNSGQNKIDYSVAAEFIKRDIENLYDVFREQIRSLGIAEYYKADMISDCFKTCGLEFALYSPKNSLMPAFKNVYKRGANLNKAYIRDKGPKETGDQGQNSYKALEEYRELTWYIEVKNNDQSYNAYKNLLQLIYYHAFLPEVRENEALITDFINRTKEWNRKETEERLNTKNNKKHKNFDENDDITVNTYRYESIPDYQGESLDDYLKVLQRKQMARAKEVNEKEEGNNNYIQFIRDVVVWAFGAYLENKLKNYKNELQPPLSKENIGLNDTLKELFPEEKVKSPFNIKCRFSISTFIDNKGKSTDNTSAEAVKTDGKEDEKDKKNIKRKDLLCFYLFLRLLDENEICKLQHQFIKYRCSLKERRFPGNRTKLEKETELLAELEELMELVRFTMPSIPEISAKAESGYDTMIKKYFKDFIEKKVFKNPKTSNLYYHSDSKTPVTRKYMALLMRSAPLHLYKDIFKGYYLITKKECLEYIKLSNIIKDYQNSLNELHEQLERIKLKSEKQNGKDSLYLDKKDFYKVKEYVENLEQVARYKHLQHKINFESLYRIFRIHVDIAARMVGYTQDWERDMHFLFKALVYNGVLEERRFEAIFNNNDDNNDGRIVKKIQNNLNNKNRELVSMLCWNKKLNKNEFGAIIWKRNPIAHLNHFTQTEQNSKSSLESLINSLRILLAYDRKRQNAVTKTINDLLLNDYHIRIKWEGRVDEGQIYFNIKEKEDIENEPIIHLKHLHKKDCYIYKNSYMFDKQKEWICNGIKEEVYDKSILKCIGNLFKFDYEDKNKSSANPKHT

SEQ ID NO: 136 - protein, 1154 aa, Paludibacter propionicigenes
MRVSKVKVKDGGKDKMVLVHRKTTGAQLVYSGQPVSNETSNILPEKKRQSFDLSTLNKTIIKFDTAKKQKLNVDQYKIVEKIFKYPKQELPKQIKAEEILPFLNHKFQEPVKYWKNGKEESFNLTLLIVEAVQAQDKRKLQPYYDWKTWYIQTKSDLLKKSIENNRIDLTENLSKRKKALLAWETEFTASGSIDLTHYHKVYMTDVLCKMLQDVKPLTDDKGKINTNAYHRGLKKALQNHQPAIFGTREVPNEANRADNQLSIYHLEVVKYLEHYFPIKTSKRRNTADDIAHYLKAQTLKTTIEKQLVNAIRANIIQQGKTNHHELKADTTSNDLIRIKTNEAFVLNLTGTCAFAANNIRNMVDNEQTNDILGKGDFIKSLLKDNTNSQLYSFFFGEGLSTNKAEKETQLWGIRGAVQQIRNNVNHYKKDALKTVFNISNFENPTITDPKQQTNYADTIYKARFINELEKIPEAFAQQLKTGGAVSYYTIENLKSLLTTFQFSLCRSTIPFAPGFKKVFNGGINYQNAKQDESFYELMLEQYLRKENFAEESYNARYFMLKLIYNNLFLPGFTTDRKAFADSVGFVQMQNKKQAEKVNPRKKEAYAFEAVRPMTAADSIADYMAYVQSELMQEQNKKEEKVAEETRINFEKFVLQVFIKGFDSFLRAKEFDFVQMPQPQLTATASNQQKADKLNQLEASITADCKLTPQYAKADDATHIAFYVFCKLLDAAHLSNLRNELIKFRESVNEFKFHHLLEIIEICLLSADVVPTDYRDLYSSEADCLARLRPFIEQGADITNWSDLFVQSDKHSPVIHANIELSVKYGTTKLLEQIINKDTQFKTTEANFTAWNTAQKSIEQLIKQREDHHEQWVKAKNADDKEKQERKREKSNFAQKFIEKHGDDYLDICDYINTYNWLDNKMHFVHLNRLHGLTIELLGRMAGFVALFDRDFQFFDEQQIADEFKLHGFVNLHSIDKKLNEVPTKKIKEIYDIRNKIIQINGNKINESVRANLIQFISSKRNYYNNAFLHVSNDEIKEKQMYDIRNHIAHFNYLTKDAADFSLIDLINELRELLHYDRKLKNAVSKAFIDLFDKHGMILKLKLNADHKLKVESLEPKKIYHLGSSAKDKPEYQYCTNQVMMAYCNMCRSLLEMKK

SEQ ID NO: 137 - protein, 1182 aa, Leptotrichia wadei
MYMKITKIDGVSHYKKQDKGILKKKWKDLDERKQREKIEARYNKQIESKIYKEFFRLKNKKRIEKEEDQNIKSLYFFIKELYLNEKNEEWELKNINLEILDDKERVIKGYKFKEDVYFFKEGYKEYYLRILFNNLIEKVQNENREKVRKNKEFLDLKEIFKKYKNRKIDLLLKSINNNKINLEYKKENVNEEIYGINPTNDREMTFYELLKEIIEKKDEQKSILEEKLDNFDITNFLENIEKIFNEETEINIIKGKVLNELREYIKEKEENNSDNKLKQIYNLELKKYIENNFSYKKQKSKSKNGKNDYLYLNFLKKIMFIEEVDEKKEINKEKFKNKINSNFKNLFVQHILDYGKLLYYKENDEYIKNTGQLETKDLEYIKTKETLIRKMAVLVSFAANSYYNLFGRVSGDILGTEVVKSSKTNVIKVGSHIFKEKMLNYFFDFEIFDANKIVEILESISYSIYNVRNGVGHFNKLILGKYKKKDINTNKRIEEDLNNNEEIKGYFIKKRGEIERKVKEKFLSNNLQYYYSKEKIENYFEVYEFEILKRKIPFAPNFKRIIKKGEDLFNNKNNKKYEYFKNFDKNSAEEKKEFLKTRNFLLKELYYNNFYKEFLSKKEEFEKIVLEVKEEKKSRGNINNKKSGVSFQSIDDYDTKINISDYIASIHKKEMERVEKYNEEKQKDTAKYIRDFVEEIFLTGFINYLEKDKRLHFLKEEFSILCNNNNNVVDFNININEEKIKEFLKENDSKTLNLYLFFNMIDSKRISEFRNELVKYKQFTKKRLDEEKEFLGIKIELYETLIEFVILTREKLDTKKSEEIDAWLVDKLYVKDSNEYKEYEEILKLFVDEKILSSKEAPYYATDNKTPILLSNFEKTRKYGTQSFLSEIQSNYKYSKVEKENIEDYNKKEEIEQKKKSNIEKLQDLKVELHKKWEQNKITEKEIEKYNNTTRKINEYNYLKNKEELQNVYLLHEMLSDLLARNVAFFNKWERDFKFIVIAIKQFLRENDKEKVNEFLNPPDNSKGKKVYFSVSKYKNTVENIDGIHKNFMNLIFLNNKFMNRKIDKMNCAIWVYFRNYIAHFLHLHTKNEKISLISQMNLLIKLFSYDKKVQNHILKSTKTLLEKYNIQINFEISNDKNEVFKYKIKNRLYSKKGKMLGKNNKFEILENEFLENVKAMLEYSE

SEQ ID NO: 138 - protein, 1224 aa, Bergeyella zoohelcum
MENKTSLGNNIYYNPFKPQDKSYFAGYFNAAMENTDSVFRELGKRLKGKEYTSENFFDAIFKENISLVEYERYVKLLSDYFPMARLLDKKEVPIKERKENFKKNFKGIIKAVRDLRNFYTHKEHGEVEITDEIFGVLDEMLKSTVLTVKKKKVKTDKTKEILKKSIEKQLDILCQKKLEYLRDTARKIEEKRRNQRERGEKELVAPFKYSDKRDDLIAAIYNDAFDVYIDKKKDSLKESSKAKYNTKSDPQQEEGDLKIPISKNGVVFLLSLFLTKQEIHAFKSKIAGFKATVIDEATVSEATVSHGKNSICFMATHEIFSHLAYKKLKRKVRTAEINYGEAENAEQLSVYAKETLMMQMLDELSKVPDVVYQNLSEDVQKTFIEDWNEYLKENNGDVGTMEEEQVIHPVIRKRYEDKFNYFAIRFLDEFAQFPTLRFQVHLGNYLHDSRPKENLISDRRIKEKITVFGRLSELEHKKALFIKNTETNEDREHYWEIFPNPNYDFPKENISVNDKDFPIAGSILDREKQPVAGKIGIKVKLLNQQYVSEVDKAVKAHQLKQRKASKPSIQNIIEEIVPINESNPKEAIVFGGQPTAYLSMNDIHSILYEFFDKWEKKKEKLEKKGEKELRKEIGKELEKKIVGKIQAQIQQIIDKDTNAKILKPYQDGNSTAIDKEKLIKDLKQEQNILQKLKDEQTVREKEYNDFIAYQDKNREINKVRDRNHKQYLKDNLKRKYPEAPARKEVLYYREKGKVAVWLANDIKRFMPTDFKNEWKGEQHSLLQKSLAYYEQCKEELKNLLPEKVFQHLPFKLGGYFQQKYLYQFYTCYLDKRLEYISGLVQQAENFKSENKVFKKVENECFKFLKKQNYTHKELDARVQSILGYPIFLERGFMDEKPTIIKGKTFKGNEALFADWFRYYKEYQNFQTFYDTENYPLVELEKKQADRKRKTKIYQQKKNDVFTLLMAKHIFKSVFKQDSIDQFSLEDLYQSREERLGNQERARQTGERNTNYIWNKTVDLKLCDGKITVENVKLKNVGDFIKYEYDQRVQAFLKYEENIEWQAFLIKESKEEENYPYVVEREIEQYEKVRREELLKEVHLIEEYILEKVKDKEILKKGDNQNFKYYILNGLLKQLKNEDVESYKVFNLNTEPEDVNINQLKQEATDLEQKAFVLTYIRNKFAHNQLPKKEFWDYCQEKYGKIEKEKTYAEYFAEVFKKEKEALIK

SEQ ID NO: 139 - protein, 1126 aa, Prevotella intermedia
MEDDKKTTDSIRYELKDKHFWAAFLNLARHNVYITVNHINKILEEGEINRDGYETTLKNTWNEIKDINKKDRLSKLIIKHFPFLEAATYRLNPTDTTKQKEEKQAEAQSLESLRKSFFVFIYKLRDLRNHYSHYKHSKSLERPKFEEGLLEKMYNIFNASIRLVKEDYQYNKDINPDEDFKHLDRTEEEFNYYFTKDNEGNITESGLLFFVSLFLEKKDAIWMQQKLRGFKDNRENKKKMTNEVFCRSRMLLPKLRLQSTQTQDWILLDMLNELIRCPKSLYERLREEDREKFRVPIEIADEDYDAEQEPFKNTLVRHQDRFPYFALRYFDYNEIFTNLRFQIDLGTYHFSIYKKQIGDYKESHHLTHKLYGFERIQEFTKQNRPDEWRKFVKTFNSFETSKEPYIPETTPHYHLENQKIGIRFRNDNDKIWPSLKTNSEKNEKSKYKLDKSFQAEAFLSVHELLPMMFYYLLLKTENTDNDNEIETKKKENKNDKQEKHKIEEIIENKITEIYALYDTFANGEIKSIDELEEYCKGKDIEIGHLPKQMIAILKDEHKVMATEAERKQEEMLVDVQKSLESLDNQINEEIENVERKNSSLKSGKIASWLVNDMMRFQPVQKDNEGKPLNNSKANSTEYQLLQRTLAFFGSEHERLAPYFKQTKLIESSNPHPFLKDTEWEKCNNILSFYRSYLEAKKNFLESLKPEDWEKNQYFLKLKEPKTKPKTLVQGWKNGFNLPRGIFTEPIRKWFMKHRENITVAELKRVGLVAKVIPLFFSEEYKDSVQPFYNYHFNVGNINKPDEKNFLNCEERRELLRKKKDEFKKMTDKEKEENPSYLEFKSWNKFERELRLVRNQDIVTWLLCMELFNKKKIKELNVEKIYLKNINTNTTKKEKNTEEKNGEEKNIKEKNNILNRIMPMRLPIKVYGRENFSKNKKKKIRRNTFFTVYIEEKGTKLLKQGNFKALERDRRLGGLFSFVKTPSKAESKSNTISKLRVEYELGEYQKARIEIIKDMLALEKTLIDKYNSLDTDNFNKMLTDWLELKGEPDKASFQNDVDLLIAVRNAFSHNQYPMRNRIAFANINPFSLSSANTSEEKGLGIANQLKDKTHKTIEKIIEIEKPIETKE

SEQ ID NO: 140 - protein, 1127 aa, Prevotella buccae
MQKQDKLFVDRKKNAIFAFPKYITIMENKEKPEPIYYELTDKHFWAAFLNLARHNVYTTINHINRRLEIAELKDDGYMMGIKGSWNEQAKKLDKKVRLRDLIMKHFPFLEAAAYEMTNSKSPNNKEQREKEQSEALSLNNLKNVLFIFLEKLQVLRNYYSHYKYSEESPKPIFETSLLKNMYKVFDANVRLVKRDYMHHENIDMQRDFTHLNRKKQVGRTKNIIDSPNFHYHFADKEGNMTIAGLLFFVSLFLDKKDAIWMQKKLKGFKDGRNLREQMTNEVFCRSRISLPKLKLENVQTKDWMQLDMLNELVRCPKSLYERLREKDRESFKVPFDIFSDDYNAEEEPFKNTLVRHQDRFPYFVLRYFDLNEIFEQLRFQIDLGTYHFSIYNKRIGDEDEVRHLTHHLYGFARIQDFAPQNQPEEWRKLVKDLDHFETSQEPYISKTAPHYHLENEKIGIKFCSAHNNLFPSLQTDKTCNGRSKFNLGTQFTAEAFLSVHELLPMMFYYLLLTKDYSRKESADKVEGIIRKEISNIYAIYDAFANNEINSIADLTRRLQNTNILQGHLPKQMISILKGRQKDMGKEAERKIGEMIDDTQRRLDLLCKQTNQKIRIGKRNAGLLKSGKIADWLVNDMMRFQPVQKDQNNIPINNSKANSTEYRMLQRALALFGSENFRLKAYFNQMNLVGNDNPHPFLAETQWEHQTNILSFYRNYLEARKKYLKGLKPQNWKQYQHFLILKVQKTNRNTLVTGWKNSFNLPRGIFTQPIREWFEKHNNSKRIYDQILSFDRVGFVAKAIPLYFAEEYKDNVQPFYDYPFNIGNRLKPKKRQFLDKKERVELWQKNKELFKNYPSEKKKTDLAYLDFLSWKKFERELRLIKNQDIVTWLMFKELFNMATVEGLKIGEIHLRDIDTNTANEESNNILNRIMPMKLPVKTYETDNKGNILKERPLATFYIEETETKVLKQGNFKALVKDRRLNGLFSFAETTDLNLEEHPISKLSVDLELIKYQTTRISIFEMTLGLEKKLIDKYSTLPTDSFRNMLERWLQCKANRPELKNYVNSLIAVRNAFSHNQYPMYDATLFAEVKKFTLFPSVDTKKIELNIAPQLLEIVGKAIKEIEKSENKN

SEQ ID NO: 141 - protein, 1135 aa, Porphyromonas gingivalis
MNTVPASENKGQSRTVEDDPQYFGLYLNLARENLIEVESHVRIKFGKKKLNEESLKQSLLCDHLLSVDRWTKVYGHSRRYLPFLHYFDPDSQIEKDHDSKTGVDPDSAQRLIRELYSLLDFLRNDFSHNRLDGTTFEHLEVSPDISSFITGTYSLACGRAQSRFAVFFKPDDFVLAKNRKEQLISVADGKECLTVSGFAFFICLFLDREQASGMLSRIRGFKRTDENWARAVHETFCDLCIRHPHDRLESSNTKEALLLDMLNELNRCPRILYDMLPEEERAQFLPALDENSMNNLSENSLDEESRLLWDGSSDWAEALTKRIRHQDRFPYLMLRFIEEMDLLKGIRFRVDLGEIELDSYSKKVGRNGEYDRTITDHALAFGKLSDFQNEEEVSRMISGEASYPVRFSLFAPRYAIYDNKIGYCHTSDPVYPKSKTGEKRALSNPQSMGFISVHDLRKLLLMELLCEGSFSRMQSDFLRKANRILDETAEGKLQFSALFPEMRHRFIPPQNPKSKDRREKAETTLEKYKQEIKGRKDKLNSQLLSAFDMDQRQLPSRLLDEWMNIRPASHSVKLRTYVKQLNEDCRLRLRKFRKDGDGKARAIPLVGEMATFLSQDIVRMIISEETKKLITSAYYNEMQRSLAQYAGEENRRQFRAIVAELRLLDPSSGHPFLSATMETAHRYTEGFYKCYLEKKREWLAKIFYRPEQDENTKRRISVFFVPDGEARKLLPLLIRRRMKEQNDLQDWIRNKQAHPIDLPSHLFDSKVMELLKVKDGKKKWNEAFKDWWSTKYPDGMQPFYGLRRELNIHGKSVSYIPSDGKKFADCYTHLMEKTVRDKKRELRTAGKPVPPDLAADIKRSFHRAVNEREFMLRLVQEDDRLMLMAINKMMTDREEDILPGLKNIDSILDEENQFSLAVHAKVLEKEGEGGDNSLSLVPATIEIKSKRKDWSKYIRYRYDRRVPGLMSHFPEHKATLDEVKTLLGEYDRCRIKIFDWAFALEGAIMSDRDLKPYLHESSSREGKSGEHSTLVKMLVEKKGCLTPDESQYLILIRNKAAHNQFPCAAEMPLIYRDVSAKVGSIEGSSAKDLPEGSSLVDSLWKKYEMIIRKILPILDPENRFFGKLLNNMSQPINDL

SEQ ID NO: 142 - protein, 1115 aa, Bacteroides pyogenes
MESIKNSQKSTGKTLQKDPPYFGLYLNMALLNVRKVENHIRKWLGDVALLPEKSGFHSLLTTDNLSSAKWTRFYYKSRKFLPFLEMFDSDKKSYENRRETAECLDTIDRQKISSLLKEVYGKLQDIRNAFSHYHIDDQSVKHTALIISSEMHRFIENAYSFALQKTRARFTGVFVETDFLQAEEKGDNKKFFAIGGNEGIKLKDNALIFLICLFLDREEAFKFLSRATGFKSTKEKGFLAVRETFCALCCRQPHERLLSVNPREALLMDMLNELNRCPDILFEMLDEKDQKSFLPLLGEEEQAHILENSLNDELCEAIDDPFEMIASLSKRVRYKNRFPYLMLRYIEEKNLLPFIRFRIDLGCLELASYPKKMGEENNYERSVTDHAMAFGRLTDFHNEDAVLQQITKGITDEVRFSLYAPRYAIYNNKIGFVRTSGSDKISFPTLKKKGGEGHCVAYTLQNTKSFGFISIYDLRKILLLSFLDKDKAKNIVSGLLEQCEKHWKDLSENLFDAIRTELQKEFPVPLIRYTLPRSKGGKLVSSKLADKQEKYESEFERRKEKLTEILSEKDFDLSQIPRRMIDEWLNVLPTSREKKLKGYVETLKLDCRERLRVFEKREKGEHPLPPRIGEMATDLAKDIIRMVIDQGVKQRITSAYYSEIQRCLAQYAGDDNRRHLDSIIRELRLKDTKNGHPFLGKVLRPGLGHTEKLYQRYFEEKKEWLEATFYPAASPKRVPRFVNPPTGKQKELPLIIRNLMKERPEWRDWKQRKNSHPIDLPSQLFENEICRLLKDKIGKEPSGKLKWNEMFKLYWDKEFPNGMQRFYRCKRRVEVFDKVVEYEYSEEGGNYKKYYEALIDEVVRQKISSSKEKSKLQVEDLTLSVRRVFKRAINEKEYQLRLLCEDDRLLFMAVRDLYDWKEAQLDLDKIDNMLGEPVSVSQVIQLEGGQPDAVIKAECKLKDVSKLMRYCYDGRVKGLMPYFANHEATQEQVEMELRHYEDHRRRVFNWVFALEKSVLKNEKLRRFYEESQGGCEHRRCIDALRKASLVSEEEYEFLVHIRNKSAHNQFPDLEIGKLPPNVTSGFCECIWSKYKAIICRIIPFIDPERRFFGKLLEQK

SEQ ID NO: 143 - protein, 668 aa, unidentified
  source 1..668: note = Description of Unknown: Cas13c sequence
MTEKKSIIFKNKSSVEIVKKDIFSQTPDNMIRNYKITLKISEKNPRVVEAEIEDLMNSTILKDGRRSARREKSMTERKLIEEKVAENYSLLANCPMEEVDSIKIYKIKRFLTYRSNMLLYFASINSFLCEGIKGKDNETEEIWHLKDNDVRKEKVKENFKNKLIQSTENYNSSLKNQIEEKEKLLRKESKKGAFYRTIIKKLQQERIKELSEKSLTEDCEKIIKLYSELRHPLMHYDYQYFENLFENKENSELTKNLNLDIFKSLPLVRKMKLNNKVNYLEDNDTLFVLQKTKKAKTLYQIYDALCEQKNGFNKFINDFFVSDGEENTVFKQIINEKFQSEMEFLEKRISESEKKNEKLKKKFDSMKAHFHNINSEDTKEAYFWDIHSSSNYKTKYNERKNLVNEYTELLGSSKEKKLLREEITQINRKLLKLKQEMEEITKKNSLFRLEYKMKIAFGFLFCEFDGNISKFKDEFDASNQEKIIQYHKNGEKYLTYFLKEEEKEKFNLEKMQKIIQKTEEEDWLLPETKNNLFKFYLLTYLLLPYELKGDFLGFVKKHYYDIKNVDFMDENQNNIQVSQTVEKQEDYFYHKIRLFEKNTKKYEIVKYSIVPNEKLKQYFEDLGIDIKYLTGSVESGEKWLGENLGIDIKYLTVEQKSEVSEEKIKKFL

SEQ ID NO: 144 - protein, 796 aa, unidentified
  source 1..796: note = Description of Unknown: Cas13c sequence
MEKDKKGEKIDISQEMIEEDLRKILILFSRLRHSMVHYDYEFYQALYSGKDFVISDKNNLENRMISQLLDLNIFKELSKVKLIKDKAISNYLDKNTTIHVLGQDIKAIRLLDIYRDICGSKNGFNKFINTMITISGEEDREYKEKVIEHFNKKMENLSTYLEKLEKQDNAKRNNKRVYNLLKQKLIEQQKLKEWFGGPYVYDIHSSKRYKELYIERKKLVDRHSKLFEEGLDEKNKKELTKINDELSKLNSEMKEMTKLNSKYRLQYKLQLAFGFILEEFDLNIDTFINNFDKDKDLIISNFMKKRDIYLNRVLDRGDNRLKNIIKEYKFRDTEDIFCNDRDNNLVKLYILMYILLPVEIRGDFLGFVKKNYYDMKHVDFIDKKDKEDKDTFFHDLRLFEKNIRKLEITDYSLSSGFLSKEHKVDIEKKINDFINRNGAMKLPEDITIEEFNKSLILPIMKNYQINFKLLNDIEISALFKIAKDRSITFKQAIDEIKNEDIKKNSKKNDKNNHKDKNINFTQLMKRALHEKIPYKAGMYQIRNNISHIDMEQLYIDPLNSYMNSNKNNITISEQIEKIIDVCVTGGVTGKELNNNIINDYYMKKEKLVFNLKLRKQNDIVSIESQEKNKREEFVFKKYGLDYKDGEINIIEVIQKVNSLQEELRNIKETSKEKLKNKETLFRDISLINGTIRKNINFKIKEMVLDIVRMDEIRHINIHIYYKGENYTRSNIIKFKYAIDGENKKYYLKQHEINDINLELKDKFVTLICNMDKHPNKNKQTINLESNYIQNVKFIIP

SEQ ID NO: 145 - protein, 897 aa, unidentified
  source 1..897: note = Description of Unknown: Cas13c sequence
MENKGNNKKIDFDENYNILVAQIKEYFTKEIENYNNRIDNIIDKKELLKYSEKKEESEKNKKLEELNKLKSQKLKILTDEEIKADVIKIIKIFSDLRHSLMHYEYKYFENLFENKKNEELAELLNLNLFKNLTLLRQMKIENKTNYLEGREEFNIIGKNIKAKEVLGHYNLLAEQKNGFNNFINSFFVQDGTENLEFKKLIDEHFVNAKKRLERNIKKSKKLEKELEKMEQHYQRLNCAYVWDIHTSTTYKKLYNKRKSLIEEYNKQINEIKDKEVITAINVELLRIKKEMEEITKSNSLFRLKYKMQIAYAFLEIEFGGNIAKFKDEFDCSKMEEVQKYLKKGVKYLKYYKDKEAQKNYEFPFEEIFENKDTHNEEWLENTSENNLFKFYILTYLLLPMEFKGDFLGVVKKHYYDIKNVDFTDESEKELSQVQLDKMIGDSFFHKIRLFEKNTKRYEIIKYSILTSDEIKRYFRLLELDVPYFEYEKGTDEIGIFNKNIILTIFKYYQIIFRLYNDLEIHGLFNISSDLDKILRDLKSYGNKNINFREFLYVIKQNNNSSTEEEYRKIWENLEAKYLRLHLLTPEKEEIKTKTKEELEKLNEISNLRNGICHLNYKEIIEEILKTEISEKNKEATLNEKIRKVINFIKENELDKVELGFNFINDFFMKKEQFMFGQIKQVKEGNSDSITTERERKEKNNKKLKETYELNCDNLSEFYETSNNLRERANSSSLLEDSAFLKKIGLYKVKNNKVNSKVKDEEKRIENIKRKLLKDSSDIMGMYKAEVVKKLKEKLILIFKHDEEKRIYVTVYDTSKAVPENISKEILVKRNNSKEEYFFEDNNKKYVTEYYTLEITETNELKVIPAKKLEGKEFKTEKNKENKLMLNNHYCFNVKIIY

SEQ ID NO: 146 - protein, 919 aa, unidentified
  source 1..919: note = Description of Unknown: Cas13c sequence
MEEIKHKKNKSSIIRVIVSNYDMTGIKEIKVLYQKQGGVDTFNLKTIINLESGNLEIISCKPKEREKYRYEFNCKTEINTISITKKDKVLKKEIRKYSLELYFKNEKKDTVVAKVTDLLKAPDKIEGERNHLRKLSSSTERKLLSKTLCKNYSEISKTPIEEIDSIKIYKIKRFLNYRSNFLIYFALINDFLCAGVKEDDINEVWLIQDKEHTAFLENRIEKITDYIFDKLSKDIENKKNQFEKRIKKYKTSLEELKTETLEKNKTFYIDSIKTKITNLENKITELSLYNSKESLKEDLIKIISIFTNLRHSLMHYDYKSFENLFENIENEELKNLLDLNLFKSIRMSDEFKTKNRTNYLDGTESFTIVKKHQNLKKLYTYYNNLCDKKNGFNTFINSFFVTDGIENTDFKNLIILHFEKEMEEYKKSIEYYKIKISNEKNKSKKEKLKEKIDLLQSELINMREHKNLLKQIYFFDIHNSIKYKELYSERKNLIEQYNLQINGVKDVTAINHINTKLLSLKNKMDKITKQNSLYRLKYKLKIAYSFLMIEFDGDVSKFKNNFDPTNLEKRVEYLDKKEEYLNYTAPKNKFNFAKLEEELQKIQSTSEMGADYLNVSPENNLFKFYILTYIMLPVEFKGDFLGFVKNHYYNIKNVDFMDESLLDENEVDSNKLNEKIENLKDSSFFNKIRLFEKNIKKYEIVKYSVSTQENMKEYFKQLNLDIPYLDYKSTDEIGIFNKNMILPIFKYYQNVFKLCNDIEIHALLALANKKQQNLEYAIYCCSKKNSLNYNELLKTFNRKTYQNLSFIRNKIAHLNYKELFSDLFNNELDLNTKVRCLIEFSQNNKFDQIDLGMNFINDYYMKKTRFIFNQRRLRDLNVPSKEKIIDGKRKQQNDSNNELLKKYGLSRTNIKDIFNKAWY

SEQ ID NO: 147 - protein, 1110 aa, unidentified
  source 1..1110: note = Description of Unknown: Cas13c sequence
MKVRYRKQAQLDTFIIKTEIVNNDIFIKSIIEKAREKYRYSFLFDGEEKYHFKNKSSVEIVKNDIFSQTPDNMIRNYKITLKISEKNPRVVEAEIEDLMNSTILKDGRRSARREKSMTERKLIEEKVAENYSLLANCPIEEVDSIKIYKIKRFLTYRSNMLLYFASINSFLCEGIKGKDNETEEIWHLKDNDVRKEKVKENFKNKLIQSTENYNSSLKNQIEEKEKLSSKEFKKGAFYRTIIKKLQQERIKELSEKSLTEDCEKIIKLYSELRHPLMHYDYQYFENLFENKENSELTKNLNLDIFKSLPLVRKMKLNNKVNYLEDNDTLFVLQKTKKAKTLYQIYDALCEQKNGFNKFINDFFVSDGEENTVFKQIINEKFQSEMEFLEKRISESEKKNEKLKKKLDSMKAHFRNINSEDTKEAYFWDIHSSRNYKTKYNERKNLVNEYTKLLGSSKEKKLLREEITKINRQLLKLKQEMEEITKKNSLFRLEYKMKIAFGFLFCEFDGNISKFKDEFDASNQEKIIQYHKNGEKYLTSFLKEEEKEKFNLEKMQKIIQKTEEEDWLLPETKNNLFKFYLLTYLLLPYELKGDFLGFVKKHYYDIKNVDFMDENQNNIQVSQTVEKQEDYFYHKIRLFEKNTKKYEIVKYSIVPNEKLKQYFEDLGIDIKYLTGSVESGEKWLGENLGIDIKYLTVEQKSEVSEEKNKKVSLKNNGMFNKTILLFVFKYYQIAFKLFNDIELYSLFFLREKSEKPFEVFLEELKDKMIGKQLNFGQLLYVVYEVLVKNKDLDKILSKKIDYRKDKSFSPEIAYLRNFLSHLNYSKFLDNFMKINTNKSDENKEVLIPSIKIQKMIQFIEKCNLQNQIDFDFNFVNDFYMRKEKMFFIQLKQIFPDINSTEKQKKSEKEEILRKRYHLINKKNEQIKDEHEAQSQLYEKILSLQKIFSCDKNNFYRRLKEEKLLFLEKQGKKKISMKEIKDKIASDISDLLGILKKEITRDIKDKLTEKFRYCEEKLLNISFYNHQDKKKEEGIRVFLIRDKNSDNFKFESILDDGSNKIFISKNGKEITIQCCDKVLETLMIEKNTLKISSNGKIISLIPHYSYSIDVKY